(19) United States
(12) Patent Application Publication
(10) Pub. No.: US 2023/0371483 A1
(43) Pub. Date: Nov. 23, 2023

(54) METHODS AND COMPOSITIONS FOR SEXING AND STERILIZATION IN DROSOPHILA SUZUKII AND AEDES AEGYPTI

(71) Applicant: The Regents of the University of California, Oakland, CA (US)

(72) Inventors: Omar Akbari, La Jolla, CA (US); Ming Li, La Jolla, CA (US); Ting Yang, La Jolla, CA (US); Nikolay Kandul, La Jolla, CA (US)

(21) Appl. No.: 17/629,348

(22) PCT Filed: Jul. 24, 2020
(86) PCT No.: PCT/US2020/043618

§ 371 Date: Jan. 21, 2022

Related U.S. Application Data
(60) Provisional application No. 62/878,642, filed on Jul. 25, 2019.
(60) Provisional application No. 62/929,480, filed on Nov. 1, 2019.

(51) Int. Cl. A01K 67/033; C12N 15/11; C12N 9/22; C12N 15/90

(52) U.S. Cl. CPC A01K 67/0339; C12N 15/11; C12N 9/22; C12N 15/902; A01K 2227/706; A01K 2217/077; C12N 2310/20

(57) ABSTRACT

Provided herein is a next-generation highly-efficient technology that can be used for biocontrol of D. suzukii and/or Aedes aegypti. The composition and technique termed precision guided SIT (pgSIT) functions by exploiting the precision and accuracy of CRISPR to simultaneously disrupt genes essential for either female viability or male fertility. It utilizes a simple breeding scheme requiring two homozygous strains—one expressing Cas9 and the other expressing double guide RNAs (dgRNAs). A single mating between these strains mechanistically results in synchronous RNA-guided dominant biallelic knockouts of both target genes throughout development, resulting in the complete penetrance of desired phenotypes in all progeny. This document provides methods and compositions relating to producing such insect eggs, insects, insect populations and uses thereof in reducing a wild-type insect population, along with methods and materials for producing genetically modified progeny of D. suzukii and/or Aedes aegypti.

### CROSS-REFERENCE TO RELATED APPLICATION

This application claims the benefit under 35 U.S.C. 119(e) of U.S. Provisional Serial Nos. 62/878,642, filed Jul. 25, 2019, and 62/929,480, filed Nov. 1, 2019, the contents of each of which are incorporated herein by reference in their entireties.

### STATEMENT AS TO FEDERALLY SPONSORED RESEARCH

This invention was made with government support under Grant No. HR0011-17-2-0047 awarded by the Defense Advanced Research Projects Agency. The Government has certain rights in the invention.

### SEQUENCE LISTING

The instant application contains a Sequence Listing which has been submitted electronically in ASCII format and is hereby incorporated by reference in its entisrety. Said ASCII copy, created on Jul. 24, 2020, is named 114198-0990_SequenceListing_ST25.txt and is 765,952 bytes in size.

### TECHNICAL FIELD

This document relates to methods and materials for producing genetically modified progeny of an Aedes aegypti mosquito or a Drosophila suzukii.

### BACKGROUND

Drosophila suzukii (Spotted Wing Drosophila, SWD) is a major invasive pest of ripening small fruit including raspberries, blueberries, strawberries, and cherries (Walsh et al. 2011; Stockton, Wallingford, and Loeb 2018). It has caused significant worldwide economic losses including significant damage in the berry- and cherry-growing industries of western North America (Stockton, Wallingford, and Loeb 2018; Ioriatti et al. 2015; Van Steenwyk and Bolda 2014; Walton et al. 2014). Achieving effective control of D. suzukii has been difficult in a number of crop systems including cherries (Van Timmeren et al. 2017; Mazzi et al. 2017), and control measures have largely relied on prophylactic application of expensive broad spectrum insecticides (Haye et al. 2016; Van Timmeren et al. 2017; Mazzi et al. 2017; Schetelig et al. 2018). This is problematic, as the repeated use of broad-spectrum insecticides has led to disruption of integrated pest management systems developed for crops such as cherries and berries, and has had a serious impact on beneficial arthropods, resulting, for example, in an increased use of miticides (Van Steenwyk and Bolda 2014). Additionally, broad use of insecticides makes it inevitable that resistance will become a major problem in the foreseeable future (Haye et al. 2016), increases the risk of residues on fruits, and arouses public concern (Van Timmeren et al. 2017). However, there are no effective alternatives to managing D. suzukii infestation, and it is likely that, unless more effective control measures are developed, this pest will continue to spread (Haye et al. 2016).

Additionally, the annual incidence of vector-borne disease exceeds 1 billion globally with mosquito-diseases comprising the majority of the global vector-borne disease burden (World Health Organization 2014). There are currently no vaccines for most mosquito-borne diseases, so prevention, mainly through vector control, is the primary method to reduce disease burden.

Chemical insecticides have historically been an important tool for mosquito control, but they have limitations, most notably their limited efficacy due to increasing vector insecticide resistance and their lack of species specificity. While many insecticide driven approaches have been successful in some disease prevention programs (Pluess et al. 2010), for a myriad of reasons, they have mixed results overall (Esu et al. 2010; George et al. 2015; Maciel-de-Freitas et al. 2014). Even in areas where sustained vector control has been achieved in the past, insecticide resistance has greatly reduced or eliminated the impact of vector control on disease transmission (Liu 2015; Hemingway, Field, and Vontas 2002; Maciel-de-Freitas et al. 2014). Due to the widespread use of insecticides and the limited number of insecticide families available to vector control programs, insecticide resistance will continue to be a barrier to insecticide-based vector control.

Sterile insect technique (SIT) is the gold standard for insect population suppression. In classic SIT, insects are irradiated with ionizing radiation to induce male sterility. The sterile males are then released to mate with wild females resulting in non-viable progeny. Over time, repeated mass releases of sterile males suppresses and can even eliminate the target population. This approach was used to eradicate the screwworm fly, Cochliomyia hominivorax, (Krafsur et al. 1986), the Mexican fruit fly, Anastrepha ludens, and the Mediterranean fruit fly, Ceratitis capitata, from regions of North America (Hendrichs et al. 2002). Notwithstanding, in mosquitoes irradiation-based SIT causes high male mortality and fitness costs. Recent field studies release of irradiated, sterile, male Aedes albopictus led to very limited population reduction (Bellini et al. 2013) likely for these reasons. While the classic irradiation-based SIT presents an environment-friendly method of a local population suppression, it is not currently feasible or scalable for the control of mosquito populations.

In recent years innovative genetic vector control methods, such as the Release of Insects Carrying a Dominant Lethal (MDL) (Thomas et al. 2000), have demonstrated large reductions in wild vector populations (Carvalho et al. 2015; Harris et al. 2012). Other novel disease or vector control methods, such as the DENV and ZIKV transmission blocking Wolbachia infected Aedes aegypti and the Wolbachia Incompatible Insect Technique (Wolbachia IIT), respectively, are currently being evaluated in the field (Schmidt et al. 2017). While effective, these methods require large numbers of mosquitoes to be raised, manually sexed and released as adults in the field, near target sites. Building mosquito mass rearing factories in local disease endemic areas is costly, labor-intensive and current procedures are error-prone (Gilles et al. 2014; Papathanos et al. 2009). Female release, even in small numbers, is particularly problematic to the Wolbachia technology as this will immunize the target population to the incompatible Wolbachia strain and ultimately lead to the failure of the approach. Some studies even indicate that in some contexts, Wolbachia enhances pathogen infection (Hughes, Rivero, and Rasgon 2014; Dodson et al. 2014) or can cause large vector fitness costs (Joshi et al. 2014). Additionally, the antibiotic drugs required for MDL mosquitoes have high male fitness-costs (˜5% that of wt male fitness, based on MDL field trials in the Cayman Islands (Harris et al. 2011) and Brazil (Carvalho et al. 2015) resulting from the loss or alteration of gut microbiome or symbiotic bacteria as well as toxicity to mitochondrial cell functions (Moullan et al. 2015; Chatzispyrou et al. 2015).

Therefore, there is still an urgent need for new vector control technologies for the suppression of wild populations of, e.g., an Aedes aegypti mosquito or a Drosophila suzukii.

### SUMMARY OF THE DISCLOSURE

This disclosure provides a mating system producing a genetically modified progeny that is a sterile male insect of Drosophila suzukii or Aedes aegypti mosquito. The method comprises, or consists essentially of, or yet further consists of a genetically modified first parental insect and a genetically modified second parental insect. In one embodiment, the first genetically modified parental insect comprises or has been transduced or transfected with at least one nucleic acid sequence that comprises, or consists essentially of, or yet further consists of (a) at least a first guide RNA targeting a female essential genomic sequence that is required for female-specific viability in Drosophila suzukii or Aedes aegypti, and (b) at least a second guide RNA targeting a male sterility genomic sequence that is required for male-specific fertility in Drosophila suzukii or Aedes aegypti. Additionally or alternatively, the second parental insect comprises or has been transduced with a nucleic acid that encodes an endonuclease under the control of a regulatory sequence that is suitable for directing protein expression in Drosophila suzukii or Aedes aegypti.

In some embodiments, the targeted sequence required for female-specific viability in Aedes aegypti is the Intersex or the labrum (Lab) gene. In another embodiment, the targeted sequence required for male-specific fertility in Aedes aegypti is a sequence of the zero population growth (Zpg)1 gene. Additionally or alternatively, the targeted sequence required for male-specific fertility in Drosophila suzukii is selected from a sequence of Cannonball (Can), meiosis I arrest (Mia), P-element induced wimpy testis (PIWI), Heterochromatin Protein 1e (HP1e), zero population growth (Zpg), male sterile (3) K81 (K81), misfire (Mfr), or sneaky (Snky). In a further embodiment, the targeted sequence is a sequence in the gene exon of the genes that disclosed herein.

In some embodiments, the targeted sequence required for female-specific viability in Drosophila suzukii is GGCGGCAGCGGCGGGAATGG (Sxl—Max Scott site, SEQ ID NO: 82). In another embodiment, the targeted sequence required for male-specific fertility in Drosophila suzukii is selected from: TGGCGGTACCGGCTCCGGAA (Btub Original site 2, SEQ ID NO: 83), CTTGAGTGTGCACCAGCTGG (Btub site 2, SEQ ID NO: 84), CAATGCGGTAACCAGATCGG (Btub site 3, SEQ ID NO: 85), GCCTCGGGGTCTAAAGATGT (Btub site 4, SEQ ID NO: 86), GCTACAGAGGAATGGCCCAG (Cannonball (Can), SEQ ID NO: 66), GGATCGGGATAACCTGCCGT (Cannonball (Can), SEQ ID NO: 67), GAGAATCCCCTTGTTGCGGG (meiosis I arrest (Mia), SEQ ID NO: 68), GAGCTCTGACCATCCGCATG (meiosis I arrest (Mia), SEQ ID NO: 69), GGTGTTGGACAGCACATCGA (P-element induced wimpy testis (PIWI), SEQ ID NO: 70), GAGCACACGTGATCGCAAGA (P-element induced wimpy testis (PIWI), SEQ ID NO: 71), CAACCTCAAgTTGTaCCAAG (Heterochromatin Protein 1e (HP1e), SEQ ID NO: 72), GGATTATCCAAAACTAAGCA (Heterochromatin Protein 1e (HP1e), SEQ ID NO: 73), GCAACTGCAAACGCATTCCG (zero population growth (Zpg), SEQ ID NO: 74), GCCCAAGTTGCACCTGCAGG (zero population growth (Zpg), SEQ ID NO: 75), GATCACAGCGAGTTCTTCGG (male sterile (3) K81 (K81), SEQ ID NO: 76), GGAGTTGGGGTCGTCGACAT male sterile (3) K81 (K81), SEQ ID NO: 77), GGCGTCAAGTTCAAGAAGCA (misfire (Mfr), SEQ ID NO: 78), GAGAACGGGACACTGGCAGG (misfire (Mfr), SEQ ID NO: 79), GACTTCTCGTAGGTGCGCAA (sneaky (Snky), SEQ ID NO: 80), or GTAGGTGCGCAATGGTAAGA (sneaky (Snky), SEQ ID NO: 81). Additionally or alternatively, the targeted sequence required for female-specific viability in Aedes aegypti is selected from: AGCGACATGCAGCCATTCTG (double sex (Dsx) 1, SEQ ID NO: 87 in the revised plasmid), CTCACAAGTAGAGCTACACG (Dsx2, SEQ ID NO: 88 in the revised plasmid), GGTAGCTGGCCGTTGCCAAA (Dsx3, SEQ ID NO: 89 in the revised plasmid), CTGTCGTCGTTTTTTTCCGG (Dsx4, SEQ ID NO: 90 in the revised plasmid), GGATATTGGTACGACCCGGG (Dsx5, SEQ ID NO: 91), GCGGGATTCGCTCTCGACGA (Intersex1, SEQ ID NO: 58), GGACAACATTTCCAAGGTTA (Intersex2, SEQ ID NO: 59), GCACTAGTGGGATATCCTGA (labrum (Lab) 5, SEQ ID NO: 60), GTCCAGGAAGCATTTGGTAT (labrum (Lab) 4, SEQ ID NO: 61), GTATTCGATGTTCTCCACC (sister of sex lethal (Slx) 4, SEQ ID NO: 92), GAAGGCATATCAAACATTCG (sister of sex lethal (Slx) 5, SEQ ID NO: 93), GTTAAGAACTCGGCCATCGA (homeotic protein Proboscipedia (Pb) 1, SEQ ID NO: 94), GCAAGTTAAGCCTGAAACAA (homeotic protein Proboscipedia (Pb) 2, SEQ ID NO: 95). In another embodiment, the targeted sequence required for male-specific fertility in Aedes aegypti is selected from: TACTACAACGAGGCTACCGG (Btub1, SEQ ID NO: 96), GTCTCCGCAATACGCCCCGG (Btub2, SEQ ID NO: 97), GATAGGAGCCAAGTTCTGGG (Btub3, SEQ ID NO: 98), CACTATGGATTCGGTCCGAG (Btub4, SEQ ID NO: 99), GTGTAGACTGGCCCTAACGG (zero population growth (Zpg)1, SEQ ID NO: 62), TCTCGTTCCCCAAACACTTG (Zpg2, SEQ ID NO: 63), TCATCAAGAACATGTTCAGG (Zpg3, SEQ ID NO: 64), ACAGTTGGAACCGAACGCTG (Zpg4, SEQ ID NO: 65).

In another aspect, provided is a mating system producing a genetically modified progeny that is a sterile male insect of Drosophila suzukii. The method comprises a first genetically modified parental insect and a second genetically modified parental insect. In one embodiment, the first genetically modified parental Drosophila suzukii comprises at least one nucleic acid sequence that comprises, or consists essentially of, or yet further consists of: (a) at least a first guide RNA targeting a female essential genomic sequence that is required for female-specific viability in Drosophila suzukii, and (b) at least a second guide RNA targeting a male sterility genomic sequence that is required for male-specific fertility in Drosophila suzukii. In a further embodiment, the second genetically modified parental insect comprises a nucleic acid that encodes an endonuclease under the control of a regulatory sequence that is suitable for directing protein expression in Drosophila suzukii. In some embodiments, the regulatory sequence comprises, or consists essentially of, or yet further consists of a promoter selected from a deadhead (Dhd) promoter or a bicoid (BicC) promoter.

In yet another aspect, provided is a method of producing a genetically modified progeny that is a sterile male insect of Drosophila suzukii or Aedes aegypti mosquito, the method comprising, or consisting essentially of, or yet further consisting of genetically crossing two parental insects of the mating system as disclosed herein, and producing and collecting the progeny of the parental insects.

Further provided is a progeny produced by a mating system as disclosed herein, or a method as disclosed herein, along with compositions (such as polynucleotides, vectors, cells, or a Drosophila suzukii or Aedes aegypti mosquito comprising any one or more of the polynucleotides, vectors, cells as disclosed herein) or kits (comprising or consisting essentially of, or yet further consisting of genetically any one or more of the compositions and an optional instruction) for use in a mating system or a method as disclosed herein. In some embodiments, the genetically modified progeny is an egg, a population of eggs, an insect, or a population of insects. In a further embodiment, the progeny is sterile male. Also provided is a method of reducing a wild-type insect population comprising, or consisting essentially of, or yet further consisting of introducing the progeny as disclosed herein to the wild-type insect population.

In one aspect, provided herein is an isolated polynucleotide Cas9 construct as described herein, its complement or an equivalent of each thereof. In one aspect, the isolated Cas9 construct is of the group of: 874Z, 874S, 874R, 874W, and 874Z1. Further provided herein is a fragment of the isolated Cas9 construct, wherein the fragment encodes a Cas9 polypeptide.

Vectors as described herein as well as vectors comprising the isolated polynucleotide the isolated Cas9 construct, wherein the fragment encodes a Cas9 polypeptide are further provided.

Yet further described is an isolated host cell comprising the isolated polynucleotide of as disclosed herein or the vector as disclosed herein, optionally wherein the host cell is an insect cell. In one aspect, the insect is an arthropod, such as a Drosophila, e.g., D. suzukii.

In another aspect, provided herein is an expression product of the polynucleotide as described herein.

Also provide is a guide RNA (gRNA) as disclosed herein, or its complement or an equivalent of each thereof. The gRNA is optionally a single guide RNA (sgRNA) or optionally a double guide RNA (dg RNA). The gRNA can be comprised within a vector.

Further provided are isolated host cells comprising the gRNA as described herein and/or a vector comprising the gRNA, optionally wherein the host cell is an insect cell. In one aspect, the insect is an arthropod, such as a Drosophila, e.g., D. suzukii.

Further provided is an isolated host cell comprising the isolated polynucleotide of this disclosure and the gRNA of this disclosure, optionally wherein the host cell is an insect cell. In one aspect, the insect is an arthropod, such as a Drosophila, e.g., D. suzukii. In one aspect, the host cell is an egg, a sperm or a zygote.

Yet further provided herein is a host cell system comprising a first host cell and a second host cell, wherein the first host cell and the second host cell are homozygous, optionally wherein the host cell is an insect cell. In one aspect, the insect is an arthropod, such as a Drosophila, e.g., D. suzukii.

Also provided is an insect comprising the isolated host cell as described herein. In one aspect, the insect is an arthropod, such as a Drosophila, e.g., D. sukii.

A mating system is provided, comprising the first host cell as described herein and the second host cell as described herein, wherein the first host cell and the second host cell are homozygous. Progeny produced by the mating system also are provided herein.

Yet further provide is a CRISPR system comprising:
(a) a Cas9 polypeptide as described herein, or an equivalent thereof, and
(b) a recombinant or synthetic guide RNA (gRNA) as described herein, or an equivalent thereof.

Yet further provided is an isolated host cell comprising this CRISPR system.

The systems, host cells, polynucleotides and vectors can be used to sterilize or induce lethality of an insect by inserting the cell containing such into the insect. The disclosed methods and compositions are prepared and used in accordance with the methods disclosed in (Kandul et al. 2019), incorporated herein by reference.

In a further aspect, this document provides methods and materials for producing genetically modified progeny of a sterile male Aedes aegypti mosquito egg.

In one aspect, a method of producing a genetically modified progeny of a sterile male Aedes aegypti mosquito egg, the method comprises, or consists essentially of, or yet further consists of: (a) introducing at least one nucleic acid sequence into a first Aedes aegypti mosquito, where the at least one nucleic acid sequence including at least a first guide RNA targeting a female essential genomic sequence that is required for female-specific viability; and at least a second guide RNA targeting a male sterility genomic sequence that is required for male-specific fertility; (b) introducing an endonuclease into a second Aedes aegypti mosquito, the second Aedes aegypti mosquito; and (c) genetically crossing the first Aedes aegypti mosquito and the second Aedes aegypti mosquito, wherein genetically crossing the first Aedes aegypti mosquito and the second Aedes aegypti mosquito produces a progeny of sterile male Aedes aegypti mosquito eggs.

In some embodiments, the female essential genomic sequence comprises, or consists essentially of, or yet further consists of a gene essential for female-specific viability or a female-specific exon essential for female-specific development and/or female-specific viability.

In some embodiments, the female-essential genomic sequence comprises, or consists essentially of, or yet further consists of transformer 2 (tra-2).

In some embodiments, the female essential genomic sequence comprises, or consists essentially of, or yet further consists of doublesex (dsx).

In some embodiments, the at least one first guide RNA comprises, or consists essentially of, or yet further consists of more than one first guide RNA each of which targets a different female-essential genomic sequence that is required for female-specific viability or targets a different region of the same female-essential genomic sequence that is required for female-specific viability.

In some embodiments, the male sterility genomic sequence comprises, or consists essentially of, or yet further consists of a gene essential for male-specific sterility.

In some embodiments, the male sterility genomic sequence is selected from a gene comprises, or consists essentially of, or yet further consists of a gene from the group of βTubulin (βTub), fuzzy onions (Fzo), protaimine A (ProtA), or spermatocyte arrest (Sa).

In some embodiments, the at least one second guide RNA comprises, or consists essentially of, or yet further consists of more than one second guide RNA each of which targets a different male sterility genomic sequence that is required for male fertility or targets a different region of the same male sterility genomic sequence that is required for male specific fertility.

In some embodiments, the endonuclease comprises, or consists essentially of, or yet further consists of a CRISPR-associated sequence 9 (Cas9) endonuclease or a variant thereof, a CRISPR-associated sequence 13 (Cas13) endonuclease or a variant thereof, a CRISPR from Prevotella and Francisella 1 (Cpf1) endonuclease or a variant thereof, a CRISPR from Microgenomates and Smithella 1 (Cms1) endonuclease or a variant thereof, or a CRISPR-associated sequence 6 (Cash) endonuclease or a variant thereof.

In some embodiments, the first Aedes aegypti is a female and the second Aedes aegypti is a male.

In some embodiments, the first Aedes aegypti is a male and the second Aedes aegypti is a female.

In some embodiments, the introducing step (a) comprises, or consists essentially of, or yet further consists of integrating the at least one nucleic acid into the genome of the first Aedes aegypti mosquito.

In some embodiments, the genetically crossing step (c) comprises, or consists essentially of, or yet further consists of producing progeny comprising the endonuclease, at least one first guide RNA, and at least one second guide RNA from which male Aedes aegypti mosquito eggs mature to adulthood.

In some embodiments, the endonuclease is operably linked to a nup50 promoter or a fragment thereof or a uniq promoter or a fragment thereof.

In another aspect the disclosure provides a progeny of Aedes aegypti mosquito eggs comprising up to 100% sterile male Aedes aegypti mosquito eggs produced by the methods described herein.

Other features and advantages of the invention will be apparent from the following detailed description, and from the claims.

### BRIEF DESCRIPTION OF FIGURES

FIGS. 1A to 1B provide a schematic diagram (FIG. 1A) and example images of Drosophila suzukii (FIG. 1B) showing breeding for mass production of sterile Drosophila suzukii males by precision guided sterile insect technique (pgSIT). The CRISPR/Cas9 system provides a way to guide Cas9 endonucleases to a specific gene locus by programming target specificity into a guide RNA (gRNA). The two components required for CRISPR/Cas9-based gene knock-out were split into separate insect lines. Each transgenic line expresses Cas9 or two gRNAs that target gene essential for female viability and male sterility genes. The Cas9 and gRNA lines can be indefinitely propagated in the laboratory. Cas9×gRNA crosses produce both components of the CRISPR/Cas9 system in the same cells and thus cause multiple mutations inside targeted genes resulting in early development knock-outs. Only sterile F1 males will emerge from the eggs. More than 10 lines were engineered targeting one or more female essential and/or male fertility genes. At least two lines demonstrated full penetrance of desired phenotypes (100%, >1,000 embryos).

FIGS. 2A to 2G provide experimental results vial utilizing various constructs comprising triple gRNAs and Cas9 to be expressed under the promoter of vas, nos or Ubiq.

FIGS. 3A to 3D provide experimental results vial utilizing triple-gRNA constructs and different Cas9 constructs (FIG. 3A, Nanos Z1; FIG. 3B, Ubiq W; FIG. 3C, W4A; and FIG. 3D, S-Dhd).

FIG. 4 provides experimental results vial utilizing single-gRNA constructs and a Cas9 to be expressed under the promoter of vas.

FIG. 5 shows production and distribution of standard genetic control technologies. FIG. 5 shows manual sexing and manual release of adults. Current genetic vector control technologies require production facilities to be in close proximity to release sites. Additionally, due to current sxing techniques only adults can be released. The disclosed technology can be developed in one centralized facility and then distributed globally in any life stage (eggs, larvae, pupae, adults), because the females are eliminated early in embryonic development. This flexibility could allow novel filed distribution methods to remote sites (e.g., egg distribution by drone).

FIG. 6 is a schematic diagram showing breeding for mass production of sterile mosquito males by precision guided sterile insect technique (pgSIT). The CRISPR/Cas9 system provides a way to guide Cas9 endonucleases to a specific gene locus by programming target specificity into a guide RNA (gRNA). The two components required for CRISPR/Cas9-based gene knock-out were split into separate insect lines. Each transgenic line expresses Cas9 or two gRNAs that target gene essential for female viability and male sterility genes. The Cas9 and gRNA lines can be indefinitely propagated in the laboratory. Cas9×gRNA crosses produce both components of the CRISPR/Cas9 system in the same cells and thus cause multiple mutations inside targeted genes resulting in early development knock-outs. Only sterile F1 males will emerge from the eggs.

FIG. 7 shows generation of double and triple mutant mosquitos. Single injections of multiplexed sgRNAs robustly generate double- and triple-mutant mosquitoes. Larva, pupae, and adult G1 phenotypes for double-mutants, including: yellow body and white eyes (yellow/white), a mixture of yellow and dark body (yellow/ebony), dark body and white eyes (ebony/white), and one triple-mutant, which is a phenotypic mixture of yellow and dark body and white eyes (yellow/ebony/white). The striking differences between wild-type and mutant larva, pupae and adult are highlighted. (Magnifications: whole-body images, 20×; Insets, 100×.) See, Li et al. (2017) PNAS.

FIGS. 8A and 8B show a schematic diagram of scalability for workflow (FIG. 8A) and workflow (FIG. 8B). FIG. 8A shows small scale manual egg production is the current procedure for mass production in most genetic control programs. FIG. 8B shows automated sex sorting procedures that incorporates the methods and compositions described herein.

FIGS. 9A to 9D provide a Cas9 construct referred herein to as vector 874Z, expressing Cas9 under a promoter of Vasa (i.e., Vasa-Cas9). In one embodiment, vector 874Z refers to pBac-vasa-Cas9-2a-eGFP-p10-UTR-Opie2-dsRed-Modified having a sequence of SEQ ID NO: 1. Its plasmid map is shown in FIG. 9A, while FIG. 9B lists features of the plasmid and the corresponding locations (i.e., starting nt and ending nt in the plasmid). In another embodiment, vector 874Z refers to pBac-vasa-Cas9-2a-eGFP-p10-UTR-Opie2-dsRed having a sequence of SEQ ID NO: 2. Its plasmid map is shown in FIG. 9C, while FIG. 9D lists features of the plasmid and the corresponding locations (i.e., starting nt and ending nt in the plasmid).

FIGS. 10A to 10B provide a Cas9 construct referred herein to as vector 874S, expressing Cas9 under a promoter of Dhd (i.e., Dhd-Cas9). In one embodiment, vector 874S refers to pBac-dmeDhd-Cas9-2a-eGFP-p10-UTR-Opie2-dsRed having a sequence of SEQ ID NO: 3. Its plasmid map is shown in FIG. 10A, while FIG. 10B lists features of the plasmid and the corresponding locations (i.e., starting nt and ending nt in the plasmid).

FIGS. 11A to 11B provide a Cas9 construct referred herein to as vector 874R, expressing Cas9 under a promoter of BicC (i.e., BicC-Cas9). In one embodiment, vector 874R refers to pBac-dmeBicC-Cas9-2a-eGFP-p10-UTR-Opie2-dsRed having a sequence of SEQ ID NO: 4. Its plasmid map is shown in FIG. 11A, while FIG. 11B lists features of the plasmid and the corresponding locations (i.e., starting nt and ending nt in the plasmid).

FIGS. 12A to 12B provide a Cas9 construct referred herein to as vector 874W, expressing Cas9 under a promoter of Ubiq (i.e., Ubiq-Cas9). In one embodiment, vector 874W refers to pBac-dmeUbiq-Cas9-2a-eGFP-p10-UTR-Opie2-dsRed having a sequence of SEQ ID NO: 5. Its plasmid map is shown in FIG. 12A, while FIG. 12B lists features of the plasmid and the corresponding locations (i.e., starting nt and ending nt in the plasmid).

FIGS. 13A to 13D provide a Cas9 construct referred herein to as vector 874Z1, expressing Cas9 under a promoter of Nanos (i.e., Nanos-Cas9). In one embodiment, vector 874Z1 refers to pBac-dmeNanos-Cas9-2a-eGFP-p10-UTR-Opie2-dsRed-Modified (i.e., pBac-nanos-Cas9-2a-eGFP-p10-UTR-Opie2-dsRed-Modified) having a sequence of SEQ ID NO: 6. Its plasmid map is shown in FIG. 13A, while FIG. 13B lists features of the plasmid and the corresponding locations (i.e., starting nt and ending nt in the plasmid). In another embodiment, vector 874Z1 refers to pBac-dmeNanos-Cas9-2a-eGFP-p10-UTR-Opie2-dsRed (i.e., pBac-nanos-Cas9-2a-eGFP-p10-UTR-Opie2-dsRed) having a sequence of SEQ ID NO: 7. Its plasmid map is shown in FIG. 13C, while FIG. 13D lists features of the plasmid and the corresponding locations (i.e., starting nt and ending nt in the plasmid).

FIGS. 14A to 14C provide a gRNA construct herein to as vector 1056C.2, comprising or expressing two sxl gRNAs. In one embodiment, vector 1056C.2 refers to pBac-Ds-HL-U63-sxln-gRNA-U63UTR-opie-mVenus-SV40-U63-sxlm-gRNA-U63UT-HR or HL-U6:3-sxl2-gRNA-UTR-opie-mVenus-SV40-U6:3-sxl1-gRNA-UTR-HR having a sequence of SEQ ID NO: 8. Its plasmid map is shown in FIG. 14A, while FIGS. 14B and 14C list features of the plasmid and the corresponding locations (i.e., starting nt and ending nt in the plasmid).

FIGS. 15A to 15C provide a gRNA construct herein to as vector 1056H, comprising or expressing two sxl gRNAs and one btub gRNA. In one embodiment, vector 1056H refers to pBac-Ds-HL-U63-sxln-gRNA-U63UTR-opie-mVenus-SV40-U63-sxlm-gRNA-U63UT-HR-U63-Btub1-UTR or HL-U6:3-sxl2-gRNA-UTR-opie-mVenus-SV40-U6:3-sxl1-gRNA-UTR-HR-U6-Btuborig2-UTR having a sequence of SEQ ID NO: 9. Its plasmid map is shown in FIG. 15A, while FIGS. 15B and 15C list features of the plasmid and the corresponding locations (i.e., starting nt and ending nt in the plasmid).

FIGS. 16A to 16C provide a gRNA construct herein to as vector 1056I, comprising or expressing two sxl gRNAs and one btub gRNA. In one embodiment, vector 1056I refers to pBac-Ds-HL-U63-sxln-gRNA-U63UTR-opie-mVenus-SV40-U63-sxlm-gRNA-U63UT-HR-U63-Btub2-UTR or HL-U6:3-sxl2-gRNA-UTR-opie-mVenus-SV40-U6:3-sxl1-gRNA-UTR-HR-U6-Btub2-UTR having a sequence of SEQ ID NO: 10. Its plasmid map is shown in FIG. 16A, while FIGS. 16B and 16C list features of the plasmid and the corresponding locations (i.e., starting nt and ending nt in the plasmid).

FIGS. 17A to 17C provide a gRNA construct herein to as vector 1056J, comprising or expressing two sxl gRNAs and one btub gRNA. In one embodiment, vector 1056J refers to pBac-Ds-HL-U63-sxln-gRNA-U63UTR-opie-mVenus-SV40-U63-sxlm-gRNA-U63UT-HR-U63-Btub3-UTR or HL-U6:3-sxl2-gRNA-UTR-opie-mVenus-SV40-U6:3-sxl1-gRNA-UTR-HR-U6-Btub3-UTR having a sequence of SEQ ID NO: 11. Its plasmid map is shown in FIG. 17A, while FIGS. 17B and 17C list features of the plasmid and the corresponding locations (i.e., starting nt and ending nt in the plasmid).

FIGS. 18A to 18C provide a gRNA construct herein to as vector 1056K, comprising or expressing two sxl gRNAs and one btub gRNA. In one embodiment, vector 1056K refers to pBac-Ds-HL-U63-sxln-gRNA-U63UTR-opie-mVenus-SV40-U63-sxlm-gRNA-U63UT-HR-U63-Btub1-UTR or HL-U6:3-sxl2-gRNA-UTR-opie-mVenus-SV40-U6:3-sxl1-gRNA-UTR-HR-U6-Btub4-UTR having a sequence of SEQ ID NO: 12. Its plasmid map is shown in FIG. 18A, while FIGS. 18B and 18C list features of the plasmid and the corresponding locations (i.e., starting nt and ending nt in the plasmid).

FIGS. 19A to 19C provide a gRNA construct herein to as vector 1104D, comprising or expressing an sxl gRNA and two Cannonball gRNAs. In one embodiment, vector 1104D refers to pBac-[U6-gRNA-Slx]-[U6-gRNA #2]-[Opie2-eGFP-p10]-[U6-gRNA #1-Can] or pBac-[U6-gRNA-Slx]-[U6-gRNA #2]-[Opie2-eGFP-p10]-[U6-gRNA #1-Can] having a sequence of SEQ ID NO: 13. Its plasmid map is shown in FIG. 19A, while FIGS. 19B and 19C list features of the plasmid and the corresponding locations (i.e., starting nt and ending nt in the plasmid). FIG. 19C further provides an amino acid sequence of the encoded eGFP (SEQ ID NO: 14).

FIGS. 20A to 20C provide a gRNA construct herein to as vector 1104E, comprising or expressing one sxl gRNA and two Mia gRNAs. In one embodiment, vector 1104E refers to pBac-[U6-gRNA-Slx]-[U6-gRNA-Mia #1]-[Opie2-eGFP-p10]-[U6-gRNA-Mia #2] having a sequence of SEQ ID NO: 15. Its plasmid map is shown in FIG. 20A, while FIGS. 20B and 20C list features of the plasmid and the corresponding locations (i.e., starting nt and ending nt in the plasmid). FIG. 20C further provides an amino acid sequence of the encoded eGFP (SEQ ID NO: 14).

FIGS. 21A to 21C provide a gRNA construct herein to as vector 1104J, comprising or expressing an sxl gRNA and two Zpg gRNAs. In one embodiment, vector 1104J refers to pBac-[U6-gRNA-Slx]-[U6-gRNA-Zpg #1]-[Opie2-eGFP-p10]-[U6-gRNA-Zpg #2] having a sequence of SEQ ID NO: 16. Its plasmid map is shown in FIG. 21A, while FIGS. 21B and 21C list features of the plasmid and the corresponding locations (i.e., starting nt and ending nt in the plasmid). FIG. 21C further provides an amino acid sequence of the encoded eGFP (SEQ ID NO: 14).

FIGS. 22A to 22C provide a gRNA construct herein to as vector 1104H, comprising or expressing an sxl gRNA and two PIWI gRNAs. In one embodiment, vector 1104H refers to pBac-[U6-gRNA-Slx]-[U6-gRNA-PIWI #1]-[Opie2-eGFP-p10]-[U6-gRNA-PIWI #2] having a sequence of SEQ ID NO: 17. Its plasmid map is shown in FIG. 22A, while FIGS. 22B and 22C list features of the plasmid and the corresponding locations (i.e., starting nt and ending nt in the plasmid). FIG. 22C further provides an amino acid sequence of the encoded eGFP (SEQ ID NO: 14).

FIGS. 23A to 23C provide a gRNA construct herein to as vector 1104I, comprising or expressing an sxl gRNA and two HP1e gRNAs. In one embodiment, vector 1104I refers to pBac-[U6-gRNA-Slx]-[U6-gRNA-HP1e #1]-[Opie2-eGFP-p10]-[U6-gRNA-HP1e #2] having a sequence of SEQ ID NO: 18. Its plasmid map is shown in FIG. 23A, while FIGS. 23B and 23C list features of the plasmid and the corresponding locations (i.e., starting nt and ending nt in the plasmid). FIG. 23C further provides an amino acid sequence of the encoded eGFP (SEQ ID NO: 14).

FIGS. 24A to 24C provide a gRNA construct herein to as vector 1104K, comprising or expressing an sxl gRNA and two K81 gRNAs. In one embodiment, vector 1104K refers to pBac-[U6-gRNA-Slx]-[U6-gRNA-K81 #1]-[Opie2-eGFP-p10]-[U6-gRNA-K81 #2] having a sequence of SEQ ID NO: 19. Its plasmid map is shown in FIG. 24A, while FIGS. 24B and 24C list features of the plasmid and the corresponding locations (i.e., starting nt and ending nt in the plasmid). FIG. 24C further provides an amino acid sequence of the encoded eGFP (SEQ ID NO: 14).

FIGS. 25A to 25C provide a gRNA construct herein to as vector 1104M, comprising or expressing an sxl gRNA and two Mfr gRNAs. In one embodiment, vector 1104M refers to pBac-[U6-gRNA-Slx]-[U6-gRNA-Mfr #1]-[Opie2-eGFP-p10]-[U6-gRNA-Mfr #2] having a sequence of SEQ ID NO: 20. Its plasmid map is shown in FIG. 25A, while FIGS. 25B and 25C list features of the plasmid and the corresponding locations (i.e., starting nt and ending nt in the plasmid). FIG. 25C further provides an amino acid sequence of the encoded eGFP (SEQ ID NO: 14).

FIGS. 26A to 26C provide a gRNA construct herein to as vector 1104N, comprising or expressing an sxl gRNA and two Snky gRNAs. In one embodiment, vector 1104N refers to pBac-[U6-gRNA-Slx]-[U6-gRNA-Snky #1]-[Opie2-eGFP-p10]-[U6-gRNA-Snky #2] having a sequence of SEQ ID NO: 21. Its plasmid map is shown in FIG. 26A, while FIGS. 26B and 26C list features of the plasmid and the corresponding locations (i.e., starting nt and ending nt in the plasmid). FIG. 26C further provides an amino acid sequence of the encoded eGFP (SEQ ID NO: 14).

FIGS. 27A to 27B provide a Cas9 construct referred herein to as vector 874L, expressing Cas9 under a promoter of Exuperentia (i.e., Exuperentia-cas9). In one embodiment, vector 874L refers to pBac-AAEL010097-Cas9-2a-eGFP-p10-UTR-Opie2-dsRed having a sequence of SEQ ID NO: 22. Its plasmid map is shown in FIG. 27A, while FIG. 27B lists features of the plasmid and the corresponding locations (i.e., starting nt and ending nt in the plasmid).

FIGS. 28A to 28B provide a Cas9 construct referred herein to as vector 874M, expressing Cas9 under a promoter of 4-nitrophenylphosphatase (i.e., 4-nitrophenylphosphatase-cas9). In one embodiment, vector 874M refers to pBac-AAEL007097-Cas9-2a-eGFP-p10-UTR-Opie2-dsRed having a sequence of SEQ ID NO: 23. Its plasmid map is shown in FIG. 28A, while FIG. 28B lists features of the plasmid and the corresponding locations (i.e., starting nt and ending nt in the plasmid).

FIGS. 29A to 29B provide a Cas9 construct referred herein to as vector 874N, expressing Cas9 under a promoter of Trunk (i.e., Trunk-cas9). In one embodiment, vector 874N refers to pBac-AAEL007584-Cas9-2a-eGFP-p10-UTR-Opie2-dsRed having a sequence of SEQ ID NO: 24. Its plasmid map is shown in FIG. 29A, while FIG. 29B lists features of the plasmid and the corresponding locations (i.e., starting nt and ending nt in the plasmid).

FIGS. 30A to 30B provide a Cas9 construct referred herein to as vector 874P, expressing Cas9 under a promoter of Nup50 (i.e., Nup50-cas9). In one embodiment, vector 874P refers to pBac-AAEL005635-Cas9-2a-eGFP-p10-UTR-Opie2-dsRed having a sequence of SEQ ID NO: 25. Its plasmid map is shown in FIG. 30A, while FIG. 30B lists features of the plasmid and the corresponding locations (i.e., starting nt and ending nt in the plasmid).

FIGS. 31A to 31B provide a Cas9 construct referred herein to as vector 874X, expressing Cas9 under a promoter of Polyubiquitin (i.e., Polyubiquitin-cas9). In one embodiment, vector 874X refers to pBac-Aedes-Ubiq-Cas9-2a-eGFP-p10-UTR-Opie2-dsRed having a sequence of SEQ ID NO: 26. Its plasmid map is shown in FIG. 31A, while FIG. 31B lists features of the plasmid and the corresponding locations (i.e., starting nt and ending nt in the plasmid).

FIGS. 32A to 32B provide a Cas9 construct referred herein to as vector 874Y, expressing Cas9 under a promoter of Ubiquitin L40 (i.e., Ubiquitin L40-cas9). In one embodiment, vector 874Y refers to pBac-Aedes-Ubiq (AAEL006511)-Cas9-2a-eGFP-p10-UTR-Opie2-dsRed having a sequence of SEQ ID NO: 27. Its plasmid map is shown in FIG. 32A, while FIG. 32B lists features of the plasmid and the corresponding locations (i.e., starting nt and ending nt in the plasmid).

FIGS. 33A to 33B provide a Cas9 construct referred herein to as vector 874Y3, expressing Cas9 under a promoter of Vasa (i.e., Vasa-cas9). In one embodiment, vector 874Y3 refers to pBac-Aedes-Vasa(AAEL004978)-Cas9-2a-eGFP-p10-UTR-Opie2-dsRed having a sequence of SEQ ID NO: 28. Its plasmid map is shown in FIG. 33A, while FIG. 33B lists features of the plasmid and the corresponding locations (i.e., starting nt and ending nt in the plasmid).

FIGS. 34A to 34C provide a gRNA construct herein to as vector 1055A, comprising or expressing two β-tubulin gRNAs. In one embodiment, vector 1055A refers to pBac-U6(AAEL017774)-sgBTub1-LongScaffold-3 UTR-U6(AAEL017763)-sgBTub2-LongScaffold-3 UTR-3xp3-tdTomato-v3 having a sequence of SEQ ID NO: 29. Its plasmid map is shown in FIG. 34A, while FIGS. 34B and 34C list features of the plasmid and the corresponding locations (i.e., starting nt and ending nt in the plasmid).

FIGS. 35A to 35C provide a gRNA construct herein to as vector 1055B, comprising or expressing two β-tubulin gRNAs. In one embodiment, vector 1055B refers to pBac-U6(AAEL017774)-sgBTub3-LongScaffold-3 UTR-U6(AAEL017763)-sgBTub4-LongScaffold-3 UTR-3xp3-tdTomato-v3 having a sequence of SEQ ID NO: 30. Its plasmid map is shown in FIG. 35A, while FIGS. 35B and 35C list features of the plasmid and the corresponding locations (i.e., starting nt and ending nt in the plasmid).

FIGS. 36A to 36C provide a gRNA construct herein to as vector 1055C, comprising or expressing two Double sex gRNAs. In one embodiment, vector 1055C refers to pBac-U6(AAEL017774)-sgDsx1-LongScaffold-3 UTR-U6(AAEL017763)-sgDsx2-LongScaffold-3 UTR-3xp3-tdTomato-v3-Revised having a sequence of SEQ ID NO: 31. Its plasmid map is shown in FIG. 36A, while FIGS. 36B and 36C list features of the plasmid and the corresponding locations (i.e., starting nt and ending nt in the plasmid).

FIGS. 37A to 37C provide a gRNA construct herein to as vector 1055D, comprising or expressing two Double sex gRNAs. In one embodiment, vector 1055D refers to pBac-U6(AAEL017774)-sgDsx3-LongScaffold-3 UTR-U6(AAEL017763)-sgDsx4-LongScaffold-3 UTR-3xp3-tdTomato-v3-Revised having a sequence of SEQ ID NO: 32. Its plasmid map is shown in FIG. 37A, while FIGS. 37B and 37C list features of the plasmid and the corresponding locations (i.e., starting nt and ending nt in the plasmid).

FIGS. 38A to 38C provide a gRNA construct herein to as vector 1055E, comprising or expressing two Zpg gRNAs. In one embodiment, vector 1055E refers to pBac-U6(AAEL017774)-sgZpg1-LongScaffold-3 UTR-U6(AAEL017763)-sgZpg2-LongScaffold-3 UTR-3xp3-tdTomato-v3 having a sequence of SEQ ID NO: 33. Its plasmid map is shown in FIG. 38A, while FIGS. 38B and 38C list features of the plasmid and the corresponding locations (i.e., starting nt and ending nt in the plasmid).

FIGS. 39A to 39C provide a gRNA construct herein to as vector 1055F, comprising or expressing two Zpg gRNAs. In one embodiment, vector 1055F refers to pBac-U6(AAEL017774)-sgZpg3-LongScaffold-3 UTR-U6(AAEL017763)-sgZpg4-LongScaffold-3 UTR-3xp3-tdTomato-v3 having a sequence of SEQ ID NO: 34. Its plasmid map is shown in FIG. 39A, while FIGS. 39B and 39C list features of the plasmid and the corresponding locations (i.e., starting nt and ending nt in the plasmid).

FIGS. 40A to 40C provide a gRNA construct herein to as vector 1055J, comprising or expressing two Intersex gRNAs. In one embodiment, vector 1055J refers to pBac-U6(AAEL017774)-gRNA=In #1-LongScaffold-3 UTR-U6(AAEL017763)-gRNA=In #2-LongScaffold-3 UTR-3xp3-tdTomato-v3 having a sequence of SEQ ID NO: 35. Its plasmid map is shown in FIG. 40A, while FIGS. 40B and 40C list features of the plasmid and the corresponding locations (i.e., starting nt and ending nt in the plasmid).

FIGS. 41A to 41B provide a gRNA construct herein to as vector 1055Y, comprising or expressing two Labrum gRNAs. In one embodiment, vector 1055Y refers to pBac-U6(AAEL017774)-sgLab5-U6(AAEL017763)-sgLab4-LongScaffold-3 UTR-3xp3-tdTomato having a sequence of SEQ ID NO: 36. Its plasmid map is shown in FIG. 41A, while FIG. 41B lists features of the plasmid and the corresponding locations (i.e., starting nt and ending nt in the plasmid).

FIGS. 42A to 42B provide a gRNA construct herein to as vector 1055Z, comprising or expressing two Sister of sex lethal gRNAs. In one embodiment, vector 1055Z refers to pBac-U6(AAEL017774)-sgSlx4-U6(AAEL017763)-sgSlx5-LongScaffold-3 UTR-3xp3-tdTomato having a sequence of SEQ ID NO: 37. Its plasmid map is shown in FIG. 42A, while FIG. 42B lists features of the plasmid and the corresponding locations (i.e., starting nt and ending nt in the plasmid).

FIGS. 43A to 43B provide a gRNA construct herein to as vector 1055T, comprising or expressing two Proboscipedia gRNAs. In one embodiment, vector 1055T refers to pBac-U6(AAEL017774)-sgPb1-LongScaffold-3 UTR-U6(AAEL017763)-sgPb2-LongScaffold-3 UTR-3xp3-tdTomato having a sequence of SEQ ID NO: 38. Its plasmid map is shown in FIG. 43A, while FIG. 43B lists features of the plasmid and the corresponding locations (i.e., starting nt and ending nt in the plasmid).

FIGS. 44A to 44C provide a gRNA construct herein to as vector 1067A, comprising or expressing four gRNAs (targeting β-tubulin). In one embodiment, vector 1067A refers to pBac-U6(AAEL017774)-sgBTub1-U6(AAEL017763)-sgBTub2-U6(AAEL017774)-sgBTub3-U6(AAEL017763)-sgBTub4-LongScaffold-3 UTR-3xp3-tdTomato-v3 having a sequence of SEQ ID NO: 39. Its plasmid map is shown in FIG. 44A, while FIGS. 44B and 44C list features of the plasmid and the corresponding locations (i.e., starting nt and ending nt in the plasmid).

FIGS. 45A to 45C provide a gRNA construct herein to as vector 1067B, comprising or expressing four gRNAs (targeting Double sex). In one embodiment, vector 1067B refers to pBac-U6(AAEL017774)-sgDsx1-U6(AAEL017763)-sgDsx2-U6(AAEL017774)-sgDsx3-U6(AAEL017763)-sgDsx4-LongScaffold-3 UTR-3xp3-tdTomato-v3 having a sequence of SEQ ID NO: 40. Its plasmid map is shown in FIG. 45A, while FIGS. 45B and 45C list features of the plasmid and the corresponding locations (i.e., starting nt and ending nt in the plasmid).

FIGS. 46A to 46C provide a gRNA construct herein to as vector 1067C, comprising or expressing four gRNAs (targeting Zpg). In one embodiment, vector 1067C refers to pBac-U6(AAEL017774)-sgZpg1-U6(AAEL017763)-sgZpg2-U6(AAEL017774)-sgZpg3-U6(AAEL017763)-sgZpg4-LongScaffold-3 UTR-3xp3-tdTomato-v3 having a sequence of SEQ ID NO: 41. Its plasmid map is shown in FIG. 46A, while FIGS. 46B and 46C list features of the plasmid and the corresponding locations (i.e., starting nt and ending nt in the plasmid).

FIGS. 47A to 47C provide a gRNA construct herein to as vector 1067D, comprising or expressing four gRNAs (targeting Dsx, Isx and βtub). In one embodiment, vector 1067D refers to pBac-U6(AAEL017774)-sgDsx1-U6(AAEL017774)-sgIsx1-U6(AAEL017774)-sgBTub3-U6(AAEL017763)-sgBTub2-LongScaffold-3 UTR-3xp3-tdTomato having a sequence of SEQ ID NO: 42. Its plasmid map is shown in FIG. 47A, while FIGS. 47B and 47C list features of the plasmid and the corresponding locations (i.e., starting nt and ending nt in the plasmid).

FIGS. 48A to 48C provide a gRNA construct herein to as vector 1067E, comprising or expressing four gRNAs (targeting Dsx and βtub). In one embodiment, vector 1067E refers to pBac-U6(AAEL017774)-sgDsx1-U6(AAEL017774)-sgDsx5-U6(AAEL017774)-sgBTub3-U6(AAEL017763)-sgBTub2-LongScaffold-3 UTR-3xp3-tdTomato having a sequence of SEQ ID NO: 43. Its plasmid map is shown in FIG. 48A, while FIGS. 48B and 48C list features of the plasmid and the corresponding locations (i.e., starting nt and ending nt in the plasmid).

FIGS. 49A to 49C provide a gRNA construct herein to as vector 1067F, comprising or expressing four gRNAs (targeting Dsx and βtub). In one embodiment, vector 1067F refers to pBac-U6(AAEL017774)-sgBTub3-U6(AAEL017763)-sgBTub2-LongScaffold-3 UTR-3xp3-tdTomato-U6(AAEL017774)-sgDsx1-U6(AAEL017763)-sgDsx1 having a sequence of SEQ ID NO: 44. Its plasmid map is shown in FIG. 49A, while FIGS. 49B and 49C list features of the plasmid and the corresponding locations (i.e., starting nt and ending nt in the plasmid).

FIGS. 50A to 50C provide a gRNA construct herein to as vector 1067G, comprising or expressing four gRNAs (targeting Dsx and Zpg). In one embodiment, vector 1067G refers to pBac-U6(AAEL017774)-sgZpg3-U6(AAEL017763)-sgZpg4-LongScaffold-3 UTR-3xp3-tdTomato-U6(AAEL017774)-sgDsx1-U6(AAEL017763)-sgDsx1 having a sequence of SEQ ID NO: 45. Its plasmid map is shown in FIG. 50A, while FIGS. 50B and 50C list features of the plasmid and the corresponding locations (i.e., starting nt and ending nt in the plasmid).

FIGS. 51A to 51C provide a gRNA construct herein to as vector 1067H, comprising or expressing six gRNAs (targeting Dsx and βtub). In one embodiment, vector 1067H refers to pBac-U6(AAEL017774)-sgDsx1-U6(AAEL017763)-sgDsx2-U6(AAEL017774)-sgDsx3-U6(AAEL017763)-sgDsx4-LongScaffold-3 UTR-3xp3-tdTomato-v3-U6(AAEL017774)-sgBTub3-U6(AAEL017763)-sgBTub2 having a sequence of SEQ ID NO: 46. Its plasmid map is shown in FIG. 51A, while FIGS. 51B and 51C list features of the plasmid and the corresponding locations (i.e., starting nt and ending nt in the plasmid).

FIGS. 52A to 52C provide a gRNA construct herein to as vector 1067L, comprising or expressing six gRNAs (targeting Dsx, Isx and βtub). In one embodiment, vector 1067L refers to pBac-pBac-U6(AAEL017774)-sgDsx1-U6(AAEL017774)-sgIsx1-U6(AAEL017774)-sgBTub3-U6(AAEL017763)-sgBTub2-LongScaffold-3 UTR-3xp3-tdTomato-v3-U6(AAEL017774)-sgDsx2-U6(AAEL017763)-sgDsx4 having a sequence of SEQ ID NO: 47. Its plasmid map is shown in FIG. 52A, while FIGS. 52B and 52C list features of the plasmid and the corresponding locations (i.e., starting nt and ending nt in the plasmid).

FIG. 53 provides illustrations of vectors 874L, 874M, 874N and 874P. Ovaries should express GFP. Body should be dsRed. There is no need for Cas9 protein/RNA/Helper plasmid co-Injection. Higher Rates of Cas9 mediated cleavage were detected.

FIG. 54 provides model-predicted impact of releases of pgSIT eggs on Aedes aegypti mosquito population density with comparison to releases of Wolbachia-based incompatible insect technique (IIT), release of insects carrying a dominant lethal gene (MDL), and female-specific MDL (fsRIDL). Releases are carried out weekly over a 6-month period with release ratios (relative to wild adults) shown in the key. Model predictions were computed using 2000 realizations of the stochastic implementation of the MGDrivE simulation framework (Sanchez et al. 2018) for a randomly mixing Ae. aegypti population of 10,000 adult females and model parameters described in Supplemental Table 10 of Kandul et al. 2019. Previous results suggested that pgSIT releases outcompete those of other suppression technologies when pgSIT mating competitiveness is estimated from lab experiments for Drosophila melanogaster (top line). However, pgSIT and fsRIDL perform similarly well when mating competitiveness is estimated from field releases of MDL strains of Aedes aegypti.

### DETAILED DESCRIPTION

Unless defined otherwise, all technical and scientific terms used herein have the same meanings as commonly understood by one of ordinary skill in the art to which this invention belongs. All nucleotide sequences provided herein are presented in the 5′ to 3′ direction. Although any methods and materials similar or equivalent to those described herein can be used in the practice or testing of the present invention, the preferred methods, devices, and materials are now described. All technical and patent publications cited herein are incorporated herein by reference in their entirety. Nothing herein is to be construed as an admission that the invention is not entitled to antedate such disclosure by virtue of prior invention.

The practice of the present technology will employ, unless otherwise indicated, conventional techniques of tissue culture, immunology, molecular biology, microbiology, cell biology, and recombinant DNA, which are within the skill of the art. See, e.g., Green and Sambrook eds. (2012) Molecular Cloning: A Laboratory Manual, 4th edition; the series Ausubel et al. eds. (2015) Current Protocols in Molecular Biology; the series Methods in Enzymology (Academic Press, Inc., N.Y.); MacPherson et al. (2015) PCR 1: A Practical Approach (IRL Press at Oxford University Press); MacPherson et al. (1995) PCR 2: A Practical Approach; McPherson et al. (2006) PCR: The Basics (Garland Science); Harlow and Lane eds. (1999) Antibodies, A Laboratory Manual; Greenfield ed. (2014) Antibodies, A Laboratory Manual; Freshney (2010) Culture of Animal Cells: A Manual of Basic Technique, 6th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Herdewijn ed. (2005) Oligonucleotide Synthesis: Methods and Applications; Hames and Higgins eds. (1984) Transcription and Translation; Buzdin and Lukyanov ed. (2007) Nucleic Acids Hybridization: Modern Applications; Immobilized Cells and Enzymes (IRL Press (1986)); Grandi ed. (2007) In Vitro Transcription and Translation Protocols, 2nd edition; Guisan ed. (2006) Immobilization of Enzymes and Cells; Perbal (1988) A Practical Guide to Molecular Cloning, 2nd edition; Miller and Calos eds, (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Lundblad and Macdonald eds. (2010) Handbook of Biochemistry and Molecular Biology, 4th edition; Herzenberg et al. eds (1996) Weir's Handbook of Experimental Immunology, 5th edition; and/or more recent editions thereof.

The terminology used in the description herein is for the purpose of describing particular embodiments only and is not intended to be limiting of the invention. All publications, patent applications, patents and other references mentioned herein are incorporated by reference in their entirety.

All numerical designations, e.g., pH, temperature, time, concentration, and molecular weight, including ranges, are approximations which are varied (+) or (−) by increments of 1.0 or 0.1, as appropriate or alternatively by a variation of +/−15%, or alternatively 10% or alternatively 5% or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

Unless the context indicates otherwise, it is specifically intended that the various features of the invention described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless explicitly indicated otherwise, all specified embodiments, features, and terms intend to include the recited embodiment, feature, or term and biological equivalents thereof.

### Definitions

As used in the specification and claims, the singular form “a”, “an” and “the” include plural references unless the context clearly dictates otherwise. For example, the term “a polypeptide” includes a plurality of polypeptides, including mixtures thereof.

The term “about,” as used herein when referring to a measurable value such as an amount or concentration and the like, is meant to encompass variations of 20%, 10%, 5%, 1%, 0.5%, or even 0.1% of the specified amount.

As used herein, the term “comprising” is intended to mean that the compositions and methods include the recited elements, but do not exclude others. “Consisting essentially of” when used to define compositions and methods, shall mean excluding other elements of any essential significance to the combination for the intended use. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

The term “protein,” “peptide,” and “polypeptide” are used interchangeably and in their broadest sense to refer to a compound of two or more subunit amino acids, amino acid analogs or peptidomimetics. The subunits may be linked by peptide bonds. In another embodiment, the subunit may be linked by other bonds, e.g., ester, ether, etc. A protein or peptide must contain at least two amino acids and no limitation is placed on the maximum number of amino acids that can comprise a protein's or peptide's sequence. As used herein the term “amino acid” refers to natural and/or unnatural or synthetic amino acids, including glycine and both the D and L optical isomers, amino acid analogs and peptidomimetics.

As used herein, the term “fusion protein” refers to a protein comprised of domains from more than one naturally occurring or recombinantly produced protein, where generally each domain serves a different function. In this regard, the term “linker” refers to a protein fragment that is used to link these domains together—optionally to preserve the conformation of the fused protein domains and/or prevent unfavorable interactions between the fused protein domains that can compromise their respective functions.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double-and single-stranded molecules. Unless otherwise specified or required, any embodiment of this invention that is a polynucleotide encompasses both the double-stranded form and each of two complementary single-stranded forms known or predicted to make up the double-stranded form.

The terms “oligonucleotide” or “polynucleotide” or “portion,” or “segment” thereof refer to a stretch of polynucleotide residues which is long enough to use in PCR or various hybridization procedures to identify or amplify identical or related parts of mRNA or DNA molecules. The polynucleotide compositions of this invention include RNA, cDNA, genomic DNA, synthetic forms, and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (e.g., methyl phosphonates, phosphotriesters, phosphoamidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

A polynucleotide is composed of a specific sequence of four nucleotide bases: adenine (A); cytosine (C); guanine (G); thymine (T); and uracil (U) for thymine when the polynucleotide is RNA. In some embodiments, the polynucleotide may comprise one or more other nucleotide bases, such as inosine (I), a nucleoside formed when hypoxanthine is attached to ribofuranose via a β-N9-glycosidic bond, resulting in the chemical structure:

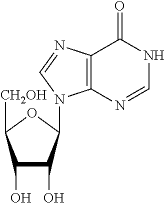

Inosine is read by the translation machinery as guanine (G).

The term “polynucleotide sequence” is the alphabetical representation of a polynucleotide molecule. This alphabetical representation can be input into databases in a computer having a central processing unit and used for bioinformatics applications such as functional genomics and homology searching.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

The terms “equivalent” or “biological equivalent” are used interchangeably when referring to a particular molecule, biological, or cellular material and intend those having minimal homology while still maintaining desired structure or functionality.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

As used herein, the term “functional” may be used to modify any molecule, biological, or cellular material to intend that it accomplish a particular, specified effect.

The compositions for the administration of the CRISPR vectors and systems can be conveniently presented in dosage unit form and can be prepared by any of the methods well known in the art.

“Administration” can be effected in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, nasal administration, injection, and topical application.

The term “effective amount” refers to a quantity sufficient to achieve a desired effect. In the context of therapeutic or prophylactic applications, the effective amount will depend on the type and severity of the condition at issue and the characteristics of the individual subject, such as general health, age, sex, body weight, and tolerance to pharmaceutical compositions. In the context of an immunogenic composition, in some embodiments the effective amount is the amount sufficient to result in a protective response against a pathogen. In other embodiments, the effective amount of an immunogenic composition is the amount sufficient to result in antibody generation against the antigen. In some embodiments, the effective amount is the amount required to confer passive immunity on a subject in need thereof. With respect to immunogenic compositions, in some embodiments the effective amount will depend on the intended use, the degree of immunogenicity of a particular antigenic compound, and the health/responsiveness of the subject's immune system, in addition to the factors described above. The skilled artisan will be able to determine appropriate amounts depending on these and other factors.

In the case of an in vitro application, in some embodiments the effective amount will depend on the size and nature of the application in question. It will also depend on the nature and sensitivity of the in vitro target and the methods in use. The skilled artisan will be able to determine the effective amount based on these and other considerations. The effective amount may comprise one or more administrations of a composition depending on the embodiment.

The term “mutation” as used herein, refers to an alteration to a nucleic acid sequence encoding a protein relative to the consensus sequence of said protein. “Missense” mutations result in the substitution of one codon for another; “nonsense” mutations change a codon from one encoding a particular amino acid to a stop codon. Nonsense mutations often result in truncated translation of proteins. “Silent” mutations are those that have no effect on the resulting protein. As used herein the term “point mutation” refers to a mutation affecting only one nucleotide in a gene sequence. “Splice site mutations” are those mutations present pre-mRNA (prior to processing to remove introns) resulting in mistranslation and often truncation of proteins from incorrect delineation of the splice site.

“Messenger RNA” or “mRNA” is a nucleic acid molecule that is transcribed from DNA and then processed to remove non-coding sections known as introns. The resulting mRNA is exported from the nucleus (or another locus where the DNA is present) and translated into a protein. The term “pre-mRNA” refers to the strand prior to processing to remove non-coding sections.

The terms “hairpin,” “hairpin loop,” “stem loop,” and/or “loop” used alone or in combination with “motif” is used in context of an oligonucleotide to refer to a structure formed in single stranded oligonucleotide when sequences within the single strand which are complementary when read in opposite directions base pair to form a region whose conformation resembles a hairpin or loop.

As used herein, the term “domain” refers to a particular region of a protein or polypeptide and is associated with a particular function. For example, “a domain which associates with an RNA hairpin motif” refers to the domain of a protein that binds one or more RNA hairpin. This binding may optionally be specific to a particular hairpin.

It is to be inferred without explicit recitation and unless otherwise intended, that when the present disclosure relates to a polypeptide, protein, polynucleotide or antibody, an equivalent or a biologically equivalent of such is intended within the scope of this disclosure. As used herein, the term “biological equivalent thereof” is intended to be synonymous with “equivalent thereof” when referring to a reference protein, antibody, polypeptide or nucleic acid, intends those having minimal homology while still maintaining desired structure or functionality. Unless specifically recited herein, it is contemplated that any polynucleotide, polypeptide or protein mentioned herein also includes equivalents thereof. For example, an equivalent intends at least about 70% homology or identity, or at least 80% homology or identity and alternatively, or at least about 85%, or alternatively at least about 90%, or alternatively at least about 95%, or alternatively 98% percent homology or identity and exhibits substantially equivalent biological activity to the reference protein, polypeptide or nucleic acid. Alternatively, when referring to polynucleotides, an equivalent thereof is a polynucleotide that hybridizes under stringent conditions to the reference polynucleotide or its complement.

Applicants have provided herein the polypeptide and/or polynucleotide sequences for use in gene and protein editing techniques described below. It should be understood, although not always explicitly stated that the sequences provided herein can be used to provide the expression product as well as substantially identical sequences that produce a protein that has the same biological properties. These “biologically equivalent” or “biologically active” polypeptides are encoded by equivalent polynucleotides as described herein. They may possess at least 60%, or alternatively, at least 65%, or alternatively, at least 70%, or alternatively, at least 75%, or alternatively, at least 80%, or alternatively at least 85%, or alternatively at least 90%, or alternatively at least 95% or alternatively at least 98%, identical primary amino acid sequence to the reference polypeptide when compared using sequence identity methods run under default conditions. Specific polypeptide sequences are provided as examples of particular embodiments. Modifications to the sequences to amino acids with alternate amino acids that have similar charge. Additionally, an equivalent polynucleotide is one that hybridizes under stringent conditions to the reference polynucleotide or its complement or in reference to a polypeptide, a polypeptide encoded by a polynucleotide that hybridizes to the reference encoding polynucleotide under stringent conditions or its complementary strand. Alternatively, an equivalent polypeptide or protein is one that is expressed from an equivalent polynucleotide.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PC reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

“Homology” or “identity” or “similarity” refers to sequence similarity between two peptides or between two nucleic acid molecules. Homology can be determined by comparing a position in each sequence that can be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present invention.

As used herein, the term “recombinant expression system” refers to a genetic construct or constructs for the expression of certain genetic material formed by recombination.

A “vector” is defined as any molecule that can carry inserted polynucleotides into a host cell. Examples of vectors are liposomes, micelles biocompatible polymers, including natural polymers and synthetic polymers; lipoproteins; polypeptides; polysaccharides; lipopolysaccharides; artificial viral envelopes; metal particles; and bacteria, or viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

A polynucleotide disclosed herein can be delivered to a cell or tissue using a gene delivery vehicle. “Gene delivery,” “gene transfer,” “transducing,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

As used herein, the terms “nucleic acid sequence” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

As used herein, a “contiguous” polynucleotide refers to nucleic acid sequence conjugated with each other directly or indirectly.

A “plasmid” is an extra-chromosomal DNA molecule separate from the chromosomal DNA which is capable of replicating independently of the chromosomal DNA. In many cases, it is circular and double-stranded. Plasmids provide a mechanism for horizontal gene transfer within a population of microbes and typically provide a selective advantage under a given environmental state. Plasmids may carry genes that provide resistance to naturally occurring antibiotics in a competitive environmental niche, or alternatively the proteins produced may act as toxins under similar circumstances.

“Plasmids” used in genetic engineering are called “plasmid vectors”. Many plasmids are commercially available for such uses. The gene to be replicated is inserted into copies of a plasmid containing genes that make cells resistant to particular antibiotics and a multiple cloning site (MCS, or polylinker), which is a short region containing several commonly used restriction sites allowing the easy insertion of DNA fragments at this location. Another major use of plasmids is to make large amounts of proteins. In this case, researchers grow bacteria containing a plasmid harboring the gene of interest. Just as the bacterium produces proteins to confer its antibiotic resistance, it can also be induced to produce large amounts of proteins from the inserted gene.

A “yeast artificial chromosome” or “YAC” refers to a vector used to clone large DNA fragments (larger than 100 kb and up to 3000 kb). It is an artificially constructed chromosome and contains the telomeric, centromeric, and replication origin sequences needed for replication and preservation in yeast cells. Built using an initial circular plasmid, they are linearized by using restriction enzymes, and then DNA ligase can add a sequence or gene of interest within the linear molecule by the use of cohesive ends. Yeast expression vectors, such as YACs, YIps (yeast integrating plasmid), and YEps (yeast episomal plasmid), are extremely useful as one can get eukaryotic protein products with posttranslational modifications as yeasts are themselves eukaryotic cells, however YACs have been found to be more unstable than BACs, producing chimeric effects.

A “viral vector” is defined as a recombinantly produced virus or viral particle that comprises a polynucleotide to be delivered into a host cell, either in vivo, ex vivo or in vitro.

Examples of viral vectors include retroviral vectors, adenovirus vectors, adeno-associated virus vectors, alphavirus vectors and the like. Infectious tobacco mosaic virus (TMV)-based vectors can be used to manufacturer proteins and have been reported to express Griffithsin in tobacco leaves (O'Keefe et al. (2009) Proc. Nat. Acad. Sci. USA 106(15):6099-6104). Alphavirus vectors, such as Semliki Forest virus-based vectors and Sindbis virus-based vectors, have also been developed for use in gene therapy and immunotherapy. See, Schlesinger & Dubensky (1999) Curr. Opin. Biotechnol. 5:434-439 and Ying et al. (1999) Nat. Med. 5(7):823-827. In aspects where gene transfer is mediated by a retroviral vector, a vector construct refers to the polynucleotide comprising the retroviral genome or part thereof, and a therapeutic gene. Further details as to modern methods of vectors for use in gene transfer may be found in, for example, Kotterman et al. (2015) Viral Vectors for Gene Therapy: Translational and Clinical Outlook Annual Review of Biomedical Engineering 17.

As used herein, “retroviral mediated gene transfer” or “retroviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. As used herein, retroviral vector refers to a viral particle capable of introducing exogenous nucleic acid into a cell through a viral or viral-like entry mechanism.

Retroviruses carry their genetic information in the form of RNA; however, once the virus infects a cell, the RNA is reverse-transcribed into the DNA form which integrates into the genomic DNA of the infected cell. The integrated DNA form is called a provirus.

In aspects where gene transfer is mediated by a DNA viral vector, such as an adenovirus (Ad) or adeno-associated virus (AAV), a vector construct refers to the polynucleotide comprising the viral genome or part thereof, and a transgene. Adenoviruses (Ads) are a relatively well characterized, homogenous group of viruses, including over 50 serotypes. Ads do not require integration into the host cell genome. Recombinant Ad derived vectors, particularly those that reduce the potential for recombination and generation of wild-type virus, have also been constructed. Such vectors are commercially available from sources such as Takara Bio USA (Mountain View, CA), Vector Biolabs (Philadelphia, PA), and Creative Biogene (Shirley, NY). Wild-type AAV has high infectivity and specificity integrating into the host cell's genome. See, Wold and Toth (2013) Curr. Gene. Ther. 13(6):421-433, Hermonat & Muzyczka (1984) Proc. Natl. Acad. Sci. USA 81:6466-6470, and Lebkowski et al. (1988) Mol. Cell. Biol. 8:3988-3996.

Vectors that contain both a promoter and a cloning site into which a polynucleotide can be operatively linked are well known in the art. Such vectors are capable of transcribing RNA in vitro or in vivo, and are commercially available from sources such as Agilent Technologies (Santa Clara, Calif) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

Gene delivery vehicles also include DNA/liposome complexes, micelles and targeted viral protein-DNA complexes. Liposomes that also comprise a targeting antibody or fragment thereof can be used in the methods disclosed herein. In addition to the delivery of polynucleotides to a cell or cell population, direct introduction of the proteins described herein to the cell or cell population can be done by the non-limiting technique of protein transfection, alternatively culturing conditions that can enhance the expression and/or promote the activity of the proteins disclosed herein are other non-limiting techniques.

As used herein, a “gene editing system” refers to refers to genetic engineering in which a pol nucleotide is inserted, deleted, modified or replaced in a cell, optionally of an insect.

As used herein, the term “helper” in reference to a virus or plasmid refers to a virus or plasmid used to provide the additional components necessary for replication and packaging of a viral particle or recombinant viral particle. The components encoded by a helper virus may include any genes required for virion assembly, encapsidation, genome replication, and/or packaging. For example, the helper virus may encode necessary enzymes for the replication of the viral genome. Non-limiting examples of helper viruses and plasmids suitable for use with AAV constructs include pHELP (plasmid), adenovirus (virus), or herpesvirus (virus).

As used herein, the term “AAV” is a standard abbreviation for adeno-associated virus. Adeno-associated virus is a single-stranded DNA parvovirus that grows only in cells in which certain functions are provided by a co-infecting helper virus. General information and reviews of AAV can be found in, for example, Carter, 1989, Handbook of Parvoviruses, Vol. 1, pp. 169-228, and Berns, 1990, Virology, pp. 1743-1764, Raven Press, (New York). It is fully expected that the same principles described in these reviews will be applicable to additional AAV serotypes characterized after the publication dates of the reviews because it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3: 1-61 (1974)). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control.

An “AAV vector” as used herein refers to a vector comprising one or more polynucleotides of interest (or transgenes) that are flanked by AAV terminal repeat sequences (ITRs). Such AAV vectors can be replicated and packaged into infectious viral particles when present in a host cell that has been transfected with a vector encoding and expressing rep and cap gene products.

Adeno-associated virus (AAV) is a replication-deficient parvovirus, the single-stranded DNA genome of which is about 4.7 kb in length including two 145 nucleotide inverted terminal repeat (ITRs). There are multiple serotypes of AAV. The nucleotide sequences of the genomes of the AAV serotypes are known. For example, the complete genome of AAV-1 is provided in GenBank Accession No. NC 002077; the complete genome of AAV-2 is provided in GenBank Accession No. NC 001401 and Srivastava et al., J. Virol., 45: 555-564 {1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(1): 67-76 (2006); and the AAV-11 genome is provided in Virology, 330(2): 375-383 (2004). The sequence of the AAV rh.74 genome is provided in U.S. Pat. No. 9,434,928, incorporated herein by reference. Cis-acting sequences directing viral DNA replication (rep), encapsidation/packaging and host cell chromosome integration are contained within the AAV ITRs. Three AAV promoters (named p5, p19, and p40 for their relative map locations) drive the expression of the two AAV internal open reading frames encoding rep and cap genes. The two rep promoters (p5 and pi 9), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is inserted as cloned DNA in plasmids, which makes construction of recombinant genomes feasible. Furthermore, because the signals directing AAV replication and genome encapsidation are contained within the ITRs of the AAV genome, some or all of the internal approximately 4.3 kb of the genome (encoding replication and structural capsid proteins, rep-cap) may be replaced with foreign DNA. To generate AAV vectors, the rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

Multiple studies have demonstrated long-term (>1.5 years) recombinant AAV-mediated protein expression in muscle. See, Clark et al., Hum Gene Ther, 8: 659-669 (1997); Kessler et al., Proc Nat. Acad Sc. USA, 93: 14082-14087 (1996); and Xiao et al., J Virol, 70: 8098-8108 (1996). See also, Chao et al., Mol Ther, 2:619-623 (2000) and Chao et al., Mol Ther, 4:217-222 (2001). Moreover, because muscle is highly vascularized, recombinant AAV transduction has resulted in the appearance of transgene products in the systemic circulation following intramuscular injection as described in Herzog et al., Proc Natl Acad Sci USA, 94: 5804-5809 (1997) and Murphy et al., Proc Natl Acad Sci USA, 94: 13921-13926 (1997). Moreover, Lewis et al., J Virol, 76: 8769-8775 (2002) demonstrated that skeletal myofibers possess the necessary cellular factors for correct antibody glycosylation, folding, and secretion, indicating that muscle is capable of stable expression of secreted protein therapeutics. Recombinant AAV (rAAV) genomes of the invention comprise a nucleic acid molecule encoding γ-sarcoglycan and one or more AAV ITRs flanking the nucleic acid molecule. AAV DNA in the rAAV genomes may be from any AAV serotype for which a recombinant virus can be derived including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13 and AAV rh74. Production of pseudotyped rAAV is disclosed in, for example, WO 01/83692. Other types of rAAV variants, for example rAAV with capsid mutations, are also contemplated. See, for example, Marsic et al., Molecular Therapy, 22(11): 1900-1909 (2014). The nucleotide sequences of the genomes of various AAV serotypes are known in the art.

The term “Cas9” refers to a CRISPR associated endonuclease referred to by this name. Non-limiting exemplary Cas9s are provided herein, e.g. the Cas9 provided for in UniProtKB G3ECR1 (CAS9_STRTR) or the Staphylococcus aureus Cas9, as well as the nuclease dead Cas9, orthologs and biological equivalents each thereof. Orthologs include but are not limited to Streptococcus pyogenes Cas9 (“spCas9”); Cas 9 from Streptococcus thermophiles, Legionella pneumophilia, Neisseria lactamica, Neisseria meningitides, Francisella novicida; and Cpf1 (which performs cutting functions analogous to Cas9) from various bacterial species including Acidaminococcus spp. and Francisella novicida U112.

As used herein, the term “CRISPR” refers to a technique of sequence specific genetic manipulation relying on the clustered regularly interspaced short palindromic repeats pathway. CRISPR can be used to perform gene editing and/or gene regulation, as well as to simply target proteins to a specific genomic location. Gene editing refers to a type of genetic engineering in which the nucleotide sequence of a target polynucleotide is changed through introduction of deletions, insertions, or base substitutions to the polynucleotide sequence. In some aspects, CRISPR-mediated gene editing utilizes the pathways of non-homologous end-joining (NHEJ) or homologous recombination to perform the edits. Gene regulation refers to increasing or decreasing the production of specific gene products such as protein or RNA.

In one embodiment, the guide polynucleotide is a gRNA. The term “gRNA” or “guide RNA” as used herein refers to the guide RNA sequences used to target specific genes for correction employing the CRISPR technique. Techniques of designing gRNAs and donor therapeutic polynucleotides for target specificity are well known in the art. For example, Doench, J., et al. Nature biotechnology 2014; 32(12):1262-7, Mohr, S. et al. (2016) FEBS Journal 283: 3232-38, and Graham, D., et al. Genome Biol. 2015; 16: 260. gRNA comprises or alternatively consists essentially of, or yet further consists of a fusion polynucleotide comprising CRISPR RNA (crRNA) and trans-activating CRISPR RNA (tracrRNA); or a polynucleotide comprising CRISPR RNA (crRNA) and trans-activating CRISPR RNA (tracrRNA). In some aspects, a gRNA is synthetic (Kelley, M. et al. (2016) J of Biotechnology 233 (2016) 74-83). As used herein, a biological equivalent of a gRNA includes but is not limited to polynucleotides or targeting molecules that can guide a Cas9 or equivalent thereof to a specific nucleotide sequence such as a specific region of a cell's genome.

A “composition” is intended to mean a combination of active polypeptide, polynucleotide or antibody and another compound or composition, inert (e.g., a detectable label) or active (e.g., a gene delivery vehicle).

As used herein, with respect to the CRISPR-based technology, the term “guide polynucleotide” refers to a polynucleotide having a “synthetic sequence” capable of binding the corresponding endonuclease enzyme protein (e.g., Cas9) and a variable target sequence capable of binding the genomic target (e.g., a nucleotide sequence found in an exon of a target gene). In some embodiments of the present disclosure, a guide polynucleotide is a guide ribonucleic acid (gRNA). In some embodiments, the variable target sequence of the guide polynucleotide is any sequence within the target that is unique with respect to the rest of the genome and is immediately adjacent to a Protospacer Adjacent Motif (PAM). The exact sequence of the PAM sequence may vary as different endonucleases require different PAM sequences.

With respect to the endonuclease enzyme protein of the CRISPR-based technology, the term “endonuclease” refers to any suitable endonuclease enzyme protein or a variant thereof that will be specifically directed by the selected guide polynucleotide to enzymatically knock-out the target sequence of the guide polynucleotide. As used herein, the term “variant thereof,” as used with respect to an endonuclease, refers to the referenced endonuclease in its enzymatically functional form expressed in any suitable host organism or expression system and/or including any modifications to enhance the enzymatic activity of the endonuclease. In some embodiments of the present disclosure, a suitable endonuclease includes a CRISPR-associated sequence 9 (Cas9) endonuclease or a variant thereof, a CRISPR-associated sequence 13 (Cas13) endonuclease or a variant thereof, CRISPR-associated sequence 6 (Cash) endonuclease or a variant thereof, a CRISPR from Prevotella and Francisella 1 (Cpf1) endonuclease or a variant thereof, or a CRISPR from Microgenomates and Smithella 1 (Cms1) endonuclease or a variant thereof. In some embodiments of the present disclosure, a suitable endonuclease includes a Streptococcus pyogenes Cas9 (SpCas9), a Staphylococcus aureus Cas9 (SaCas9), a Francisella novicida Cas9 (FnCas9), or a variant thereof. Variants may include a protospacer adjacent motif (PAM) SpCas9 (xCas9), high fidelity SpCas9 (SpCas9-FIF1), a high fidelity SaCas9, or a high fidelity FnCas9. In other embodiments of the present disclosure, the endonuclease comprises a Cas fusion nuclease comprising a Cas9 protein or a variant thereof fused with a Fok1 nuclease or variant thereof. Variants of the Cas9 protein of this fusion nuclease include a catalytically inactive Cas9 (e.g., dead Cas9). In some embodiments of the present disclosure, the endonuclease may be a Cas9, Cas13, Cas6, Cpf1, CMS1 protein, or any variant thereof that is derived or expressed from Methanococcus maripaludis C7, Corynebacterium diphtheria, Corynebacterium efficiens YS-314, Corynebacterium glutamicum (ATCC 13032), Corynebacterium glutamicum (ATCC 13032), Corynebacterium glutamicum R, Corynebacterium kroppenstedtii (DSM 44385), Mycobacterium abscessus (ATCC 19977), Nocardia farcinica IFM1 0 152, Rhodococcus erythropolis PR4, Rhodococcus jostii RFIA1, Rhodococcus opacus B4 (uid36573), Acidothermus cellulolyticus 11B, Arthrobacter chlorophenolicus A6, Kribbella flavida (DSM 17836, uid43465), Thermomonospora curvata (DSM431 83), Bifidobacterium dentium Bd1, Bifidobacterium longum DJO10A, Slackia heliotrinireducens (DSM 20476), Persephonella marina EX H1, Bacteroides fragilis NCTC 9434, Capnocytophaga ochracea (DSM 7271), Flavobacterium psychrophilum JIP02 86, Akkermansia muciniphila (ATCC BAA 835), Roseiflexus castenholzii (DSM 13941), Roseiflexus RS1, Synechocystis PCC6803, Elusimicrobium minutum Pei191, uncultured Termite group 1 bacterium phylotype Rs D 17, Fibrobacter succinogenes S85, Bacillus cereus (ATCC 10987), Listeria innocua, Lactobacillus casei, Lactobacillus rhamnosus GG, Lactobacillus salivarius UCC1 18, Streptococcus agalactiae-5-A909, Streptococcus agalactiae NEM316, Streptococcus agalactiae 2603, Streptococcus dysgalactiae equisimilis GGS 124, Streptococcus equi zooepidemicus MGCS1 0565, Streptococcus gallolyticus UCN34 (uid46061), Streptococcus gordonii Challis subst CH1, Streptococcus mutans NN2025 (uid46353), Streptococcus mutans, Streptococcus pyogenes M 1 GAS, Streptococcus pyogenes MGAS5005, Streptococcus pyogenes MGAS2096, Streptococcus pyogenes MGAS9429, Streptococcus pyogenes MGAS 10270, Streptococcus pyogenes MGAS61 80, Streptococcus pyogenes MGAS31 5, Streptococcus pyogenes SSI-1, Streptococcus pyogenes MGAS1 0750, Streptococcus pyogenes NZ1 3 1, Streptococcus thermophiles CNRZ1 066, Streptococcus thermophiles LMD-9, Streptococcus thermophiles LMG 1831 1, Clostridium botulinum A3 Loch Maree, Clostridium botulinum B Eklund 17B, Clostridium botulinum Ba4 657, Clostridium botulinum F Langeland, Clostridium cellulolyticum H 10, Finegoldia magna (ATCC 29328), Eubacterium rectale (ATCC 33656), Mycoplasma gallisepticum, Mycoplasma mobile 163K, Mycoplasma penetrans, Mycoplasma synoviae 53, Streptobacillus moniliformis (DSM 121 12), Bradyrhizobium BTAil, Nitrobacter hamburgensis X14, Rhodopseudomonas palustris BisB1 8, Rhodopseudomonas palustris BisB5, Parvibaculum lavamentivorans DS-1, Dinoroseobacter shibae. DFL 12, Gluconacetobacter diazotrophicus Pal 5 FAPERJ, Gluconacetobacter diazotrophicus Pal 5 JGI, Azospirillum B51 0 (uid46085), Rhodospirillum rubrum (ATCC 11170), Diaphorobacter TPSY (uid29975), Verminephrobacter eiseniae EF01-2, Neisseria meningitides 053442, Neisseria meningitides alpha14, Neisseria meningitides Z2491, Desulfovibrio salexigens DSM 2638, Campylobacter jejuni doylei 269 97, Campylobacter jejuni 8 1116, Campylobacter jejuni, Campylobacter lari RM21 00, Helicobacter hepaticus, Wolinella succinogenes, Tolumonas auensis DSM 9 187, Pseudoalteromonas atlantica T6c, Shewanella pealeana (ATCC 700345), Legionella pneumophila Paris, Actinobacillus succinogenes 130Z, Pasteurella multocida, Francisella tularensis novicida U112, Francisella tularensis holarctica, Francisella tularensis FSC 198, Francisella tularensis, Francisella tularensis WY96-3418, or Treponema denticola (ATCC 35405).

The term “cell” as used herein may refer to either a prokaryotic or eukaryotic cell, optionally obtained from a subject or a commercially available source. In one embodiment, the cell is an insect cell.

“Eukaryotic cells” comprise all of the life kingdoms except monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. Unless specifically recited, the term “host” includes a eukaryotic host, including, for example, yeast, higher plant, insect and mammalian cells. Non-limiting examples of eukaryotic cells or hosts include simian, bovine, porcine, murine, rat, avian, reptilian and human, e.g., HEK293 cells, Chinese Hamster Ovary (CHO) cells and 293T cells.

“Prokaryotic cells” that usually lack a nucleus or any other membrane-bound organelles and are divided into two domains, bacteria and archaea. In addition to chromosomal DNA, these cells can also contain genetic information in a circular loop called an episome. Bacterial cells are very small, roughly the size of an animal mitochondrion (about 1-2 μm in diameter and 10 μm long). Prokaryotic cells feature three major shapes: rod shaped, spherical, and spiral. Instead of going through elaborate replication processes like eukaryotes, bacterial cells divide by binary fission. Examples include but are not limited to Bacillus bacteria, E. coli bacterium, and Salmonella bacterium.

As used herein, the terms “engineered” “modified” and like terms refers to the introduction of a heterologous recombinant nucleic acid sequence into the target, such as another nucleic acid sequence, chromosome, cell or insect egg, or insect. As would be understood by one of ordinary skill in the art, techniques for genetic modification of insects are known and described, for example in Cockburn et al., Biotechnology and Genetic Engineering Reviews, 2: 68-99, (1984), the entire contents of which are incorporate herein by reference. In one embodiment, the term “engineered” “integrated” “modified” or the like may refer to the integration of recombinant nucleic acid sequence into the genome of the target insect. The genome of the target insect includes at least one chromosome of the target insect, but may include all relevant chromosome copies. As such, integration into the genome may be heterozygous or homozygous.

As used herein, the term “female-essential genomic sequence” encompasses any genomic sequence or gene specific to the female insect. Examples of a female-essential genomic sequence include a sex-determination gene or a female-specific splice variant thereof, a gene or splice variant of a gene not found in the male, a gene or splice variant of a gene essential for female gonadal development, and/or a gene or splice variant of a gene not essential for male viability. Non-limiting examples of female-essential genomic sequences include the female-specific exons in the sex-determination genes Sex-lethal (Sxl), sister of sex lethal (Slx), transformer (Tra), transformer-2 (tra-2), Intersex, Proboscipedia (Pb), labrum (Lab), and double sex (Dsx) including homologs, orthologs, and paralogs thereof. In one embodiment, the Intersex gene is the gene encoding mediator of RNA polymerase II transcription subunit 29. As used herein, the term “homolog” refers to the comparable gene of an organism found in another organism conferring the same function. As used herein, the terms “orthologs” and “paralogs” refer to types of homologs. Orthologs are corresponding genes in different lineages and are a result of speciation, and paralogs result from a gene duplication. See, for example, WO 2019/103982. In one embodiment, these sequences are sequences of ans Aedes aegypti mosquito. In another embodiment, these sequences are sequences of a Drosophila suzukii.

As used herein, the term “male sterility genomic sequence” refers to any male-specific genomic sequence (such as a gene) required for male fertility in an insect which does not affect the development of the male insect or the viability of the male insect. Non-limiting examples of a male-specific genomic sequence required for male fertility in an insect include the genes pTubulin 85D (PTub), beta tubulin (betatub), beta2 tubulin (beta2tub), fuzzy onions (Fzo), protamine A (ProtA), zero population growth (Zpg)1, Cannonball (Can), meiosis I arrest (Mia), P-element induced wimpy testis (PIWI), Heterochromatin Protein 1e (HP1e), zero population growth (Zpg), male sterile (3) K81 (K81), misfire (Mfr), sneaky (Snky), boule (bol), growth arrest specific protein 8 (gas 8), No-hitter (nht), Poly(A) RNA polymerase gld-2 homolog A (Gld2), Nix and spermatocyte arrest (Sa) and homologs, orthologs, and paralogs thereof. In one embodiment, the male sterility genomic sequence also refers to a genomic sequence (such as a gene) required for male mating barrier (such as male sexual behavior or male mating competitiveness, competing with a wild type). Non-limiting examples are fruitless (fru) or Myosin Heavy chain. See, for example, Aryan et al. 2020. In some embodiments, the nucleic acid sequence construct includes one or more second guide polynucleotides targeting one or more male-specific genomic sequence required for male fertility. The functional conservation of pTubulin 85D including Anopheles and Aedes aegypti is described in Catteruccia et al., Nat. Biotechnol. 23, 1414-141 7 (2005) and Smith et al., Insect Mol. Biol. 16, 61-71 (2007), the entire contents of both of which are incorporated herein by reference. Also, see, for example, WO 2019/103982. In one embodiment, these sequences are sequences of ans Aedes aegypti mosquito. In another embodiment, these sequences are sequences of a Drosophila suzukii.

In some embodiments of the present disclosure, the genetically modified insects and methods for generating the genetically modified insects include insects from the Order Diptera, Lepidoptera, or Coleoptera. In some embodiments of the present disclosure, the genetically modified insects and methods for generating the genetically modified insects include an insect selected from a mosquito of the genera Stegomyia, Aedes, Anopheles, or Culex. Of these genera, example mosquito species include Aedes aegypti, Aedes albopictus, Ochlerotatus triseriatus (Aedes triseriatus), Anopheles stephensi, Anopheles albimanus, Anopheles gambiae, Anopheles quadrimaculatus, Anopheles freeborni, Culex species, or Culiseta melanura.

In some embodiments, the genetically modified insects and methods for generating the genetically modified insects include any insect selected from one of the following: tephritid fruit fly selected from Medfly (Ceratitis capitata), Mexfly (Anastrepha ludens), Oriental fruit fly (Bactrocera dorsalis), Olive fruit fly (Bactrocera oleae), Melon fly (Bactrocera cucurbitae), Natal fruit fly (Ceratitis rosa), Cherry fruit fly (Rhagoletis cerasi), Queensland fruit fly (Bactrocera tyroni), Peach fruit fly (Bactrocera zonata), Caribbean fruit fly (Anastrepha suspensa), Oriental Fruit Fly (Bactrocera dorsalis), West Indian fruit fly (Anastrepha obliqua), the New World screwworm (Cochliomyia hominivorax), the Old World screwworm (Chrysomya bezziana), Australian sheep blowfly/greenbottle fly (Lucilia cuprina), the pink bollworm (Pectinophora gossypiella), the European Gypsy moth (Lymantria dispar), the Navel Orange Worm (Amyelois transitella), the Peach Twig Borer (Anarsia lineatella), the rice stem borer (Tryporyza incertulas), the noctuid moths, Heliothinae, the Japanese beetle (Papilla japonica), White-fringed beetle (Graphognatus spp.), Boll weevil (Anthonomous grandis), the Colorado potato beetle (Leptinotarsa decern lineata), the vine mealybug (Planococcus ficus), Asian citrus psyllid (Diaphorina citri), Spotted wing drosophila (drosophila suzukii), Bluegreen sharpshooter (Graphocephala atropunctata), Glassy winged sharpshooter (Homalodisca vitripennis), Light brown apple moth (Epiphyas postvittana), Bagrada bug (Bagrada hilaris), Brown marmorated stink bug (Halyomorpha halys), Asian Gypsy Moth selected from the group of Lymantria dispar asiatica, Lymantria dispar japonica, Lymantria albescens, Lymantria umbrosa, and Lymantria postalba, Asian longhorned beetle (Anoplophora glabripennis), Coconut Rhinoceros Beetle (Oryctes rhinoceros), Emerald Ash Borer (Agrilus planipennis), European Grapevine Moth (Lobesia botrana), European Gypsy Moth (Lymantria dispar), False Codling Moth (Thaumatotibia leucotreta), fire ants selected from Solenopsis invicta Buren, and S. richteri Forel, Old World Bollworm (Helicoverpa armigera), Spotted Lanternfly (Lycorma delicatula), Africanized honeybee (Apis mellifera scutellata), Fruit and shoot borer (Leucinodes orbonalis), corn root worm (Diabrotica spp.), Western corn rootworm (Diabrotica virgifera), Whitefly (Bemisia tabaci), House Fly (Musca Domestica), Green Bottle Fly (Lucilia cuprina), Silk Moth (Bombyx mori), Red Scale (Aonidiella aurantia), Dog heartworm (Dirofilaria immitis), Southern pine beetle (Dendroctonus frontalis), Avocado thrip (Thysanoptera Spp.), Botfly selected from Oestridae spp. and Dermatobia hominis), Horse Fly (Tabanus sulcifrons), Horn Fly (Haematobia irritans), Screwworm Fly selected from Cochliomyia macellaria (C. macellaria), C. hominivorax, C. aldrichi, or C. minima, Tsetse Fly (Glossina spp.), Warble Fly selected from Hypoderma bovis or Hypoderma lineatum, Spotted lanternfly (Lycorma delicatula), Khapra beetle (Trogoderma granarium), Honeybee mite (Varroa destructor), Termites (Coptotermes formosanus), Hemlock woolly adelgid (Adelges tsugae), Walnut twig beetle (Pityophthorus juglandis), European wood wasp (Sirex noctilio), Pink-spotted bollworm (Pectinophora scutigera), Two spotted spider mite (Tertanychus urticae), Diamondback moth (Plutella xylostella), Taro caterpillar (Spodoptera litura), Red flour beetle (Tribolium castaneum), Green peach aphid (Myzus persicae), Cotton Aphid (Aphis gossypii), Brown planthopper (Nilaparvata lugens), Beet armyworm (Spodotera exigua), Western flower thrips (Frankliniella occidentalis), Codling moth (Cydia pomonella), Cowpea weevil (Callosobruchus maculatus), Pea aphid (Acyrthosiphon pisum), Tomato leafminer (Tuta absoluta), Onion thrips (Thrips tabaci), and Cotton bollworm (Helicoverpa armigera).

The term “isolated” as used herein refers to molecules or biologicals or cellular materials being substantially free from other materials.

As used herein, the term “detectable marker” refers to at least one marker capable of directly or indirectly, producing a detectable signal. A non-exhaustive list of this marker includes enzymes which produce a detectable signal, for example by colorimetry, fluorescence, luminescence, such as horseradish peroxidase, alkaline phosphatase, β-galactosidase, glucose 6-phosphate dehydrogenase, chromophores such as fluorescent, luminescent dyes, groups with electron density detected by electron microscopy or by their electrical property such as conductivity, amperometry, voltammetry, impedance, detectable groups, for example whose molecules are of sufficient size to induce detectable modifications in their physical and/or chemical properties, such detection may be accomplished by optical methods such as diffraction, surface plasmon resonance, surface variation, the contact angle change or physical methods such as atomic force spectroscopy, tunnel effect, or radioactive molecules such as ³²P, ³⁵S or ¹²⁵I.

As used herein, the term “purification marker” or “selectable marker” refers to at least one marker useful for purification or identification. A non-exhaustive list of this marker includes His, lacZ, GST, maltose-binding protein, NusA, BCCP, c-myc, CaM, FLAG, GFP, YFP, cherry, thioredoxin, poly(NANP), V5, Snap, HA, chitin-binding protein, Softag 1, Softag 3, Strep, or S-protein. Suitable direct or indirect fluorescence marker comprise FLAG, GFP, YFP, RFP, dTomato, cherry, Cy3, Cy 5, Cy 5.5, Cy 7, DNP, AMCA, Biotin, Digoxigenin, Tamra, Texas Red, rhodamine, Alexa fluors, FITC, TRITC or any other fluorescent dye or hapten.

As used herein, the term “progeny” refers to a descendant or the descendants. In one embodiment, the progeny is an insect egg or a population thereof. In another embodiment, the progeny is an insect or a population thereof. In yet another embodiment, the progeny is one or more of the following: an insect egg, an insect, or a population thereof.

As used herein, the term “nuclear localization signal” or “NLS” refers to an amino acid sequence that ‘tags’ a protein for import into the cell nucleus by nuclear transport. Typically, this signal consists of one or more short sequences of positively charged lysines or arginines exposed on the protein surface. Different nuclear localized proteins may share the same NLS. An NLS has the opposite function of a nuclear export signal (NES), which targets proteins out of the nucleus.

As used herein, the term “regulatory sequence” or “expression control sequence” or the like refers to a segment of a nucleic acid molecule which is capable of increasing or decreasing the expression of specific genes within an organism. Expression control or regulatory sequences may include, e.g., include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A promoter may be selected from amongst a constitutive promoter, a tissue-specific promoter, or a cell-specific promoter.

### MODES FOR CARRYING OUT THE DISCLOSURE

Efficient sexing is an essential component to mosquito sterile insect technique (SIT) programs, since females bite and transmit disease. Additionally, the predilection for assortative mating among sterile insects and higher cost of multi-sex releases further contribute to the need for genetic sexing tools (Knipling 1955). There have been many sexing methods as disclosed: biological (e.g. sex are separated based on pupal dimorphism, female blood feeding preference, or male swarming behaviors); classical chemically/radiation induced male-specific selectable traits; or genetic/transgenic, synthetic male-specific or female lethal traits (as reviewed in Papathanos et al. 2009; Gilles et al. 2014; Catteruccia, Crisanti, and Wimmer 2009). However, there is no current technology that couples genetic sexing with a mechanism for male sterility in early development. The approach as disclosed herein drastically simplifies the SIT technology, making it more feasible and scalable than any existing vector control technology. Moreover, these technologies can be incorporated into any genetic vector and can be used without gene drive technology.

The Sterile Insect Technique (SIT) is an alternative, highly promising approach that could complement existing control methods. SIT involves the mass-production and release of sterile males, and has historically been used to control, and eradicate, insect pest populations dating back to the mid-1930s (Knipling 1955; Bushland, Lindquist, and Knipling 1955; Klassen and Curtis 2005; Vanderplank 1944; Dyck, Hendrichs, and Robinson 2005). Traditional SIT methodologies have relied on DNA-damaging agents for sterilization, substantially reducing overall fitness and mating competitiveness of released males. To overcome these issues, modern genetic SIT-like systems such as the Release of Insects carrying a Dominant Lethal (MDL) (Thomas et al. 2000; Slade and Morrison 2014), and other methodologies to release fertile males that genetically kill females such as female-specific MDL (fsRIDL) (Fu et al. 2010), and autosomal-linked X-chromosome shredders have been developed (Windbichler, Papathanos, and Crisanti 2008; Alphey 2016). While these first-generation genetic SIT technologies represent significant advances, the use of tetracycline known to ablate the microbiota (Wilkinson 1998) compromises the fitness of RIDL/fsRIDL males, and X-chromosome shredders can in principle only be developed in species with heterogametic sex chromosomes, thereby limiting wide applicability to other species. Furthermore, none of these technologies has been developed for D. suzukii or Aedes aegypti.

A next-generation highly-efficient technology that can be used for biocontrol of D. suzukii or Aedes aegypti is precision guided SIT (pgSIT). PgSIT functions by exploiting the precision and accuracy of CRISPR to simultaneously disrupt genes essential for either female viability or male fertility. It utilizes a simple breeding scheme requiring two homozygous strains—one expressing Cas9 and the other expressing double guide RNAs (dgRNAs). A single mating between these strains mechanistically results in synchronous RNA-guided dominant biallelic knockouts of both target genes throughout development, resulting in the complete penetrance of desired phenotypes in all progeny. pgSIT in Drosophila melanogaster, a model organism, was previously built (WO 2019/103982, Kandul et al. 2018 and Kandul et al. 2019). It is shown that it is extremely robust at genetically sexing and simultaneously sterilizing resulting progeny reproducibly with 100% efficiency, and that pgSIT sterile males are fit and can compete for mates. pgSIT organism that is closely related to D. suzukii.

A. D. suzukii

Applicant developed several functional prototype pgSIT systems in D. suzukii informed by the strategy and validated sequences from the work in D. melanogaster (WO 2019/103982, Kandul et al. 2018 and Kandul et al. 2019). For example, applicant built and validated multiple Cas9 expression lines in D. suzukii: nanos (nos-Cas9), vasa (vas-Cas9), bicoid (BicC-Cas9), ubiquitin (ubi-Cas9) and deadhead (Dhd-Cas9). All of these lines have demonstrated up to 100% efficiency when coupled with a gRNA targeting the white eye color gene and the Sxl guide RNA.

To disrupt female viability, applicant targeted several sex-specifically alternatively spliced sex-determination genes including sex lethal (Sxl), transformer (tra), and doublesex (dsxF), as well as zero population growth (zpg), a germline-specific gap junction gene. The sxl dgRNA line has achieved 100% female lethality in the preliminary crosses to Cas9 lines. To disrupt male fertility, applicant targeted βTub, a gene active during spermatogenesis. Some of these lines, such as sxl and βTub, were combined into a double gRNA (dgRNA) or triple gRNA (tgRNA) lines and resulted in 100% sterile transheterozygous males progeny upon crossing.

In order to be easily implemented, the pgSIT approach also requires the ability to efficiently separate animals by sex to set up appropriate crosses for sterile male generation (WO 2019/103982, Kandul et al. 2018 and Kandul et al. 2019). Therefore, applicant developed a sex-specific fluorescent reporter transgene that facilitates automated sex sorting. Specifically, applicant designed a transgene that contains a fluorescent marker (dsRed) under the control of a ubiquitous promoter; this transgene includes a female-specific intron that should mediate successful splicing of the dsRed only in females. Applicant obtained transgenic insects with the above transgene and demonstrated that only females express the red marker.

Here is a non-limiting list of constructs for pgSIT as disclosed herein. Corresponding sequences, features of the sequences and plasmid maps can be found in the drawings while the sequences are provided in the sequence listing.
Cas9 constructs
   Vasa-Cas9: 874Z (pBac-vasa-Cas9-2a-eGFP-p10-UTR-Opie2-dsRed)
   Dhd-Cas9: 874S (pBac-dmeDhd-Cas9-2a-eGFP-p10-UTR-Opie2-dsRed)
   BicC-Cas9: 874R (pBac-dmeBicC-Cas9-2a-eGFP-p10-UTR-Opie2-dsRed)
   Ubiq-Cas9: 874W (pBac-dmeUbiq-Cas9-2a-eGFP-p10-UTR-Opie2-dsRed)
   Nanos-Cas9: 874Z1 (pBac-dmeNanos-Cas9-2a-eGFP-p10-UTR-Opie2-dsRed)
Double gRNA constructs
   Initial construct with two sxl gRNA: 1056C.2
      (HL-U6:3-sxl2-gRNA-UTR-opie-mVenus-SV40-U6:3-sxl1-gRNA-UTR-HR)
   Four constructs with two sxl, one btub:
      1056H: (HL-U6:3-sxl2-gRNA-UTR-opie-mVenus-SV40-U6:3-sxl1-gRNA-UTR-HR-U6-Btuborig2-UTR)
      1056I: (HL-U6:3-sxl2-gRNA-UTR-opie-mVenus-SV40-U6:3-sxl1-gRNA-UTR-HR-U6-Btub2-UTR)
      1056J: (HL-U6:3-sxl2-gRNA-UTR-opie-mVenus-SV40-U6:3-sxl1-gRNA-UTR-HR-U6-Btub3-UTR)
      1056K: (HL-U6:3-sxl2-gRNA-UTR-opie-mVenus-SV40-U6:3-sxl1-gRNA-UTR-HR-U6-Btub4-UTR)
   Others:
      1104D (Sxl and two Cannonball gRNA): pBac-[U6-gRNA-Slx]-[U6-gRNA #2]-[Opie2-eGFP-p10]-[U6-gRNA #1-Can]
      1104E (Sxl and two Mia gRNA): pBac-[U6-gRNA-Slx]-[U6-gRNA-Mia #1]-[Opie2-eGFP-p10]-[U6-gRNA-Mia #2]
      1104J (Sxl and two Zpg gRNA): pBac-[U6-gRNA-Slx]-[U6-gRNA-Zpg #1]-[Opie2-eGFP-p10]-[U6-gRNA-Zpg #2]
      1104H (Sxl and two PIWI gRNA): pBac-[U6-gRNA-Slx]-[U6-gRNA-PIWI #1]-[Opie2-eGFP-p10]-[U6-gRNA-PIWI #2]
      1104I (Sxl and two HP1e gRNA): pBac-[U6-gRNA-Slx]-[U6-gRNA-HP1e #1]-[Opie2-eGFP-p10]-[U6-gRNA-HP1e #2]
      1104K (Sxl and two K81 gRNA): pBac-[U6-gRNA-Slx]-[U6-gRNA-K81 #1]-[Opie2-eGFP-p10]-[U6-gRNA-K81 #2]
      1104M (Sxl and two Mfr gRNA): pBac-[U6-gRNA-Slx]-[U6-gRNA-Mfr #1]-[Opie2-eGFP-p10]-[U6-gRNA-Mfr #2]
      1104N (Sxl and two Snky gRNA): pBac-[U6-gRNA-Slx]-[U6-gRNA-Snky #1]-[Opie2-eGFP-p10]-[U6-gRNA-Snky #2]
         B. Aedes aegypti.

The disclosed technology described herein generates a precision guided SIT (pgSIT) technology which simultaneously knock-outs female viability (e.g. sex determinate) and male fertility genes using a binary CRISPR/Cas9 system in the dengue, Zika and yellow fever vector, Ae. aegypti. Importantly, as the proof-of-principle, the efficiency of pgSIT in Drosophila melanogaster was demonstrated (WO 2019/103982, Kandul et al. 2018 and Kandul et al. 2019). Specifically, D. melanogaster lines were created each expressing either Cas9 endonuclease or double guide RNAs (gRNAs), targeting one of the female-specific variants of sex determination genes sex lethal (sxl), transformer (tra), or doublesex (dsx) and a male fertility gene, beta tubulin (βTub). A simple genetic cross between Cas9 and gRNAs homozygous strains resulted in simultaneous Cas9/gRNA-based knockouts of both female viability and male sterility genes, and caused 100% male sterility and depending on which gRNAs combination was used, female lethality or masculinization in F₁ progeny. Notably, the longevity and competitiveness of pgSIT males were not compromised (WO 2019/103982, Kandul et al. 2018 and Kandul et al. 2019). As disclosed herein, constructs, compositions and methods were applied to the primary disease vector, Ae. aegypti.

Here is a non-limiting list of constructs for pgSIT as disclosed herein. Corresponding sequences, features of the sequences and plasmid maps can be found in the drawings while the sequences are provided in the sequence listing.
Cas9 constructs
   Exuperentia-cas9 (874L)
   4-nitrophenylphosphatase-cas9 (874M)
   Trunk-cas9 (874N)
   Nup50-cas9 (874P)
   Polyubiquitin-cas9 (874X)
   Ubiquitin L40-cas9 (874Y)
   Vasa-cas9 (874Y3)
gRNA constructs/plasmids
   2 gRNAs
      β-tubulin (1055A)
      β-tubulin (1055B)
      Double sex (1055C)
      Double sex (1055D)
      Zpg (1055E)
      Zpg (1055F)
      Intersex (1055J)
      Labrum (1055Y)
      Sister of sex lethal (1055Z)
      Proboscipedia (1055T)
   4 gRNAs
      β-tubulin (1067A)
      Double sex (1067B)
      Zpg (1067C)
      Dsx+Isx+βtub (1067D)
      Dsx+βtub (1067E)
      Dsx+βtub (1067F)
      Dsx+Zpg (1067G)
   6 gRNAs
      Dsx+βtub (1067H)
      Dsx+Isx+βtub (1067L)

### C. Non-Limiting Advantages

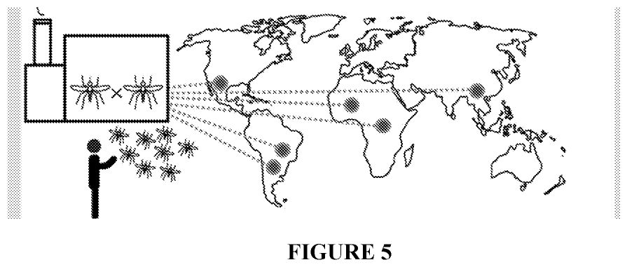

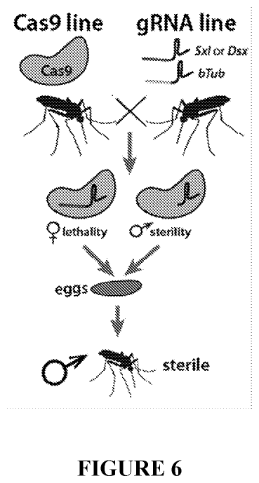

The technology as disclosed herein is an innovative improvement upon current SIT and female-killing approaches to suppress disease vector populations. The main competitor technology for this approach is the Oxitec's RIDL system, which involves the expression of a tetracycline-repressible transactivator fusion protein (tTa), which binds to the tetracycline-responsive element (tRe), driving expression of a toxin in the absence of the drug tetracycline. In the presence of tetracycline, Oxitec's RIDL system is silenced, the toxin is not produced, and progeny survive. Therefore, to mass rear insects carrying the RIDL system, progeny must be provided a diet supplemented with large amounts of tetracycline. Growth on tetracycline has numerous unwanted side effects on insects, such as loss of the gut microbiome, loss of symbiotic bacteria, and effects on mitochondrial function (Moullan et al. 2015; Chatzispyrou et al. 2015). Thus, not surprisingly, the feeding of tetracycline has recently been shown to impose a large fitness cost in insects (Zeh et al. 2012). Moreover, the RIDL system requires labor intensive manual sexing of the final product, and manual release of adult males at the release site. In order to generate these males for release, factories need to be constructed near release sites, due to the fact that adult male mosquitoes are very fragile and cannot be easily transported to remote locations. These restrictions make the RIDL system expensive and difficult to scale and service globally (FIG. 5). Consequently, the demonstrated release sites for the RIDL technologies have been relatively small and limited to regions near the production factories—primarily due to scalability factors (i.e. fitness costs, requirement for manual sexing of product, manual adult release near factory site) (for example, Carvalho et al. 2015). Together, these observations indicate that while Oxitec's RIDL system is able to suppress wild populations, a genetic system with a similar outcome, that does not impose a major antibiotic based fitness cost on the sterile males generated and is highly scalable would be advantageous. More importantly, the final product is genetically sexed early in development, so eggs can be released into the environment (requiring only one factory to service the entire world!), which is a significant advancement and improvement to the current technology (WO 2019/103982, Kandul et al. 2018 and Kandul et al 2019). Additionally, for SIT to be very effective—scalability is a must—the more scalable the system the greater number of sterile insects that can generated and released into the environment thereby resulting in a quicker population suppression effect that can be maintained.

In the approach as disclosed herein, eggs can be hatched directly into the environment (e.g. sprayed via drones), or alternatively a simple hatching strategy could be devised consisting of uniform egg packets, water containers and food packets—10 days later all the surviving mosquitoes will be sterile males that can be released directly into the environment. These hatching procedures require few resources and limited training, which simplifies application at any site, especially those in resource limited areas. Moreover, given that eggs can be distributed (as opposed to very fragile adult males) this enables one of skill in the art to reach remote locations around the globe, and eggs also facilitate the scalability in the environment (i.e. simply release more eggs every generation) which could lead to complete eradication of mosquito vectors or fly vectors, such as D. suzukii or Aedes aegypti. The simplicity of this system is among its biggest strengths. Additionally, the technology allows release of any mosquito or fly life stage, which makes this an essential technology if the expected entomological or epidemiological impact is predicted to be highest from the release of another life stage. For instance, high egg-stage mortality and high density dependent mortality of Ae. aegypti makes release of larval stage mosquitoes optimal in some scenarios (Yakob et al. 2017); however, with the approach as disclosed herein the sexing is done in an early embryonic stage allowing release at any stage. Therefore, in contrast to current technologies, the disclosed technology is highly: (1) adaptable, (2) efficient and reproducible, (3) scalable, (4) production efficient, and (5) environment-friendly.

Adaptable: The CRISPR/Cas9 system was successfully applied to a large number of insect species, including disease vectors (Kistler, Vosshall, and Matthews 2015; Gantz et al. 2015; Reid and O'Brochta 2016; Hammond et al. 2015), agricultural pests (Y. Li et al. 2016; Awata et al. 2015; Huang et al. 2016), beneficial insects (Ma et al. 2014; X. Li et al. 2015) and valuable research species (Bassett et al. 2013; Gratz et al. 2013) as well as a large number of non-insect species. Additionally, this binary approach only requires two components: Cas9 and gRNA, which greatly simplifies its application to vector control in comparison to the current RIDL (Thomas et al. 2000) and Wolbachia systems. The modular design of the system also permits easy modification of the components. To target different genes, the 23 bp gRNA target for CRISPR/Cas9 can be trivially replaced with a new gRNA targeting a different gene. The genes targeted for knockout can be interspecific, targeting a gene highly conserved between vector species, or species specific. Moreover, other CRISPR endonucleases, like Cpf1a and crRNA (Zetsche et al. 2015) can be added to the technology and bring more specificity and/or stronger knockout effect. Likewise, this technology does not require drugs or chemicals, such as in the RIDL (Thomas et al. 2000), to rear insects in the laboratory, which are known to have a negative effect on mitochondria (Moullan et al. 2015; Chatzispyrou et al. 2015) and insect gut microflora (Kalghatgi et al. 2013).

Efficient and Reproducible: The efficiency of pgSIT relies on the high cleavage rate of wt alleles by Cas9/gRNA and the inability of insect to rescue mosaic loss-of-function phenotype at the organismic level. Any CRISPR-based gene-drive system requires two actions: (1) cleavage of a target locus by Cas9/gRNA, and (2) homology directed repair (HDR) of the cut locus to integrate a transgene carrying the Cas9/gRNA system. The Cas9/gRNA-based cleavage is highly efficient process and results in numerous specific DNA cuts (WO 2019/103982, Kandul et al. 2018, Kandul et al. 2019; Oberhofer, Ivy, and Hay 2018) that can be repaired by HDR or non-homologous end joining (NHEJ) or other DNA repair pathways. NHEJ is the most efficient and common DNA repair pathways, while HDR is less efficient and limited to germ cells. Nevertheless, products of HDR carry an integrated transgene precisely at the target locus and thus are not recognized by Cas9/gRNA and selected for by a CRISPR gene drive (60%-85% of wt alleles are converted, Chu et al. 2015; Liang et al. 2017; Maruyama et al. 2015), while the precise ligation of cut alleles by NHEJ reconstitutes the target site that will be cut again by Cas9/gRNA. In short, this is how any CRISPR gene drive operates. However, rare imprecise ligations of the cut allele by NHEJ insert deletions or insertions and thus also become resistant (aka. non-complementary) to Cas9/gRNA system. In fact, any CRISPR-based gene-drive actively creates resistance alleles that are immune to further cleavage as well as samples and propagates naturally occurring resistance alleles. Accumulation of both types of resistance alleles slows down and eventually blocks a CRISPR gene drive (KaramiNejadRanjbar et al. 2018; Oberhofer, Ivy, and Hay 2018; Gantz et al. 2015; Champer et al. 2017). pgSIT relies on the high cleavage efficiency by the Cas9/gRNA system and NHEJ proficiency to cause mosaic mutations (aka. indels) at conserved loci and achieve loss-of-function phenotypes at the organismic level. The observations of multiple pgSIT systems engineered in D. melanogaster suggest that Cas9/gRNA knockouts in somatic and germ tissues early during insect development ensures the complete penetrance of loss-of-function phenotypes in an adult insect (WO 2019/103982, Kandul et al. 2018 and Kandul et al. 2019). Moreover, given that pgSIT sterile males are developed using only laboratory strains with known sequences at targeted loci in a factory, then released, the genetic diversity in the wild will NOT affect the pgSIT technology, i.e. the males are already sterile. Therefore, the significant advantage of the disclosed technology over any gene drive systems is that pgSIT is NOT creating resistance alleles and NOT sampling natural occurring alleles.

Scalable: A remarkable benefit of the pgSIT technology is that the homozygous Cas9 (A/A) and homozygous gRNA (BB) lines can be healthily maintained separately and then crossed in a central facility and the resulting F1 trans-heterozygous CRISPR/Cas9 (AB) sterile male eggs, can be distributed to any location for immediate hatching and release (WO 2019/103982, Kandul et al. 2018 and Kandul et al. 2019). pgSIT would eliminate the high cost (and error) associated with rearing and sexing mosquitoes near release locations as well as have the benefit of improved quality control and supply chain management from centralized manufacturing, especially for applications in resource limited locations. This feature makes the technology as disclosed herein especially attractive for licensing by companies for insect control and population suppression. The technology is especially beneficial for population control of Ae. aegypti, whose eggs require a diapause for a normal development and can remain viable as dried eggs for a year (Diniz et al. 2017) making the egg distribution extremely tractable.

Production efficient: The pgSIT technology provides a simple procedure to mass-produce sterile mosquito or fly males, preferred for release as the male mosquitos do not bite or transmit disease. Currently, there are no highly efficient, non-labor-intensive genetic methods to kill all female mosquitoes early in development (Papathanos et al. 2009). Sexing at the pupal or adult stages is highly labor-intensive, imprecise, or not feasible for large scale production. Many mosquito production systems that are currently available are prohibitively expensive for most target disease endemic countries and they require infrastructure that is simply not reliable in resource-limited countries. For example, the Debug program at Verily has generated a state-of-the-art automated mosquito rearing and sexing facility that achieves essentially 100% sexing accuracy for their population suppression Wolbachia strains. However, this technology requires large and expensive facilities and since mosquitoes are still sexed at the adult stage, they cannot be shipped great distances and therefore need to be produced near the release site. Many of the areas with the highest need for vector-borne disease interventions lack reliable electricity, water, supply chains and other resources that are required to reliably produce mosquitoes with this technology as well as maintain these facilities. Furthermore, deviations in these resources would likely lead to sexing errors or at a minimum slow down the production process. Therefore, this technology is likely only feasible in developed countries, which have less epidemiological need for these technologies. Other less intensive sexing methods, such as sexing mosquitoes by size are inaccurate resulting in the undesired release of females. In this approach as disclosed herein, the simple pgSIT system either kills or masculinizes all females, rendering all viable progeny to be sterile males that can be released as eggs (WO 2019/103982, Kandul et al. 2018 and Kandul et al. 2019).

Environment-friendly: The technology is an environment-friendly SIT, which is species-specific and would fit into the standard SIT legal framework. Therefore, the permits and procedures established for mosquitoes and SIT insects in multiple countries, like Brazil, US, Cayman Islands etc., can be easily adopted for the CRISPR/Cas9 based SIT approach as disclosed herein enabling this system to be moved to the field quickly.

The presently disclosed pgSIT methods of male sexing and methods of male sexing and sterility use the precision and accuracy of CRISPR-based technology to disrupt genes essential for female viability (for male sexing) or concurrently or simultaneously disrupt genes essential for female viability and male fertility. The pgSIT methods of the present disclosure utilize a simple breeding scheme requiring two insect strains (a first parent strain and a second parental strain), one expressing an endonuclease (e.g., Cas9) and the other expressing a nucleic acid sequence construct having at least one guide polynucleotide directed to the gene or genes to be disrupted. A single mating between these two parental strains mechanistically results in synchronous polynucleotide-guided (e.g., RNAguided) dominant allelic or dominant biallelic knockouts of the target gene or genes throughout development.

CRISPR technology refers to clustered regularly interspaced short palindromic repeats and has been extensively studied and modified for genome editing in most studied organisms as disclosed in Sternberg and Doudna, Mol. Cell 58, 568-574 (201 5), the entire contents of which are herein incorporated by reference.

As used herein, with respect to the CRISPR-based technology, the term “guide RNA” refers to a polynucleotide having a “synthetic sequence” capable of binding the corresponding endonuclease enzyme protein (e.g., Cas9) and a variable target sequence capable of binding the genomic target (e.g., a nucleotide sequence found in an exon of a target gene). In some embodiments of the present disclosure, a guide polynucleotide is a guide ribonucleic acid (gRNA). In some embodiments, the variable target sequence of the guide polynucleotide is any sequence within the target that is unique with respect to the rest of the genome and is immediately adjacent to a Protospacer Adjacent Motif (PAM). The exact sequence of the PAM sequence may vary as different endonucleases require different PAM sequences. As used herein, the expression “single heterologous construct having two different single guide RNAs (sgRNAs)” refers to a double guide RNA (dgRNA).

As used herein, the term “introducing an endonuclease” into a target insect refers to the recombinant introduction of an endonuclease into the insect such that the endonuclease is present in the insect. Introduction of an endonuclease into an insect does not require genomic integration, but may include genomic integration.

Table 1 lists non-limiting candidate female lethality genes and candidate male sterility genes.

TABLE 1
Rationally selected candidate female lethality or male sterility genes

| | | | |
|---|---|---|---|
| Gene | Function(s) | Selection Parameters | Refs. |

Potential Female Lethality Genes

| | | | |
|---|---|---|---|
| double sex (dsx) | sexually dimorphic | main sex determination gene | Many, (Kyrou et al. 2018) |
| | morphology, | across insects | |
| | physiology, and | female lethality in the | |
| | behavior | previous work; Female | |
| | | sterility or intersex physiology | |
| | | in A, gambiae | |
| transformer-2 | sexually dimorphic | A. aegypti-kd resulted in | (Hoang et al. 2016) |
| (tra-2) | morphology, | increased female lethality | |
| | physiology, and | | |
| | behavior | | |

Potential Male Sterility Genes

| | | | |
|---|---|---|---|
| beta tubulin beta | testis-specific | A. aegypti and Anopheles- | (Hoyle et al. 1995; |
| 2 isoform | | testis specific | Catteruccia, Benton, |
| (β2Tub) | | Drosophila-kd induced male | and Crisanti 2005) |
| | | sterility | |
| boule (bol) | Testis-specific; | A. aegypti-transient kd in | (Whyard et al. 2015; |
| | spermatogenesis and | larvae resulted in high male | Sekiné, Furusawa, |
| | germ cell development | sterility | and Hatakeyama |
| | | | 2015) |
| Gld2 | sperm maturation | sperm maturation in D. | (Sartain et al. 2011) |
| | | melanogaster and putative | |
| | | orthologs in Aedes and | |
| | | Anopheles spp. | |
| growth arrest | testis specific | A. aegypti-transient kd in | (Whyard et al. 2015) |
| specific protein | | larvae resulted in high male | |
| 8 (gas 8) | | sterility | |
| fuzzy onions | mitochondrial fusion | A. aegypti-transient kd in | (Yaffe 1997; Hwa et |
| | factor during sperm | larvae resulted in high male | al. 2002) |
| | formation | sterility | |
| Nix | male development | feminization or genital | (Hall et al. 2015) |
| | | deformities in A. aegypti, but | |
| | | not present in other species | |
| No-hitter (nht) | spermatid | A. aegypti-transient kd in | (Whyard et al. 2015) |
| | differentiation | larvae resulted in high male | |
| | | sterility | |
| zero population | male fertility | An. gambiae-transient kd in | (Thailayil et al. |
| growth (zpg) | | embryo increased male | 2011; Whyard et al. |
| | | sterility; A. aegypti-transient | 2015) |
| | | kd resulted in high male | |
| | | sterility | |

Potential Male Mating Barrier Genes

| | | | |
|---|---|---|---|
| fruitless (fru) | male sexual behavior, | Drosophila, anophelines and | (Salvemini et al. |
| | male muscle | A. aegypti | 2013) |
| | differentiation | | |

### Compositions and Methods

In one aspect, provided is a mating system producing a genetically modified progeny that is a sterile male insect of Drosophila suzukii or Aedes aegypti mosquito. The method comprises, or consists essentially of, or yet further consists of genetically a first parental insect and a second parental insect. In one embodiment, the first parental insect comprises, or consists essentially of, or yet further consists of at least one nucleic acid sequence that comprises, or consists essentially of, or yet further consists of: (a) at least a first guide RNA targeting a female essential genomic sequence that is required for female-specific viability in Drosophila suzukii or Aedes aegypti, and (b) at least a second guide RNA targeting a male sterility genomic sequence that is required for male-specific fertility in Drosophila suzukii or Aedes aegypti.

In some aspects and/or embodiments of the disclosure herein, the guide polynucleotide targets one or more of doublesex (DSX), IS, Myosin Heavy chain, and Beta2tub. In some aspects and/or embodiments of the disclosure herein, the guide polynucleotide targeting a sex-specifically alternatively spliced sex-determination gene optionally selected from: sex lethal (Sxl), transformer (tra), doublesex (dsxF), or those listed in Table 1 and/or disclosed herein. In some aspects and/or embodiments of the disclosure herein, the guide polynucleotide targets a gene active during spermatogenesis optionally selected from βTubulin 85D (βTub), fuzzy onions (fzo), protamine A (ProtA), or spermatocyte arrest. In some aspects and/or embodiments of the disclosure herein, the guide polynucleotide targets one or more of those identified in Table 1 or otherwise disclosed herein.

In a further embodiment, the targeted sequence required for female-specific viability in Aedes aegypti is selected from a sequence of the Intersex (mediator of RNA polymerase II transcription subunit 29) or labrum (Lab) gene. In another embodiment, the targeted sequence required for male-specific fertility in Aedes aegypti is a sequence of the zero population growth (Zpg)1 gene.

In some embodiments, the targeted sequence is a sequence in the gene exon. The targeted sequence or a fragment thereof hybridizes to the corresponding gRNA. In one embodiment, the targeted sequence hybridizes to the corresponding gRNA without any mismatches. In another embodiment, the targeted sequence hybridizes to the corresponding gRNA with 1, 2, 3, 4, 5, 6, 7, 8, 9, 10 or more mismatches. Based on the targeted sequence, the gRNA sequence can be determined. In one embodiment, a gRNA comprises, or consists essentially of, or yet further consists of a sequence complement to a targeted sequence, such as those as disclosed herein, or an equivalent that is capable of binding to the same targeted sequence but comprises 1, 2, 3, 4, 5, 6, 7, 8, 9, 10 or more mismatches. In another embodiment, a gRNA comprises, or consists essentially of, or yet further consists of a sequence reverse-complement to a targeted sequence, such as those as disclosed herein, or an equivalent that is capable of binding to the same targeted sequence but comprises 1, 2, 3, 4, 5, 6, 7, 8, 9, 10 or more mismatches. In yet another embodiment, a gRNA comprises, or consists essentially of, or yet further consists of a sequence reverse to a targeted sequence, such as those as disclosed herein, or an equivalent that is capable of binding to the same targeted sequence but comprises 1, 2, 3, 4, 5, 6, 7, 8, 9, 10 or more mismatches.

In some embodiments, the targeted sequence required for male-specific fertility in Drosophila suzukii is selected from a sequence of Cannonball (Can), meiosis I arrest (Mia), P-element induced wimpy testis (PIWI), Heterochromatin Protein 1e (HP1e), zero population growth (Zpg), male sterile (3) K81 (K81), misfire (Mfr), or sneaky (Snky). In a further embodiment, the targeted sequence is a sequence in the gene exon.

In some embodiments, the second parental insect comprises, or consists essentially of, or yet further consists of a nucleic acid that encodes an endonuclease under the control of a regulatory sequence that is suitable for directing protein expression in Drosophila suzukii or Aedes aegypti.

In some embodiments, the targeted sequence required for female-specific viability in Aedes aegypti comprises, or consists essentially of, or yet further consists of a sequence selected from:

(Intersex1, SEQ ID NO: 58)
GCGGGATTCGCTCTCGACGA,
(Intersex2, SEQ ID NO: 59)
GGACAACATTTCCAAGGTTA,
(labrum (Lab) 5, SEQ ID NO: 60)
GCACTAGTGGGATATCCTGA,
or
(labrum (Lab) 4, SEQ ID NO: 61)
GTCCAGGAAGCATTTGGTAT.

In some embodiments, the targeted sequence required for male-specific fertility in Aedes aegypti comprises, or consists essentially of, or yet further consists of a sequence selected from:

(Zpg1, SEQ ID NO: 62)
GTGTAGACTGGCCCTAACGG,
(Zpg2, SEQ ID NO: 63)
TCTCGTTCCCCAAACACTTG,
(Zpg3, SEQ ID NO: 64)
TCATCAAGAACATGTTCAGG,
or
(Zpg4, SEQ ID NO: 65)
ACAGTTGGAACCGAACGCTG.

In some embodiments, the targeted sequence required for male-specific fertility in Drosophila suzukii comprises, or consists essentially of, or yet further consists of a sequence selected from:

(Cannonball (Can), SEQ ID NO: 66)
GCTACAGAGGAATGGCCCAG,
(Cannonball (Can), SEQ ID NO: 67)
GGATCGGGATAACCTGCCGT,
(meiosis I arrest (Mia), SEQ ID NO: 68)
GAGAATCCCCTTGTTGCGGG,
(meiosis I arrest (Mia), SEQ ID NO: 69)
GAGCTCTGACCATCCGCATG,
(P-element induced wimpy testis (PIWI),
SEQ ID NO: 70)
GGTGTTGGACAGCACATCGA,
(P-element induced wimpy testis (PIWI),
SEQ ID NO: 71)
GAGCACACGTGATCGCAAGA,
(Heterochromatin Protein 1e (HP1e), SEQ ID NO: 72)
CAACCTCAAgTTGTaCCAAG,
(Heterochromatin Protein 1e (HP1e), SEQ ID NO: 73)
GGATTATCCAAAACTAAGCA,
(zero population growth (Zpg), SEQ ID NO: 74)
GCAACTGCAAACGCATTCCG,
(zero population growth (Zpg), SEQ ID NO: 75)
GCCCAAGTTGCACCTGCAGG,
(male sterile (3) K81 (K81), SEQ ID NO: 76)
GATCACAGCGAGTTCTTCGG,
(male sterile (3) K81 (K81), SEQ ID NO: 77)
GGAGTTGGGGTCGTCGACAT,
(misfire (Mfr), SEQ ID NO: 78)
GGCGTCAAGTTCAAGAAGCA,
(misfire (Mfr), SEQ ID NO: 79)
GAGAACGGGACACTGGCAGG,
(sneaky (Snky), SEQ ID NO: 80)
GACTTCTCGTAGGTGCGCAA,
and
(sneaky (Snky), SEQ ID NO: 81)
GTAGGTGCGCAATGGTAAGA.

In some embodiments, the regulatory sequence that is suitable for directing protein expression in Drosophila suzukii comprises, or consists essentially of, or yet further consists of a promoter. In a further embodiment, the promoter comprises, or consists essentially of, or yet further consists of a sequence selected from: nt 4360 to nt 6582 of SEQ ID NO: 1 or nt 4360 to nt 6600 of SEQ ID NO: 2 (874Z promoter sequence), nt 4366 to nt 4873 of SEQ ID NO: 3 (874S promoter sequence), nt 4366 to nt 7196 of SEQ ID NO: 4 (874R promoter sequence), nt 4366 to nt 6233 of SEQ ID NO: 5 (874W promoter sequence), or nt 4360 to nt 5291 of SEQ ID NO: 6 (874Z1 promoter sequence). In one embodiment, the promoter comprises, or consists essentially of, or yet further consists of a sequence of nt 4366 to nt 6233 of SEQ ID NO: 5 (874W promoter sequence).

In some embodiments, the regulatory sequence suitable for directing protein expression in Aedes aegypti mosquito comprises, or consists essentially of, or yet further consists of a promoter comprises, or consists essentially of, or yet further consists of a sequence selected from nt 4366 to nt 6497 of SEQ ID NO: 22 (874L promoter sequence), nt 4366 to nt 6865 of SEQ ID NO: 23 or a fragment thereof comprising, or consists essentially of, or yet further consists of an AAEL007097 promoter (874M promoter sequence), nt 4366 to nt 7404 of SEQ ID NO: 24 (874N promoter sequence), nt 4366 to nt 7399 of SEQ ID NO: 25 (874P promoter sequence), or nt 4366 to nt 5751 of SEQ ID NO: 26 (874X promoter sequence), or nt 4366 to nt 4786 of SEQ ID NO: 27 (874Y promoter sequence) or nt 4363 to nt 7162 of SEQ ID NO: 28 (874Y3 promoter sequence). In one embodiment, the promoter comprises, or consists essentially of, or yet further consists of a sequence of nt 4366 to nt 7399 of SEQ ID NO: 25 (874P promoter sequence). In another embodiment, the promoter comprises, or consists essentially of, or yet further consists of a sequence of nt 4366 to nt 4786 of SEQ ID NO: 27 (874Y promoter sequence).

In another aspect, provided is a mating system producing a genetically modified progeny that is a sterile male insect of Drosophila suzukii or Aedes aegypti mosquito, comprising, or consists essentially of, or yet further consists of a first parental insect and a second parental insect. In some embodiments, the first parental insect comprises, or consists essentially of, or yet further consists of at least one nucleic acid sequence that comprises, or consists essentially of, or yet further consists of: (a) at least a first guide RNA targeting a female essential genomic sequence that is required for female-specific viability in Drosophila suzukii or Aedes aegypti, and (b) at least a second guide RNA targeting a male sterility genomic sequence that is required for male-specific fertility in Drosophila suzukii or Aedes aegypti.

In some embodiments, the targeted sequence required for female-specific viability in Drosophila suzukii comprises, or consists essentially of, or yet further consists of a sequence of GGCGGCAGCGGCGGGAATGG (Sxl—Max Scott site, SEQ ID NO: 82). In another embodiments, the targeted sequence required for male-specific fertility in Drosophila suzukii comprises, or consists essentially of, or yet further consists of a sequence selected from:

(Btub Original site 2, SEQ ID NO: 83)
TGGCGGTACCGGCTCCGGAA,
(Btub site 2, SEQ ID NO: 84)
CTTGAGTGTGCACCAGCTGG,
(Btub site 3, SEQ ID NO: 85)
CAATGCGGTAACCAGATCGG,
(Btub site 4, SEQ ID NO: 86)
GCCTCGGGGTCTAAAGATGT,
(Cannonball (Can), SEQ ID NO: 66)
GCTACAGAGGAATGGCCCAG,
(Cannonball (Can), SEQ ID NO: 67)
GGATCGGGATAACCTGCCGT,
(meiosis I arrest (Mia), SEQ ID NO: 68)
GAGAATCCCCTTGTTGCGGG,
(meiosis I arrest (Mia), SEQ ID NO: 69)
GAGCTCTGACCATCCGCATG,
(P-element induced wimpy testis (PIWI),
SEQ ID NO: 70)
GGTGTTGGACAGCACATCGA,
(P-element induced wimpy testis (PIWI),
SEQ ID NO: 71)
GAGCACACGTGATCGCAAGA,
(Heterochromatin Protein 1e (HP1e), SEQ ID NO: 72)
CAACCTCAAgTTGTaCCAAG,
(Heterochromatin Protein 1e (HP1e), SEQ ID NO: 73)
GGATTATCCAAAACTAAGCA,
(zero population growth (Zpg), SEQ ID NO: 74)
GCAACTGCAAACGCATTCCG,
(zero population growth (Zpg), SEQ ID NO: 75)
GCCCAAGTTGCACCTGCAGG,
(male sterile (3) K81 (K81), SEQ ID NO: 76)
GATCACAGCGAGTTCTTCGG,
male sterile (3) K81 (K81), SEQ ID NO: 77)
GGAGTTGGGGTCGTCGACAT,
(misfire (Mfr), SEQ ID NO: 78)
GGCGTCAAGTTCAAGAAGCA,
(misfire (Mfr), SEQ ID NO: 79)
GAGAACGGGACACTGGCAGG,
(sneaky (Snky), SEQ ID NO: 80)
GACTTCTCGTAGGTGCGCAA,
or
(sneaky (Snky), SEQ ID NO: 81)
GTAGGTGCGCAATGGTAAGA.

In some embodiments, the targeted sequence required for female-specific viability in Aedes aegypti comprises, or consists essentially of, or yet further consists of a sequence selected from: AGCGACATGCAGCCATTCTG (double sex (Dsx) 1, SEQ ID NO: 87 in the revised plasmid), CTCACAAGTAGAGCTACACG (Dsx2, SEQ ID NO: 88 in the revised plasmid), GGTAGCTGGCCGTTGCCAAA (Dsx3, SEQ ID NO: 89 in the revised plasmid), CTGTCGTCGTTTTTTTCCGG (Dsx4, SEQ ID NO: 90 in the revised plasmid), GGATATTGGTACGACCCGGG (Dsx5, SEQ ID NO: 91), GCGGGATTCGCTCTCGACGA (Intersex1, SEQ ID NO: 58), GGACAACATTTCCAAGGTTA (Intersex2, SEQ ID NO: 59), GCACTAGTGGGATATCCTGA (labrum (Lab) 5, SEQ ID NO: 60), GTCCAGGAAGCATTTGGTAT (labrum (Lab) 4, SEQ ID NO: 61), GTATTCGATGTTCTCCACC (sister of sex lethal (Slx) 4, SEQ ID NO: 92), GAAGGCATATCAAACATTCG (sister of sex lethal (Slx) 5, SEQ ID NO: 93), GTTAAGAACTCGGCCATCGA (homeotic protein Proboscipedia (Pb) 1, SEQ ID NO: 94), GCAAGTTAAGCCTGAAACAA (homeotic protein Proboscipedia (Pb) 2, SEQ ID NO: 95). In another embodiment, the targeted sequence required for male-specific fertility in Aedes aegypti comprises, or consists essentially of, or yet further consists of a sequence selected from: TACTACAACGAGGCTACCGG (Btub1, SEQ ID NO: 96), GTCTCCGCAATACGCCCCGG (Btub2, SEQ ID NO: 97), GATAGGAGCCAAGTTCTGGG (Btub3, SEQ ID NO: 98), CACTATGGATTCGGTCCGAG (Btub4, SEQ ID NO: 99), GTGTAGACTGGCCCTAACGG (zero population growth (Zpg)1, SEQ ID NO: 62), TCTCGTTCCCCAAACACTTG (Zpg2, SEQ ID NO: 63), TCATCAAGAACATGTTCAGG (Zpg3, SEQ ID NO: 64), ACAGTTGGAACCGAACGCTG (Zpg4, SEQ ID NO: 65).

In some embodiments, the second parental insect comprises, or consists essentially of, or yet further consists of a nucleic acid that encodes an endonuclease under the control of a regulatory sequence that is suitable for directing protein expression in Drosophila suzukii or Aedes aegypti.

In some embodiments, the regulatory sequence that is suitable for directing protein expression in Drosophila suzukii comprises, or consists essentially of, or yet further consists of a promoter that comprises, or consists essentially of, or yet further consists of a sequence of selected from: nt 4360 to nt 6582 of SEQ ID NO: 1 or nt 4360 to nt 6600 of SEQ ID NO: 2 (874Z promoter sequence), nt 4366 to nt 4873 of SEQ ID NO: 3 (874S promoter sequence), nt 4366 to nt 7196 of SEQ ID NO: 4 (874R promoter sequence), nt 4366 to nt 6233 of SEQ ID NO: 5 (874W promoter sequence), or nt 4360 to nt 5291 of SEQ ID NO: 6 (874Z1 promoter sequence). In one embodiment, the promoter comprises, or consists essentially of, or yet further consists of a sequence of nt 4366 to nt 6233 of SEQ ID NO: 5 (874W promoter sequence).

In some embodiments, the regulatory sequence suitable for directing protein expression in Aedes aegypti mosquito comprises, or consists essentially of, or yet further consists of a promoter that comprises, or consists essentially of, or yet further consists of a sequence selected from nt 4366 to nt 6497 of SEQ ID NO: 22 (874L promoter sequence), nt 4366 to nt 6865 of SEQ ID NO: 23 or a fragment thereof comprising, or consists essentially of, or yet further consists of an AAEL007097 promoter (874M promoter sequence), nt 4366 to nt 7404 of SEQ ID NO: 24 (874N promoter sequence), nt 4366 to nt 7399 of SEQ ID NO: 25 (874P promoter sequence), or nt 4366 to nt 5751 of SEQ ID NO: 26 (874X promoter sequence), or nt 4366 to nt 4786 of SEQ ID NO: 27 (874Y promoter sequence) or nt 4363 to nt 7162 of SEQ ID NO: 28 (874Y3 promoter sequence). In one embodiment, the promoter comprises, or consists essentially of, or yet further consists of a sequence of nt 4366 to nt 7399 of SEQ ID NO: 25 (874P promoter sequence). In another embodiment, the promoter comprises, or consists essentially of, or yet further consists of a sequence of nt 4366 to nt 4786 of SEQ ID NO: 27 (874Y promoter sequence).

In yet another aspect, provided is a mating system producing a genetically modified progeny that is a sterile male insect of Drosophila suzukii, comprising, or consists essentially of, or yet further consists of a first parental insect and a second parental insect as disclosed herein. In some embodiments, the first parental Drosophila suzukii comprises, or consists essentially of, or yet further consists of at least one nucleic acid sequence that comprises, or consists essentially of, or yet further consists of: (a) at least a first guide RNA targeting a female essential genomic sequence that is required for female-specific viability in Drosophila suzukii, and (b) at least a second guide RNA targeting a male sterility genomic sequence that is required for male-specific fertility in Drosophila suzukii. In some embodiments, the second parental insect comprises, or consists essentially of, or yet further consists of a nucleic acid that encodes an endonuclease under the control of a regulatory sequence that is suitable for directing protein expression in Drosophila suzukii. In a further embodiment, the regulatory sequence comprises, or consists essentially of, or yet further consists of a promoter selected from a deadhead (Dhd) promoter or a bicoid (BicC) promoter, or any other promoter as disclosed herein.

In some embodiments, the targeted sequence required for male-specific fertility in Drosophila suzukii is selected from a sequence of Cannonball (Can), meiosis I arrest (Mia), P-element induced wimpy testis (PIWI), Heterochromatin Protein 1e (HP1e), zero population growth (Zpg), male sterile (3) K81 (K81), misfire (Mfr), or sneaky (Snky).

In some embodiments, the targeted sequence required for female-specific viability in Drosophila suzukii comprises, or consists essentially of, or yet further consists of a sequence of GGCGGCAGCGGCGGGAATGG (Sxl—Max Scott site, SEQ ID NO: 82). In another embodiment, the targeted sequence required for male-specific fertility in Drosophila suzukii comprises, or consists essentially of, or yet further consists of a sequence selected from:

(Btub Original site 2, SEQ ID NO: 83)
TGGCGGTACCGGCTCCGGAA,
(Btub site 2, SEQ ID NO: 84)
CTTGAGTGTGCACCAGCTGG,
(Btub site 3, SEQ ID NO: 85)
CAATGCGGTAACCAGATCGG,
(Btub site 4, SEQ ID NO: 86)
GCCTCGGGGTCTAAAGATGT,
(Cannonball (Can), SEQ ID NO: 66)
GCTACAGAGGAATGGCCCAG,
(Cannonball (Can), SEQ ID NO: 67)
GGATCGGGATAACCTGCCGT,
(meiosis I arrest (Mia), SEQ ID NO: 68)
GAGAATCCCCTTGTTGCGGG,
(meiosis I arrest (Mia), SEQ ID NO: 69)
GAGCTCTGACCATCCGCATG,
(P-element induced wimpy testis (PIWI),
SEQ ID NO: 70)
GGTGTTGGACAGCACATCGA,
(P-element induced wimpy testis (PIWI),
SEQ ID NO: 71)
GAGCACACGTGATCGCAAGA,
(Heterochromatin Protein 1e (HP1e), SEQ ID NO: 72)
CAACCTCAAgTTGTaCCAAG,
(Heterochromatin Protein 1e (HP1e), SEQ ID NO: 73)
GGATTATCCAAAACTAAGCA,
(zero population growth (Zpg), SEQ ID NO: 74)
GCAACTGCAAACGCATTCCG,
(zero population growth (Zpg), SEQ ID NO: 75)
GCCCAAGTTGCACCTGCAGG,
(male sterile (3) K81 (K81), SEQ ID NO: 76)
GATCACAGCGAGTTCTTCGG,
male sterile (3) K81 (K81), SEQ ID NO: 77)
GGAGTTGGGGTCGTCGACAT,
(misfire (Mfr), SEQ ID NO: 78)
GGCGTCAAGTTCAAGAAGCA,
(misfire (Mfr), SEQ ID NO: 79)
GAGAACGGGACACTGGCAGG,
(sneaky (Snky), SEQ ID NO: 80)
GACTTCTCGTAGGTGCGCAA,
or
(sneaky (Snky), SEQ ID NO: 81)
GTAGGTGCGCAATGGTAAGA.

In some embodiments, the at least one nucleic acid sequence of the first parental Drosophila suzukii comprises, or consists essentially of, or yet further consists of a sequence of GATTGTCAACTACTTGCCCC (Sex lethal (Sxl)—Nikolai site 1, SEQ ID NO: 100).

In some embodiments, the at least one nucleic acid sequence of the first parental Aedes aegypti comprises, or consists essentially of, or yet further consists of a set of guide RNAs. In one embodiment, the targeted sequences are Dsx1, intersex1, BTub3 and BTub2. In another embodiment, the targeted sequences are Dsx1, Dsx5, BTub3, and BTub2. In yet another embodiment, the targeted sequences are Zpg3, Zpg4, Dsx1, and Dsx1. In one embodiment, the targeted sequences are Btub3, Btub2, Dsx1, and Dsx1. In another embodiment, the targeted sequences are Dsx1, Dsx2, Dsx3, Dsx4, BTub3, and BTub2. In yet another embodiment, the targeted sequences are Dsx1, intersex1, BTub3, BTub2, Dsx2, and Dsx4+.

In some embodiments, the endonuclease comprises, or consists essentially of, or yet further consists of a CRISPR-associated sequence 9 (Cas9) endonuclease or a variant thereof, a CRISPR-associated sequence 13 (Cas13) endonuclease or a variant thereof, a CRISPR from Prevotella and Francisella 1 (Cpf1) endonuclease or a variant thereof, a CRISPR from Microgenomates and Smithella 1 (Cms1) endonuclease or a variant thereof, or a CRISPR-associated sequence 6 (Cash) endonuclease or a variant thereof.

In some embodiments, the first insect is a female and the second insect is a male. In another embodiment, the first insect is a male and the second insect is a female. In a further embodiment, either or both of the following of the mating system further comprises, or consists essentially of, or yet further consists of a selection marker: the at least one nucleic acid sequence in the first parental insect or the nucleic acid that encodes an endonuclease under the control of a regulatory sequence of the second parental insect.

In one aspect, provided is a method of producing a genetically modified progeny that is a sterile male insect of Drosophila suzukii or Aedes aegypti mosquito. The method comprises, or consists essentially of, or yet further consists of genetically crossing two parental insects of the mating system as disclosed herein, and producing and collecting the progeny of the parental insects. In a further embodiment, the method further comprises an introduction step prior to the genetically crossing step, and the introduction step comprises, or consists essentially of, or yet further consists of one or both of the following:
Introducing the at least one nucleic acid sequence to the first parental insect, or
Introducing the nucleic acid that encodes an endonuclease under the control of a regulatory sequence to the second parental insect, and
Optionally wherein either or both of the introduction integrates the sequence into the insect genome, and optionally wherein the progeny comprising, or consisting essentially of, or yet further consists of the endonuclease, at least one first guide RNA, and at least one second guide RNA.

Additionally provided is a progeny produced by the mating system as disclosed herein, or the method as disclosed herein. In one embodiment, the progeny is an egg, a population of eggs, an insect, or a population of insects. In one embodiment, the progeny may be at any development stage of its life cycle, such as egg, larva or adult. In one embodiment, the progeny is sterile male.

In one aspect, provided is a progeny of the insect egg or the insect or the insect population or a progeny of each thereof of as disclosed. In a further aspect, one or more of the following is provided: a genetically modified insect egg or a progeny thereof, a genetically modified insect or a progeny thereof, or an insect population comprising, or consists essentially of, or yet further consists of at least one genetically modified insect or a progeny thereof as disclosed herein. In some aspects and/or embodiments of the disclosure herein, the progeny is the first, second, third, fourth, fifth, sixth, seventh, eighth, ninth, tenth, or more generations of the insect egg or insect or insect population.

In some aspects and/or embodiments of the disclosure herein, the insect egg or the insect or the insect population or a progeny of each thereof of, comprises or consists essentially of, or yet further consists of at least about 50%, or at least about 75%, or at least about 80%, or at least about 85%, or at least about 90%, or at least about 95%, or at least about 96%, or at least about 97%, or at least about 98%, or at least about 99%, or up to 100% of the fitness or the mating competitiveness of a wild type.

In some aspects and/or embodiments of the disclosure herein, a progeny of the insect egg or the insect or the insect population or a progeny of each thereof as disclosed herein, comprises or consists essentially of, or yet further consists of at least about 80%, or at least about 85%, or at least about 90%, or at least about 95%, or at least about 96%, or at least about 97%, or at least about 98%, or at least about 99%, or up to 100% sterile male.

Also provided is a method of reducing a wild-type insect population. In one embodiment, the method comprises, or consists essentially of, or yet further consists of introducing an insect egg or an insect or an insect population or a progeny of each thereof as disclosed herein, or the progeny as disclosed herein, to the wild-type insect population. In one embodiment, the insect is Drosophila suzukii. In another embodiment, the insect is Aedes aegypti.

In another aspect, provided is a polynucleotide comprising, or consisting essentially of, or yet further consisting of a sequence encoding a Cas9 under the control of a deadhead (Dhd) promoter or a bicoid (BicC) promoter or any other promoter as disclosed herein. In one embodiment, the polynucleotide is isolated or engineered. In a further embodiment, the polynucleotide is for use in a mating system as disclosed herein, or a method as disclosed herein.

Additionally or alternatively, provided is a polynucleotide or a sequence complementary thereto, or reverse thereto, or reverse complementary thereto, or complement thereto, or reverse complement thereto. The polynucleotide comprises, or consists essentially of, or yet further consists of one or both of:
(a) at least a first guide RNA targeting a female essential genomic sequence that is required for female-specific viability in Drosophila suzukii or Aedes aegypti, and
(b) at least a second guide RNA targeting a male sterility genomic sequence that is required for male-specific fertility in Drosophila suzukii or Aedes aegypti.

In one embodiment, the polynucleotide is isolated or engineered.

In some embodiments, the targeted sequence required for female-specific viability in Aedes aegypti is selected from a sequence of the Intersex or labrum (Lab) gene. In another embodiment, the targeted sequence required for male-specific fertility in Aedes aegypti is a sequence of the zero population growth (Zpg)1 gene. In a further embodiment, the targeted sequence is a sequence in the gene exon, and

In some embodiments, the targeted sequence required for male-specific fertility in Drosophila suzukii is selected from a sequence of Cannonball (Can), meiosis I arrest (Mia), P-element induced wimpy testis (PIWI), Heterochromatin Protein 1e (HP1e), zero population growth (Zpg), male sterile (3) K81 (K81), misfire (Mfr), or sneaky (Snky). In a further embodiment, the targeted sequence is a sequence in the gene exon. In yet a further embodiment, the polynucleotide is for use in a mating system as disclosed herein, or a method as disclosed herein.

Additionally provided is a polynucleotide or a sequence complementary thereto. Such polynucleotide comprises, or consists essentially of, or yet further consists of one or both of:
(a) a first guide RNA targeting a female essential genomic sequence that is required for female-specific viability in Drosophila suzukii or Aedes aegypti, and
(b) a second guide RNA targeting a male sterility genomic sequence that is required for male-specific fertility in Drosophila suzukii or Aedes aegypti.

In one embodiment, the polynucleotide is isolated or engineered.

In some embodiments, the targeted sequence required for female-specific viability in Drosophila suzukii is GGCGGCAGCGGCGGGAATGG (Sxl—Max Scott site, SEQ ID NO: 82). In another embodiment, the targeted sequence required for male-specific fertility in Drosophila suzukii comprises, or consists essentially of, or yet further consists of a sequence selected from: TGGCGGTACCGGCTCCGGAA (Btub Original site 2, SEQ ID NO: 83), CTTGAGTGTGCACCAGCTGG (Btub site 2, SEQ ID NO: 84), CAATGCGGTAACCAGATCGG (Btub site 3, SEQ ID NO: 85), GCCTCGGGGTCTAAAGATGT (Btub site 4, SEQ ID NO: 86), GCTACAGAGGAATGGCCCAG (Cannonball (Can), SEQ ID NO: 66), GGATCGGGATAACCTGCCGT (Cannonball (Can), SEQ ID NO: 67), GAGAATCCCCTTGTTGCGGG (meiosis I arrest (Mia), SEQ ID NO: 68), GAGCTCTGACCATCCGCATG (meiosis I arrest (Mia), SEQ ID NO: 69), GGTGTTGGACAGCACATCGA (P-element induced wimpy testis (PIWI), SEQ ID NO: 70), GAGCACACGTGATCGCAAGA (P-element induced wimpy testis (PIWI), SEQ ID NO: 71), CAACCTCAAgTTGTaCCAAG (Heterochromatin Protein 1e (HP1e), SEQ ID NO: 72), GGATTATCCAAAACTAAGCA (Heterochromatin Protein 1e (HP1e), SEQ ID NO: 73), GCAACTGCAAACGCATTCCG (zero population growth (Zpg), SEQ ID NO: 74), GCCCAAGTTGCACCTGCAGG (zero population growth (Zpg), SEQ ID NO: 75), GATCACAGCGAGTTCTTCGG (male sterile (3) K81 (K81), SEQ ID NO: 76), GGAGTTGGGGTCGTCGACAT male sterile (3) K81 (K81), SEQ ID NO: 77), GGCGTCAAGTTCAAGAAGCA (misfire (Mfr), SEQ ID NO: 78), GAGAACGGGACACTGGCAGG (misfire (Mfr), SEQ ID NO: 79), GACTTCTCGTAGGTGCGCAA (sneaky (Snky), SEQ ID NO: 80), or GTAGGTGCGCAATGGTAAGA (sneaky (Snky), SEQ ID NO: 81), and

In some embodiments, the targeted sequence required for female-specific viability in Aedes aegypti comprises, or consists essentially of, or yet further consists of a sequence selected from: AGCGACATGCAGCCATTCTG (double sex (Dsx) 1, SEQ ID NO: 87 in the revised plasmid), CTCACAAGTAGAGCTACACG (Dsx2, SEQ ID NO: 88 in the revised plasmid), GGTAGCTGGCCGTTGCCAAA (Dsx3, SEQ ID NO: 89 in the revised plasmid), CTGTCGTCGTTTTTTTCCGG (Dsx4, SEQ ID NO: 90 in the revised plasmid), GGATATTGGTACGACCCGGG (Dsx5, SEQ ID NO: 91), GCGGGATTCGCTCTCGACGA (Intersex1, SEQ ID NO: 58), GGACAACATTTCCAAGGTTA (Intersex2, SEQ ID NO: 59), GCACTAGTGGGATATCCTGA (labrum (Lab) 5, SEQ ID NO: 60), GTCCAGGAAGCATTTGGTAT (labrum (Lab) 4, SEQ ID NO: 61), GTATTCGATGTTCTCCACC (sister of sex lethal (Slx) 4, SEQ ID NO: 92), GAAGGCATATCAAACATTCG (sister of sex lethal (Slx) 5, SEQ ID NO: 93), GTTAAGAACTCGGCCATCGA (homeotic protein Proboscipedia (Pb) 1, SEQ ID NO: 94), GCAAGTTAAGCCTGAAACAA (homeotic protein Proboscipedia (Pb) 2, SEQ ID NO: 95), or wherein the targeted sequence required for male-specific fertility in Aedes aegypti is selected from: TACTACAACGAGGCTACCGG (Btub1, SEQ ID NO: 96), GTCTCCGCAATACGCCCCGG (Btub2, SEQ ID NO: 97), GATAGGAGCCAAGTTCTGGG (Btub3, SEQ ID NO: 98), CACTATGGATTCGGTCCGAG (Btub4, SEQ ID NO: 99), GTGTAGACTGGCCCTAACGG (zero population growth (Zpg)1, SEQ ID NO: 62), TCTCGTTCCCCAAACACTTG (Zpg2, SEQ ID NO: 63), TCATCAAGAACATGTTCAGG (Zpg3, SEQ ID NO: 64), ACAGTTGGAACCGAACGCTG (Zpg4, SEQ ID NO: 65);

In one embodiment, the polynucleotide is for use in a mating system as disclosed herein, or a method as disclosed herein.

In one aspect, provided is a vector comprising, or alternatively consisting essentially of, or yet further consisting of one or more of the polynucleotide(s) as disclosed herein. In one embodiment, provided is a vector comprising, or consisting essentially of, or yet further consisting of a polynucleotide as disclosed herein or its complement or an equivalent of each thereof. Such equivalent hybridize to the same targeted sequence or encodes the same protein. In one embodiment, a polynucleotide or a vector as provided herein may further comprises another sequence, such as one or more of a sequence listed as a feature in the drawings. Non-limiting examples of the sequence that may be further comprised include a sequence encoding a detectable marker (such as nt 6430 to nt 7146 of SEQ ID NO: 20 encoding an enhanced GFP having an amino acid sequence of SEQ ID NO: 14), a regulatory sequence (such as a promoter, a terminator, a polyA, an enhancer, or an intron), a polynucleotide sequence of a nuclear localization sequence or a polynucleotide sequence encoding a nuclear localization peptide (both of which are referred to herein as NLS) optionally located at the 5′ end of the sequence encoding a protein (such as Cas9 or a protein detectable marker), a polynucleotide sequence encoding a self-cleaving peptide (such as T2A) and/or any other cleavage site (such as a glycine-Proline cleavage site) optionally located at the 5′ end of the sequence encoding a detectable marker, or a gRNA scaffold located at the 3′ end of gRNA (such as nt 4514 to nt 4589 of SEQ ID NO: 17 or nt 5083 to nt 5168 of SEQ ID NO: 44).

In a further embodiment, the vector is selected from the group of 874Z, 874S, 874R, 874W, 874Z1, 874L, 874M, 874N, 874P, 874X, 874Y, 874Y3, 1056C.2, 1056H, 1056I, 1056J, 1056K, 1104D, 1104E, 1104H, 1104I, 1104J, 1104K, 1104M, 1104N, 1055A, 1055B, 1055C, 1055D, 10555E, 1055F, 1055I, 1055T, 1055Y, 1055Z, 1067A, 1067B, 1067C, 1067D, 1067E, 1067F, 1067G, 1067H, or 1067L. In yet a further embodiment, the vector is for use in the method as disclosed herein.

Also provided is an isolated or engineered host cell comprising any one or more of the polynucleotides as disclosed herein and/or any one or more of the vectors as disclosed herein. In one embodiment, the host cell produces the polynucleotide(s) and/or the vector(s). Additionally or alternatively, the host cell is an insect cell. In one embodiment, the host cell is selected from an egg, a sperm, a zygote, or a germline cell. In yet a further embodiment, the cell is an insect germline cell. In a further embodiment, the cell is a Drosophila suzukii or Aedes aegypti mosquito cell. Additionally or alternatively, the progeny may be at any development stage of its life cycle, such as egg, larva or adult.

In some embodiments, the polynucleotide is engineered to one or more of the chromosome(s) or chromosome sites of the host cell. In one embodiment, the host cell comprises homozygous polynucleotides. In another embodiment, the host cell comprises a heterozygous polynucleotide. In some aspects and/or embodiments of the disclosure herein, the polynucleotide is engineered to one or more of the chromosome(s) or chromosome site(s) of the insect egg or the insect.

In another aspect, provided is a Drosophila suzukii or Aedes aegypti mosquito comprising any one or more of: a polynucleotide as disclosed herein, a vector as disclosed herein, or a cell as disclosed herein.

In yet another aspect, provided is a kit comprising, or consisting essentially of, or yet further consisting of one or more of: a polynucleotide as disclosed herein, a vector as disclosed herein, a cell as disclosed herein, or A Drosophila suzukii or Aedes aegypti mosquito as disclosed herein.

In some aspects and/or embodiments of the disclosure herein, the cell is an insect cell. In one embodiment, the host cell is selected from an egg, a sperm, a zygote, or a germline cell. In one embodiment, the progeny is an egg, a population of eggs, an insect, or a population of insects. In one embodiment, the progeny may be at any development stage of its life cycle, such as egg, larva or adult.

In some aspects and/or embodiments of the disclosure herein and unless specified, the insect is Drosophila suzukii. In some aspects and/or embodiments of the disclosure herein and unless specified, the insect is Aedes aegypti.

In some aspects and/or embodiments of the disclosure herein, the regulatory sequences directing expression of a gRNA comprises or consists essentially of, or yet further consists of a RNA pol III promoter. In a further embodiment, the RNA pol II promoter is selected from the group consisting of H1, U6, and U6.3.

In some aspects and/or embodiments of the disclosure herein, any of the composition as disclosed herein, such as a polynucleotide, a vector, a cell, or a Drosophila suzukii or Aedes aegypti mosquito may further comprises a detectable or selectable marker.

The following examples are included to demonstrate some embodiments of the disclosure. However, those of skill in the art should, in light of the present disclosure, appreciate that many changes can be made in the specific embodiments which are disclosed and still obtain a like or similar result without departing from the spirit and scope of the invention.

### Examples

Experiment No. 1—Precision Guided Sterile Insect Technique to Control a Major Worldwide Human Disease Vector, Aedes aegypti

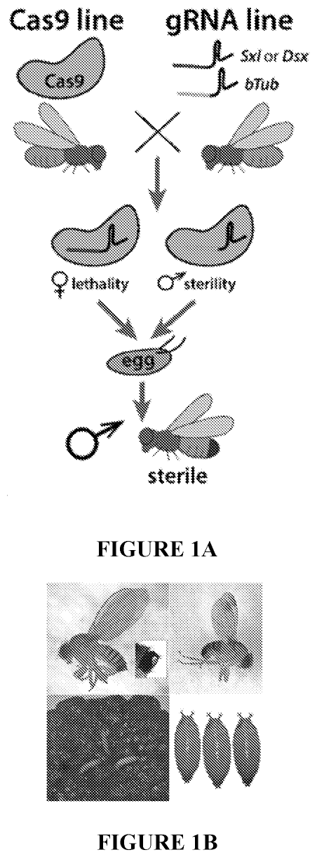

In D. melanogaster, it was demonstrated that a ubiquitously expressed or maternally deposited Cas9 endonuclease guided by a specific gRNA can efficiently mutate a target gene locus in both somatic and germ cells and cause the loss-of-function phenotype in 100% of F₁ progeny. To apply this highly efficient Cas9/gRNA-directed cleavage for the precision guided SIT (pgSIT), applicant decided to maintain a Cas9 endonuclease and a gRNA as separate component line of the CRISPR/Cas9 system and cross them when the loss-of-function phenotype is required (FIG. 1A). The gRNA line was designed to express two independent gRNAs: one to cause female lethality and the other to induce male sterility. The Cas9 line expressed the Cas9 endonuclease either ubiquitously or just in the germline. Both lines were continuously propagated and healthily expanded separately in the laboratory, while their mutual progeny developed into fit sterile males (WO 2019/103982, Kandul et al. 2018 and Kandul et al. 2019). Applicant demonstrated 100%±0% heritable cleavage of the white gene in Ae. aegypti when optimized under the expression of two separate promoters (nup50-Cas9, uniq-Cas9) and similar high cleavage rates have also been obtained in D. melanogaster (Port et al. 2014; Bassett and Liu 2014) and A. aegypti (M. Li et al. 2017) in other studies. Furthermore, through a mechanism called lethal biallelic mosaicism (LBM), 100% sterile male phenotypes are achieved even though the allelic conversion in the whole organism was less than 100% (WO 2019/103982, Kandul et al. 2018 and Kandul et al. 2019).

Lethal Biallelic Mocaisism Ensures 100% Efficiency of pgSIT Technology

As described above, pgSIT relies exclusively on highly efficient CRISPR-mediated DNA cleavage and NHEJ-based repair to cause mosaic mutations (aka. indels) at conserved loci to achieve a full loss-of-function (sterile male) phenotype. The pgSIT system built in D. melanogaster demonstrated that zygotic activity of the Cas9/gRNA complexes ensures continuous biallelic mosaicism of targeted alleles throughout development and when engineered to target female essential genes and male fertility genes resulted in complete and consistent sterile male phenotypes (WO 2019/103982, Kandul et al. 2019 and Kandul et al. 2018). Moreover, paternal inheritance of dgRNAs along with maternal deposition of Cas9 in developing embryos, in the absence of Cas9 inherited as a gene, was also sufficient to induce detectable biallelic mosaicism for all genes targeted (βTub, dsx, tra, sxl). For example, maternal deposition of Cas9 alone was sufficient to induce intersex phenotypes (dsx, tra), however it was insufficient to phenotypically ensure male sterility (βTub), and depended on the strength of the promoter maternally depositing Cas9 to ensure female death (sxl) via lethal biallelic mosaicism (WO 2019/103982, Kandul et al. 2019 and Kandul et al. 2018). Taken together, these observations suggest that rates of biallelic mosaicism, which ensure the desired phenotype, depend exclusively on whether components (i.e. Cas9 and gRNA) are inherited as genes or maternally deposited.

Further, the diversity of promoters and genetic elements in the disclosed toolbox allows us to achieve strong, consistent and timely expression of these components in Ae. aegypti and with further development, other vectors. Additionally, regardless of how the components are inherited, if rates of biallelic mosaicism are over a critical threshold, which is specific to each gene targeted, then by targeting a female essential gene and male fertility, a 100% male sterile phenotype was achieved. Mechanistically, this technology demonstrates a fundamental advance in genetics by which somatic biallelic disruptions in essential genes, that previously conferred recessive phenotypes, get simultaneously converted by pgSIT in many somatic cells resulting in dominant fully penetrant phenotypes in a single generation (WO 2019/103982, Kandul et al. 2019 and Kandul et al. 2018).

General Experimental Approach

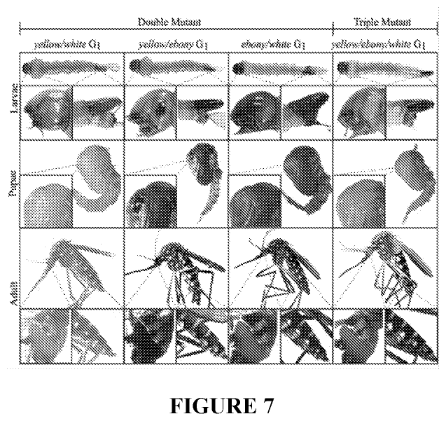

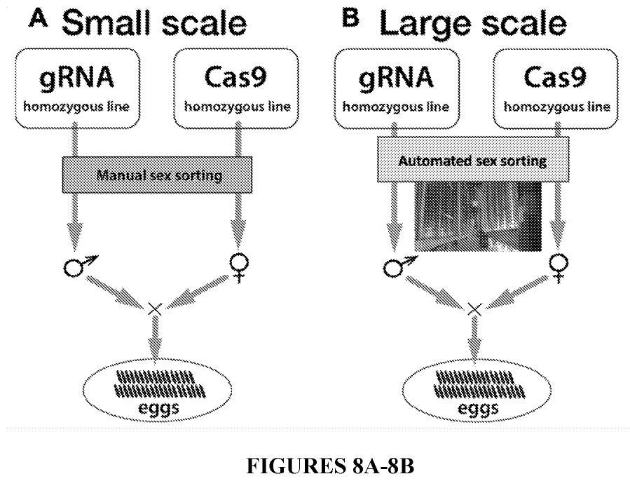

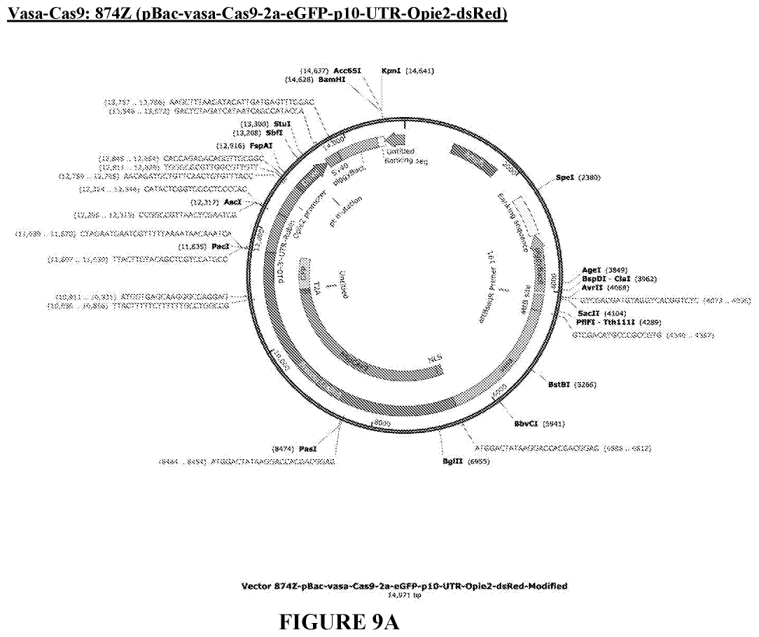

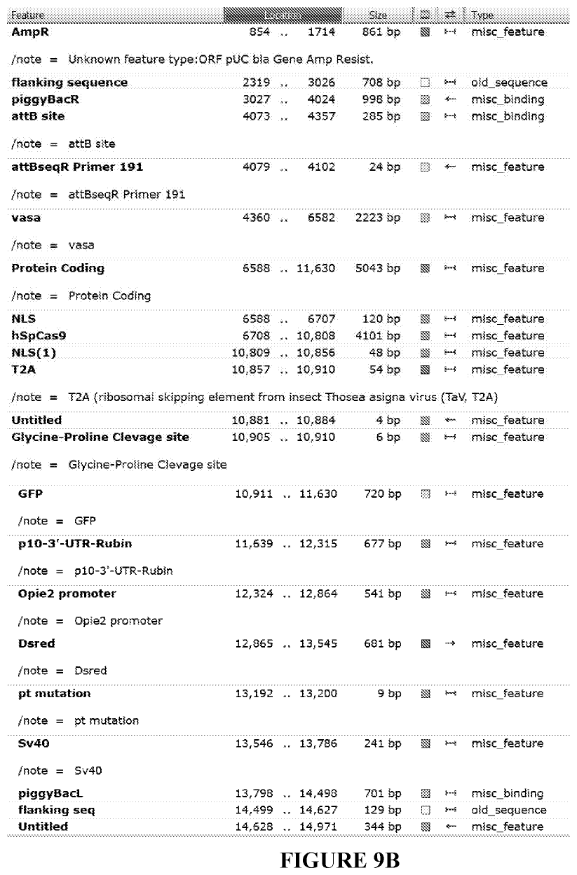

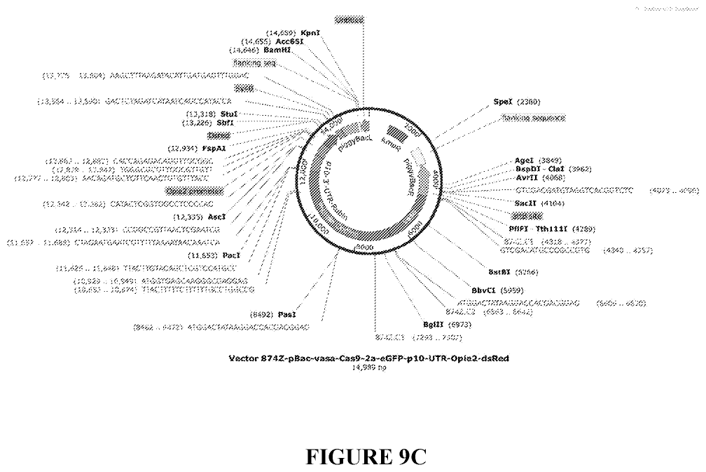

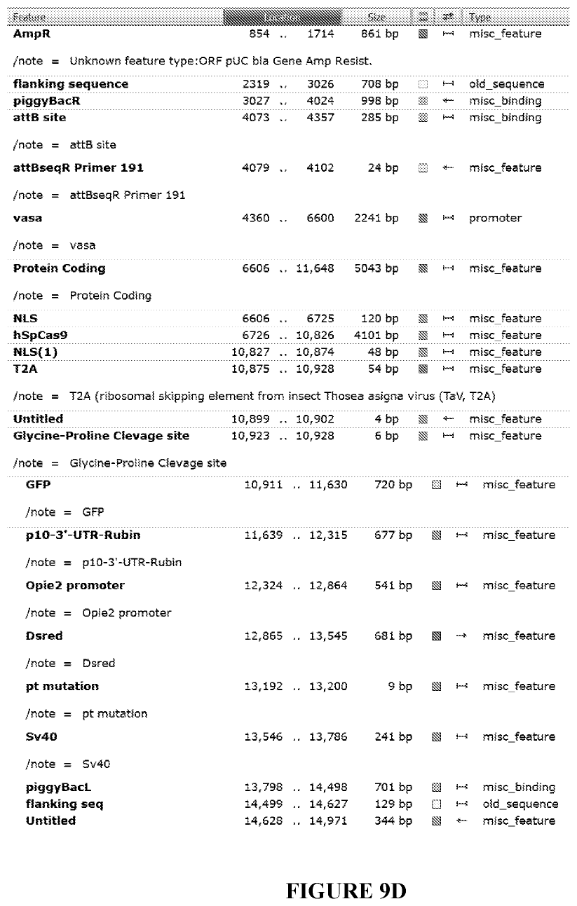

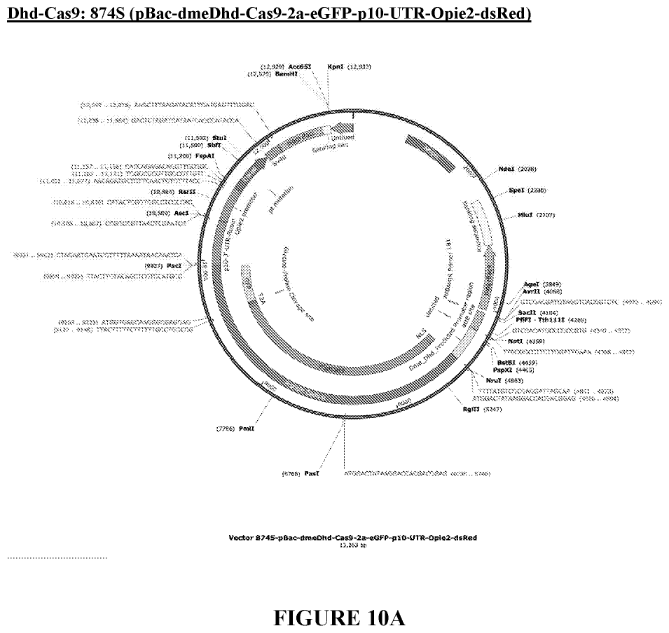

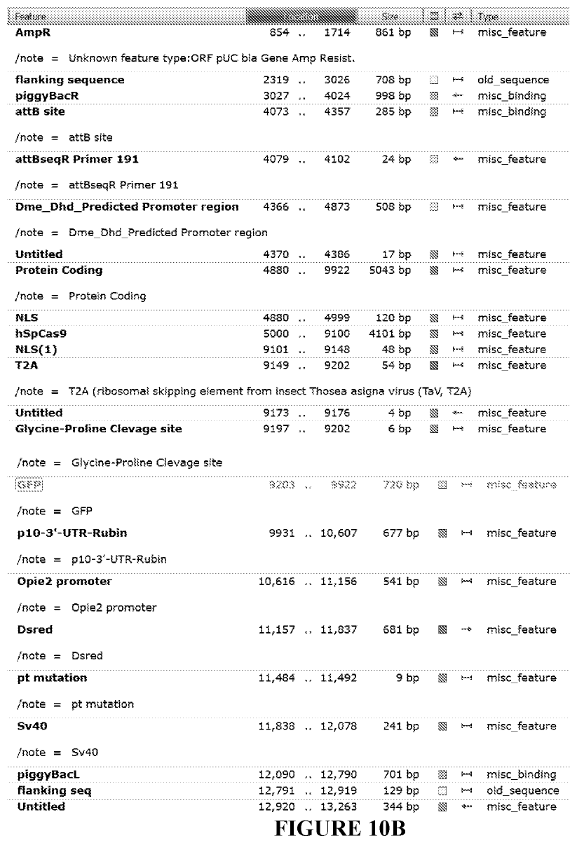

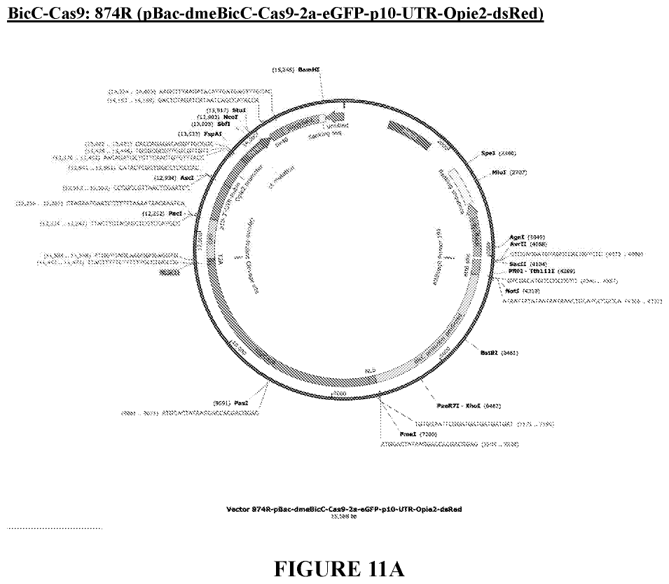

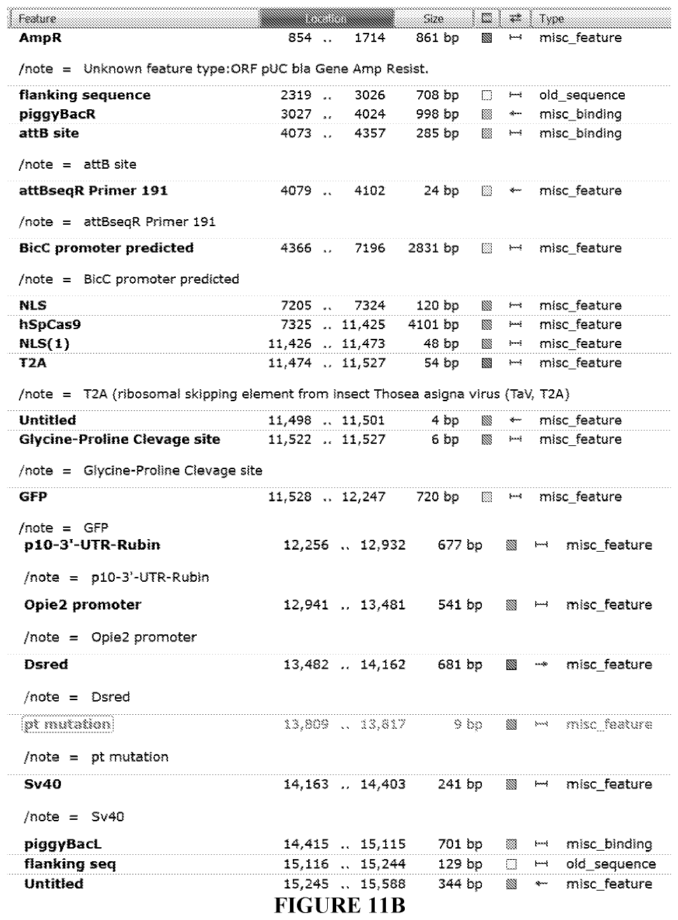

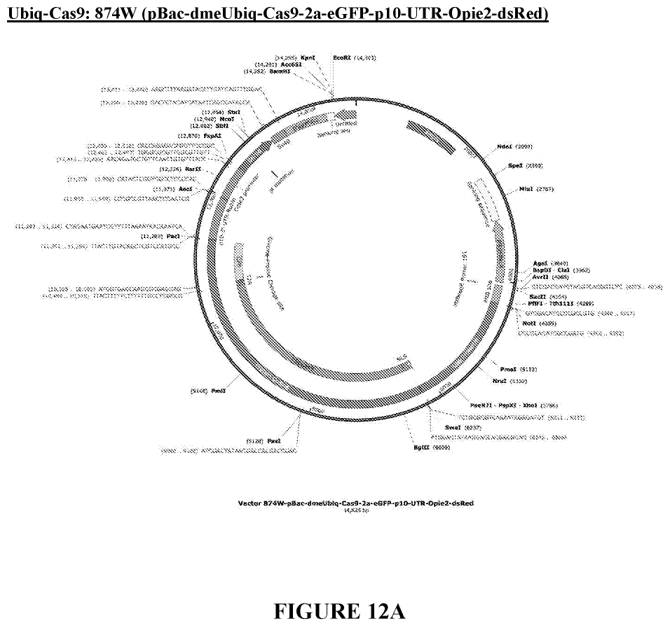

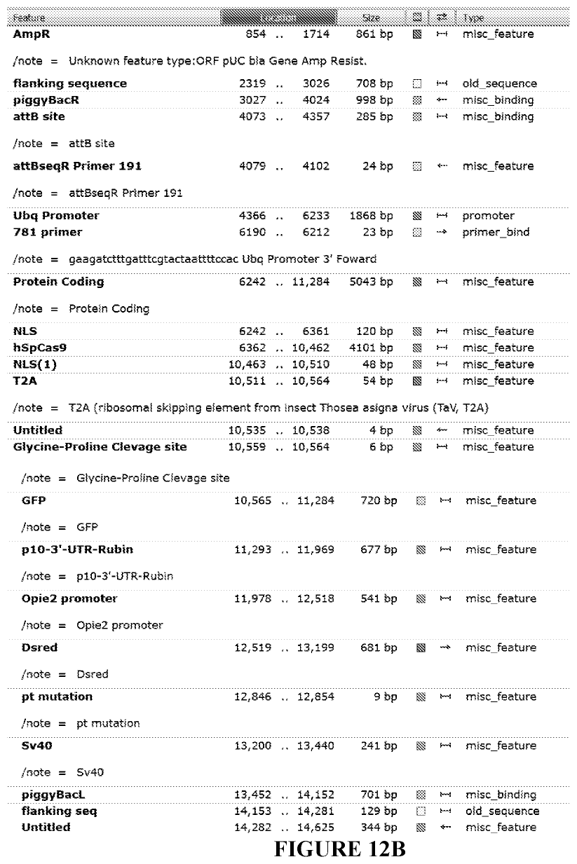

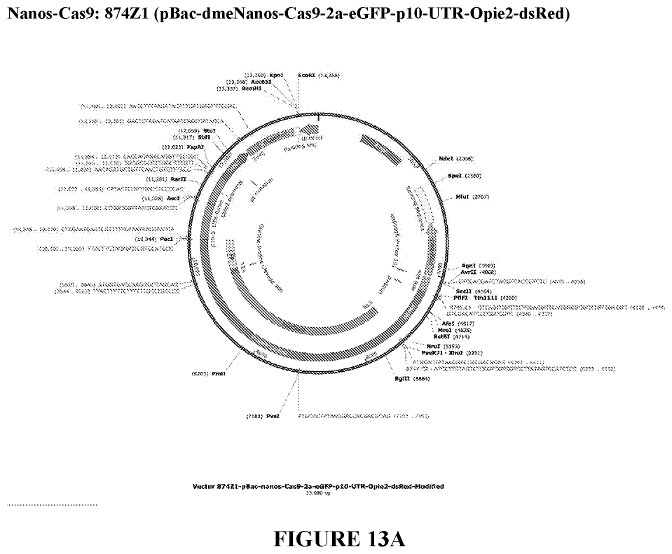

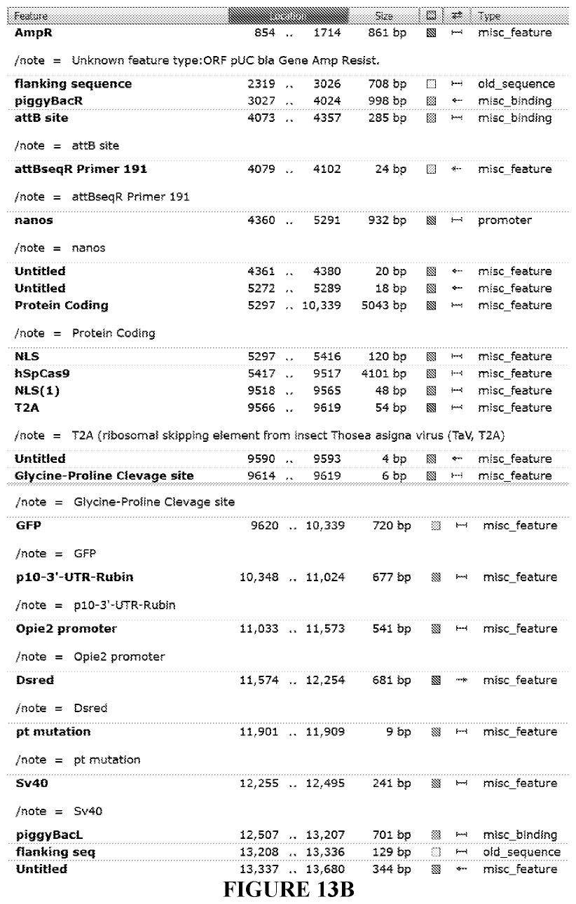

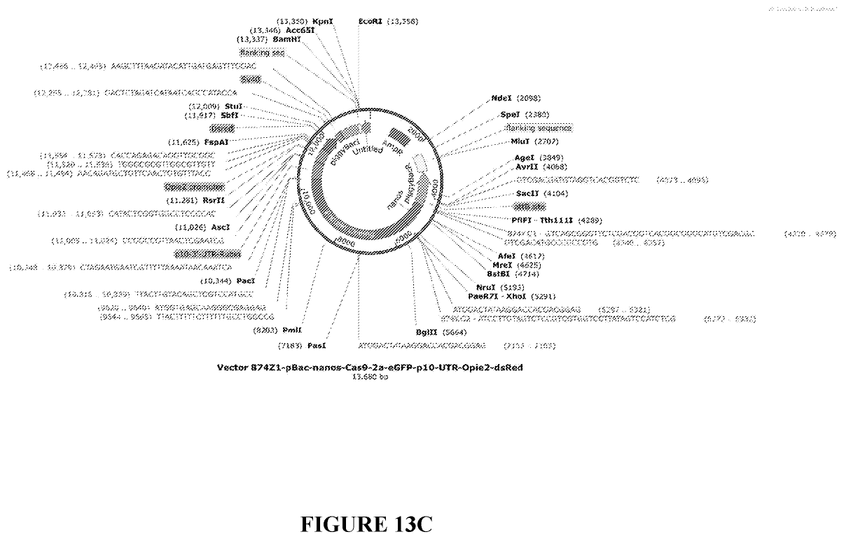

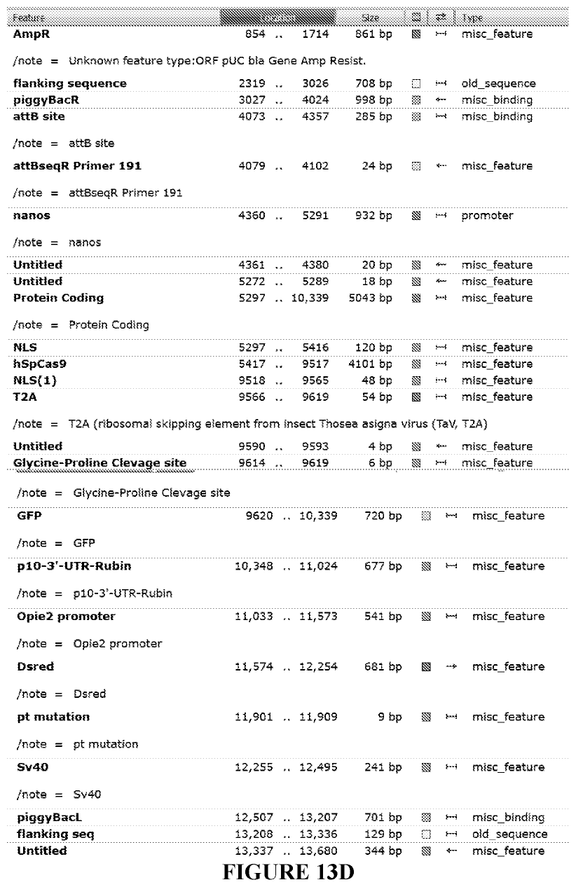

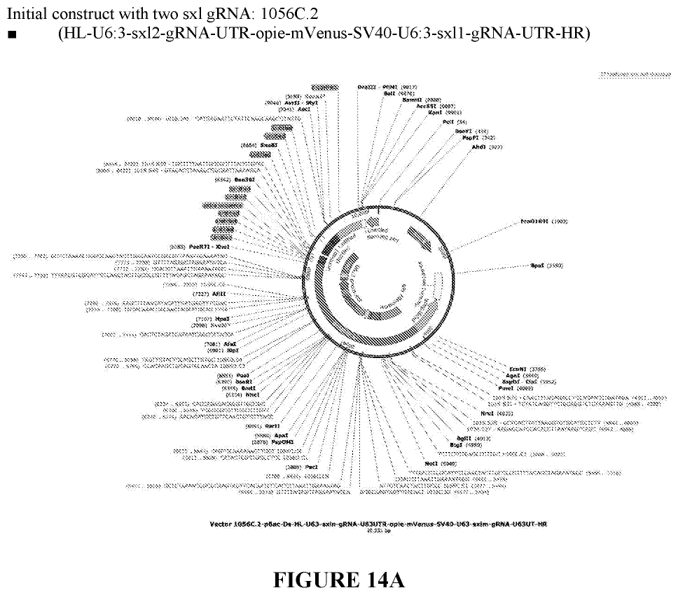

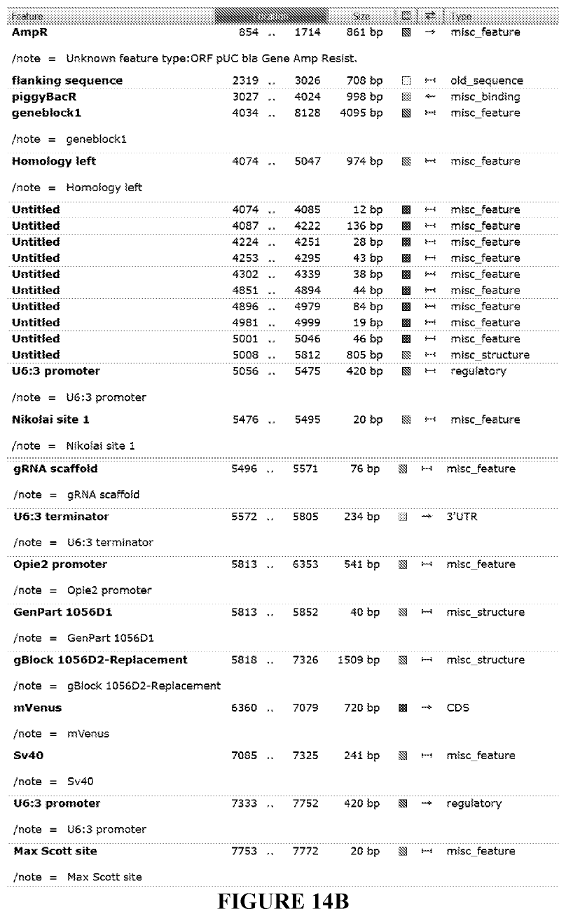

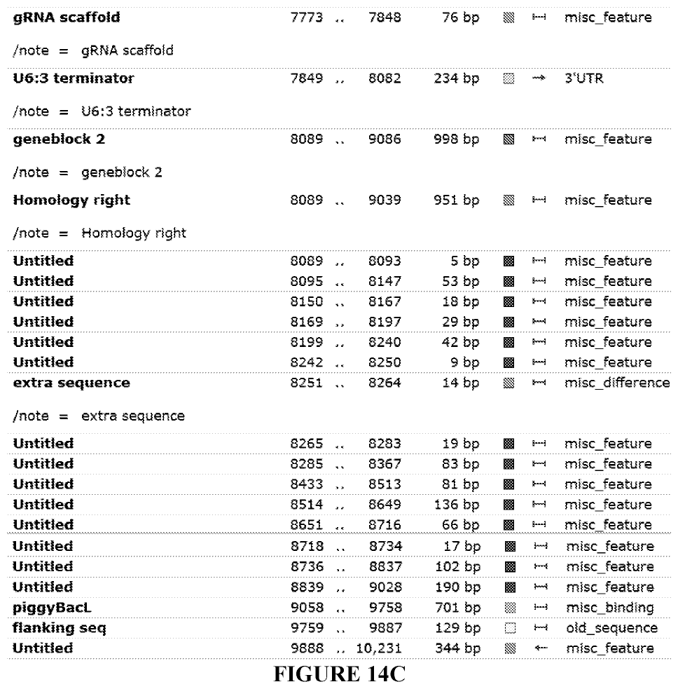

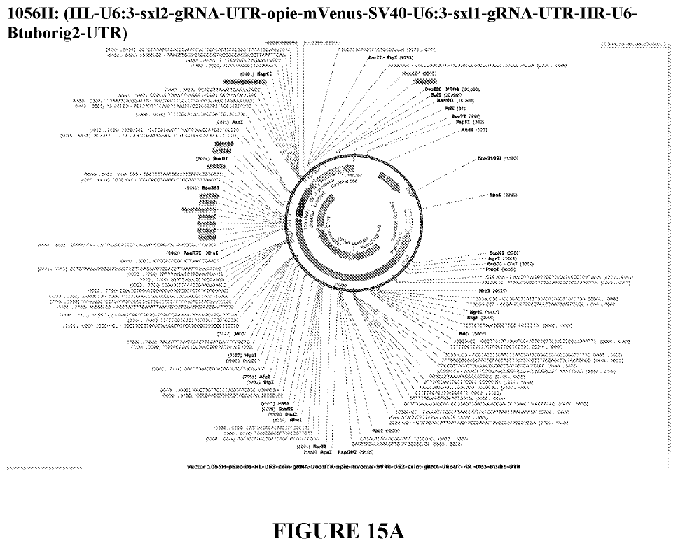

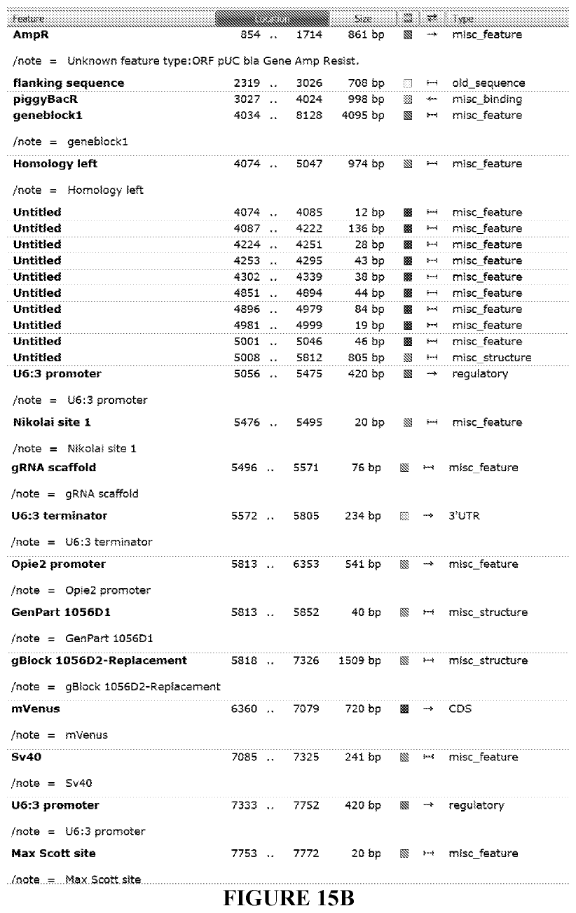

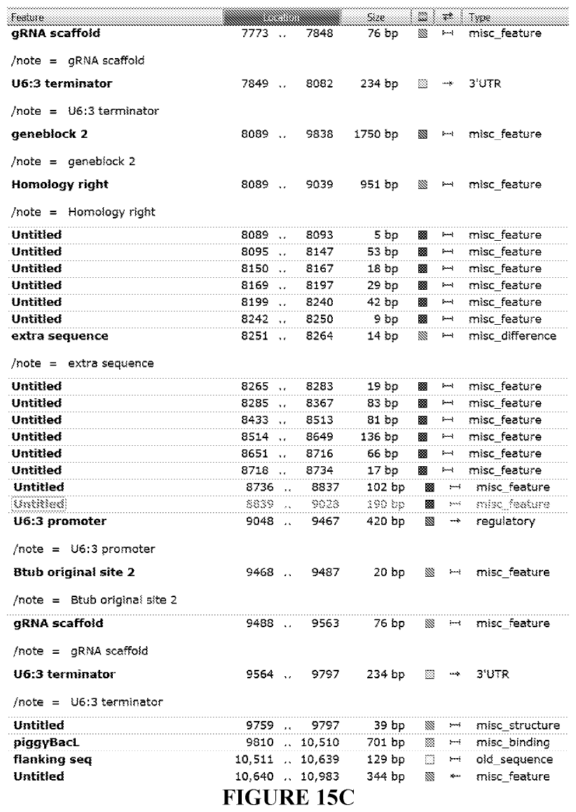

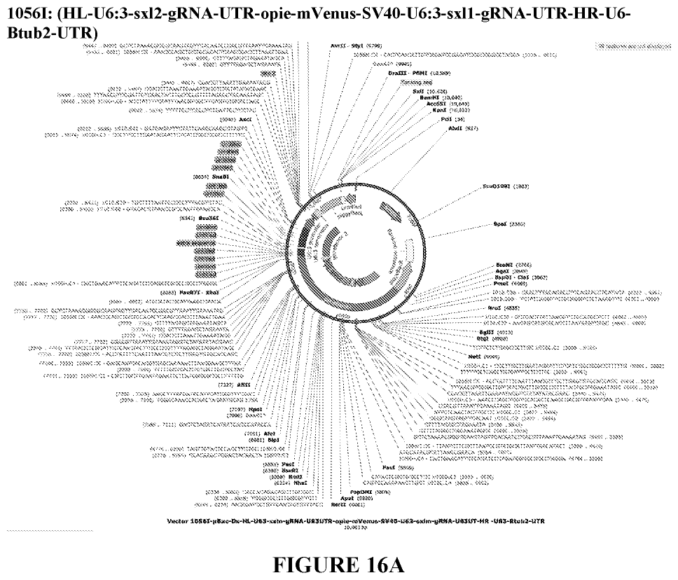

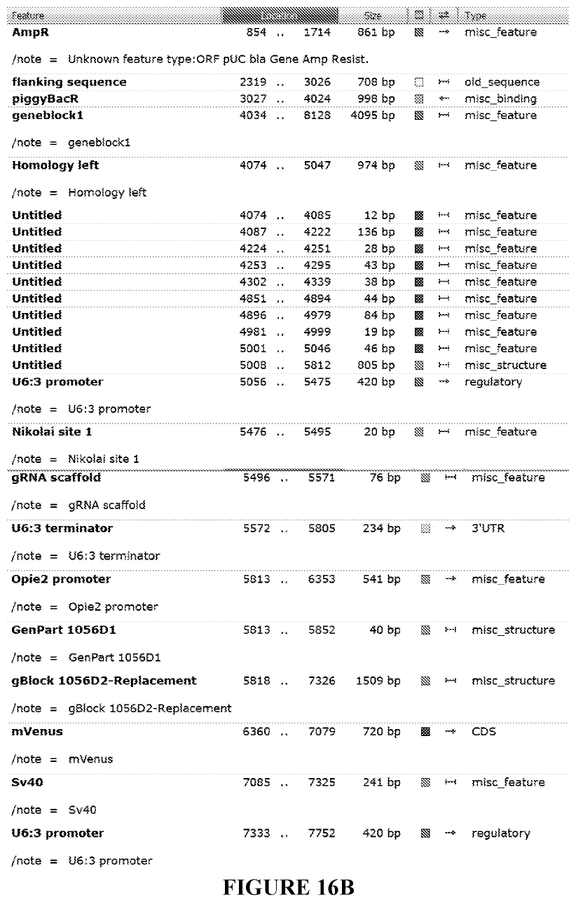

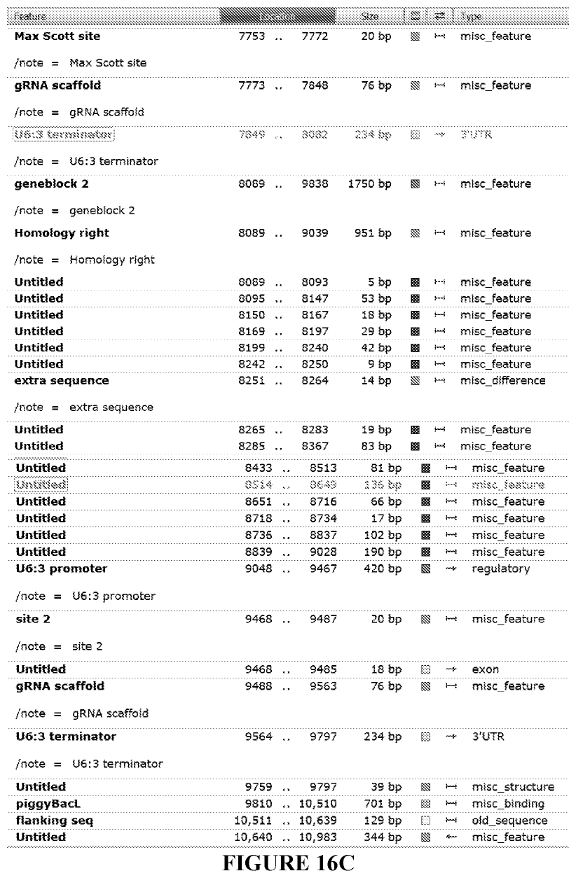

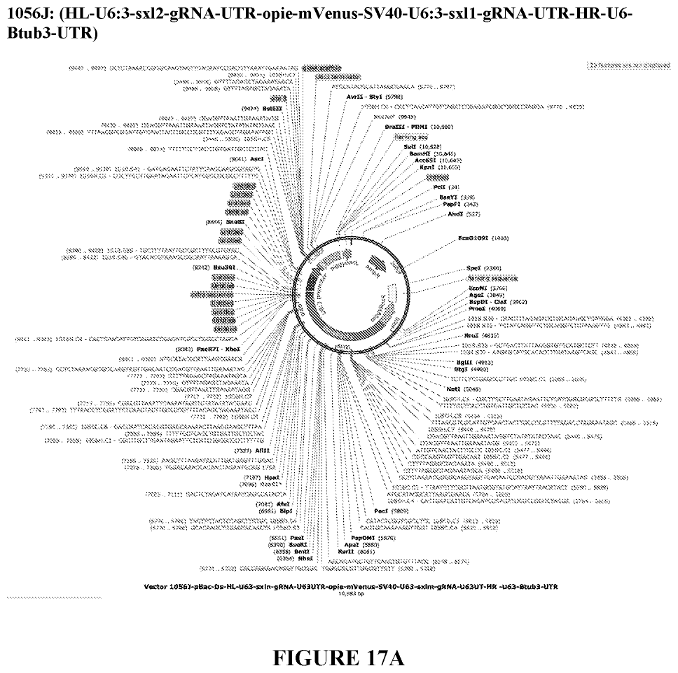

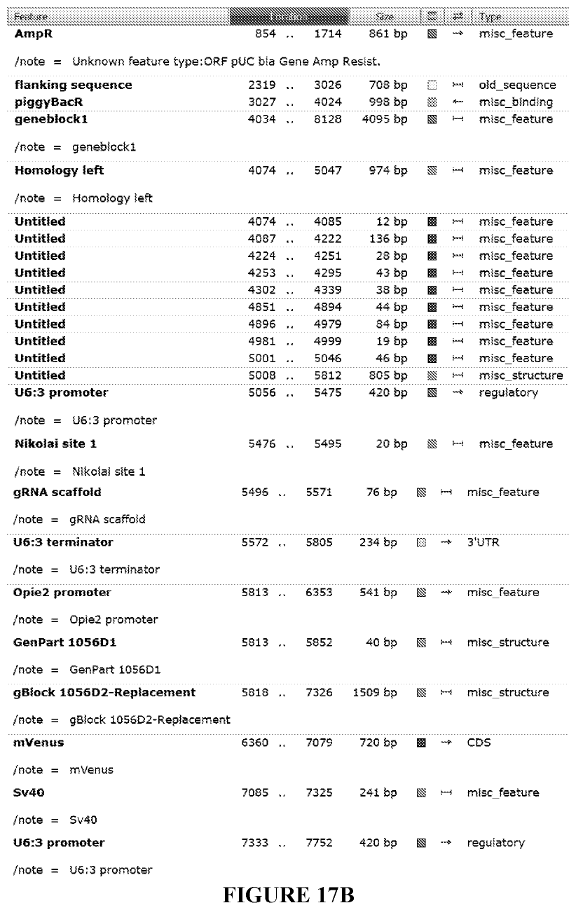

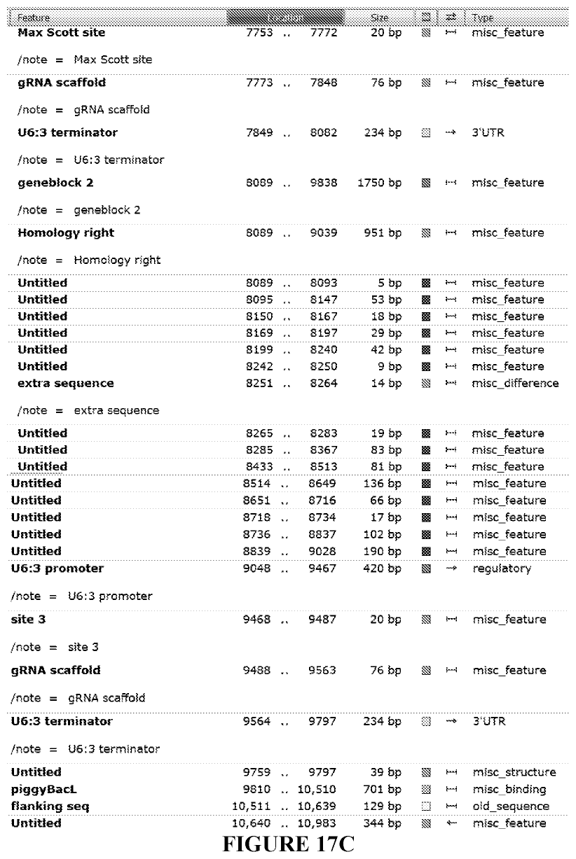

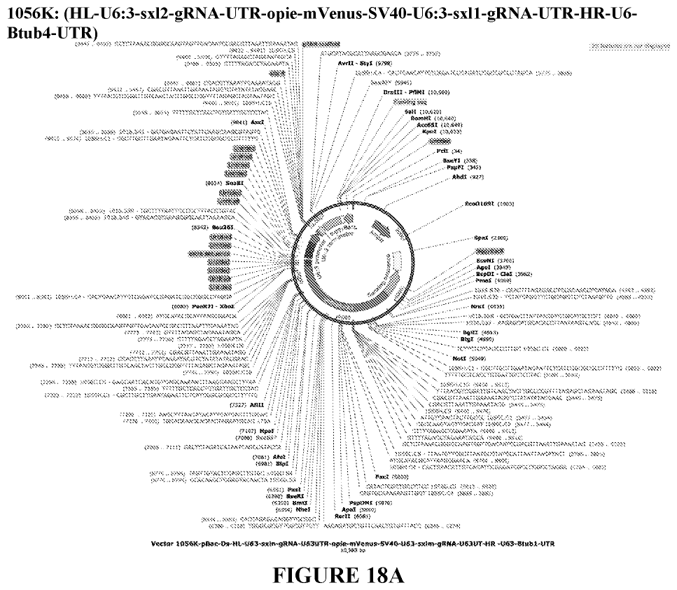

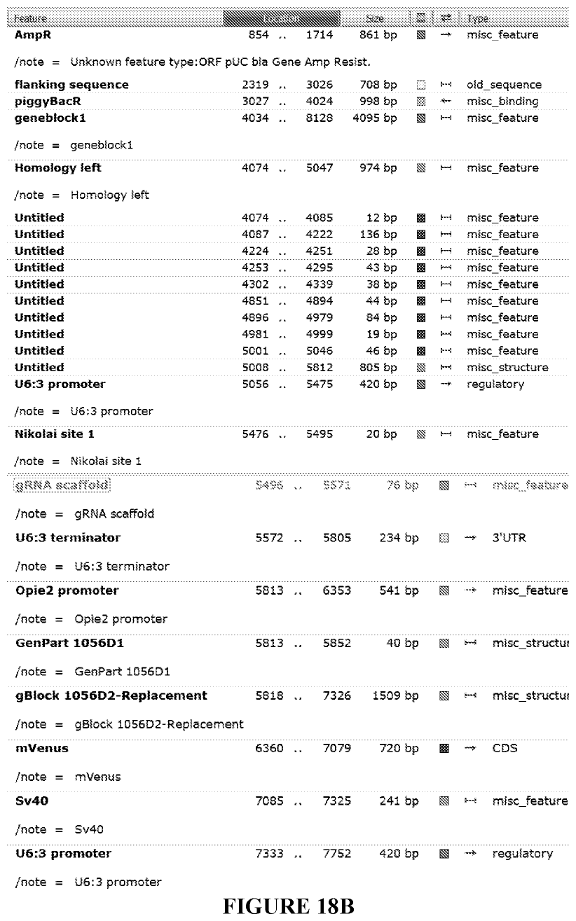

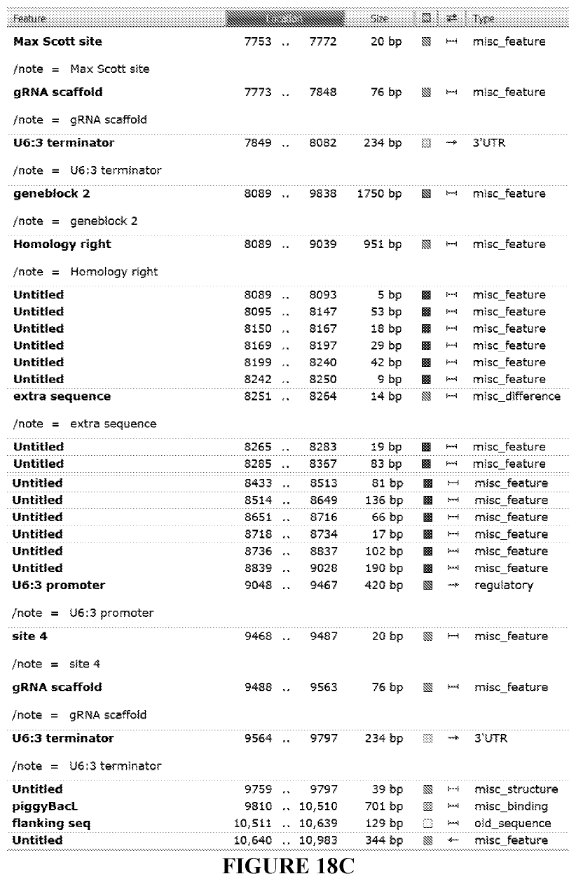

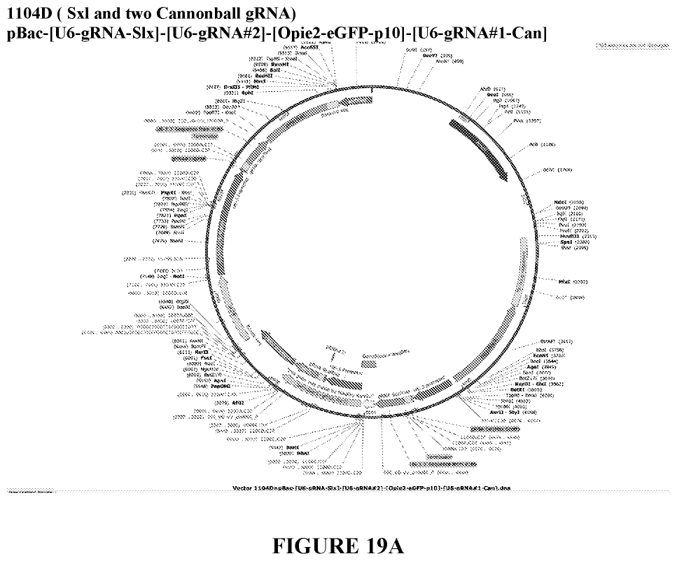

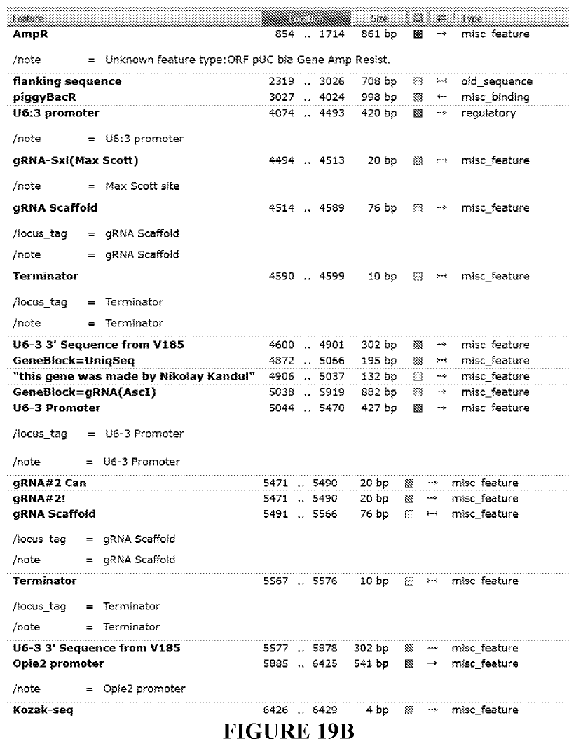

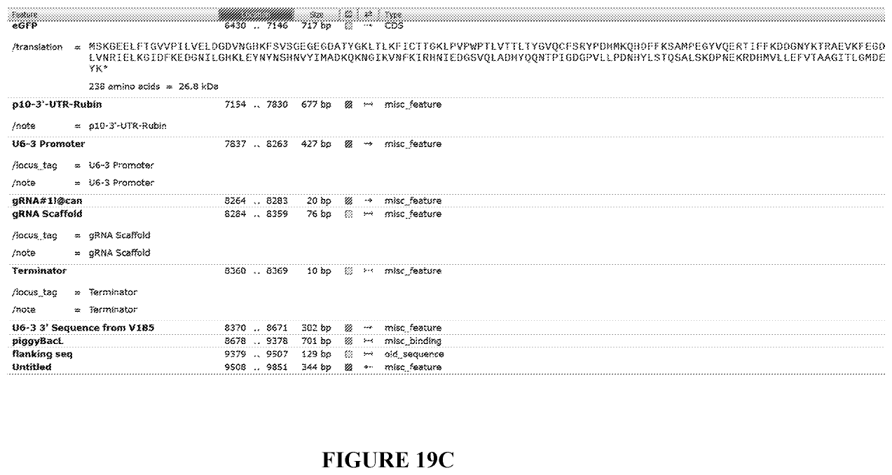

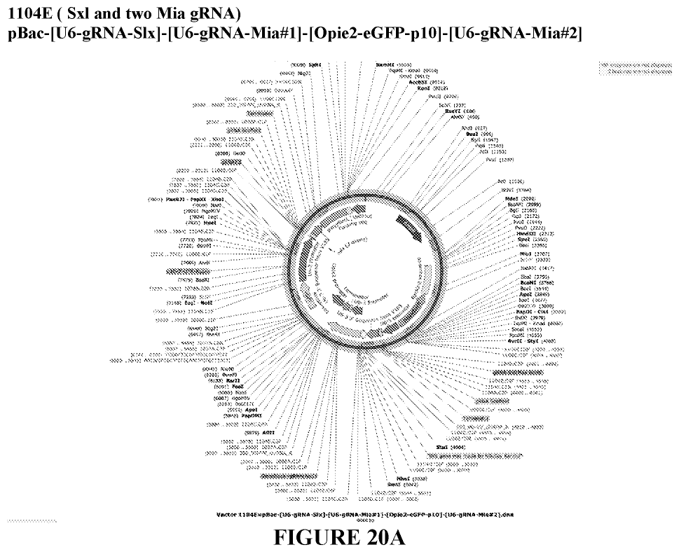

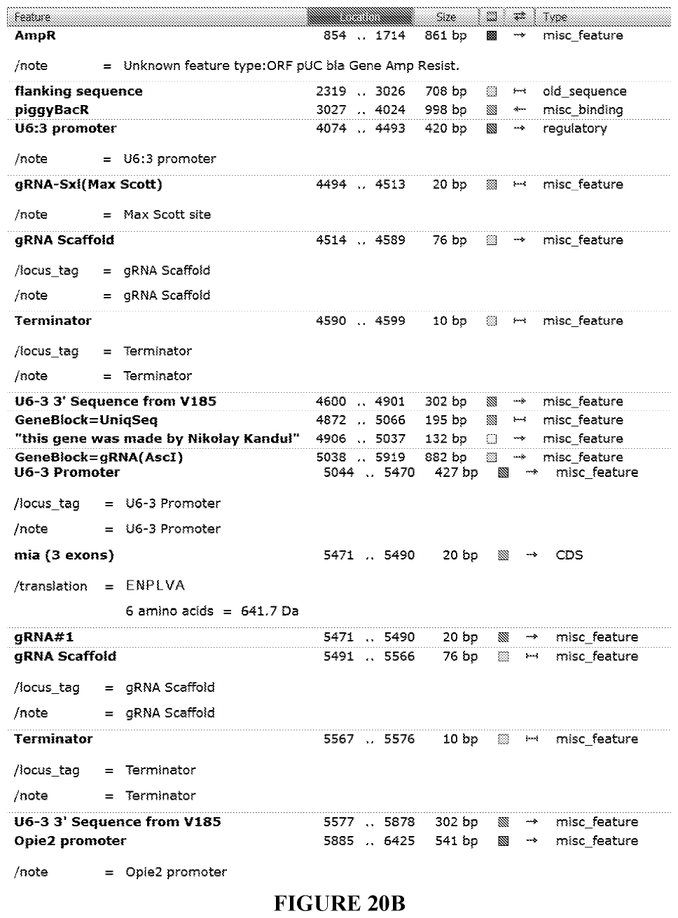

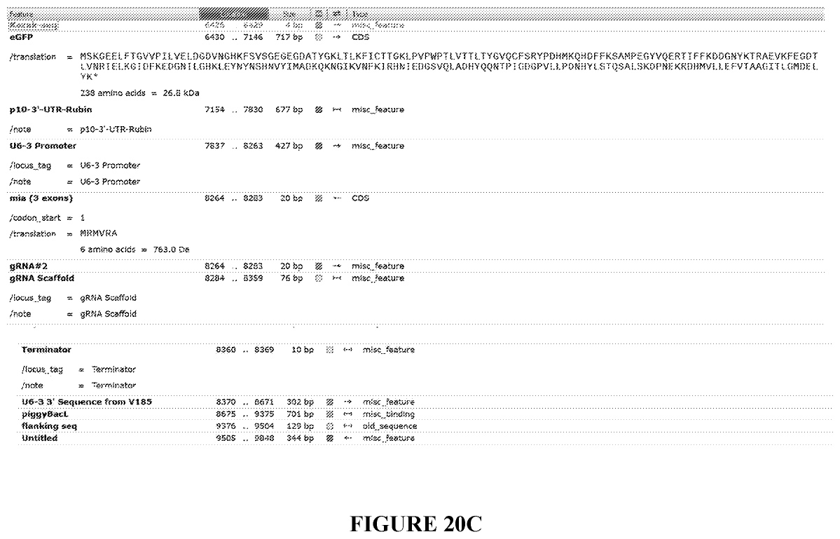

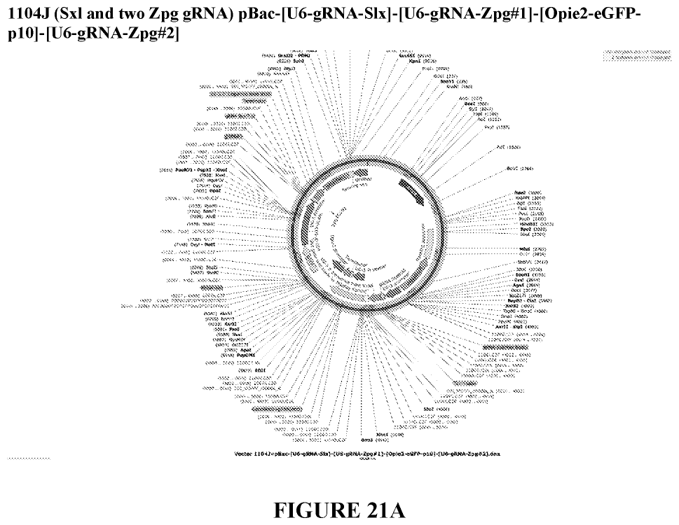

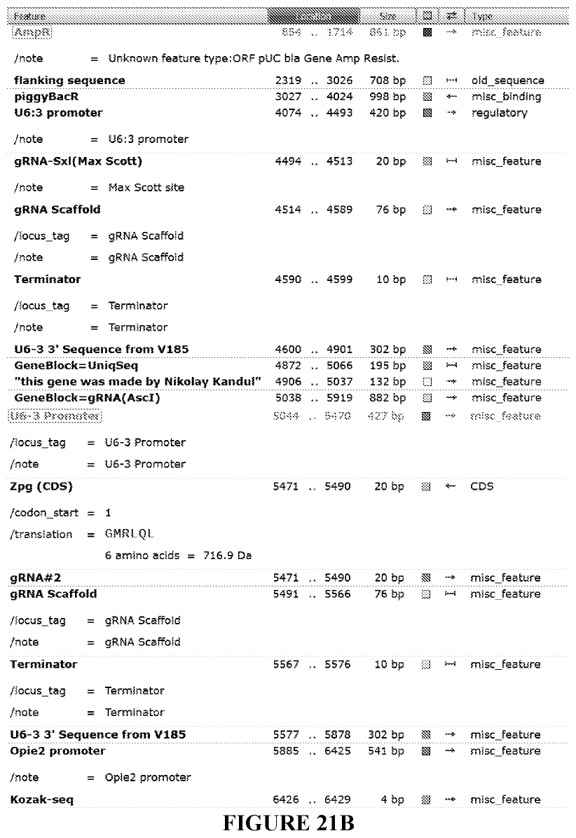

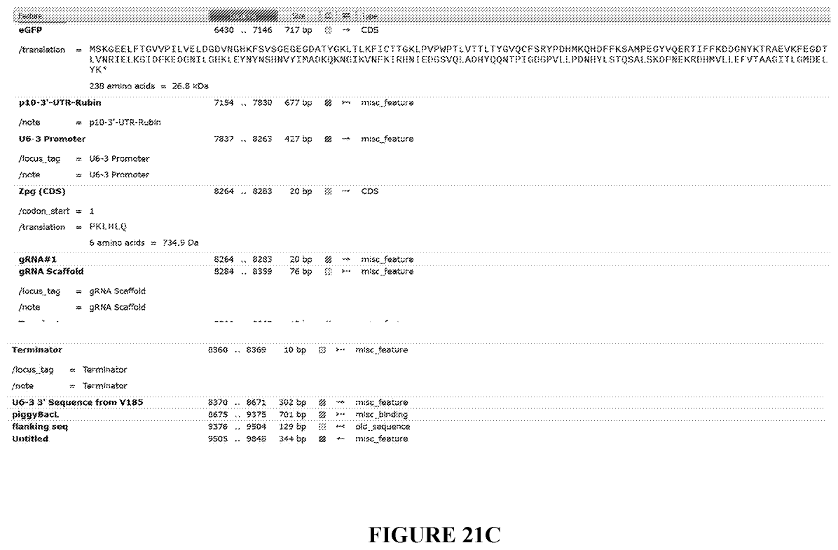

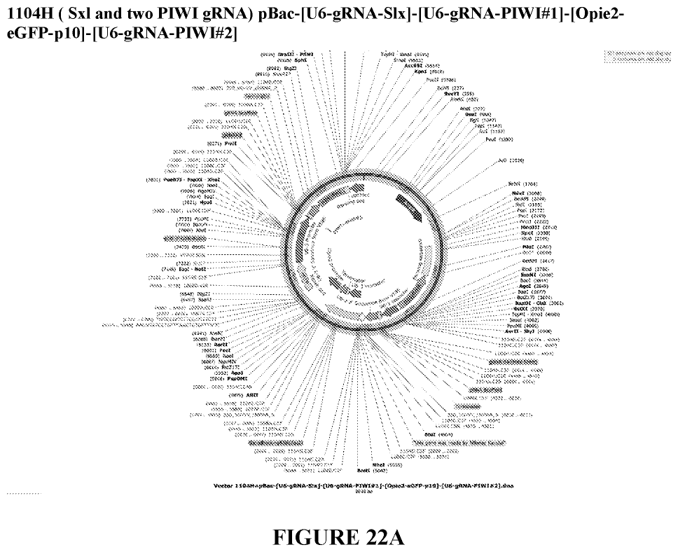

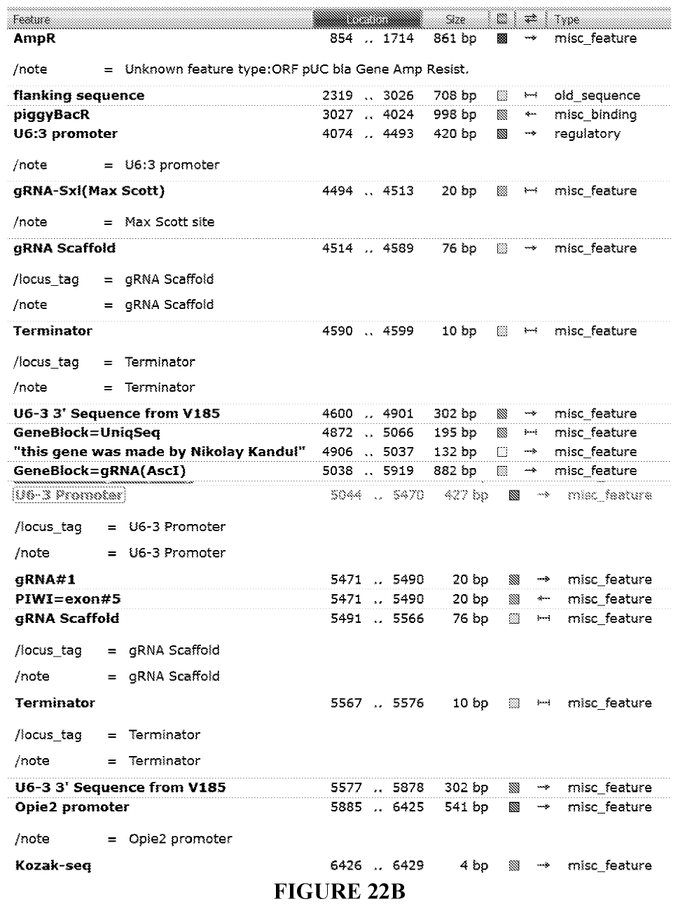

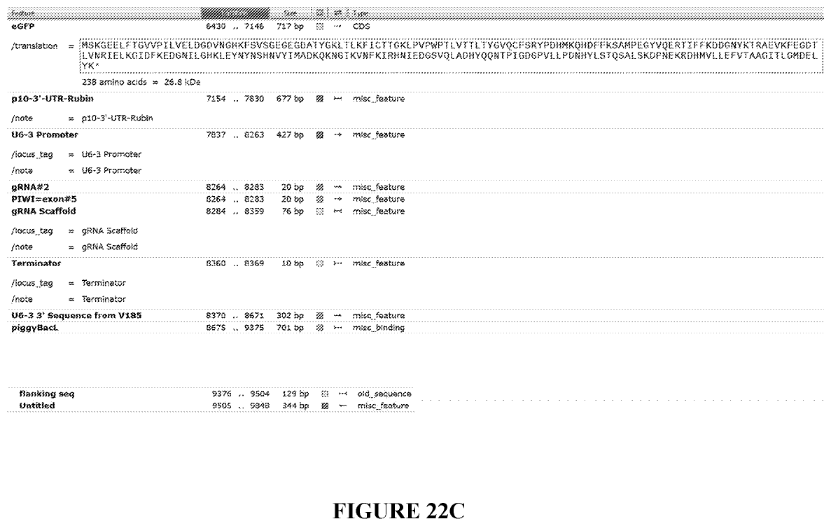

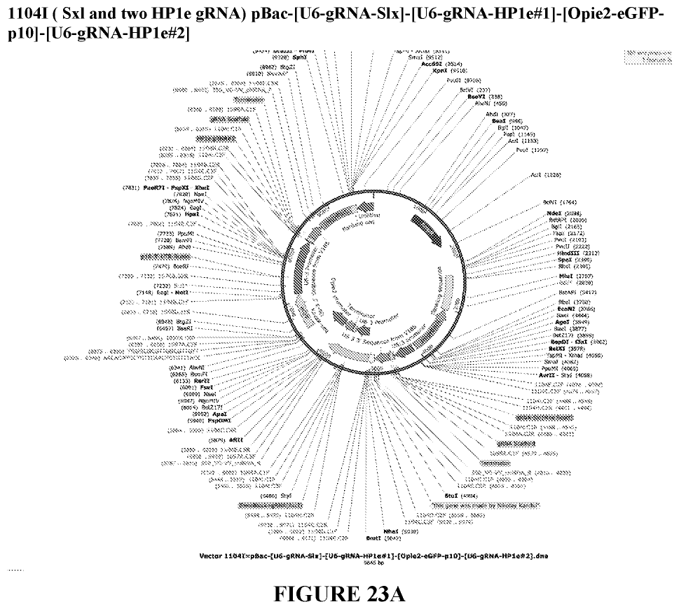

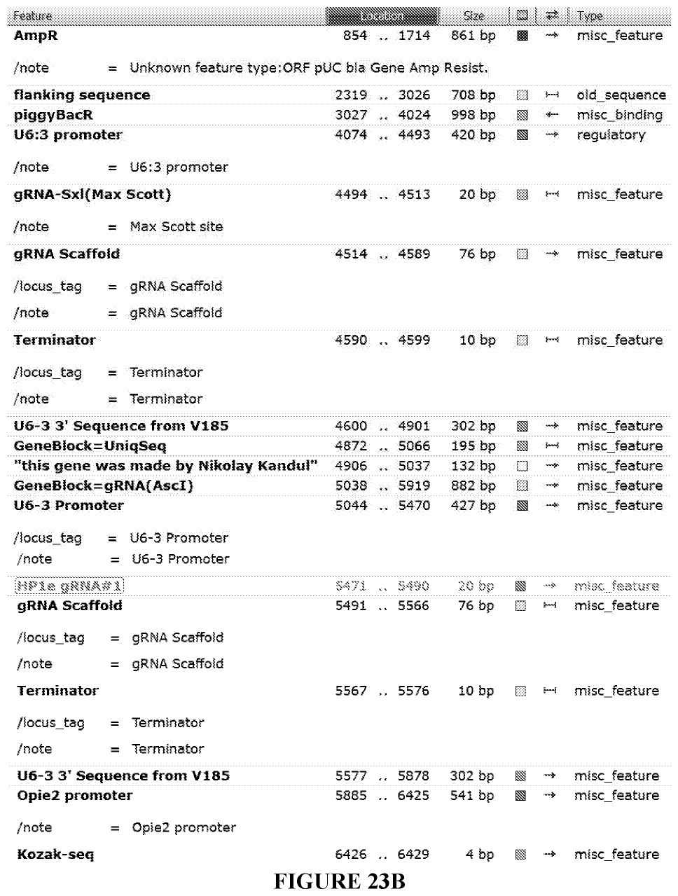

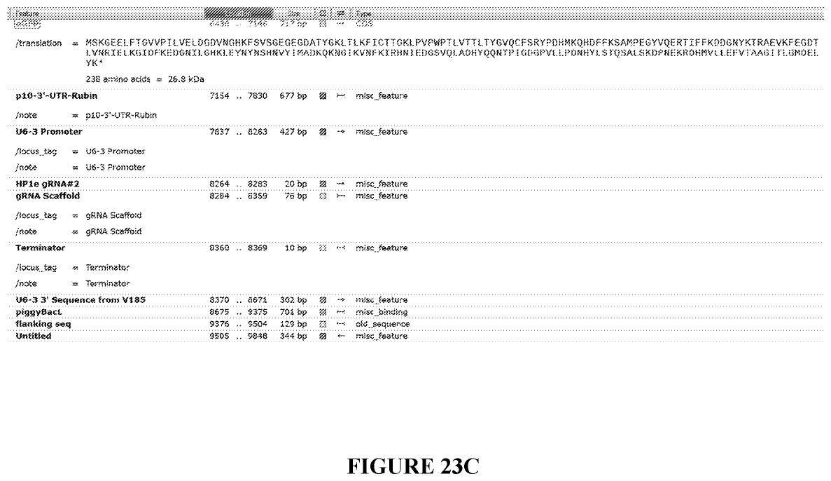

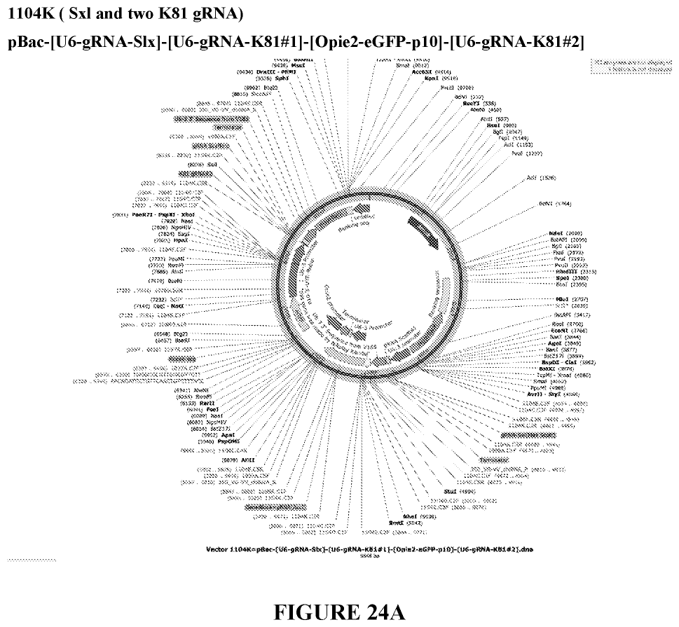

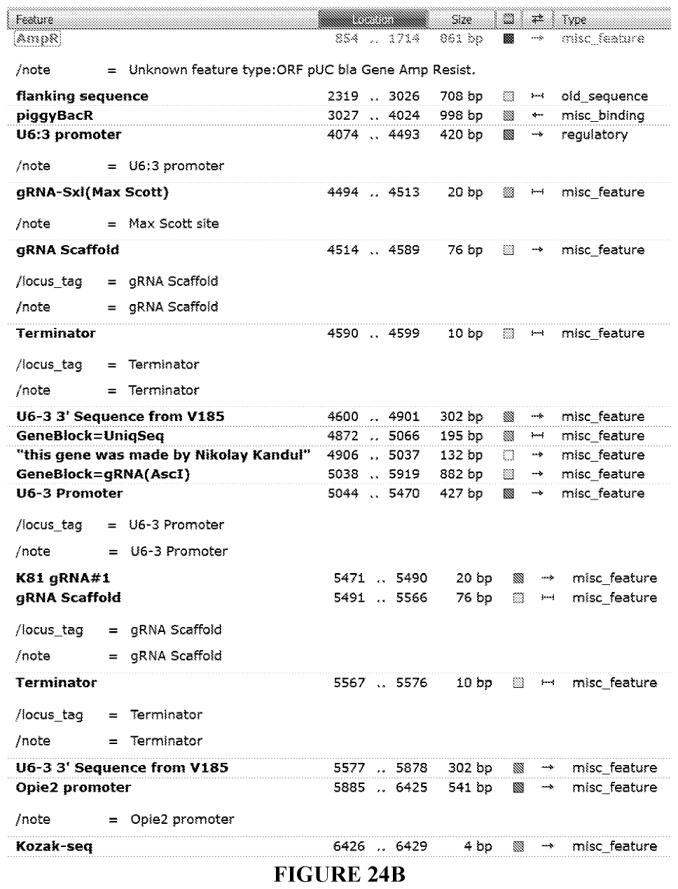

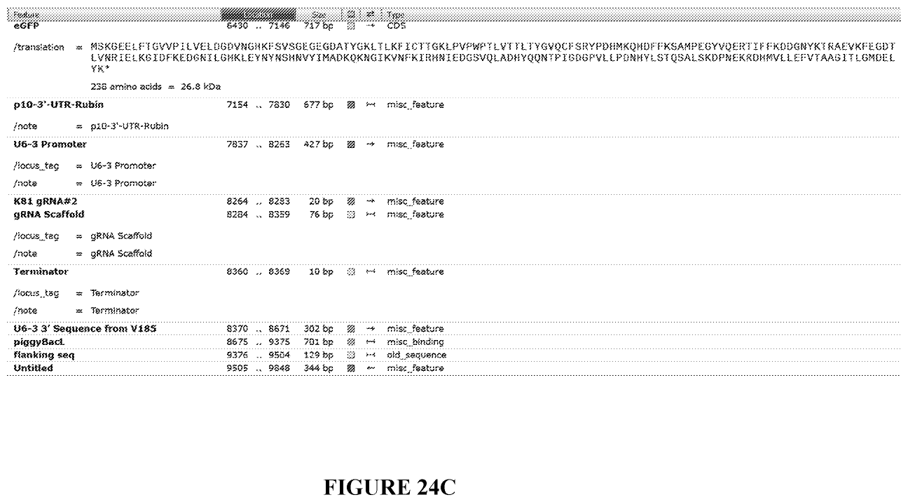

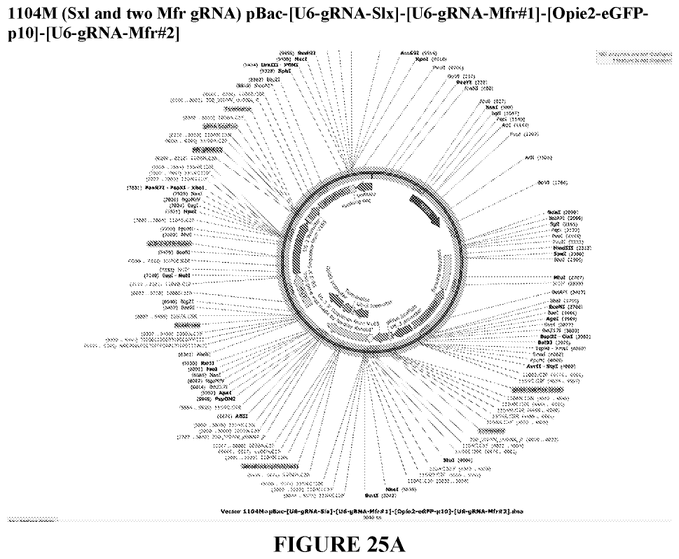

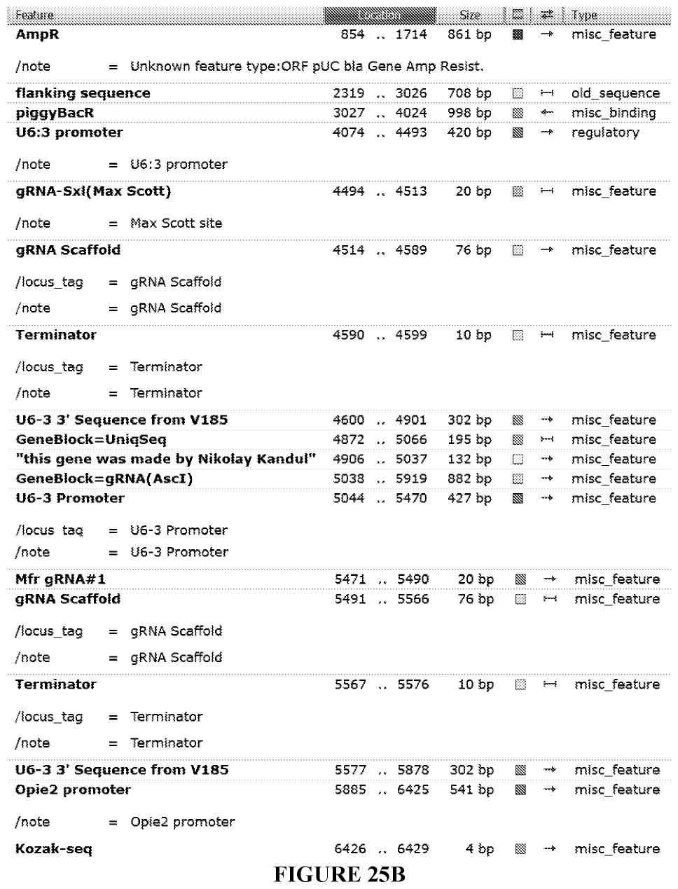

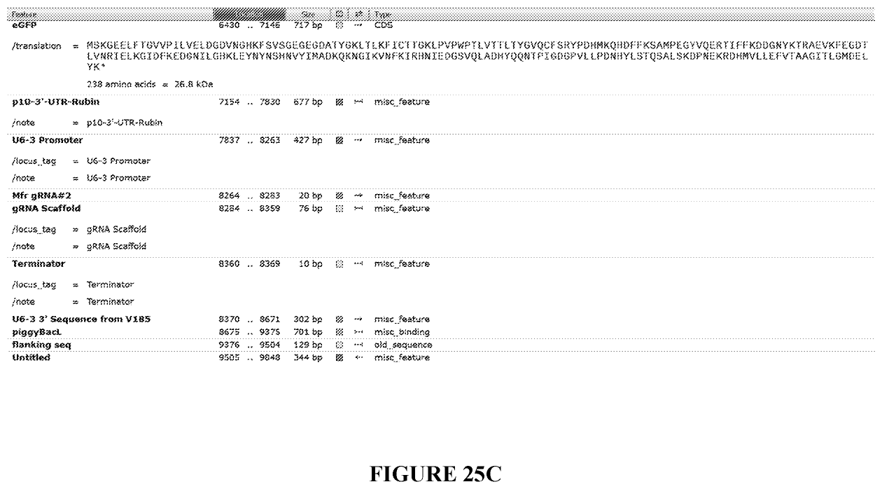

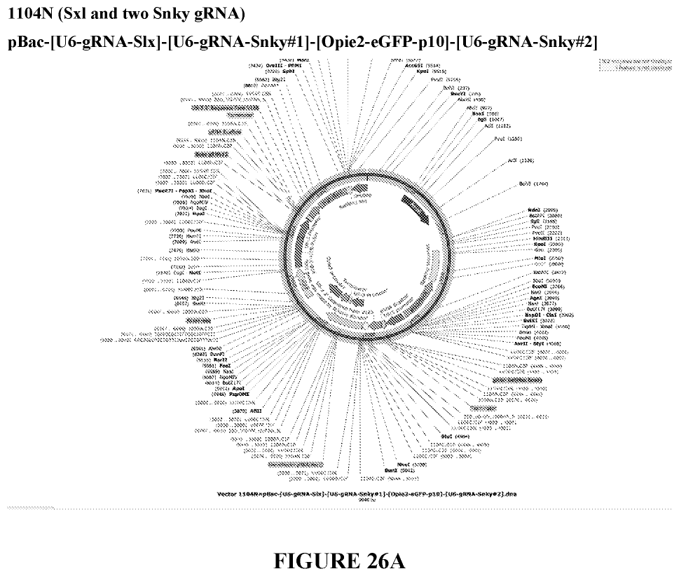

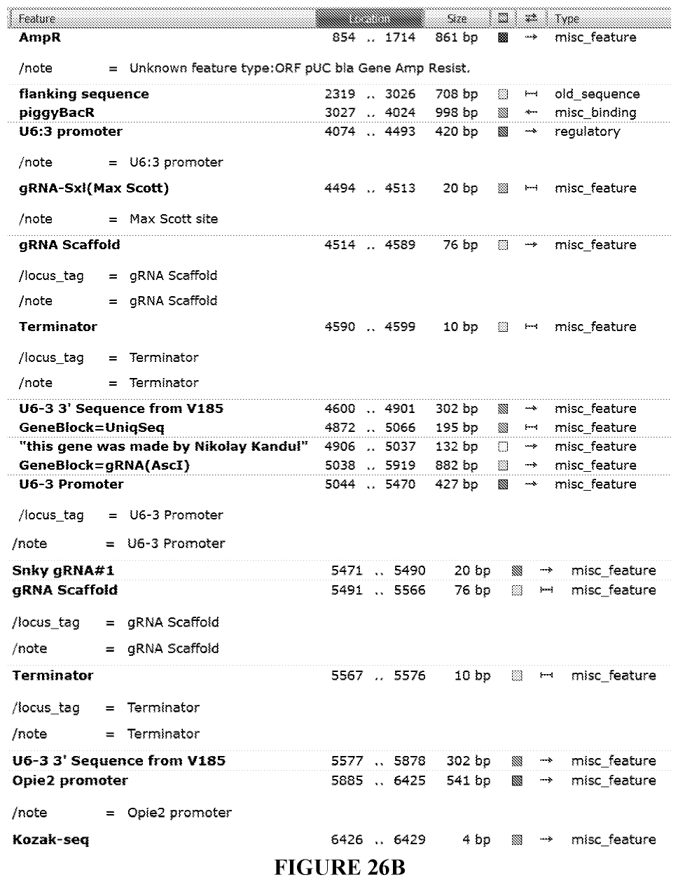

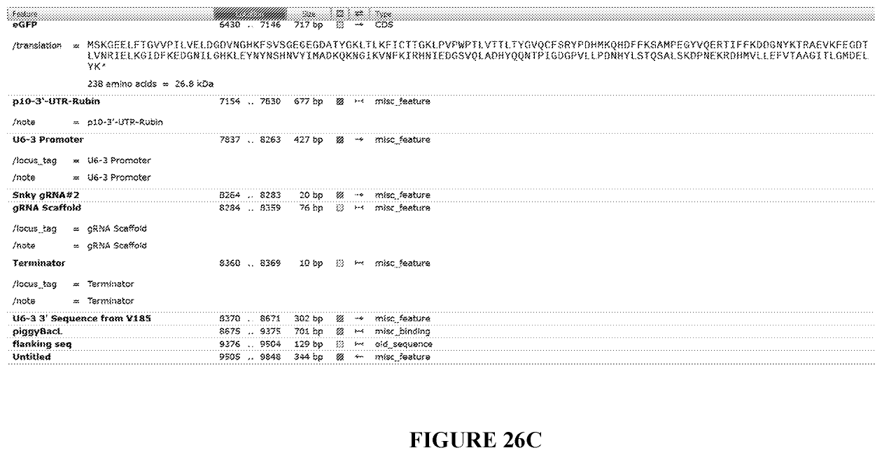

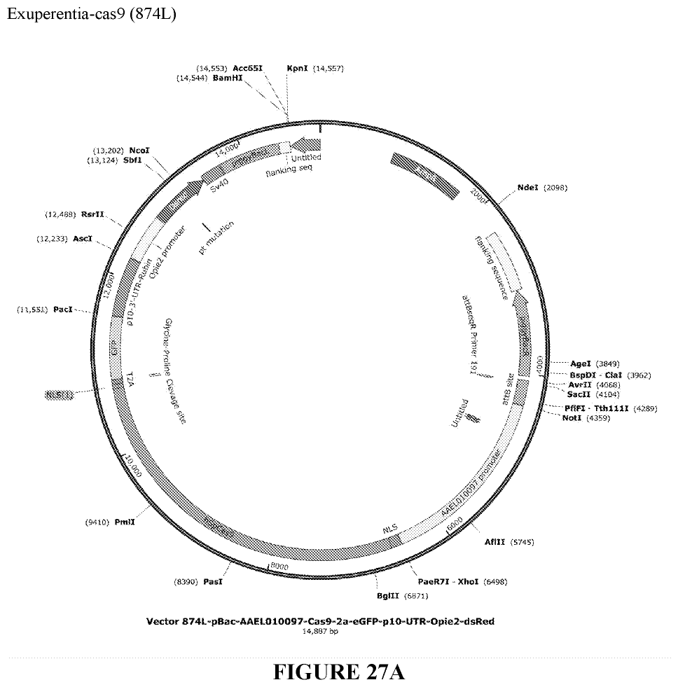

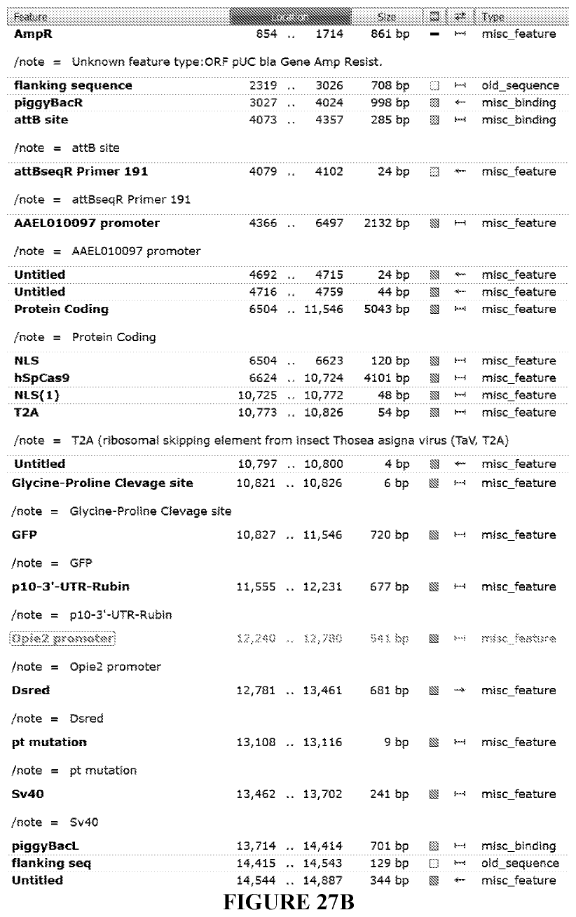

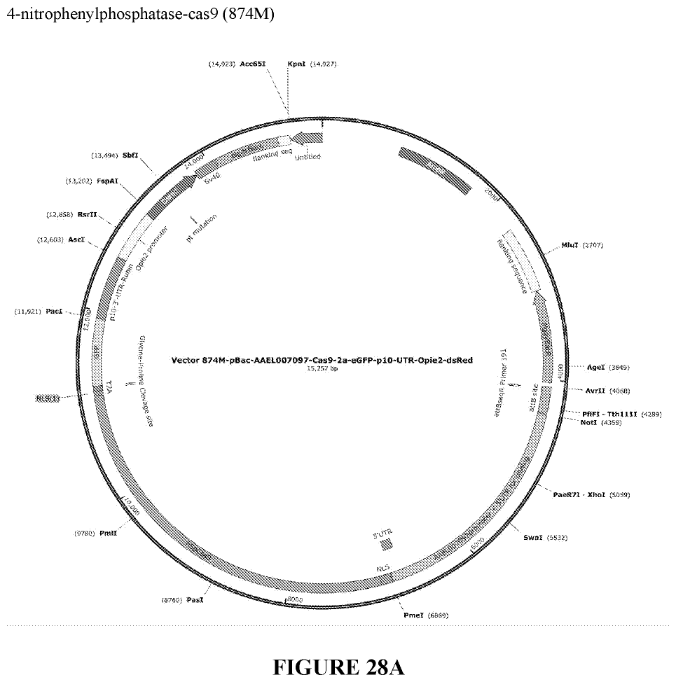

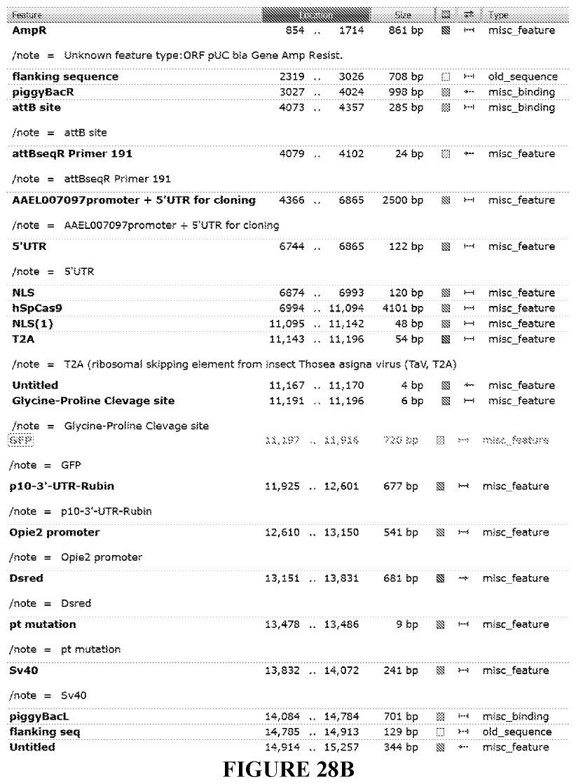

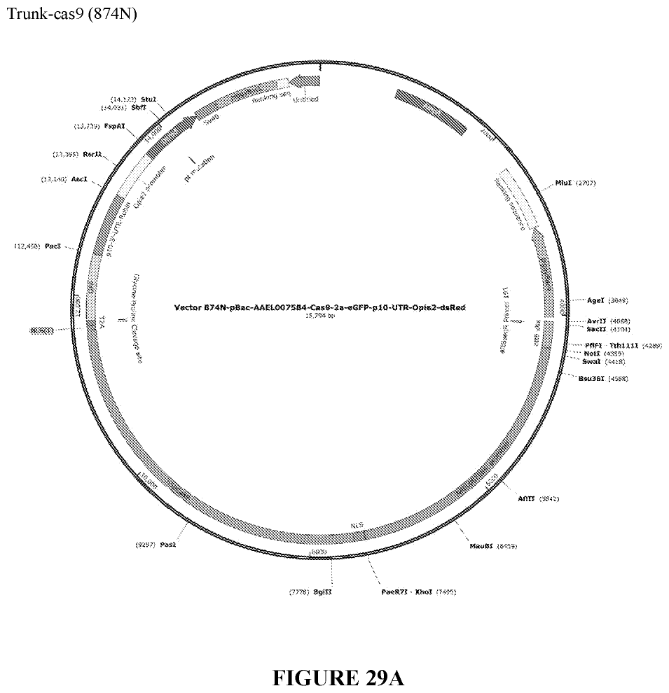

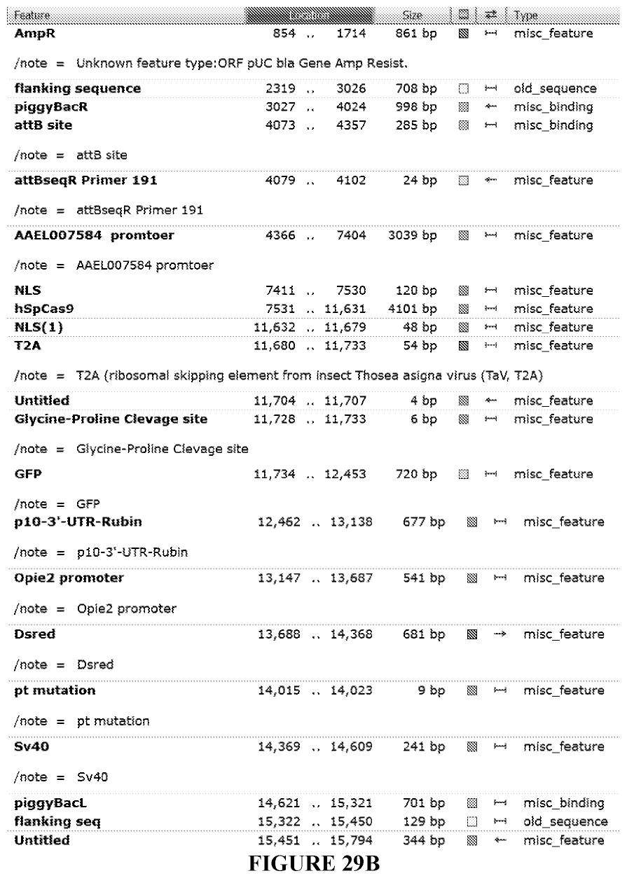

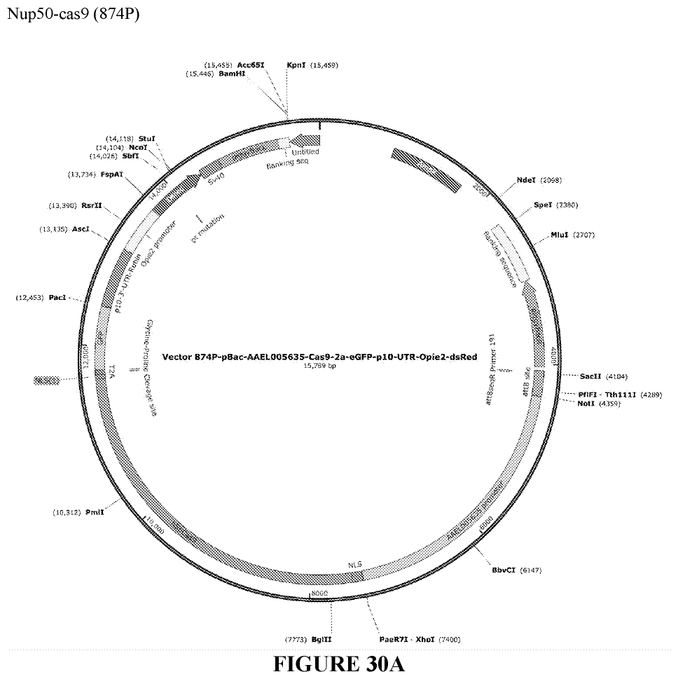

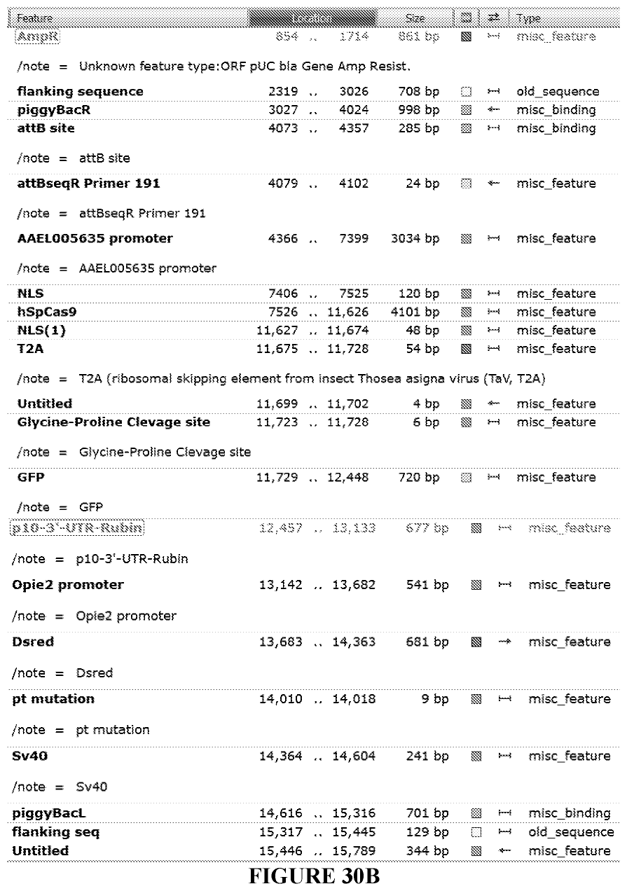

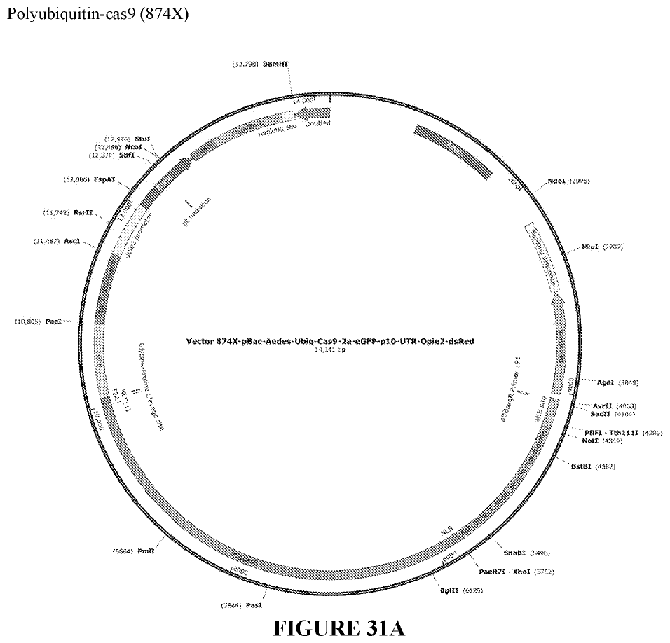

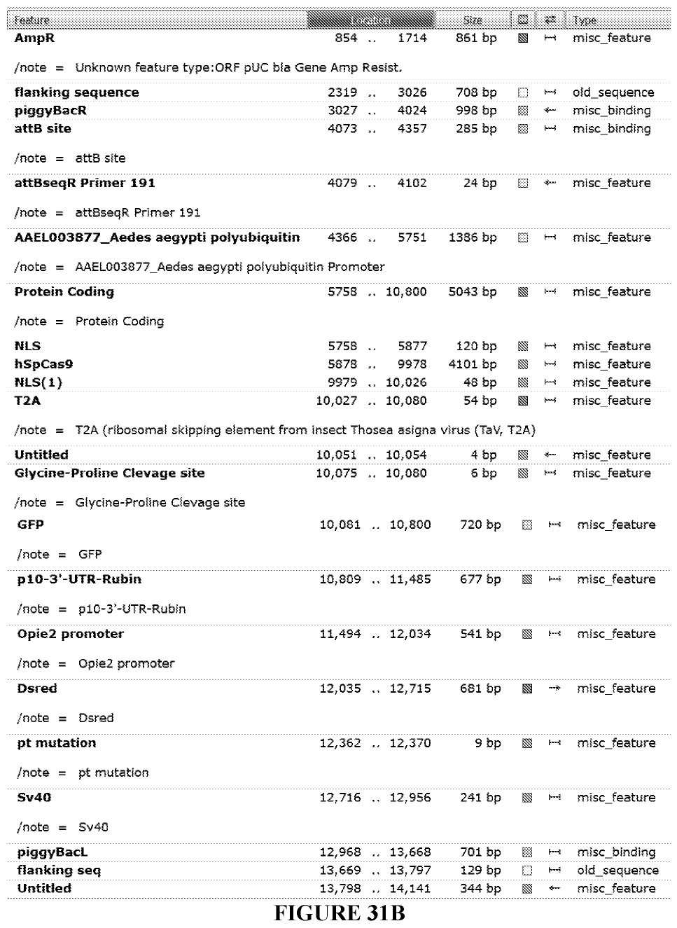

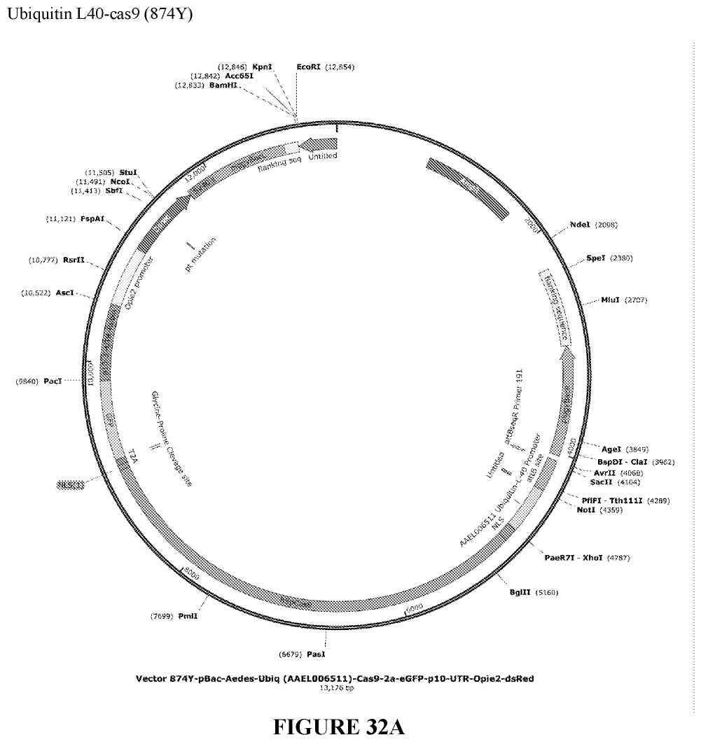

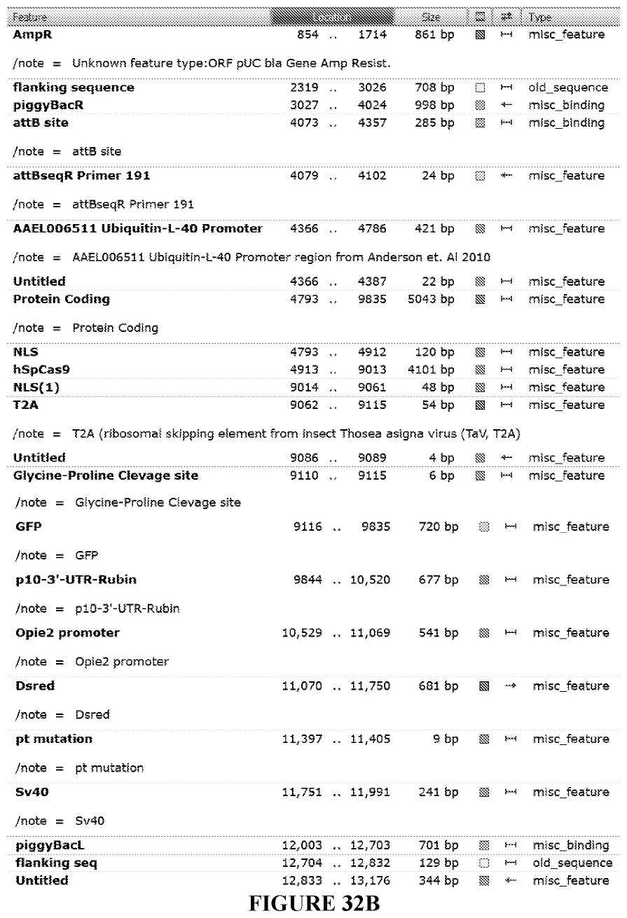

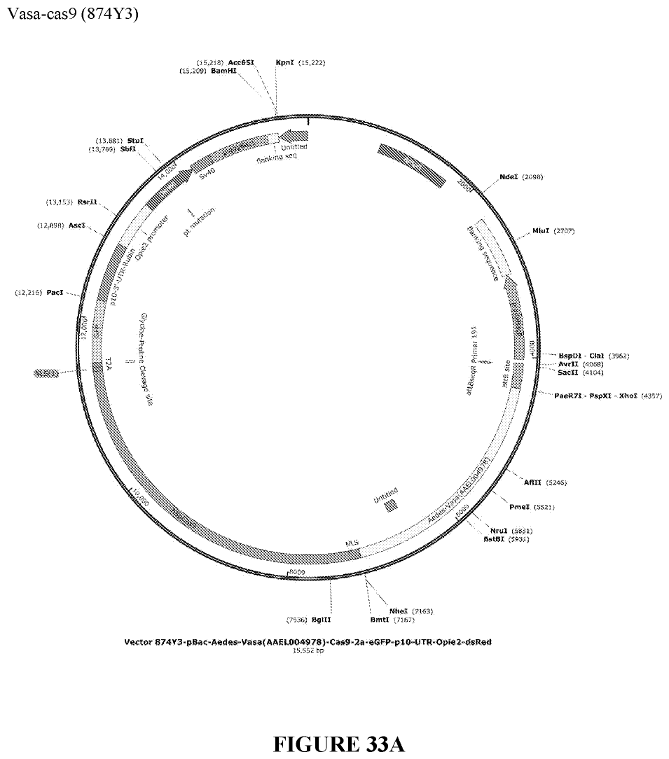

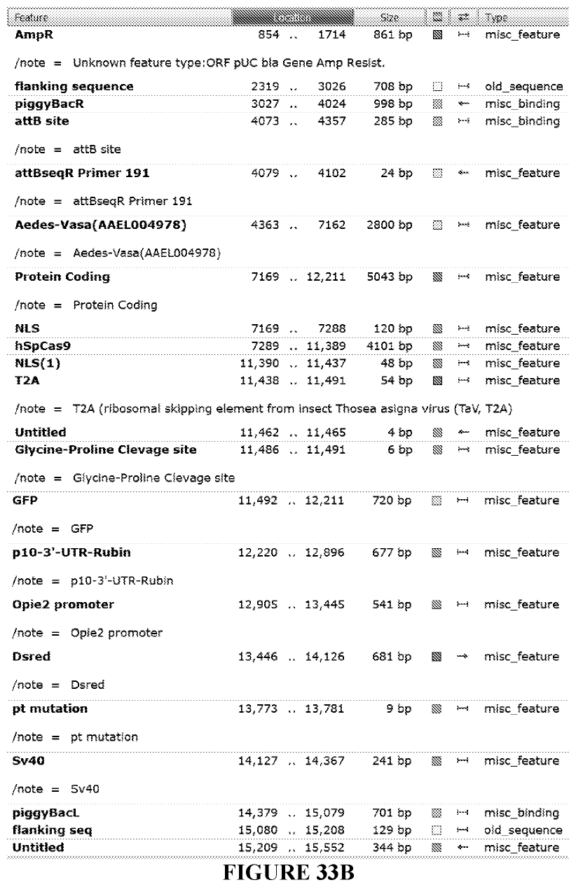

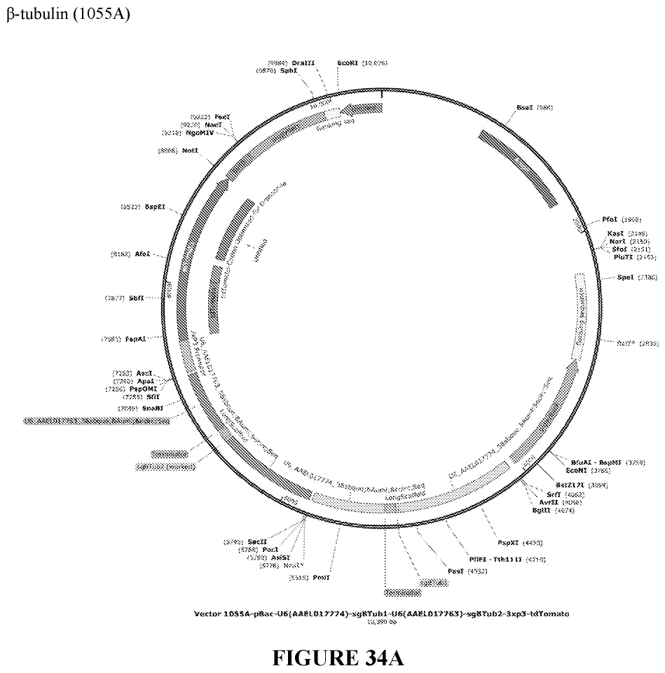

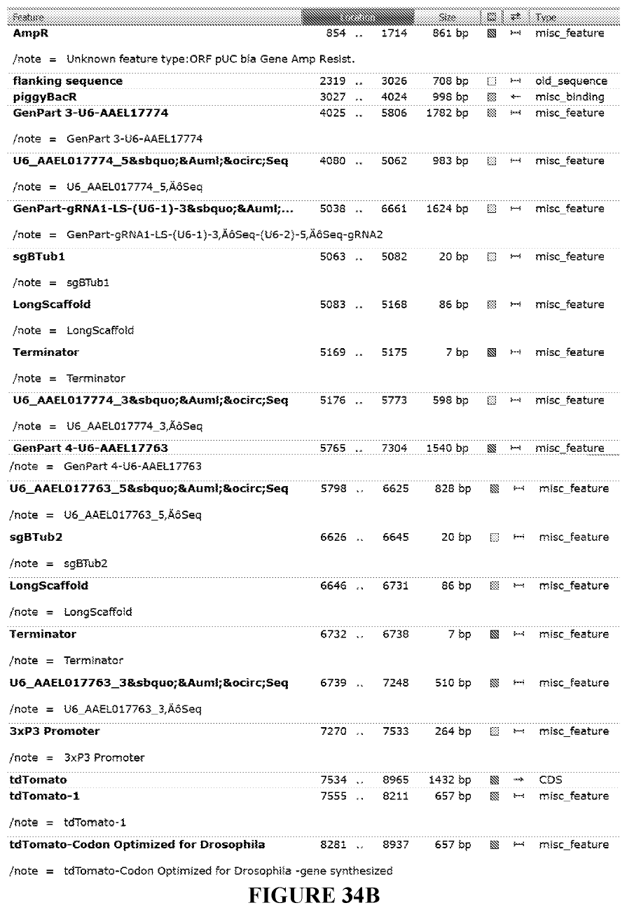

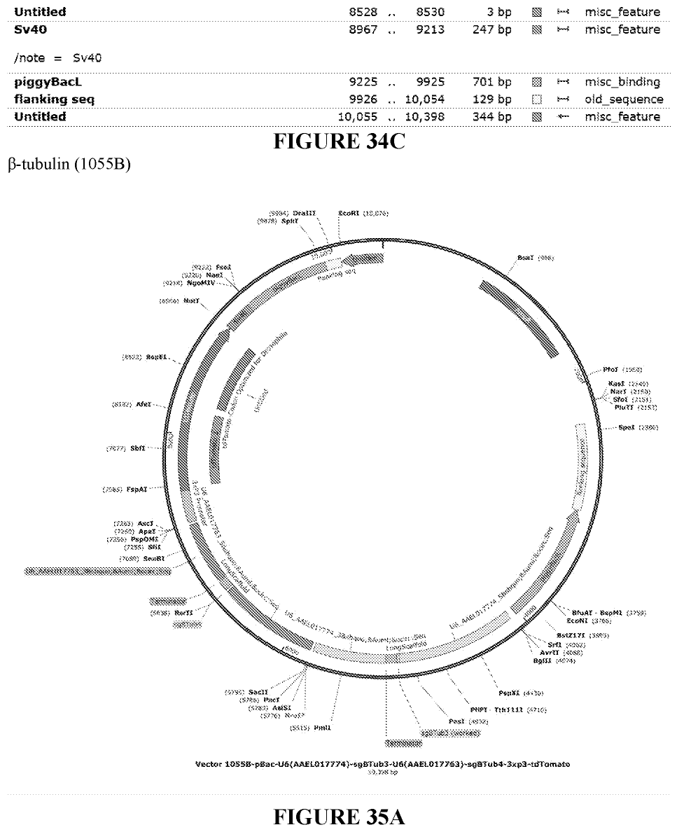

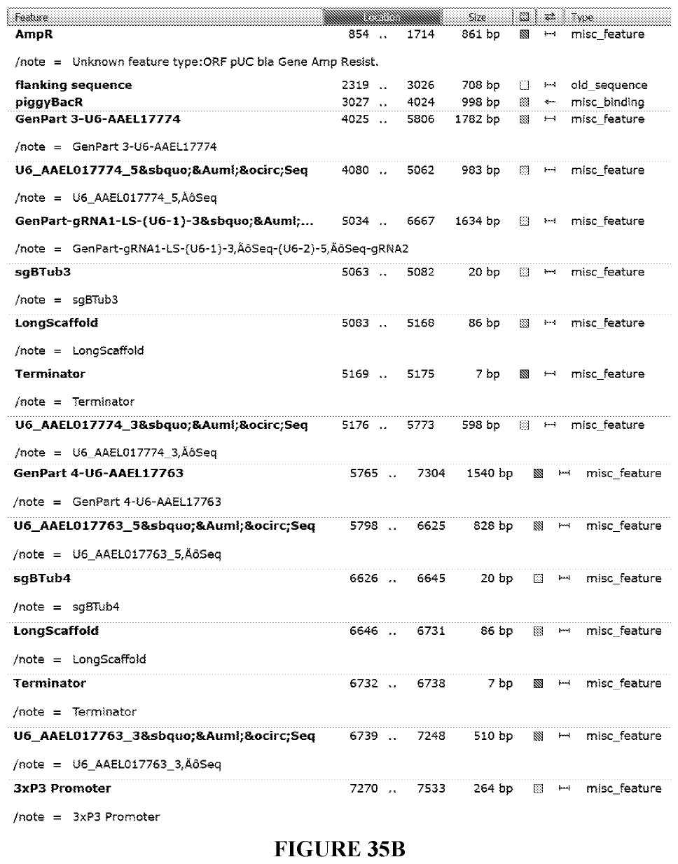

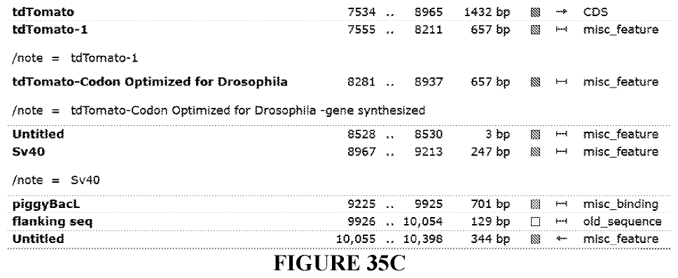

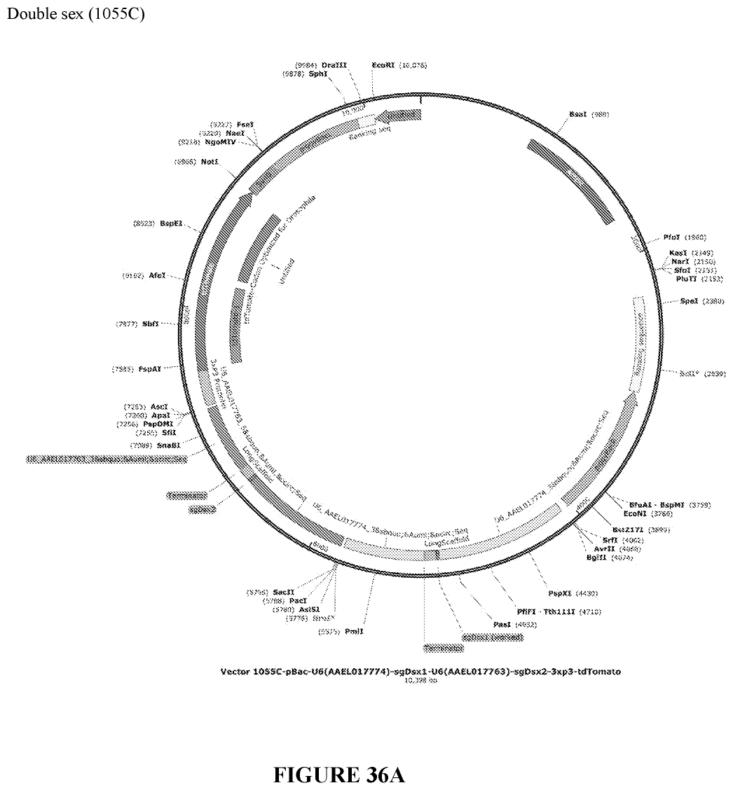

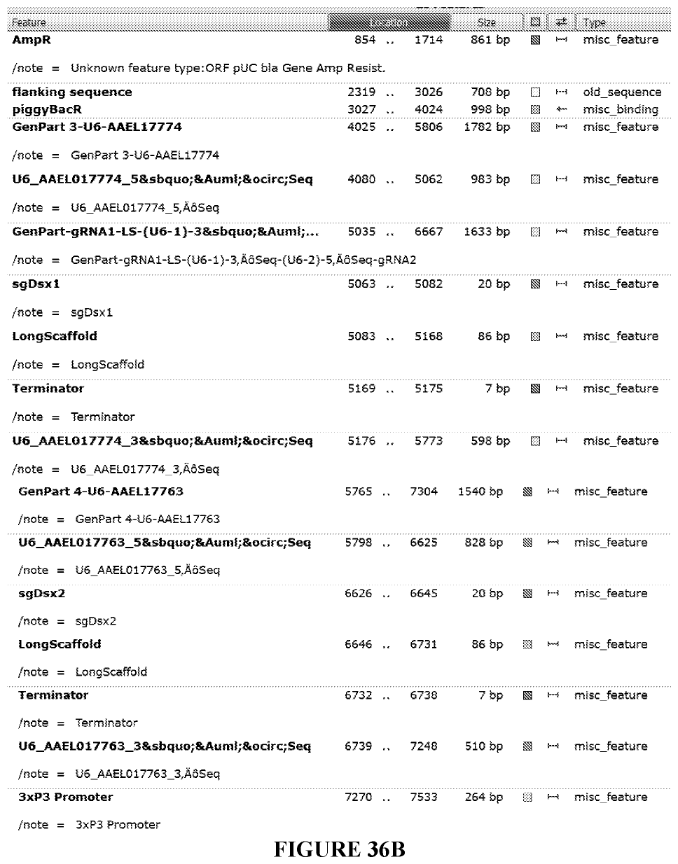

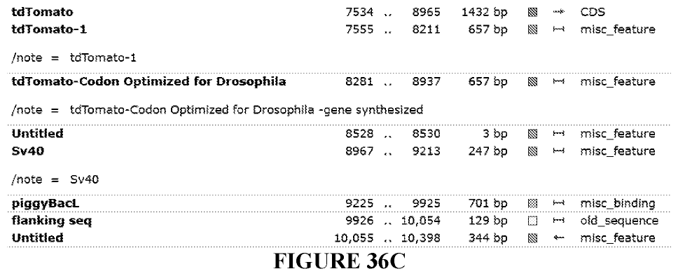

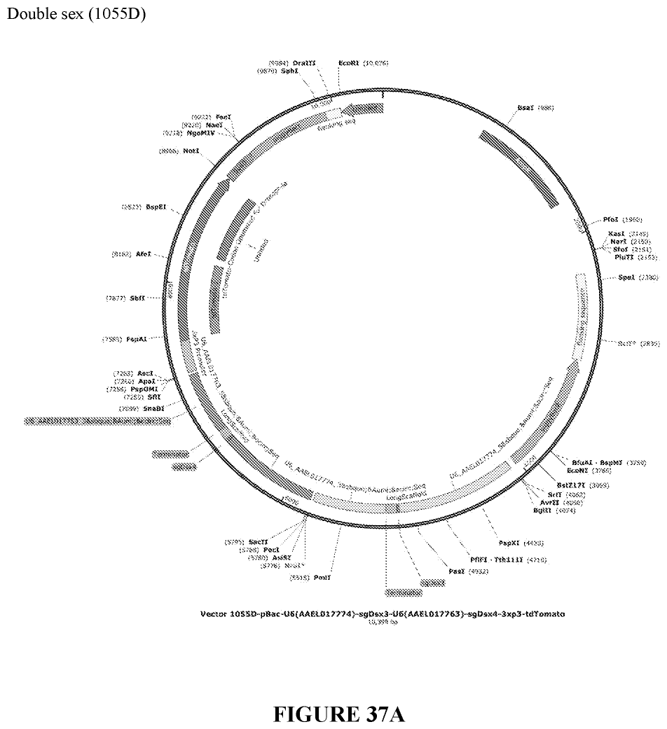

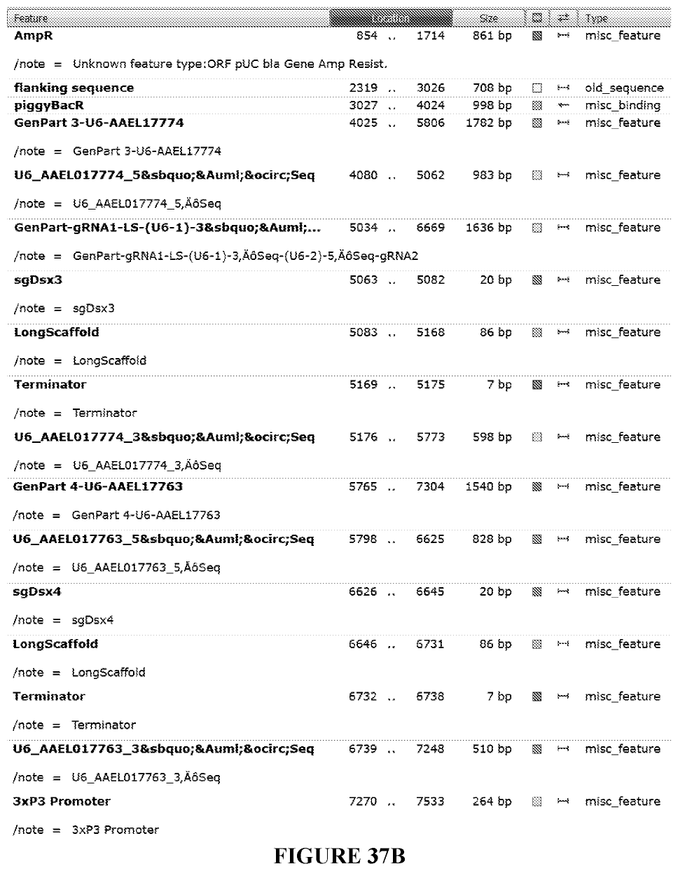

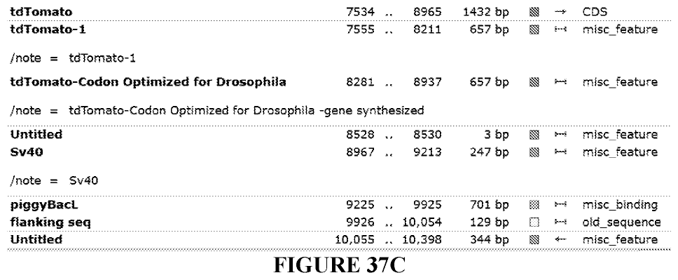

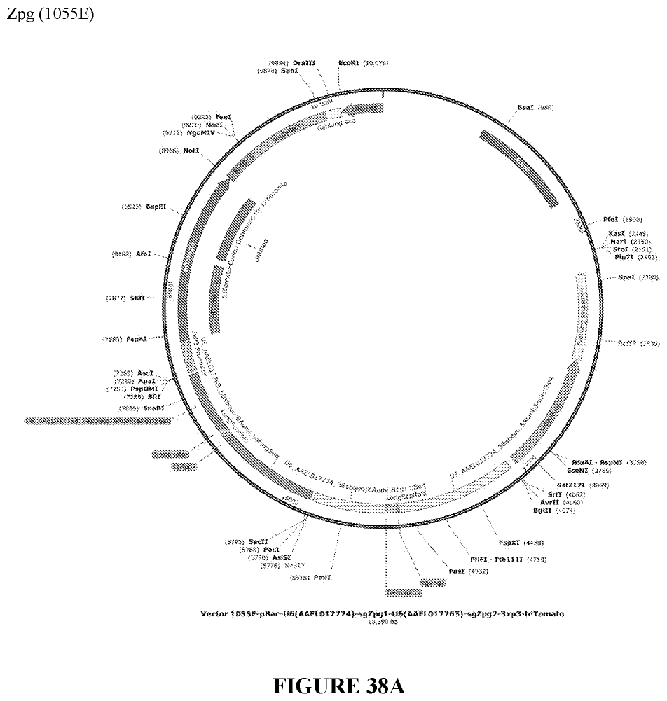

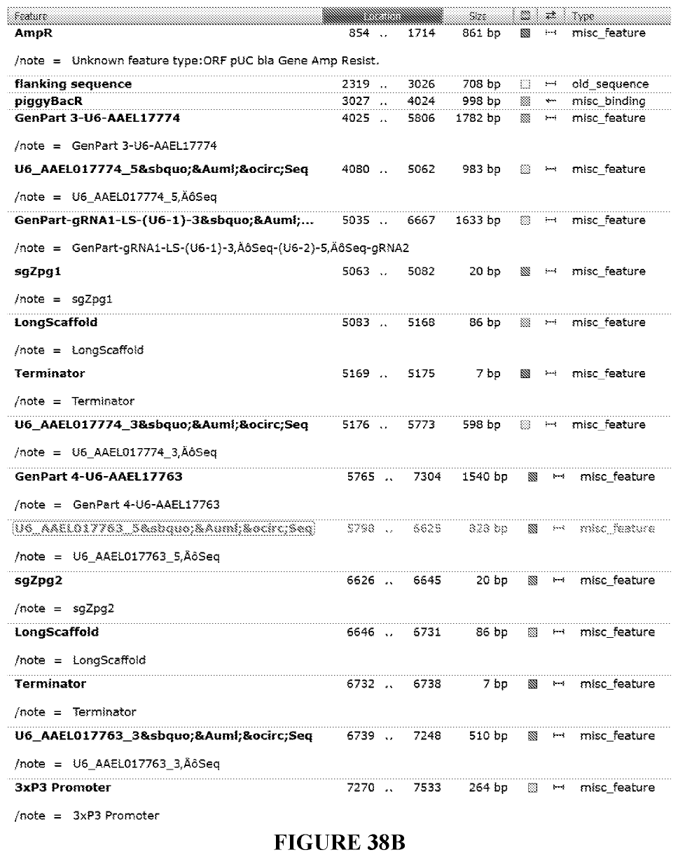

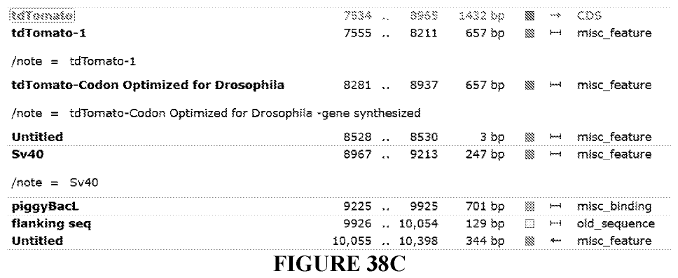

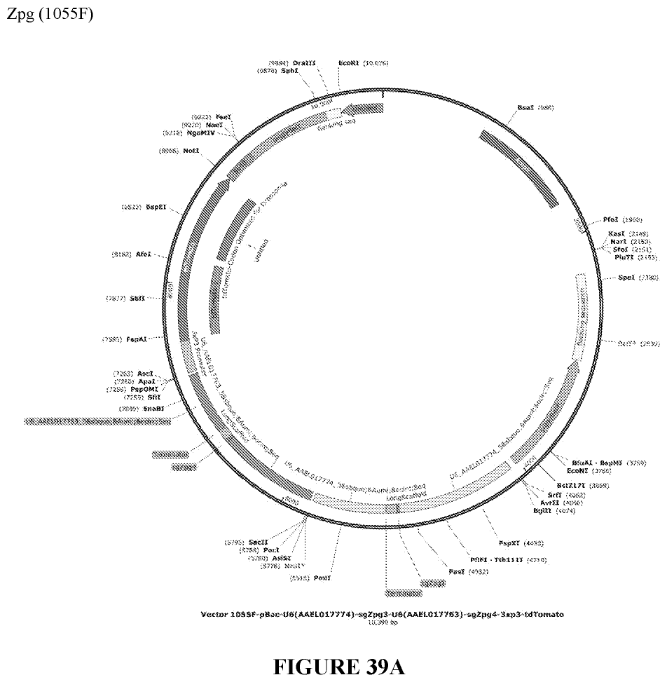

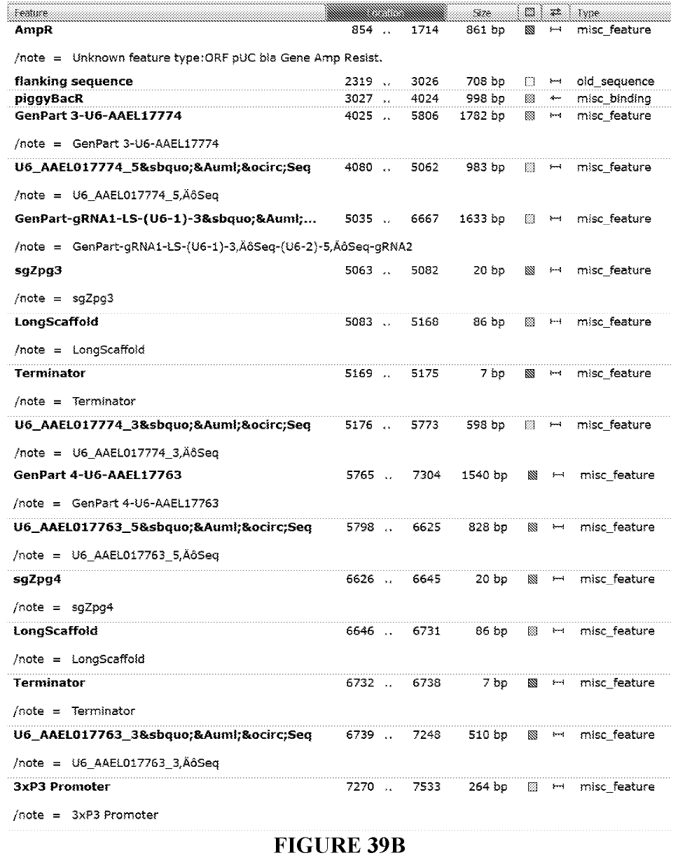

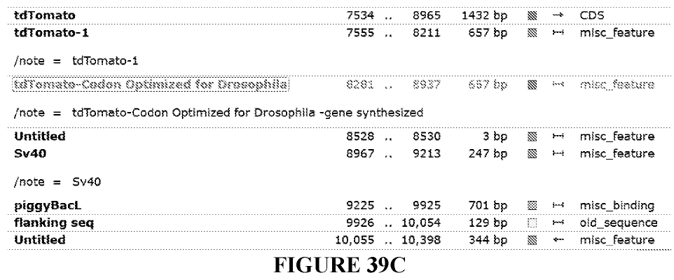

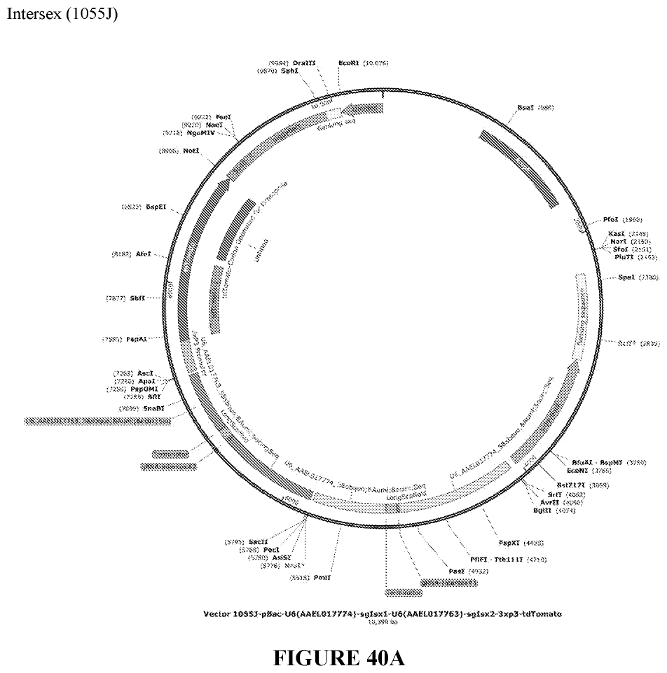

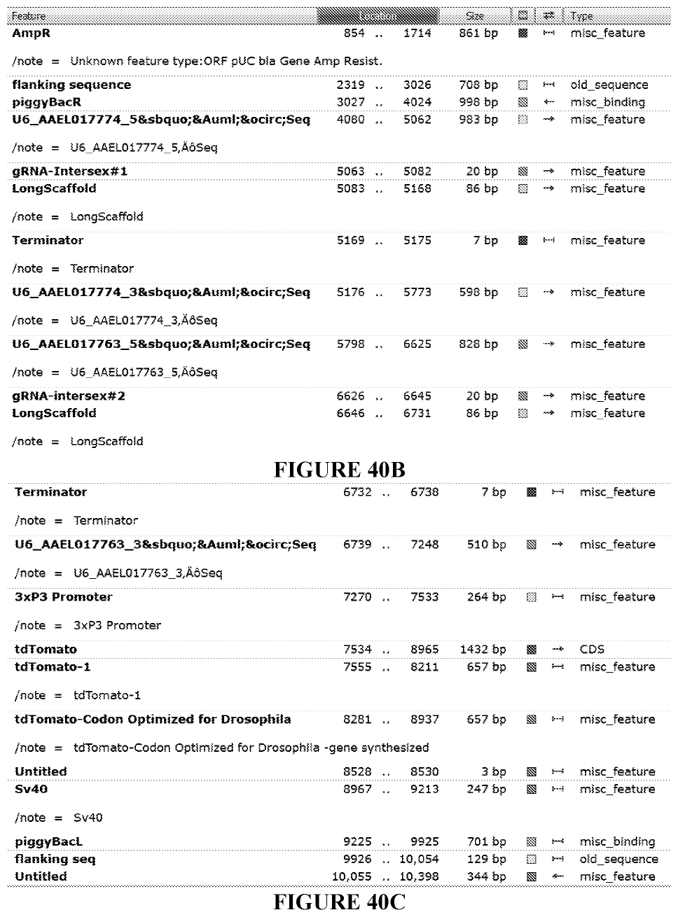

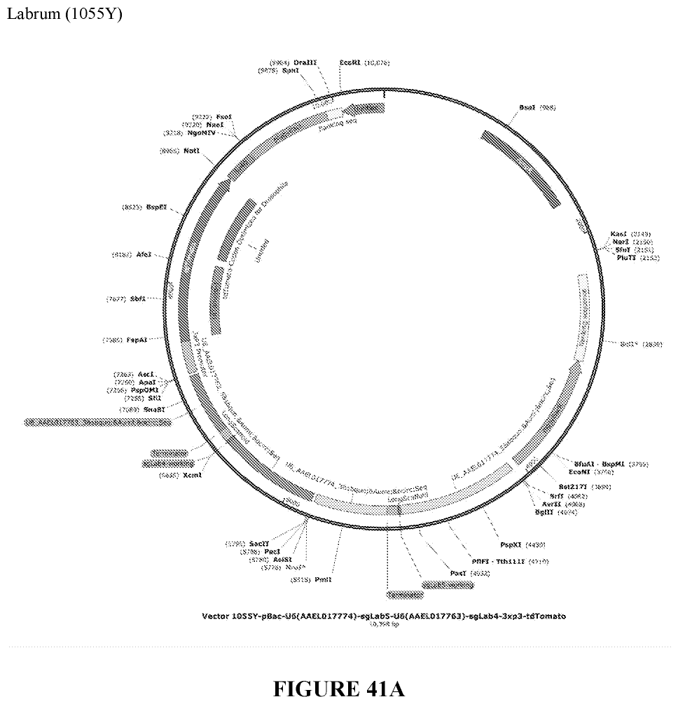

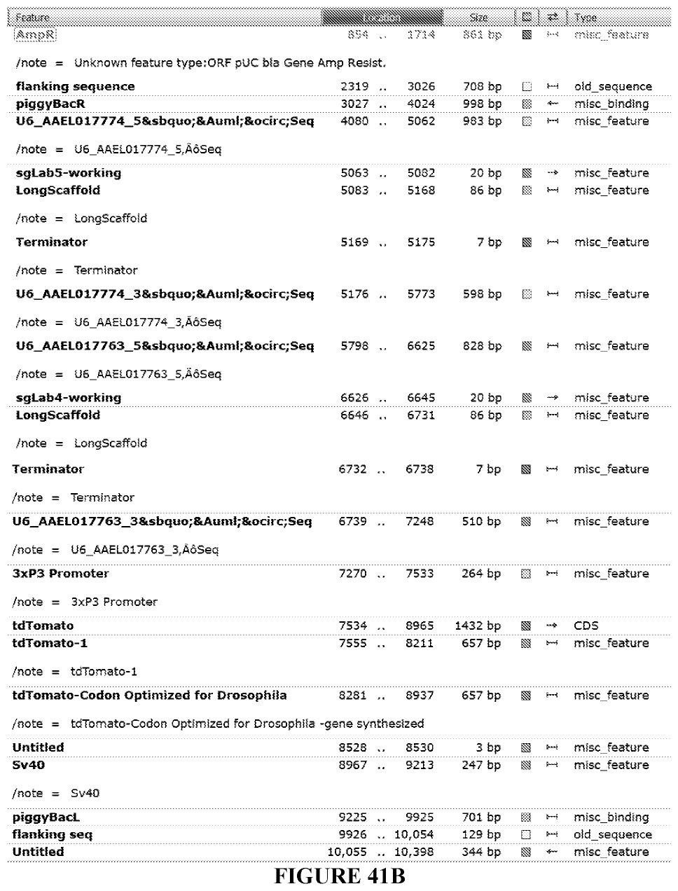

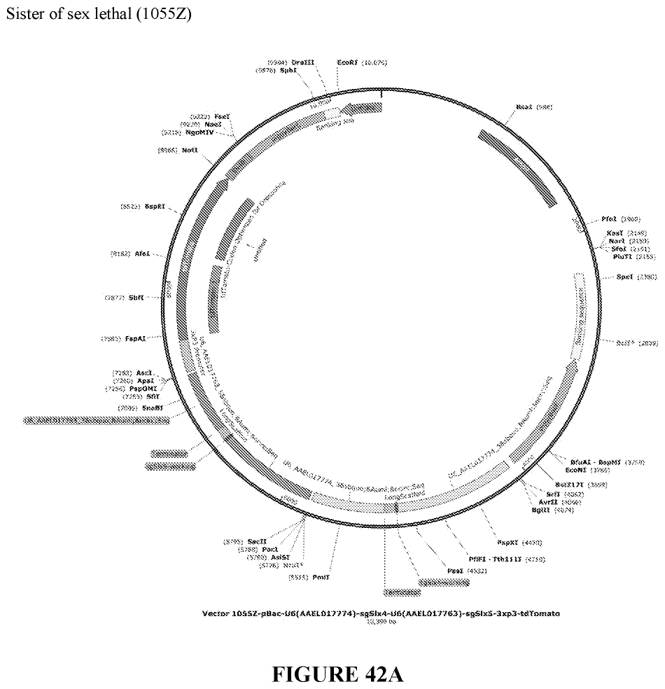

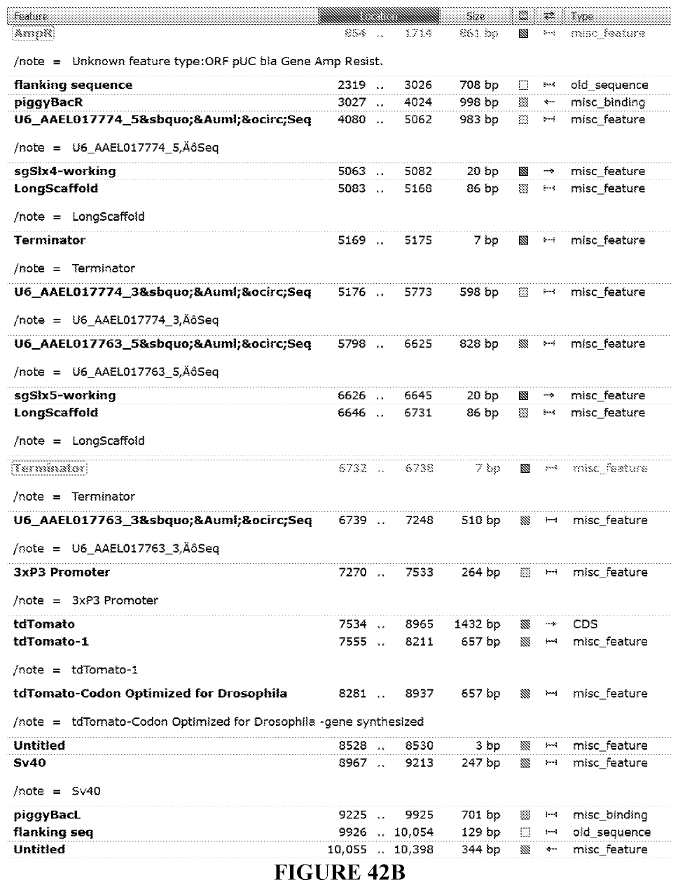

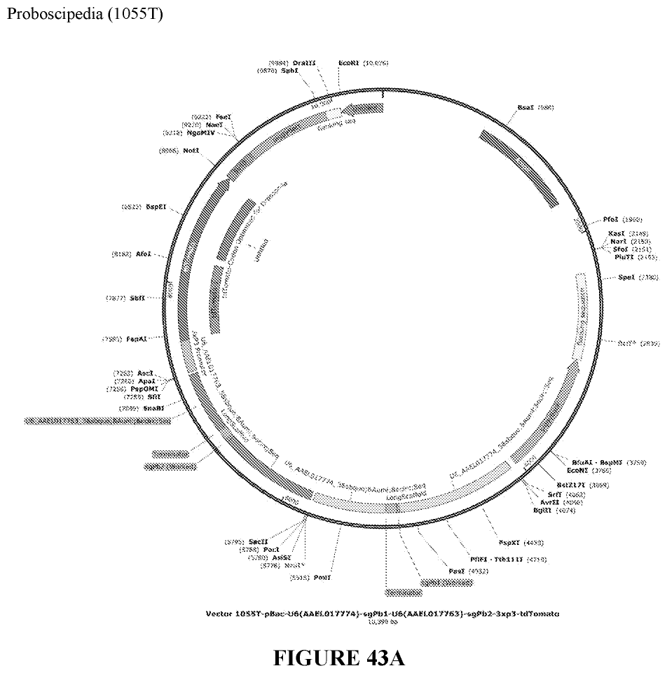

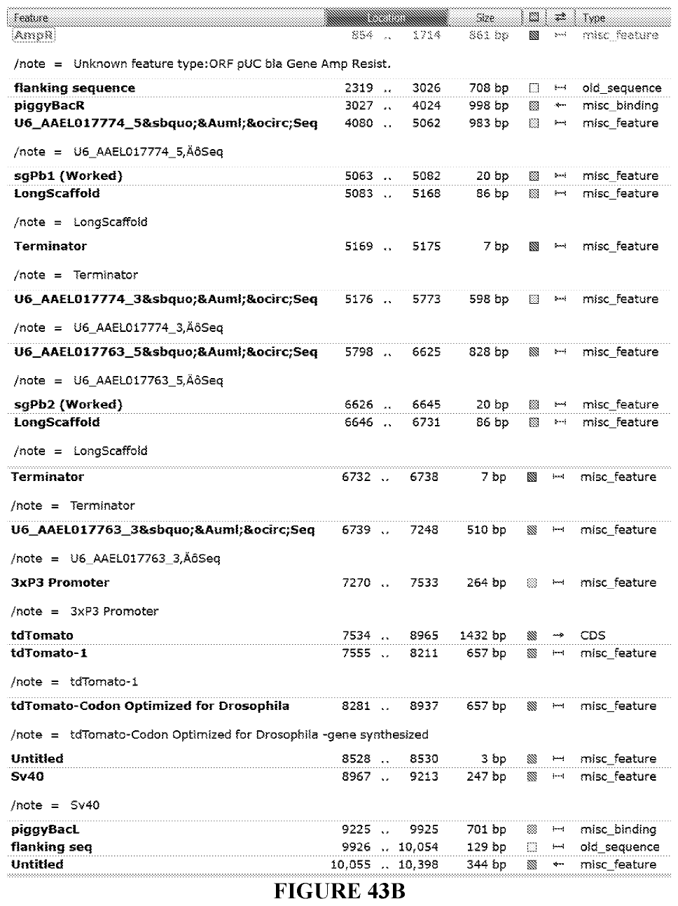

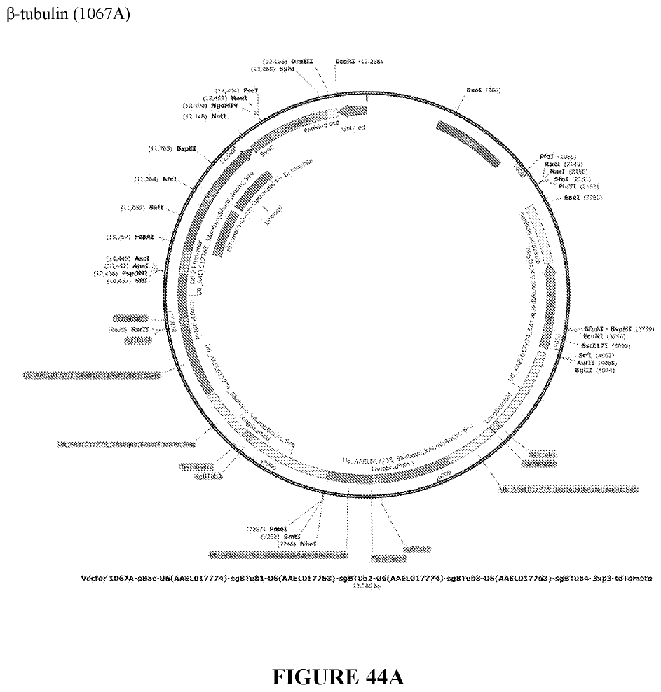

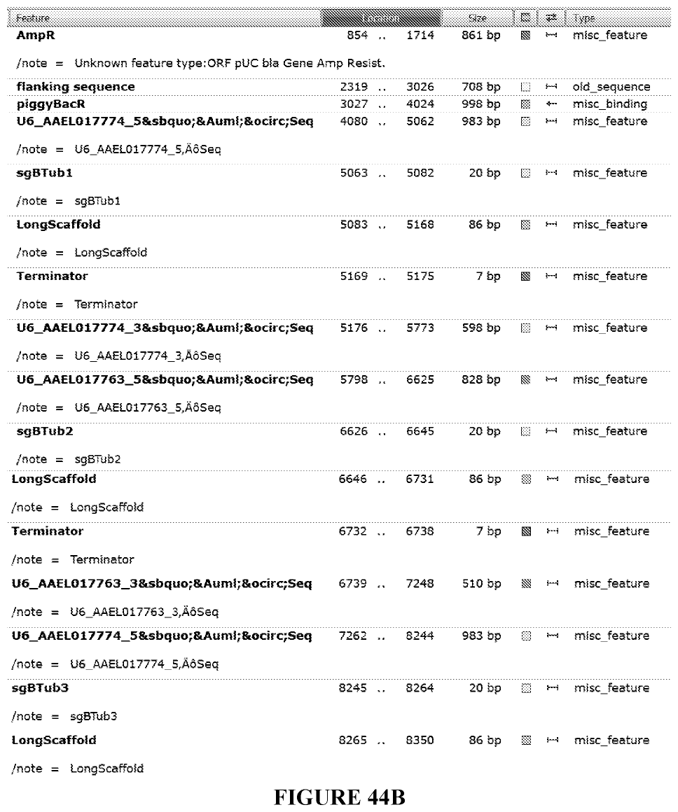

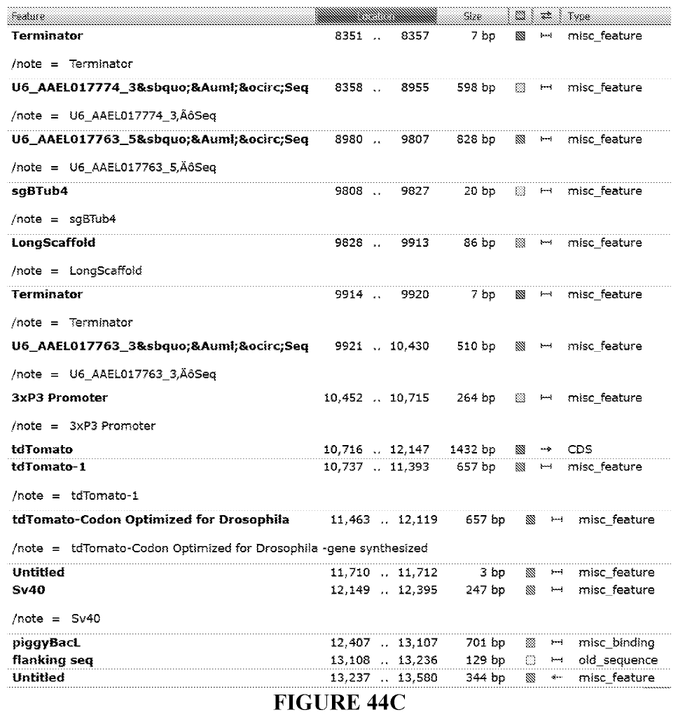

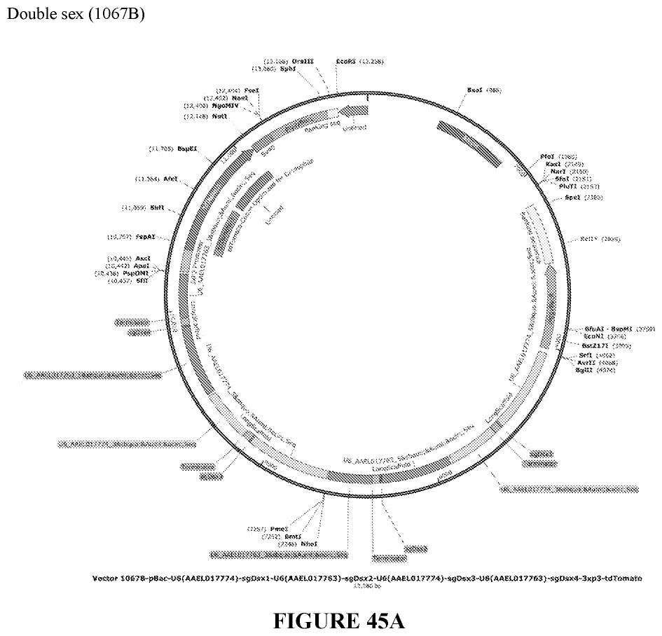

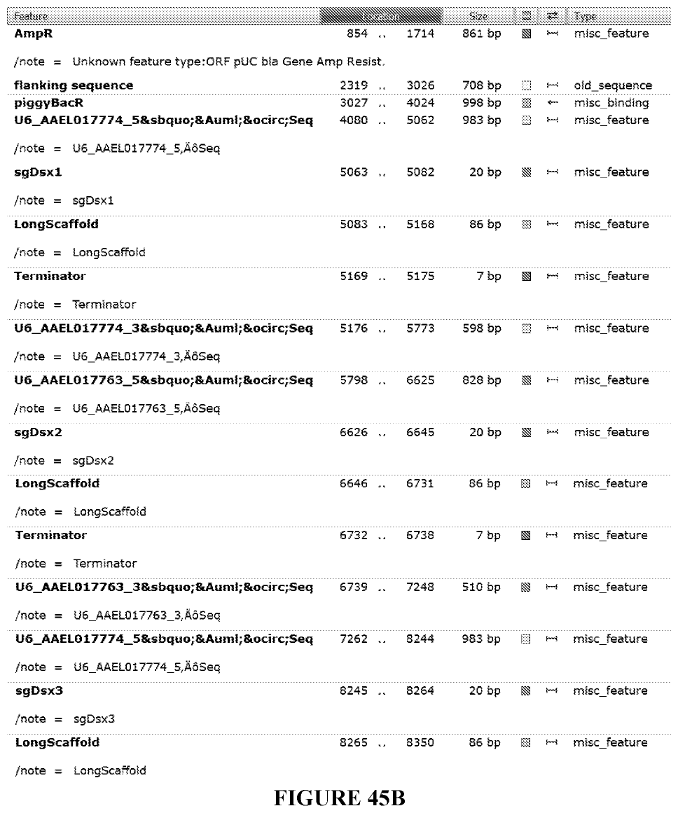

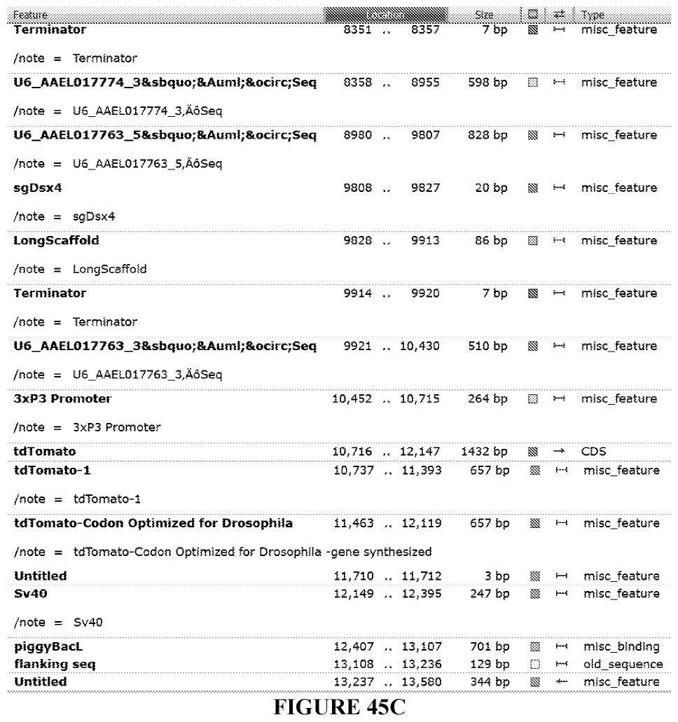

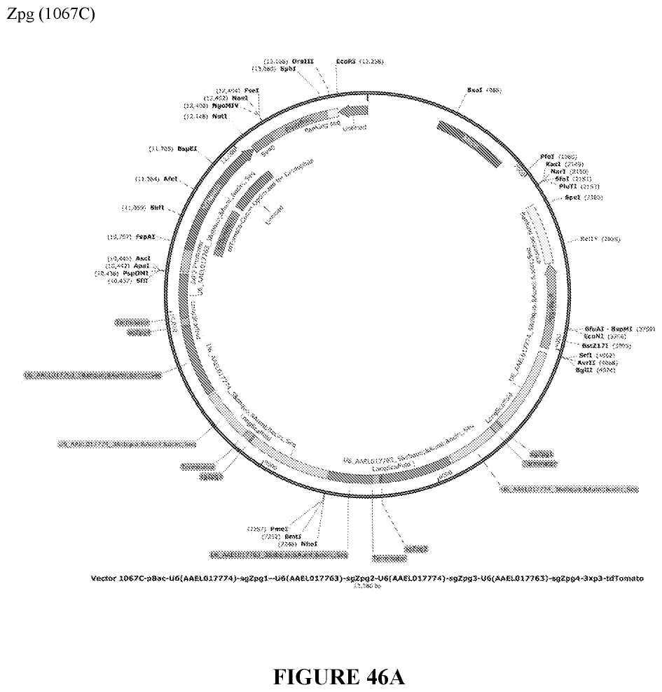

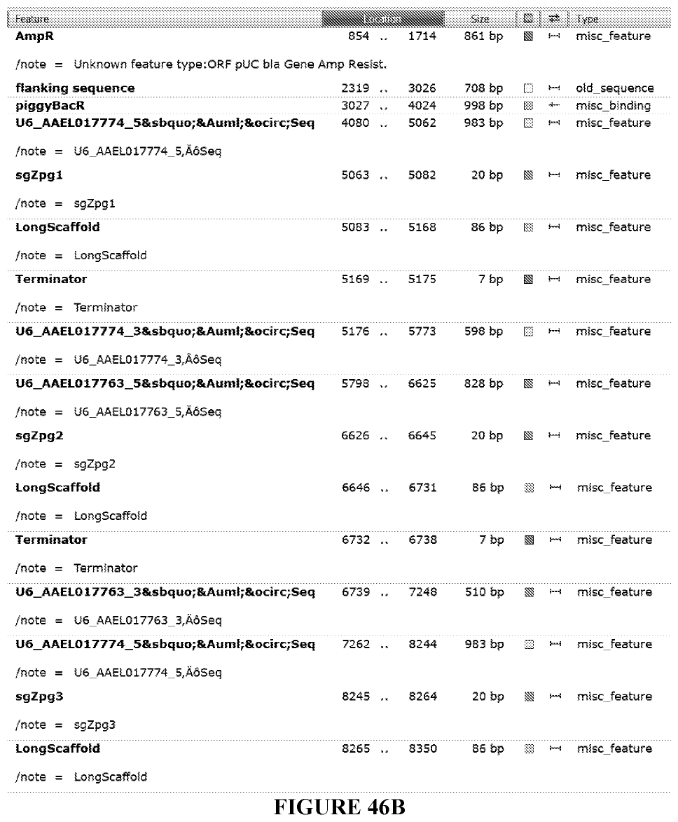

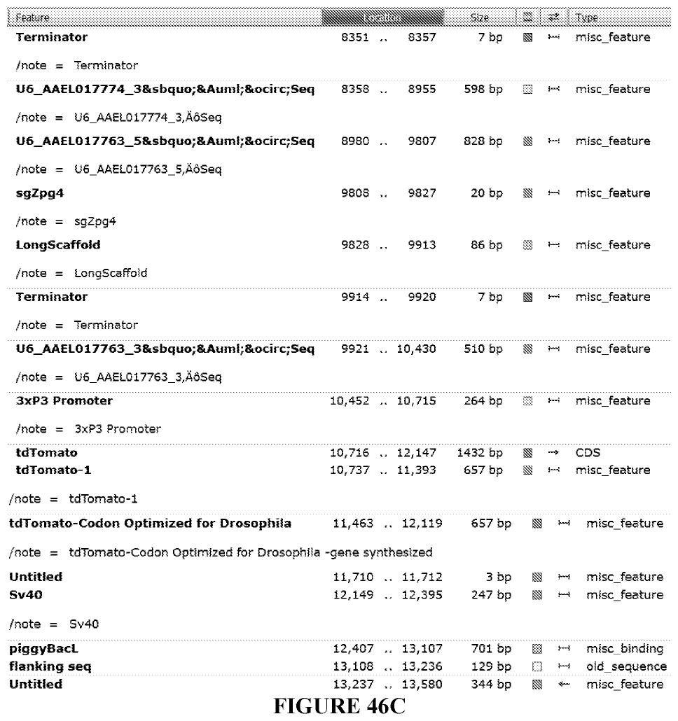

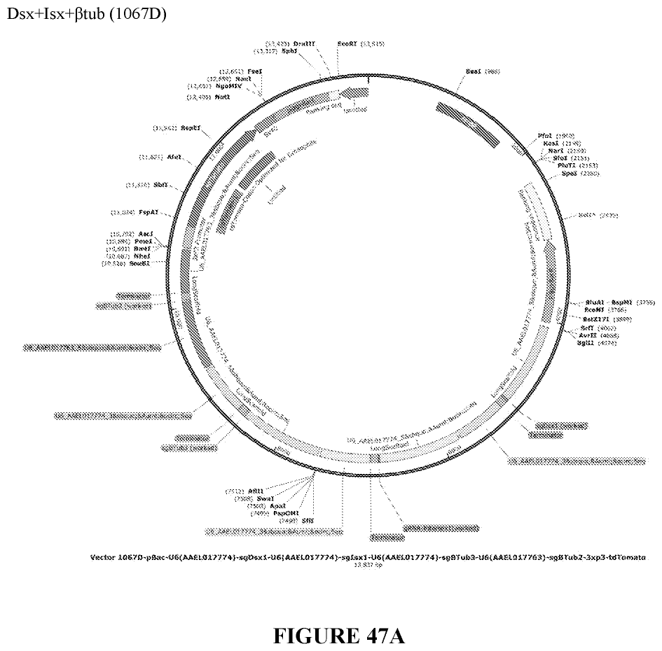

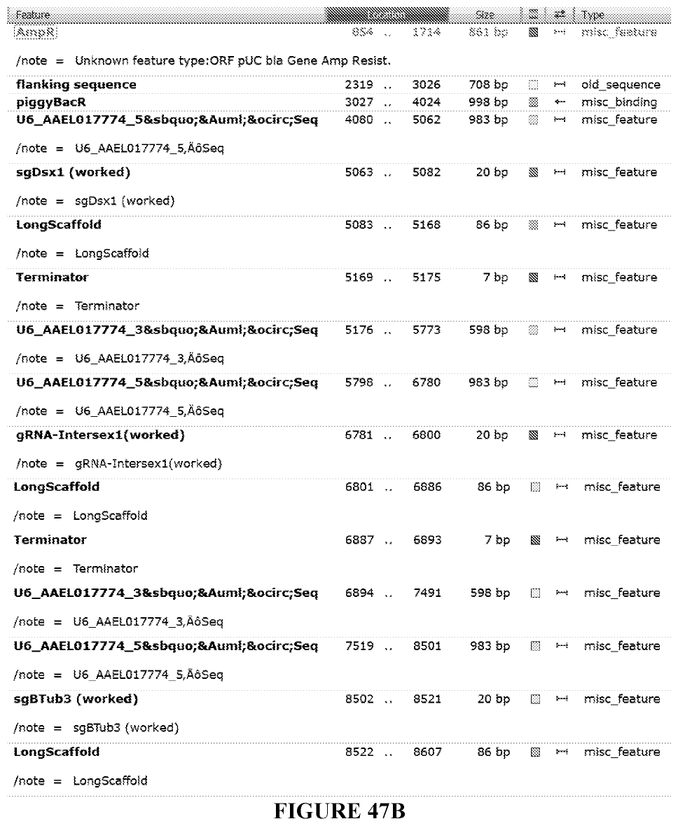

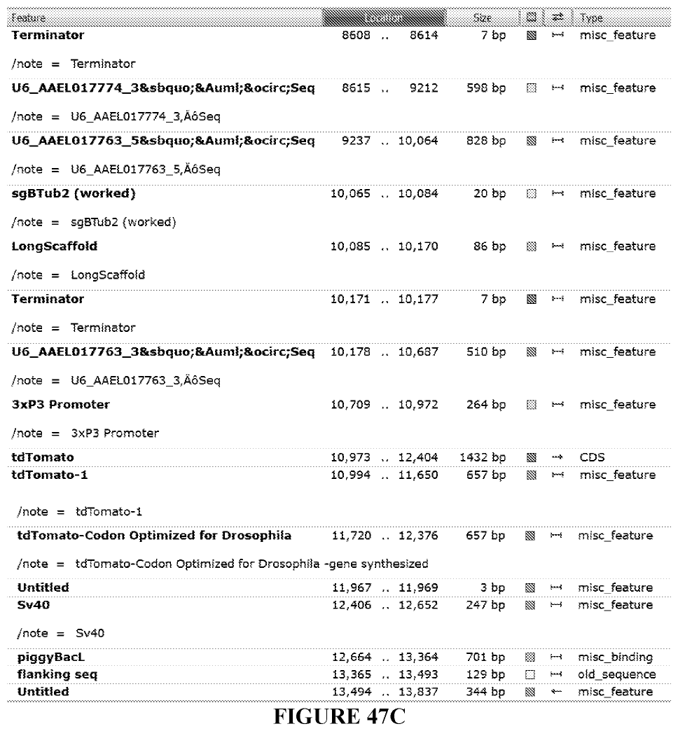

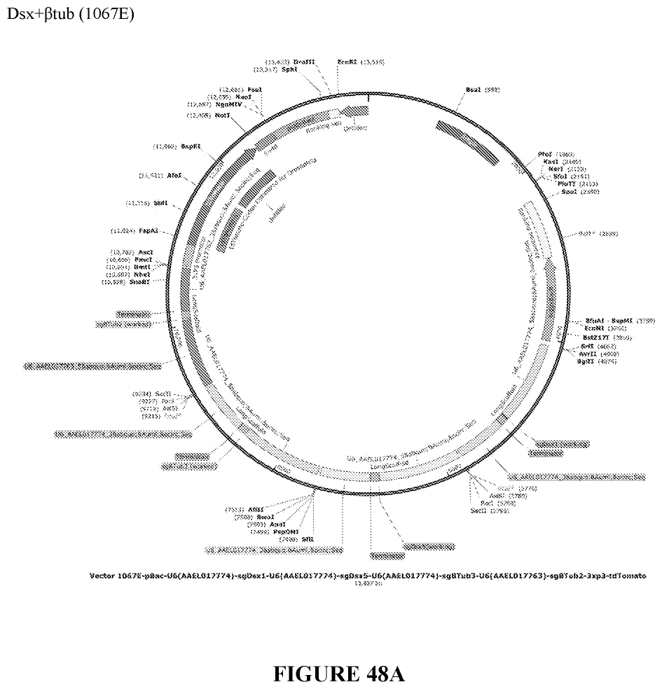

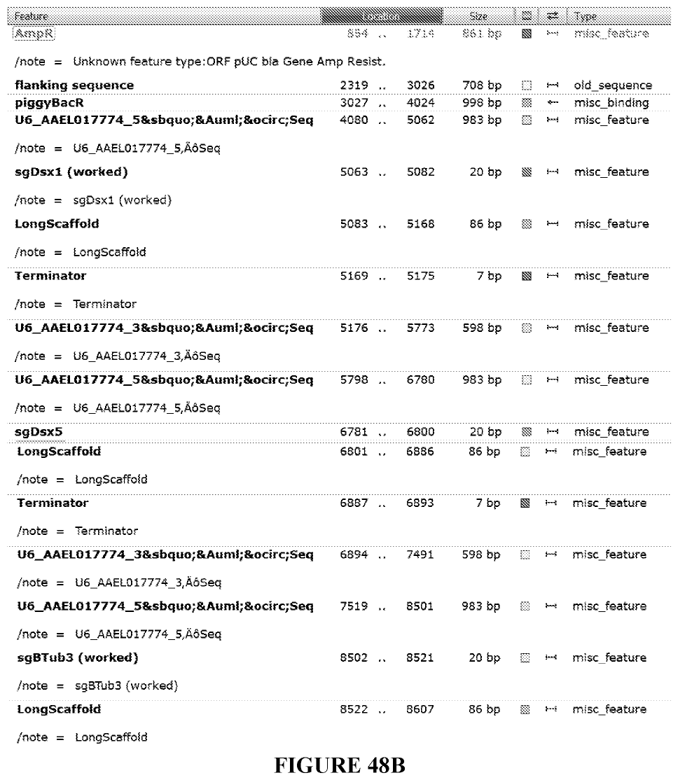

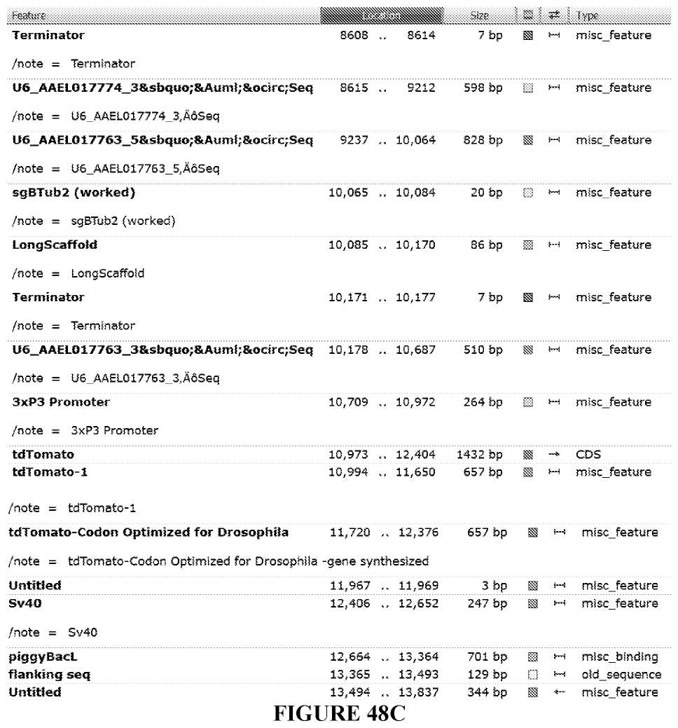

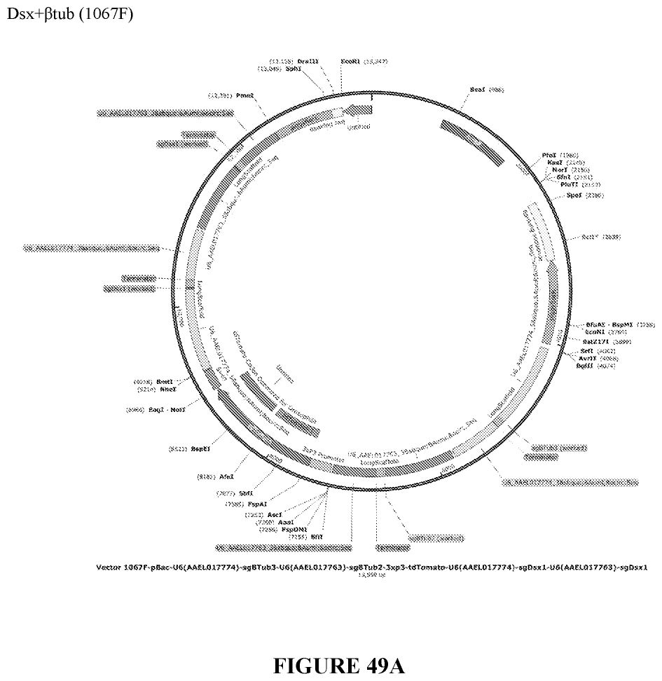

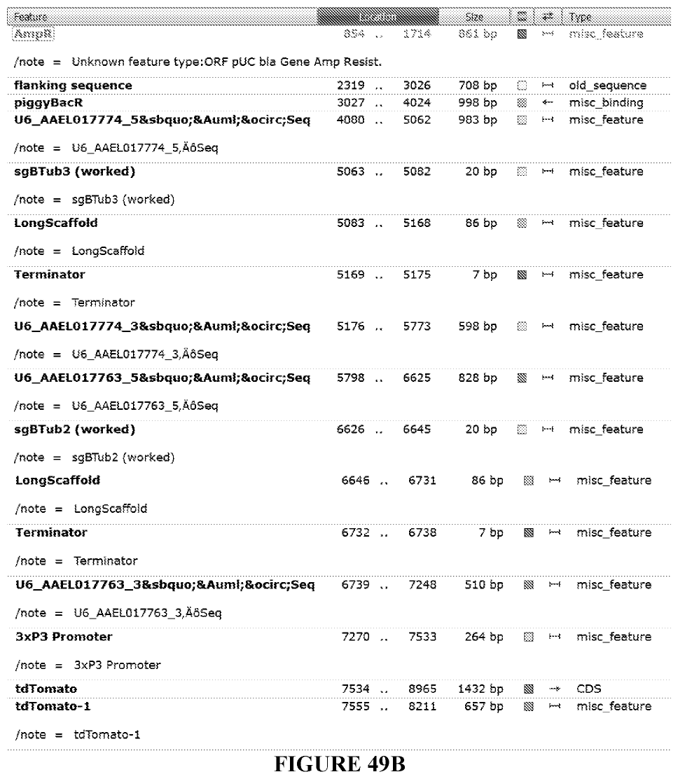

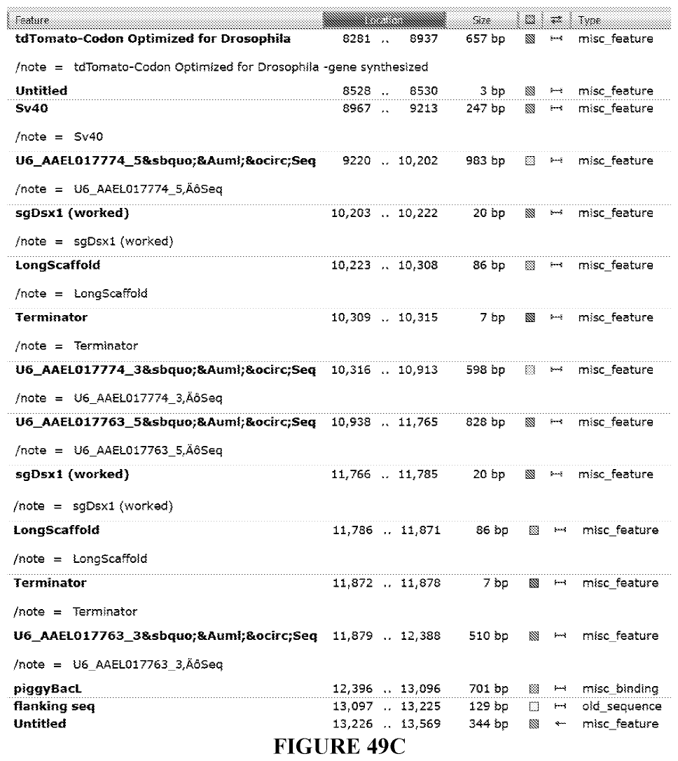

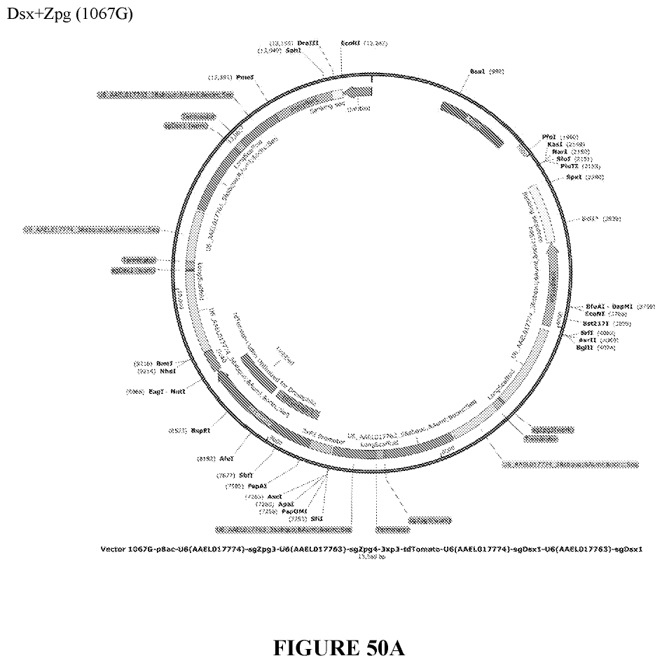

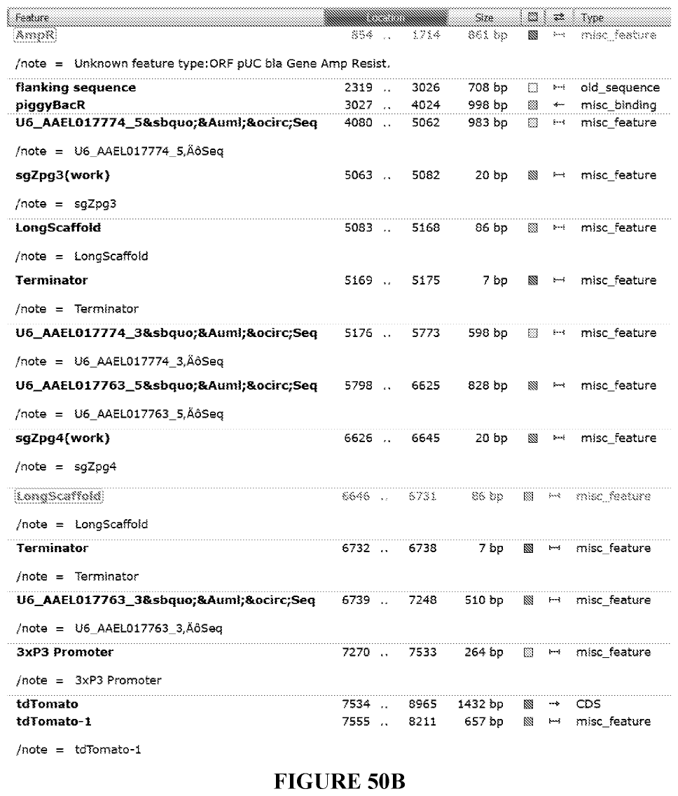

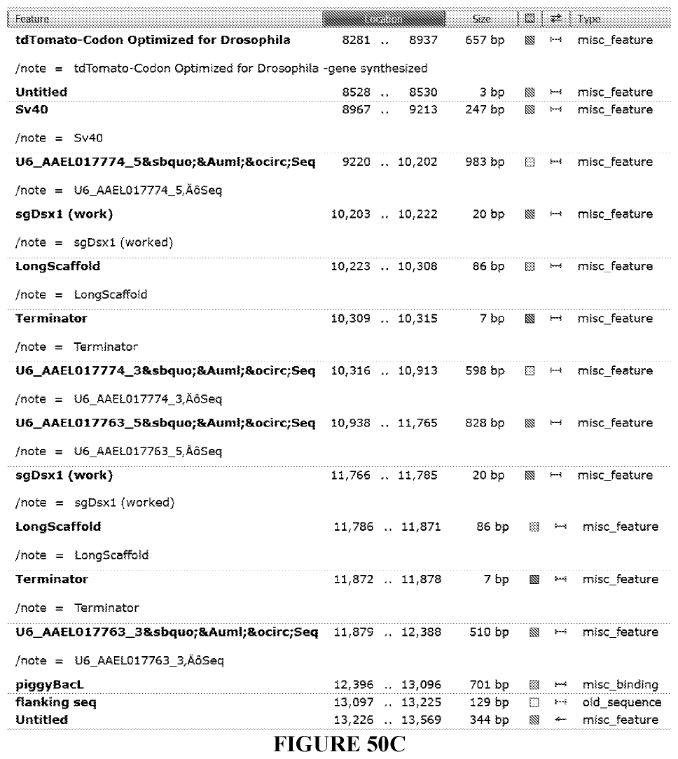

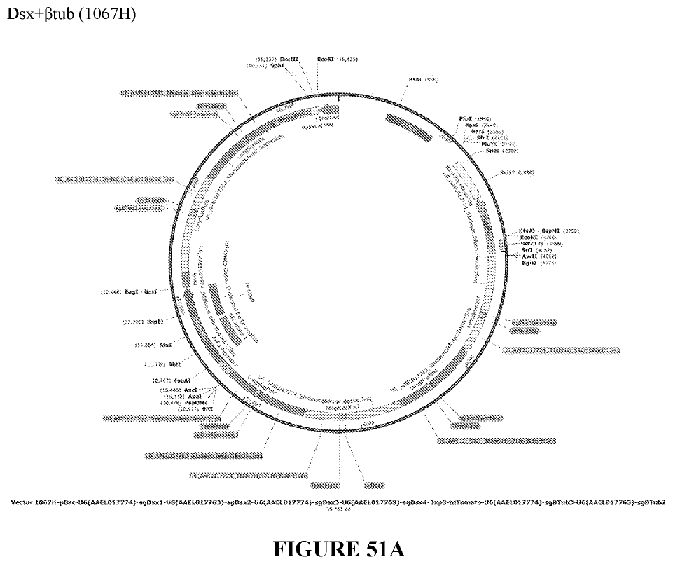

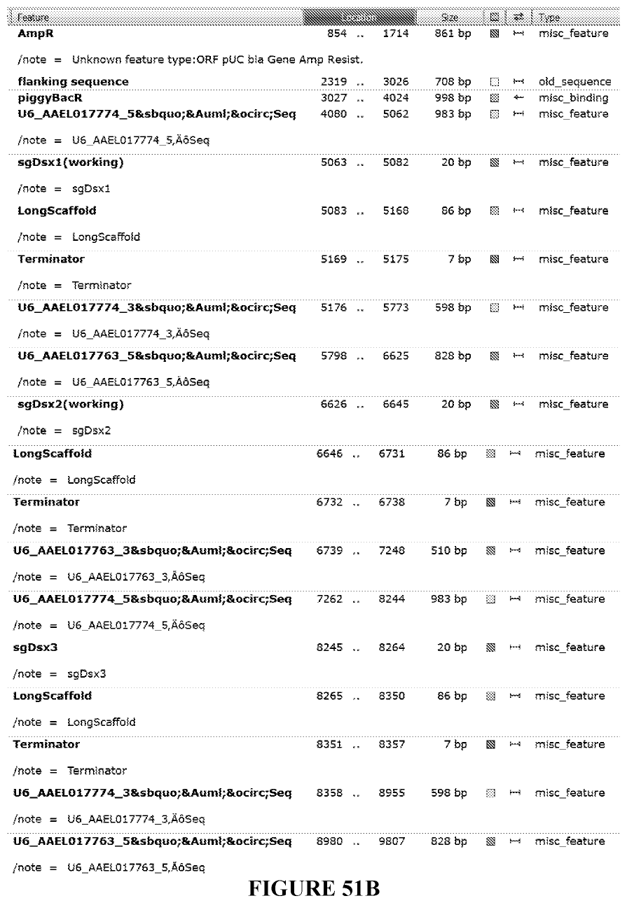

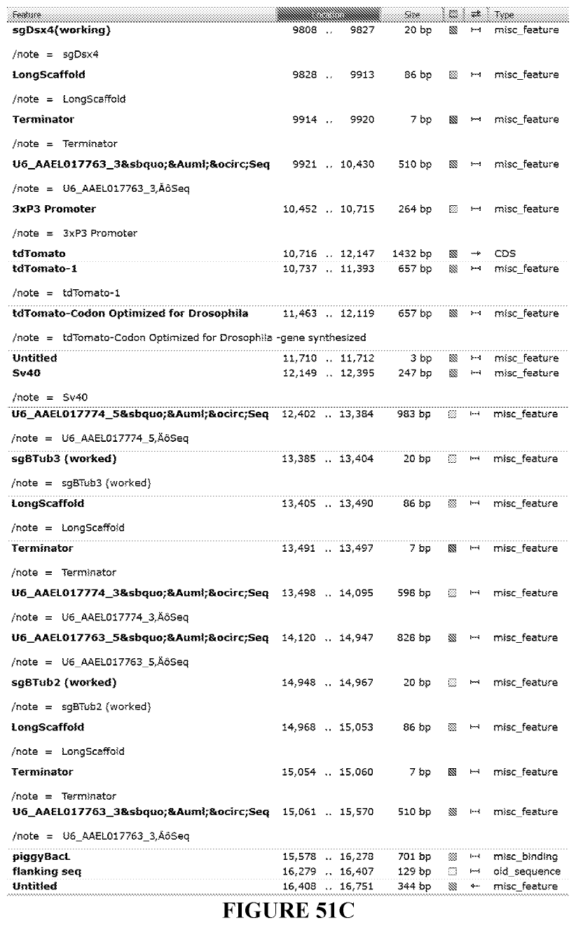

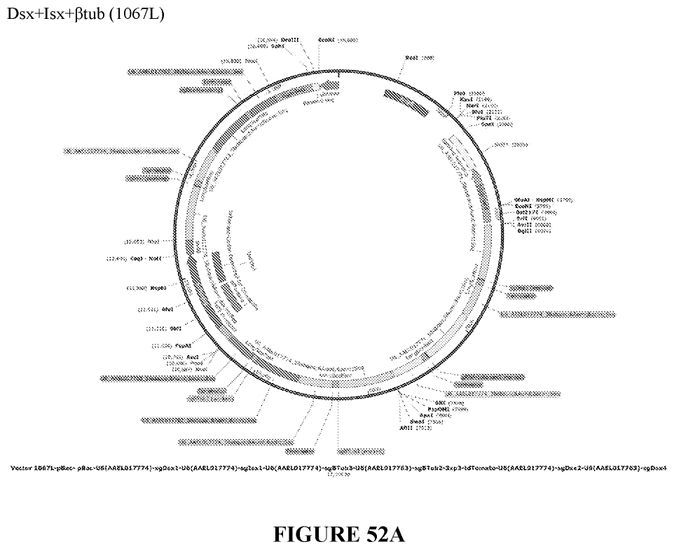

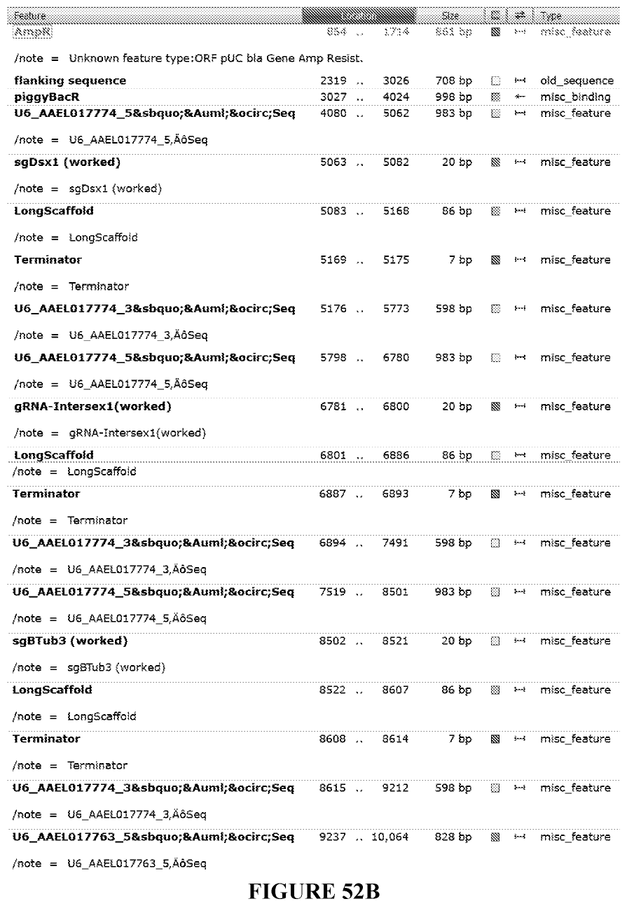

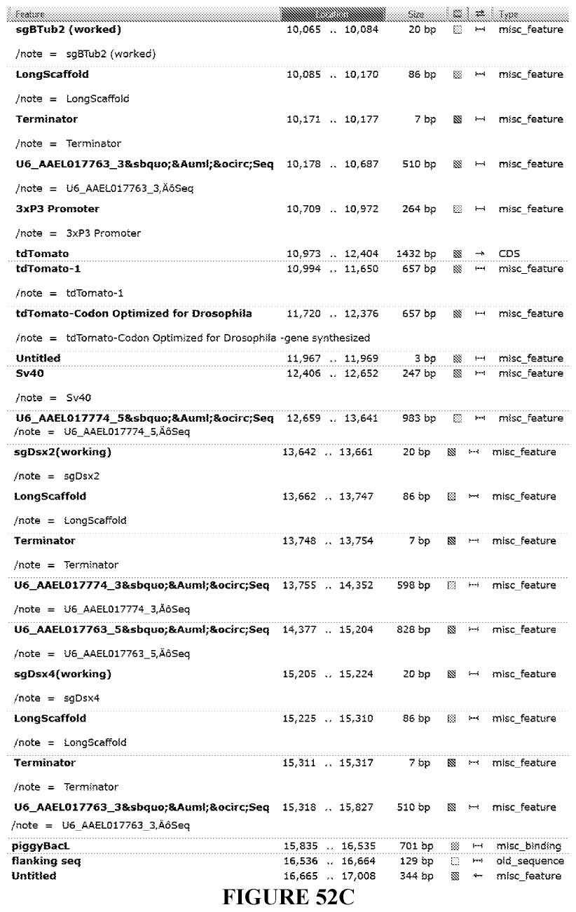

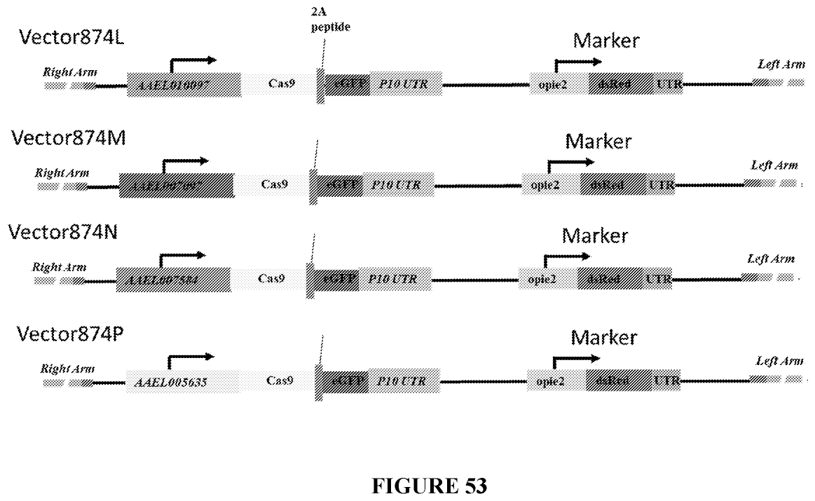

Applicant applied novel genetic sexing and pgSIT technologies to disease vector species, specifically the primary vector of dengue and Zika virus, Ae. aegypti and then adapt this technology to another vector. In preliminary experiments, applicant created six Ae. aegypti lines which endogenously express Cas9 (AaCas9). These AaCas9 lines were shown to facilitate the efficient knock-out of multiple genes when co-injected with short-guide RNAs (sgRNAs) (M. Li et al. 2017) (FIG. 7). These genes were knocked out as single, double or triple mutants making these lines amenable the multi-knock out system disclosed herein. Additional gene disruption experiments with genes known to have other visible phenotypes: ebony, deformed, vestigial, and sine oculus, further demonstrates the robustness of this approach (M. Li et al. 2017). These experiments demonstrate that applicant can generate fit, healthy, homozygous viable and efficient Cas9 strains in Ae. aegypti and applicant can use these strains to target and disrupt more than one gene at a time—a very important prerequisite for the pgSIT systems as disclosed herein. Furthermore, this technology can be edited and adapted for any transgenic competent insect. The resulting technology could revolutionize the field of vector control and mass production of sterile mosquito males to release for suppression of vector-borne diseases worldwide by shipping eggs to remote locations (FIG. 5). This technology is expanded by creating Ae. aegypti lines which express gRNAs to target female essential genes and male fertility genes and then demonstrate that this technology can be scaled and adapted to other mosquito species.

### Engineering Female Lethality

To kill all female progeny, any female essential gene can be targeted with the disclosed approach. As proof of principle gRNAs were used targeting key female-specific sex determination genes in D. melanogaster (WO 2019/103982, Kandul et al. 2018 and Kandul et al. 2019). Applicant targeted female essential genes including sex-specific alternatively spliced sex-determination genes including sex lethal (Sxl, sgRNA^{Sxl}), transformer (tra, sgRNA^{Tra}), or the highly conserved doublesex (dsxF, sgRNA^{DsxF}) gene (WO 2019/103982, Kandul et al. 2018 and Kandul et al. 2019). Sex determination in insects is an evolutionarily conserved phenomenon requiring key-regulatory genes: sxl, tra, and dsx (Sanchez 2008; Price, Egizi, and Fonseca 2015b; Bopp, Saccone, and Beye 2014). In Drosophila, sex-specific alternative splicing of sxl induces a splicing cascade of the downstream tra and dsx genes leading to production of female and male specific versions of Dsx proteins (dsx^{F} and dsx^{M}) that cause further activation of sex-specific genes. Notably, the cues that initiate a sex-specific alternative splicing of sxl may vary, even in related insects, while the core sex-determination pathway, sxl, tra, and dsx, are highly conserved among insects and other invertebrates. In Ae. aegypti, a dominant male-determining factor (Nix) activates male specific splicing of the same key genes (Hall et al. 2015); therefore it is highly likely that similar results can be achieved in this species. Transient knockdown of the dsx in Ae. aegypti during blood feeding resulted in reduced fertility in male offspring (Whyard et al. 2015). The dsx gene has also been linked to male fertility in other mosquito vector species (Price, Egizi, and Fonseca 2015a; Scali et al. 2005), so this gene may be a suitable target across many vector species. In fact, a recent study demonstrated that dsx knockouts in Anopheles gambiae could consistently generate sterile or intersex females (Kyrou et al. 2018). However, while there are many similarities between the D. melanogaster and Ae. aegypti sex determination pathways, their differences will warrant examination of multiple candidate female lethality genes. For example, the tra homologue, required for fruitless (fru), a sex-linked behavior gene, is not present in Ae. aegypti, but transformer 2 (tra-2), a member of the transformer splicing complex, is present (Salvemini et al. 2013; Nene et al. 2007). In fact, tra-2 knock down in tephritid flies causes masculinization of females (Sarno et al. 2010), while transient knock-down of tra-2 in Ae. aegypti caused female lethality (Hoang et al. 2016). Moreover, the Ae. aegypti dsx and fru orthologous have sex specific splicing (Salvemini et al. 2013, 2011). Therefore, tra-2 and potentially dsx are excellent candidates for the approach, but they need to be evaluated along with other potential genes. Finally, many D. melanogaster genes that conserved in Ae. aegypti have interesting female-specific loss-of-function phenotypes; for example, hermaphrodite, intersex, virilizer, and etc. These genes can also be tested one gene, or multiple genes, at the time to cause Ae. aegypti female-specific lethality or masculinization.

### Engineering Male Sterility

In the pgSIT, any male fertility gene can be targeted to create sterile male progeny. In the proof of concept studies conducted with D. melanogaster, gRNAs to disrupt male fertility were designed, including genes active during spermatogenesis, such as βTubulin 85D (βTub, sgRNA^{βTub}), fuzzy onions (fzo, sgRNA^{Fzo}), protamine A (ProtA, sgRNA^{ProtA}), or spermatocyte arrest (sa, sgRNA^{Sa}). Many of these genes a have homolog in Ae. aegypti. Crosses between Cas9 expression lines with nanos promoters to lines targeting the βTub gene (gRNAβTubulin and Sxl) resulted in 100% sterile male progeny (WO 2019/103982, Kandul et al. 2018 and Kandul et al. 2019). Notably, sterile males resulting from SIT technologies are only effective if they can competitively court and mate with females, and the compromised fitness of sterile males generated by a classic SIT has been the weakest point of this technology. Without wishing to be bound by the theory, because a single gene required only for later spermatid maturation was mutated, the fitness of pgSIT sterile males were not be significantly reduced. Indeed, applicant found that the pgSIT-generated males were able to compete effectively for mates with wt counterparts and survive as long or longer that wt males (WO 2019/103982, Kandul et al. 2018 and Kandul et al. 2019).

Relating to the translation of the precision-guided sterile insect technique (pgSIT), developed for Drosophila melanogaster (Kandul et al. 2019), to other species, in particular Aedes aegypti. A number of assumptions were made by Kandul et al. 2019 in extending the implications of the findings to other species. Here, applicant explored the implications of less generous assumptions regarding the field performance of pgSIT in Ae. aegypti, and confirmed that it is still a competitive technology under these conditions. Applicant also clarified logistical concerns regarding the distribution of eggs, sex sorting of adults, and the potential for mutations to occur that could disrupt field implementation.

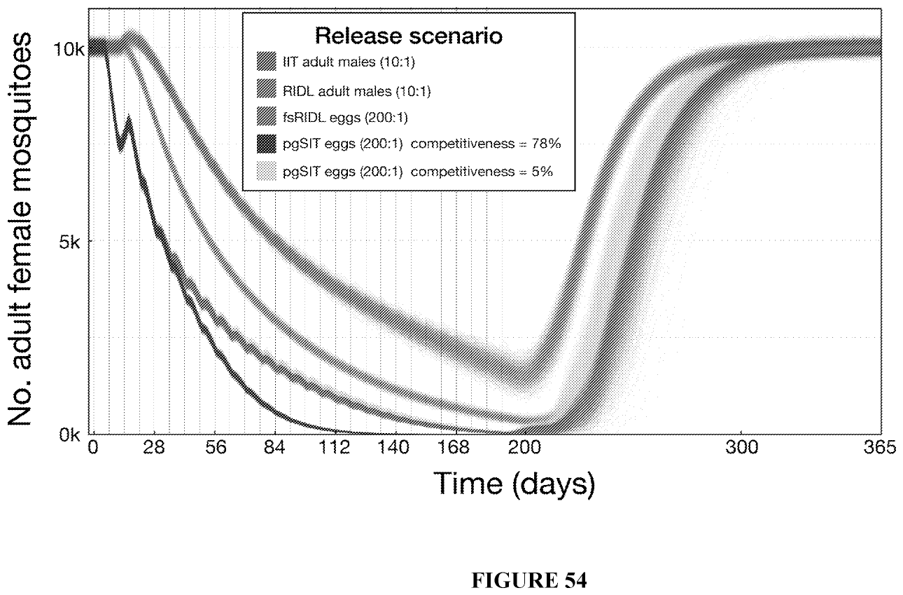

In Kandul et al. 2019, for the simulations of releases of insects carrying a dominant lethal (RIDL) and female-specific RIDL (fsRIDL), the mating competitiveness parameter was taken from the field performance of an Ae. aegypti RIDL strain (Carvalho et al. 2015 and Harris et al. 2011); whereas for pgSIT, it was taken from the lab performance of a D. melanogaster strain. Further investigation was performed. In the absence of field data for pgSIT, Applicant repeated the pgSIT simulations with the mating competitiveness parameter used for RIDL, in which released adult males having the construct had their mating competitiveness reduced by 95% as compared to wild males in the field (Carvalho et al. 2015 and Harris et al. 2011). Results from these simulations suggest a very similar performance between pgSIT and fsRIDL when identical parameters are used (FIG. 54). Without wishing to be bound by the theory, this finding may be explained as both are self-limiting population suppression technologies that involve releases at the egg stage.

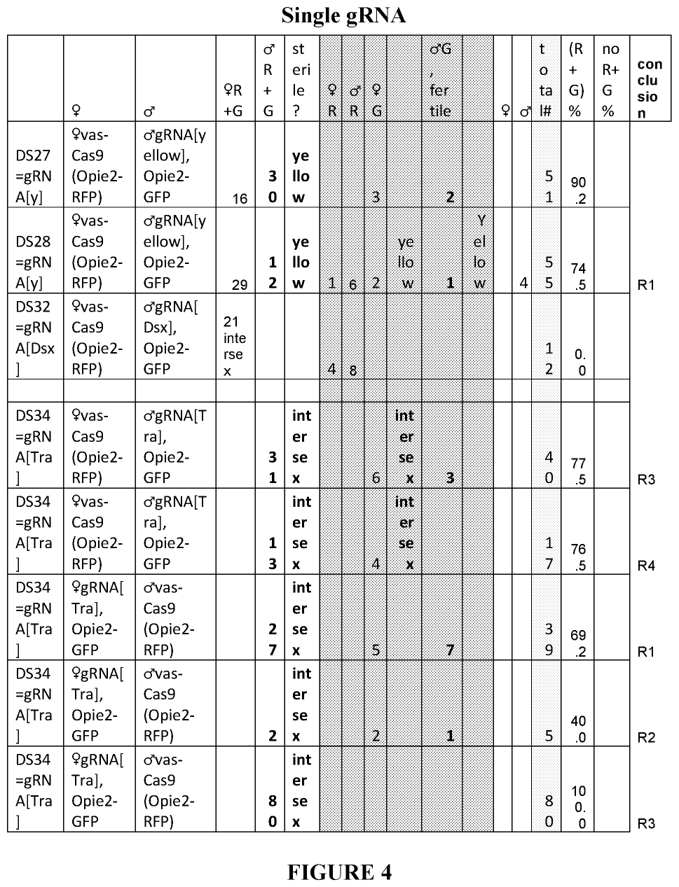

Further several lines of evidence suggest that parameters estimated for the MDL system may be conservative for the pgSIT system, and hence that pgSIT may have a greater potential to eliminate local Ae. aegypti populations than current published versions of the MDL system. First, applicant used a conservative estimate of an 18% lifespan reduction associated with the pgSIT construct, based on an estimate for the MDL system in the lab (Massonnet-Bruneel et al. 2013), despite measuring a lifespan increase associated with the pgSIT system in D. melanogaster (FIG. 4c of Kandul et al. 2019). The cause of this still needs to be investigated; however it displays the opposite trend as that seen for RIDL in Ae. aegypti. Second, the fitness of MDL mosquitoes is likely impacted by tetracycline exposure and leaky toxin expression of the dominant lethal gene—two factors that do not affect the pgSIT system. That said, there may be unforeseen fitness consequences of the pgSIT system in Ae. aegypti.

Relating to the logistics of pgSIT field implementation for Ae. aegypti: i) sex sorting of adults to produce pgSIT eggs, ii) distribution of eggs in the environment and survival to the adult stage, and iii) dispersal and mixing of pgSIT adults with wild mosquitoes. First, regarding sex sorting, without wishing to be bound by the theory, this is not to be an issue given the precent set by the Debug Project of Verily who routinely and efficiently sort female and male mosquitoes with 99.99% accuracy. This readily-available sex-sorting technology enables the separation of adult females and males from both strains to generate pgSIT eggs in quantities sufficient for wide-scale field implementation. Second, regarding egg distribution and survival, despite the difficulty of distributing eggs to small containers and breeding sites in the environment (Bouyer et al. 2019), the disclosure enables releases of pgSIT eggs into defined, artificial breeding containers in the field so that mosquitoes may then emerge into the environment with high yield survival from egg to adult. Third, regarding mixing of pgSIT males with wild mosquitoes, these artificial breeding containers would be placed carefully to ensure adequate mixing with the wild mosquito population. If the technology proceeds to field implementation, preliminary ecological assessments would be carried out at field sites, along with mathematical modeling, to design release schemes that would address this concern.

Third, relating to the potential for mutations to occur that could inactivate the Cas9 or gRNA cassettes, as a byproduct of mass rearing millions of insects on a weekly basis. It is true that mutations may occur that inactivate the pgSIT system, however these are unlikely to be detrimental to field implementation. By comparison, RIDL technology has been successfully implemented in the field despite being likely impacted more frequently by mutations that inactivate the system due to basal expression of the VP16 toxin that ecodes dominant lethality, whereas pgSIT is a binary system and is inactive until crossed. That said; assays for detecting inactive pgSIT mutants would be developed as part of the factory protocol.

Fourth, regarding to releasing eggs is not an option for insect pests such as the fruit fly, as disclosed herein applicant is actively building pgSIT in the fruit fly Drosophila suzukii, a major crop pest that lays eggs in fruit, and have plans to deploy eggs by releasing artificial food substrate in which they have been laid. Finally, in terms of regulatory limitations, without wishing to be bound by the theory, pgSIT would be regulated in a similar category to comparable insect technologies (e.g. RIDL), which has been approved for release in many countries and is being used in the field today. The pgSIT system in D. melanogaster are highly encouraging for application to other species, in particular Ae. aegypti, but also including several insect agricultural crop pests. The disclosure herein addressed the need for further lab and field studies to demonstrate the performance of the system in these species as required by Kandul et al. 2019.

### Construction of Plasmids—Cloning Vectors 1055

Dual-U6-gRNAs (such as, Vector 1055A˜1055F) were designed and constructed. Briefly, Vector 1051v3 was cut with BmtI-HF and PmeI (CutSmart 37° C., 1548 bp drop-off, 8.8 Kb backbone) and Onestep GenPartA-F was applied respectively to make Vector 1055A-1055F.

Another example is Vector 1055T-pBac-U6(AAEL017774)-sgPb1-LongScaffold-3′UTR-U6(AAEL017763)-sgPb2-LongScaffold-3′UTR-3xp3-tdTomato. It was constructed via cutting Vector 1051v3 with BmtI-HF and PmeI (CutSmart 37° C., 1548 bp drop-off, 8.8 Kb backbone), performing PCR of sgPb1-LongScaffold-3′UTR-U6(AAEL017763)-sgPb2 with primers 1055T.C1/C2 from 1051.v3 (1627 bp) and conducting Onestep according to the manufacturer protocol. For diagnostic, restriction enzymes PacI/AscI/PmeI (1.5 Kb+8.9 Kb) were used and 1051.v3 is a control (0.6 Kb+0.85 Kb+8.9 Kb).

Additionally, Vector 1055Y-pBac-U6(AAEL017774)-sgLab5-U6(AAEL017763)-sgLab4-LongScaffold-3′UTR-3xp3-tdTomato was constructed via cutting Vector 1051v3 with BmtI-HF and PmeI (CutSmart 37° C., 1548 bp drop-off, 8.8 Kb backbone), performing PCR of sgMHC3-LongScaffold-3′UTR-U6(AAEL017763)-sgMHC4 with primers 1055Y.C1/C2 from 1051.v3 (1627 bp) at 61° C. and conducting Onestep reaction according to the manufacturer protocol. For diagnostic, restriction enzymes PacI/AscI/PmeI (1.5 Kb+8.9 Kb) were used and 1051.v3 performed as a control (0.6 Kb+0.85 Kb+8.9 Kb).

Relating to constructing Vector 1055Z-pBac-U6(AAEL017774)-sgSlx4-U6(AAEL017763)-sgSlx5-LongScaffold-3′UTR-3xp3-tdTomato, Vector 1051v3 was cut with BmtI-HF and PmeI (CutSmart 37° C., 1548 bp drop-off, 8.8 Kb backbone). PCR reaction was performed on sgMHC3-LongScaffold-3′UTR-U6(AAEL017763)-sgMHC4 with primers 1055Z.C1/C2 from 1051.v3 (1627 bp) at 61° C., followed by Onestep reaction conducted according to the manufacturer protocol. For diagnostic, restriction enzymes PacI/AscI/PmeI (1.5 Kb+8.9 Kb) were used while 1051.v3 performed as a control (0.6 Kb+0.85 Kb+8.9 Kb).

Sequencing of the constructed vectors was also performed for verification purpose.

### Construction of Plasmids—Cloning Vectors 1067

Quad-U6-gRNAs were designed and constructed. For example, Vector 1067A-pBac-U6(AAEL017774)-sgBTub1-U6(AAEL017763)-sgBTub2-U6(AAEL017774)-sgBTub3-U6(AAEL017763)-sgBTub4-LongScaffold-3′UTR-3xp3-tdTomato-v3 was constructed via cutting Vector 1055B with BglII (NEBuffer 3.1, 37° C., no drop-off, 10.4 Kb backbone) a. CIP/Ant. Phosphatase, PCRing Dual-U6-gRNAs with 1067C1/1067C2 from 1055A (3249 bp) and conducting Onestep reaction conducted according to the manufacturer protocol.

Vector 1067B-pBac-U6(AAEL017774)-sgDsx1-U6(AAEL017763)-sgDsx2-U6(AAEL017774)-sgDsx3-U6(AAEL017763)-sgDsx4-LongScaffold-3′UTR-3xp3-tdTomato-v3 was constructed via cutting Vector 1055D with BglII (NEBuffer 3.1, 37° C., no drop-off, 10.4 Kb backbone) a. CIP/Ant. Phosphatase; PCRing Dual-U6-gRNAs with 1067C1/1067C2 from 1055C (3249 bp); and Onestep.

Vector 1067C-pBac-U6(AAEL017774)-sgZpg1-U6(AAEL017763)-sgZpg2-U6(AAEL017774)-sgZpg3-U6(AAEL017763)-sgZpg4-LongScaffold-3′UTR-3xp3-tdTomato-v3 was constructed via cutting Vector 1055F with BglII (NEBuffer 3.1, 37° C., no drop-off, 10.4 Kb backbone) a. CIP/Ant. Phosphatase; PCRing Dual-U6-gRNAs with 1067C1/1067C2 from 1055E (3249 bp); and Onestep.

Vector 1067D-pBac-U6(AAEL017774)-sgDsx1-U6(AAEL017774)-sgIsx1-U6(AAEL017774)-sgBTub3-U6(AAEL017763)-sgBTub2-LongScaffold-3′UTR-3xp3-tdTomato was constructed via cutting Vector 1055CJ with ApaI and AscI (Digest in CutSmart at 25° C. with ApaI, then add AscI and raise temperature to 37° C., no dropoff, 10.4 Kb backbone); PCRing U6(AAEL017774)-sgBTub3-U6(AAEL017763)-sgBTub2 with 1067C3/1067C4 from 1055BA (3230 bp); cutting PCR product with ApaI and AscI (Digest in CutSmart at 25° C. with ApaI, then add AscI and raise temperature to 37° C., keep 3199 bp); and subcloning. For diagnostic, restriction enzymes ApaI/AscI (3.4 Kb+10.4 Kb) were used, while 1055BA performed as control (10.4 Kb).

Vector 1067E-pBac-U6(AAEL017774)-sgDsx1-U6(AAEL017774)-sgDsx5-U6(AAEL017774)-sgBTub3-U6(AAEL017763)-sgBTub2-LongScaffold-3′UTR-3xp3-tdTomato was constructed via cutting Vector 1055C1 with ApaI and AscI (Digest in CutSmart at 25° C. with ApaI, then add AscI and raise temperature to 37° C., no dropoff, 10.4 Kb backbone); PCRing U6(AAEL017774)-sgBTub3-U6(AAEL017763)-sgBTub2 with 1067C3/1067C4 from 1055BA (3230 bp); cutting PCR product with ApaI and AscI (Digest in CutSmart at 25° C. with ApaI, then add AscI and raise temperature to 37° C., keep 3199 bp); and subcloning. For diagnostic, restriction enzymes ApaI/AscI (3.4 Kb+10.4 Kb) were used, while 1055C1 and 1055BA served as control (10.4 Kb).

Vector 1067F.v3-pBac-U6(AAEL017774)-sgBTub3-U6(AAEL017763)-sgBTub2-LongScaffold-3′UTR-3xp3-tdTomato-U6(AAEL017774)-sgDsx1-U6(AAEL017763)-sgDsx1 was constructed via cutting Vector 1055BA with FseI (Stored at −80° C., Digest in CutSmart at 37° C., no dropoff, 10.4 Kb backbone), followed by CIP; PCRing U6(AAEL017774)-sgDsx1-U6(AAEL017763)-sgDsx1 with 1067C5/1067C6 from 1055C2 (3238 bp); and onestep. For diagnostic, restriction enzymes BmtI (or NheI) and PmeI (3.2 Kb+10.4 Kb) were used.

Vector 1067G-pBac-U6(AAEL017774)-sgZpg3-U6(AAEL017763)-sgZpg4-LongScaffold-3′UTR-3xp3-tdTomato-U6(AAEL017774)-sgDsx1-U6(AAEL017763)-sgDsx1 was constructed via cutting Vector 1055F with FseI (Stored at −80° C., Digest in CutSmart at 37° C., no dropoff, 10.4 Kb backbone), followed by CIP; PCRing U6(AAEL017774)-sgDsx1-U6(AAEL017763)-sgDsx1 with 1067C5/1067C6 from 1055C2 (3238 bp); and onestep. For diagnostic, restriction enzymes BmtI (or NheI) and PmeI (3.2 Kb+10.4 Kb) were used.

Vector 1067H-pBac-U6(AAEL017774)-sgDsx1-U6(AAEL017763)-sgDsx2-U6(AAEL017774)-sgDsx3-U6(AAEL017763)-sgDsx4-LongScaffold-3′UTR-3xp3-tdTomato-v3-U6(AAEL017774)-sgBTub3-U6(AAEL017763)-sgBTub2 was constructed via cutting Vector 1067B with FseI (Stored at −80° C., Digest in CutSmart at 37° C., no dropoff, 13.6 Kb backbone), followed by CIP; PCRing U6(AAEL017774)-sgBTub3-U6(AAEL017763)-sgBTub2 with 1067C5/1067C6 from 1055BA (3238 bp); and onestep. For diagnostic, restriction enzymes BmtI (or NheI) (5.2 Kb+11.6 Kb) were used, while 1067B served as control (linear 13.6 Kb).

Sequencing of the constructed vectors was also performed for verification purpose.

Generation of Sterile Males in F Progeny—Development of 100% Sterile Males from Cas9 and Dual Female Viability and Male Fertility Deficient gRNA Crosses:

The goal of pgSIT is to disrupt genes essential for male fertility/female viability simultaneously to ensure that all surviving F₁ offspring are sterile males. To achieve this feat, applicant generated three homozygous strains expressing multiplexed double gRNA (dgRNA) combinations, including dgRNA^{βTub,Sxl}, dgRNA^{βTub,Tra}, and dgRNA^{βTub,DsxF}. To genetically assess the activity of these pgSIT strains, Applicant bidirectionally crossed each line to wt, or homozygous Cas9 (either nos-Cas9, vas-Cas9, or Ubi-Cas9) (WO 2019/103982, Kandul et al. 2018 and Kandul et al. 2019). As expected, the WT crosses produced no significant gender deviations or compromised fertility. Interestingly however, the crosses between dgRNA^{βTub,Sxl} with each Cas9 strain resulted in 100% female lethality due to disruption of sxl, in addition to 100% male sterility due to simultaneous disruption of βTub (n=2521, N=24). Moreover, 100% of females from crosses between each Cas9 strain and dgRNA^{βTub,Tra} (n=1697, N=24) or dgRNA^{βTub,DsxF} (n=1791, N=24) were masculinized into sterile intersexes due to disruption of either tra or dsx, and 100% male offspring were sterile due to simultaneous disruption of βTub (n=4231, N=48). These findings demonstrate that the ability to form highly active Cas9-gRNA complexes was not saturated by dgRNAs and the pgSIT approach works reproducibly with unprecedented efficiency (WO 2019/103982, Kandul et al. (2018) and Kandul et al. (2019)). Targets listed as disclosed herein as well as the constructed vectors are tested.

### Experiment No. 2—Precision Guided Sterile Insect Technique to Control Drosophila Suzukii.

### Construction of Plasmids—Cloning Vectors 1056

1056C.2 (HL-U6:3-sxl2-gRNA-UTR-opie-mVenus-SV40-U6:3-sxl1-gRNA-UTR-HR) was constructed via cutting 1056C with PacI and AflII (CutSmart 37° C., 1.5 Kb dropout); PCRing opie-mVenus-SV40 with primers 1056C.C5/1056C.C6 from 1056.X (Tm 55C, 1.5 kb) and Onestep.

1056H (HL-U6:3-sxl2-gRNA-UTR-opie-mVenus-SV40-U6:3-sxl1-gRNA-UTR-HR-U6-Btub1-UTR) was constructed via cutting 1056C.2 with AscI+AvrII (CutSmart 37° C., no dropout); PCRing U63 promoter from vector 1056X with primers 1056H.C1/1056H.C2 (477 bp, 60-65° C.); PCRing scaffold-UTR- from vector 1056X with primers 1056H.C3/1056H.C4 (2340 bp, 50˜55° C.); and Onestep.

1056H.C1:
(SEQ ID NO: 48)
CGCTTGCTTGAATAGAATTCTCATCGGCGCGCCTTTTTGCTCACCT
GTGATTGCTCCTAC
1056H.C2:
(SEQ ID NO: 49)
AAACTTCCGGAGCCGGTACCGCCACGACGTTAAATTGAAAATAGGT
CTATATATACGAAC
1056H.C3:
(SEQ ID NO: 50)
ACCTATTTTCAATTTAACGTCGTGGCGGTACCGGCTCCGGAAGTTT
TAGAGCTAGAAATA
1056H.C4:
(SEQ ID NO: 51)
CACTGAACATTGTCAGATCCGAGATCGGCCGGCCTAGGATGCATAC
GCATTAAGCGAACA

1056I (HL-U6:3-sxl2-gRNA-UTR-opie-mVenus-SV40-U6:3-sxl1-gRNA-UTR-HR-U6-Btub2-UTR) was constructed via cutting 1056C.2 with AscI+AvrII (CutSmart 37° C., no dropout); PCRing U63 promoter from vector 1056X with primers 1056H.C1/1056H.C5 (477 bp, 60-65° C.); PCRing scaffold-UTR- from vector 1056X with primers 1056H.C6/1056H.C4 (2340 bp, 50˜55° C.); and Onestep.

1056J (HL-U6:3-sxl2-gRNA-UTR-opie-mVenus-SV40-U6:3-sxl1-gRNA-UTR-HR-U6-Btub3-UTR) was constructed via cutting 1056C.2 with AscI+AvrII (CutSmart 37° C., no dropout); PCRing U63 promoter from vector 1056X with primers 1056H.C1/1056H.C7 (477 bp, 60-65° C.); PCRing scaffold-UTR- from vector 1056X with primers 1056H.C8/1056H.C4 (2340 bp, 50˜55° C.); and Onestep.

1056K (HL-U6:3-sxl2-gRNA-UTR-opie-mVenus-SV40-U6:3-sxl1-gRNA-UTR-HR-U6-Btub4-UTR) was constructed via cutting 1056C.2 with AscI+AvrII (CutSmart 37° C., no dropout); PCRing U63 promoter from vector 1056X with primers 1056H.C1/1056H.C9 (477 bp, 60-65° C.); PCRing scaffold-UTR- from vector 1056X with primers 1056H.C10/1056H.C4 (2340 bp, 50˜55° C.); and Onestep.

1056H.C5:
(SEQ ID NO: 52)
AAACCCAGCTGGTGCACACTCAAGCGACGTTAAATTGAAAATAGGT
CTATATATACGAAC
1056H.C6:
(SEQ ID NO: 53)
ACCTATTTTCAATTTAACGTCGCTTGAGTGTGCACCAGCTGGGTTT
TAGAGCTAGAAATA
1056H.C7:
(SEQ ID NO: 54)
AAACCCGATCTGGTTACCGCATTGCGACGTTAAATTGAAAATAGGT
CTATATATACGAAC
1056H.C8:
(SEQ ID NO: 55)
ACCTATTTTCAATTTAACGTCGCAATGCGGTAACCAGATCGGGTTT
TAGAGCTAGAAATA
1056H.C9:
(SEQ ID NO: 56)
AAACACATCTTTAGACCCCGAGGCCGACGTTAAATTGAAAATAGGT
CTATATATACGAAC
1056H.C10:
(SEQ ID NO: 57)
ACCTATTTTCAATTTAACGTCGGCCTCGGGGTCTAAAGATGTGTTT
TAGAGCTAGAAATA

Development of Sterile Males from Cas9 and Dual Female Viability and Male Fertility Deficient gRNA Crosses

Targets listed as disclosed herein as well as the constructed vectors were tested.

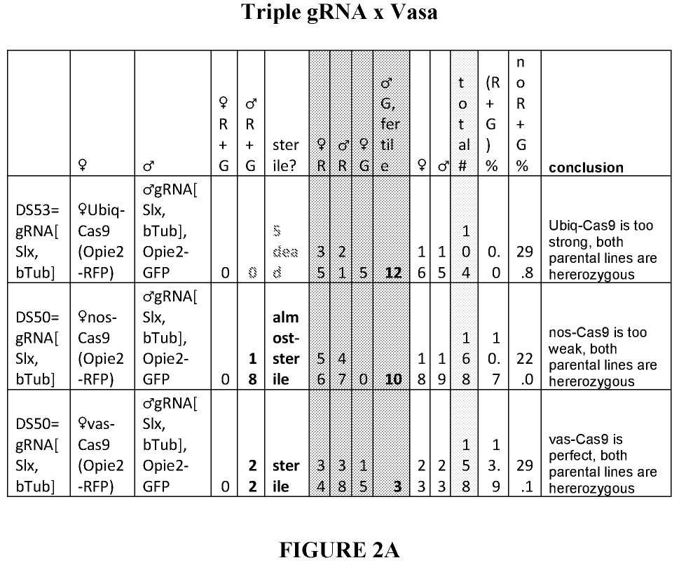

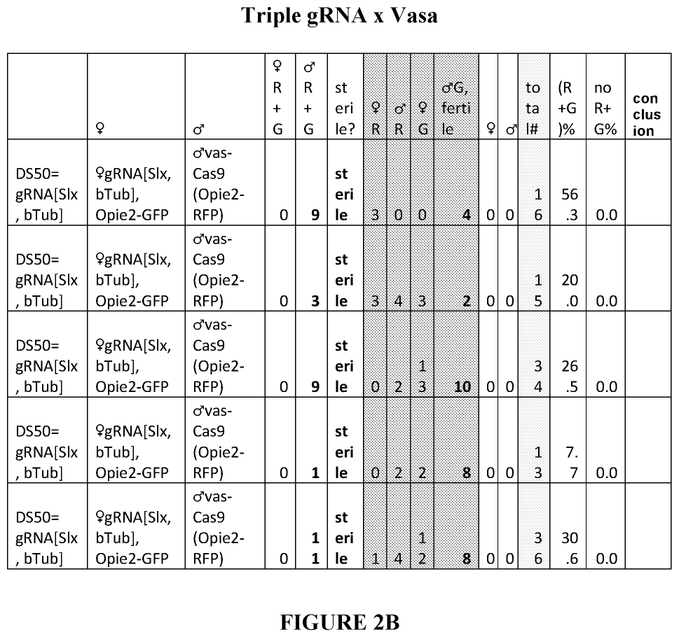

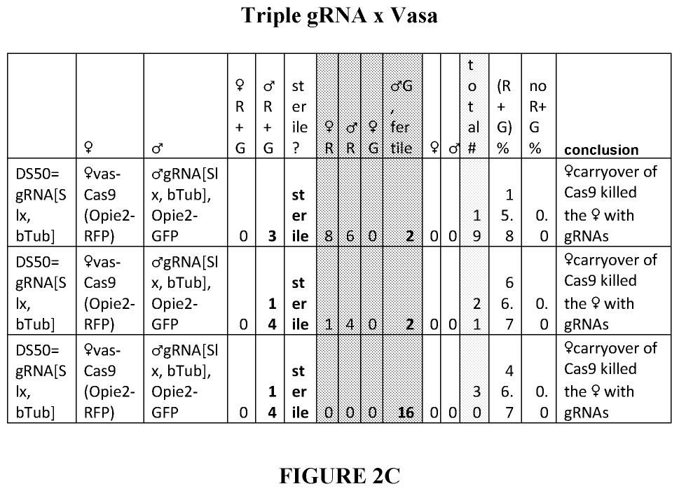

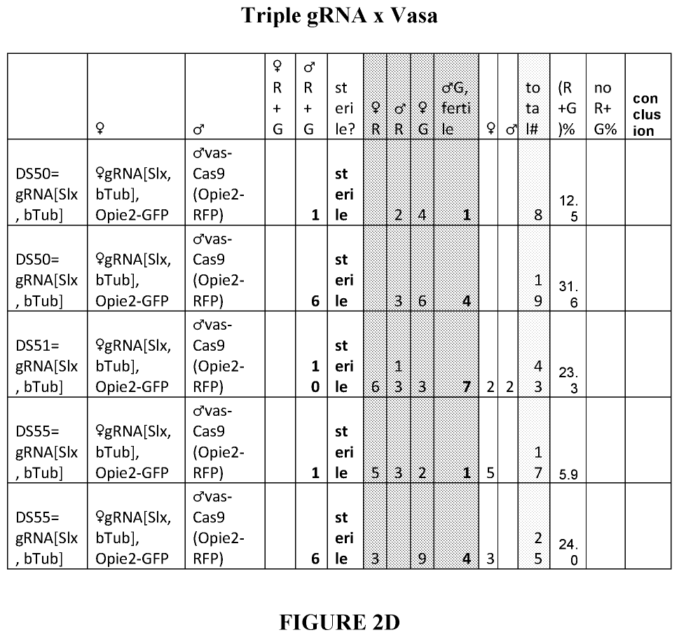

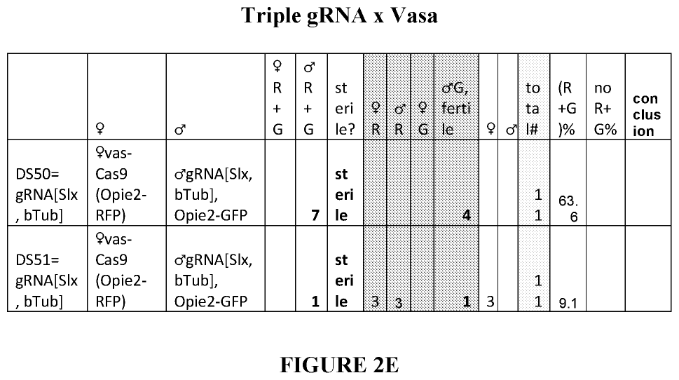

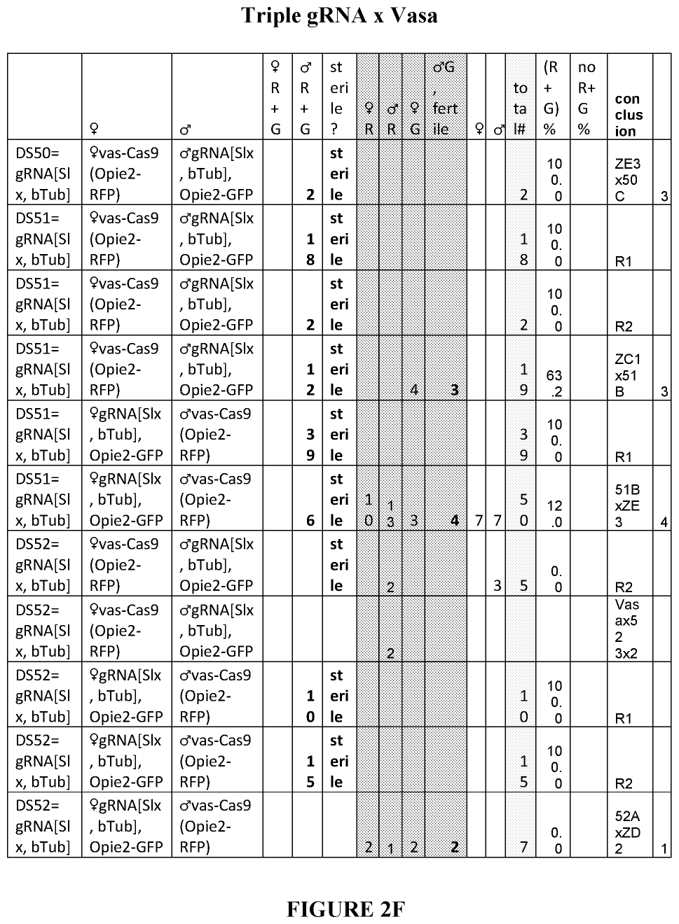

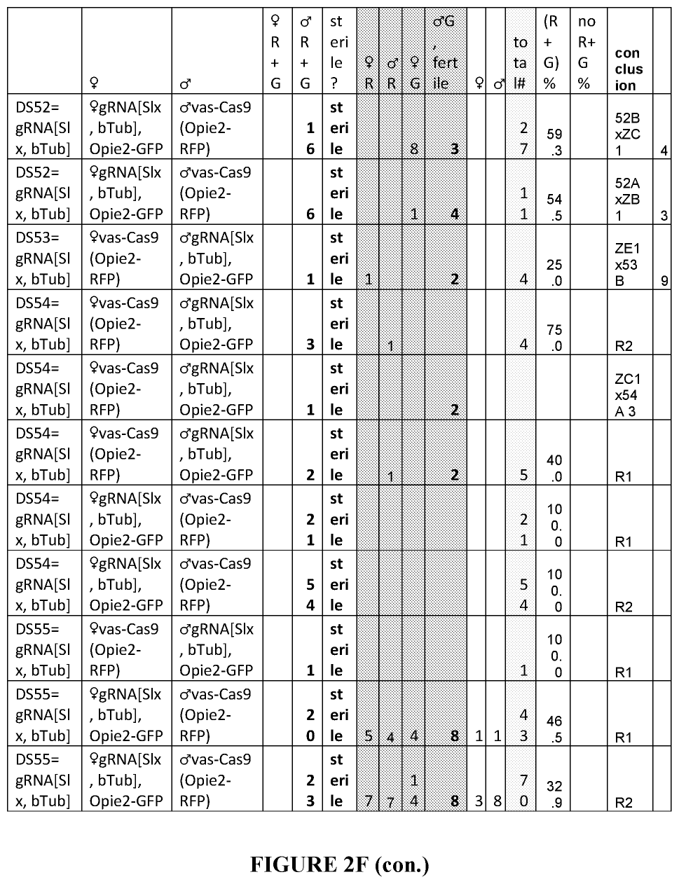

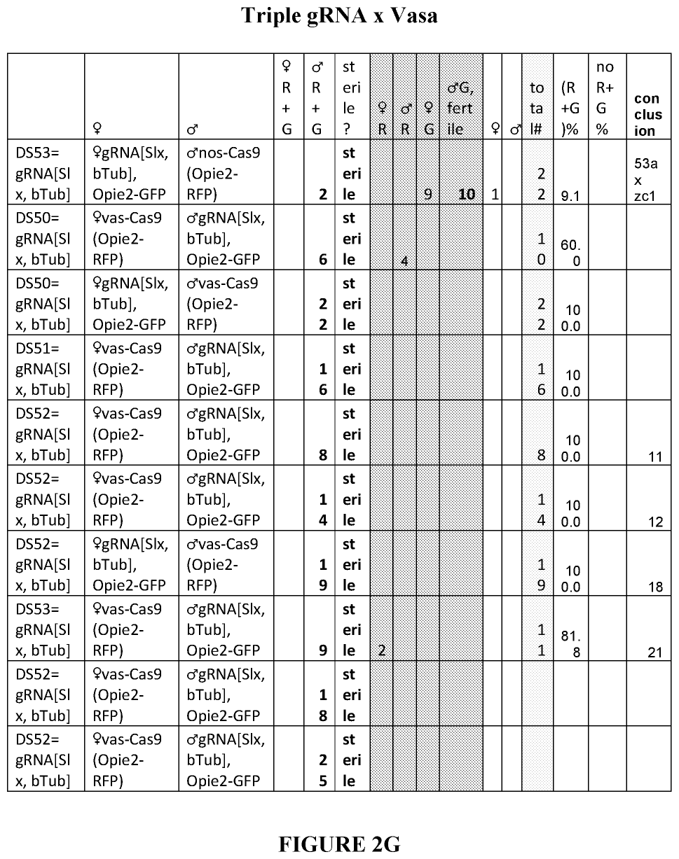

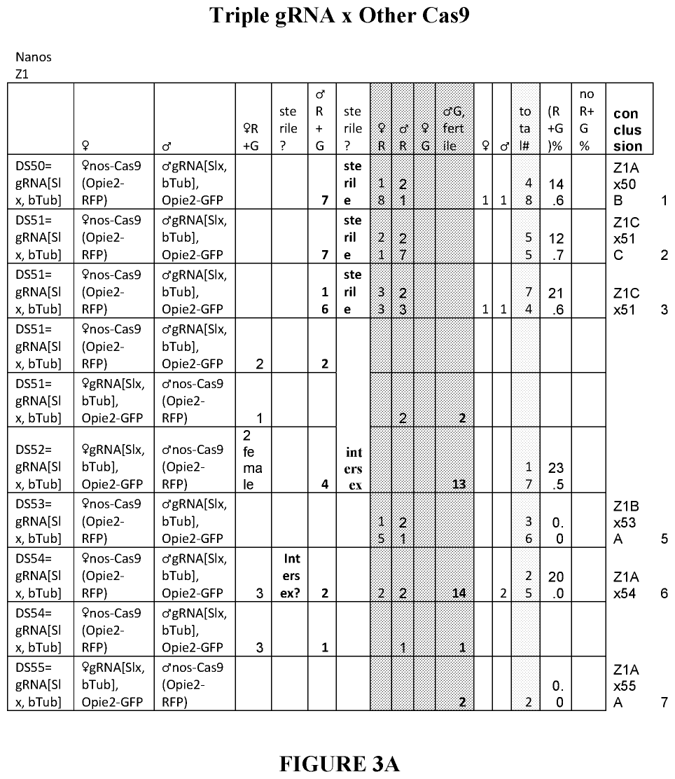

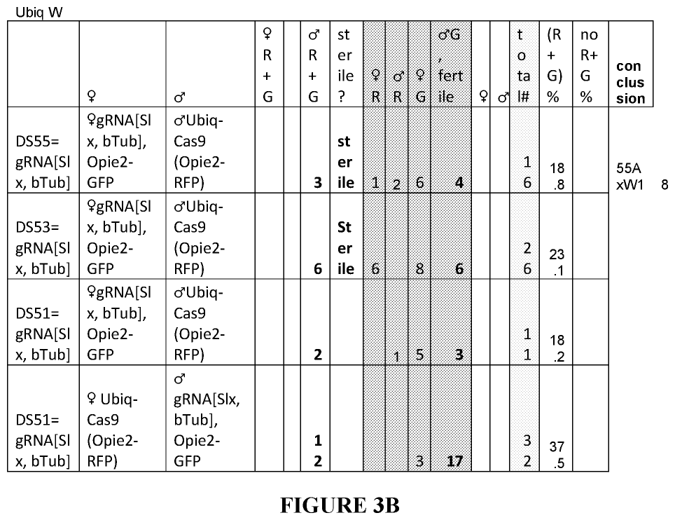

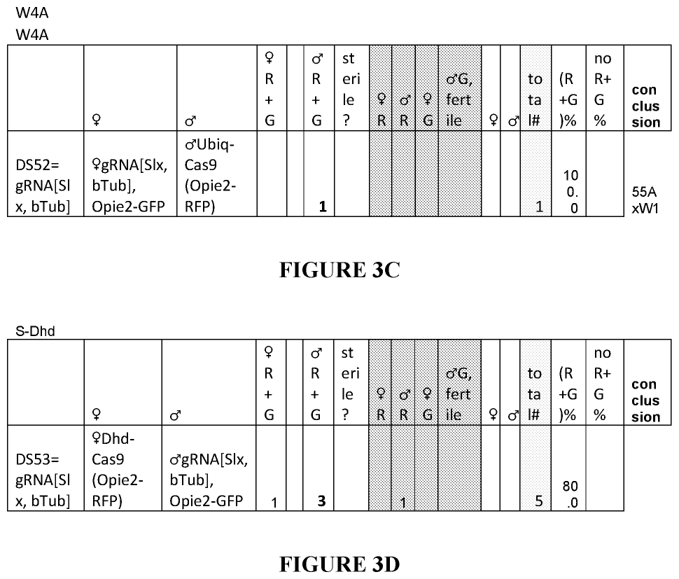

Firstly, the Cas9 and the gRNA were labelled with different fluorescent markers, such as Red Fluorescent Protein (RFP) or Green Fluorescent Protein (GFP). The insects successfully expressing Cas9 or gRNA also showed the corresponding fluorescent signal (R, red; or G, green). The insect gender and the male fertility were then determined. Results are provided in FIGS. 2A-4. As shown in FIG. 2A, use of vas-Cas9 and gRNAs targeting Slx and bTub provides optimal results, such as (1) female Drosophila suzukii having both Cas9 and gRNAs are alive; (2) male Drosophila suzukii having both Cas9 and gRNAs are sterile; and (3) male and female Drosophila suzukii having either Cas9 or gRNAs are alive and fertile. Without wishing to be bound by the theory, appropriate expression level contributes to such optimal result, for example, too strong expression of Cas9 kills female as well as male Drosophila suzukii having both Cas9 and gRNA, while too weak expression of Cas9 leads to generation of fertile male having both Cas9 and gRNA. Such appropriate expression level may be adjusted by using various the regulatory sequences, such as promoters.

### Non-Limiting Embodiments of the Disclosure

Embodiment 1. An isolated polynucleotide Cas9 construct as described herein, its complement or an equivalent of each thereof.

Embodiment 2. The isolated Cas9 construct of Embodiment 1, wherein the construct is of the group of: 874Z, 874S, 874R, 874W, and 874Z1.

Embodiment 3. A fragment of the isolated Cas9 construct of Embodiment 2, wherein the fragment encodes a Cas9 polypeptide.

Embodiment 4. A vector comprising the isolated polynucleotide of Embodiment 3.

Embodiment 5. An isolated host cell comprising the isolated polynucleotide of any of Embodiments 1-3 or the vector of Embodiment 4, optionally wherein the host cell is an insect cell.

Embodiment 6. An expression product of the polynucleotide of any of Embodiments 1-3 or the vector of Embodiment 4.

Embodiment 7. A guide RNA (gRNA) as disclosed herein, or its complement or an equivalent of each thereof.

Embodiment 8. The gRNA of Embodiment 7, wherein the gRNA is a single guide RNA (sgRNA) or a double guide RNA (dg RNA).

Embodiment 9. A vector comprising the gRNA of Embodiment 7 or 8.

Embodiment 10. An isolated host cell comprising the gRNA of Embodiment 7 or 8, or the vector of Embodiment 9, optionally wherein the host cell is an insect cell.

Embodiment 11. An isolated host cell comprising the isolated polynucleotide of any of Embodiments 1-3 and the gRNA of Embodiment 7 or 8, optionally wherein the host cell is an insect cell.

Embodiment 12. The isolated host cell of any of Embodiments 5, 10, or 11, wherein the host cell is an egg, a sperm or a zygote.

Embodiment 13. A host cell system comprising a first host cell of Embodiment 5 and a second host cell of Embodiment 10, wherein the first host cell and the second host cell are homozygous, optionally wherein the host cell is an insect cell.

Embodiment 14. An insect comprising the isolated host cell of any of Embodiments 10-12.

Embodiment 15. A mating system comprising a first host cell of Embodiment 5 and a second host cell of Embodiment 10, wherein the first host cell and the second host cell are homozygous.

Embodiment 16. A progeny produced by the mating system of Embodiment 15.

Embodiment 17. A CRISPR system comprising:
(a) a Cas9 polypeptide as described herein, or an equivalent thereof, and
(b) a recombinant or synthetic guide RNA (gRNA) as described herein, or an equivalent thereof.

Embodiment 18. An isolated host cell comprising the CRISPR system of Embodiment 17.

Embodiment 19. A method to sterilize or induce lethality of an insect comprising inserting the cell of Embodiment 18 into the insect.

Embodiment 20. A method of producing a genetically modified progeny of a sterile male Aedes aegypti mosquito egg, the method comprising:
(a) introducing at least one nucleic acid sequence into a first Aedes aegypti mosquito, wherein the at least one nucleic acid sequence comprising
   at least a first guide RNA targeting a female essential genomic sequence that is required for female-specific viability; and
   at least a second guide RNA targeting a male sterility genomic sequence that is required for male-specific fertility;
(b) introducing an endonuclease into a second Aedes aegypti mosquito, the second Aedes aegypti mosquito; and
(c) genetically crossing the first Aedes aegypti mosquito and the second Aedes aegypti mosquito, wherein genetically crossing the first Aedes aegypti mosquito and the second Aedes aegypti mosquito produces a progeny of sterile male Aedes aegypti mosquito eggs.

Embodiment 21. The method of Embodiment 20, wherein the female essential genomic sequence comprises a gene essential for female-specific viability or a female-specific exon essential for female-specific development and/or female-specific viability.

Embodiment 22. The method of Embodiment 21, wherein the female-essential genomic sequence is transformer 2 (tra-2).

Embodiment 23. The method of Embodiment 21, wherein the female essential genomic sequence is doublesex (dsx).

Embodiment 24. The method of any one of Embodiments 20-23, wherein the at least one first guide RNA comprises more than one first guide RNA each of which targets a different female-essential genomic sequence that is required for female-specific viability or targets a different region of the same female-essential genomic sequence that is required for female-specific viability.

Embodiment 25. The method of any one of Embodiments 20-24, wherein the male sterility genomic sequence comprises a gene essential for male-specific sterility.

Embodiment 26. The method of Embodiment 25, wherein the male sterility genomic sequence is selected from the group consisting of βTubulin (βTub), fuzzy onions (Fzo), protaimine A (ProtA), or spermatocyte arrest (Sa).

Embodiment 27. The method of Embodiment 25 or 26, wherein the at least one second guide RNA comprises more than one second guide RNA each of which targets a different male sterility genomic sequence that is required for male fertility or targets a different region of the same male sterility genomic sequence that is required for male specific fertility.

Embodiment 28. The method of any one of Embodiments 20-27, wherein the endonuclease comprises a CRISPR-associated sequence 9 (Cas9) endonuclease or a variant thereof, a CRISPR-associated sequence 13 (Cas13) endonuclease or a variant thereof, a CRISPR from Prevotella and Francisella 1 (Cpf1) endonuclease or a variant thereof, a CRISPR from Microgenomates and Smithella 1 (Cms1) endonuclease or a variant thereof, or a CRISPR-associated sequence 6 (Cash) endonuclease or a variant thereof.

Embodiment 29. The method of Embodiment 20, wherein the first Aedes aegypti is a female and the second Aedes aegypti is a male.

Embodiment 30. The method of Embodiment 20, wherein the first Aedes aegypti is a male and the second Aedes aegypti is a female.

Embodiment 31. The method of Embodiment 20, wherein the introducing step (a) comprises integrating the at least one nucleic acid into the genome of the first Aedes aegypti mosquito.

Embodiment 32. The method of Embodiment 20, wherein the genetically crossing step (c) comprises producing progeny comprising the endonuclease, at least one first guide RNA, and at least one second guide RNA from which male Aedes aegypti mosquito eggs mature to adulthood.

Embodiment 33. The method of any one of Embodiments 20-32, wherein the endonuclease is operably linked to a nup50 promoter or a fragment thereof or a uniq promoter or a fragment thereof.

Embodiment 34. A progeny of Aedes aegypti mosquito eggs comprising up to 100% sterile male Aedes aegypti mosquito eggs produced by the method of any one of Embodiments 20-33.

### EQUIVALENTS

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

It should be understood that although the present invention has been specifically disclosed by certain aspects, embodiments, and optional features, modification, improvement and variation of such aspects, embodiments, and optional features can be resorted to by those skilled in the art, and that such modifications, improvements and variations are considered to be within the scope of this disclosure.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

All publications, patent applications, patents, and other references mentioned herein are expressly incorporated by reference in their entirety, to the same extent as if each were incorporated by reference individually. In case of conflict, the present specification, including definitions, will control.

Other aspects are set forth within the following claims.

### REFERENCES

1. Alphey, Luke. 2016. “Genetic Control of Mosquitoes.” New Biotechnology 33 (July): S30.
2. Aryan A, Anderson M A E, Biedler J K, et al. Nix alone is sufficient to convert female Aedes aegypti into fertile males and myo-sex is needed for male flight [published online ahead of print, 2020 Jul. 13]. Proc Natl Acad Sci USA. (2020); 202001132. doi:10.1073/pnas.2001132117
3. Awata, Hiroko, Takahito Watanabe, Yoshitaka Hamanaka, Taro Mito, Sumihare Noji, and Makoto Mizunami. 2015. “Knockout Crickets for the Study of Learning and Memory: Dopamine Receptor Dop1 Mediates Aversive but Not Appetitive Reinforcement in Crickets.” Scientific Reports 5 (November): 15885.
4. Bassett, Andrew R., and Ji-Long Liu. 2014. “CRISPR/Cas9 and Genome Editing in Drosophila.” Journal of Genetics and Genomics=Yi Chuan Xue Bao 41 (1): 7-19.
5. Bassett, Andrew R., Charlotte Tibbit, Chris P. Ponting, and Ji-Long Liu. 2013. “Highly Efficient Targeted Mutagenesis of Drosophila with the CRISPR/Cas9 System.” Cell Reports 4 (1): 220-28.
6. Bellini, R., A. Medici, A. Puggioli, F. Balestrino, and M. Carrieri. 2013. “Pilot Field Trials with Aedes Albopictus Irradiated Sterile Males in Italian Urban Areas.” Journal of Medical Entomology 50 (2): 317-25.
7. Bopp, D., G. Saccone, and M. Beye. 2014. “Sex Determination in Insects: Variations on a Common Theme.” Sexual Development: Genetics, Molecular Biology, Evolution, Endocrinology, Embryology, and Pathology of Sex Determination and Differentiation 8 (1-3): 20-28.
8. Bouyer, J., Vreysen, M. J. B. Concerns about the feasibility of using “precision guided sterile males” to control insects. Nat Commun 10, 3954 (2019). https://doi.org/10.1038/s41467-019-11616-9
9. Bushland, R. C., A. W. Lindquist, and E. F. Knipling. 1955. “Eradication of Screw-Worms through Release of Sterilized Males.” Science 122 (3163): 287-88.
10. Carvalho, Danilo O., Andrew R. McKemey, Luiza Garziera, Renaud Lacroix, Christl A. Donnelly, Luke Alphey, Aldo Malavasi, and Margareth L. Capurro. 2015. “Suppression of a Field Population of Aedes Aegypti in Brazil by Sustained Release of Transgenic Male Mosquitoes.” PLoS Neglected Tropical Diseases 9 (7): e0003864.
11. Catteruccia, Flaminia, Andrea Crisanti, and Ernst A. Wimmer. 2009. “Transgenic Technologies to Induce Sterility.” Malaria Journal 8 Suppl 2 (November): S7.
12. Catteruccia, Flaminia, Jason P. Benton, and Andrea Crisanti. 2005. “An Anopheles Transgenic Sexing Strain for Vector Control.” Nature Biotechnology 23 (11): 1414-17.
13. Champer, Jackson, Riona Reeves, Suh Yeon Oh, Chen Liu, Jingxian Liu, Andrew G. Clark, and Philipp W. Messer. 2017. “Novel CRISPR/Cas9 Gene Drive Constructs Reveal Insights into Mechanisms of Resistance Allele Formation and Drive Efficiency in Genetically Diverse Populations.” PLoS Genetics 13 (7): e1006796.
14. Chatzispyrou, Iliana A., Ntsiki M. Held, Laurent Mouchiroud, Johan Auwerx, and Riekelt H. Houtkooper. 2015. “Tetracycline Antibiotics Impair Mitochondrial Function and Its Experimental Use Confounds Research.” Cancer Research 75 (21): 4446-49.
15. Chu, Van Trung, Timm Weber, Benedikt Wefers, Wolfgang Wurst, Sandrine Sander, Klaus Rajewsky, and Ralf Kuhn. 2015. “Increasing the Efficiency of Homology-Directed Repair for CRISPR-Cas9-Induced Precise Gene Editing in Mammalian Cells.” Nature Biotechnology 33 (March): 543.
16. Diniz, Diego Felipe Araujo, Cleide Maria Ribeiro de Albuquerque, Luciana Oliveira Oliva, Maria Alice Varjal de Melo-Santos, and Constância Flávia Junqueira Ayres. 2017. “Diapause and Quiescence: Dormancy Mechanisms That Contribute to the Geographical Expansion of Mosquitoes and Their Evolutionary Success.” Parasites & Vectors 10 (1): 310.
17. Dodson, Brittany L., Grant L. Hughes, Oluwatobi Paul, Amy C. Matacchiero, Laura D. Kramer, and Jason L. Rasgon. 2014. “Wolbachia Enhances West Nile Virus (WNV) Infection in the Mosquito Culex tarsalis.” PLoS Neglected Tropical Diseases 8 (7): e2965.
18. Dyck, V. A., J. Hendrichs, and A. S. Robinson, eds. 2005. Sterile Insect Technique: Principles and Practice in Area-Wide Integrated Pest Management.
19. Esu, Ekpereonne, Audrey Lenhart, Lucy Smith, and Olaf Horstick. 2010. “Effectiveness of Peridomestic Space Spraying with Insecticide on Dengue Transmission; Systematic Review.” Tropical Medicine & International Health: TM & IH 15 (5): 619-31.
20. Fu, Guoliang, Rosemary S. Lees, Derric Nimmo, Diane Aw, Li Jin, Pam Gray, Thomas U. Berendonk, et al. 2010. “Female-Specific Flightless Phenotype for Mosquito Control.” Proceedings of the National Academy of Sciences of the United States of America 107 (10): 4550-54.
21. Gantz, Valentino M., Nijole Jasinskiene, Olga Tatarenkova, Aniko Fazekas, Vanessa M. Macias, Ethan Bier, and Anthony A. James. 2015. “Highly Efficient Cas9-Mediated Gene Drive for Population Modification of the Malaria Vector Mosquito Anopheles Stephensi.” Proceedings of the National Academy of Sciences of the United States of America 112 (49): E6736-43.
22. George, Leyanna, Audrey Lenhart, Joao Toledo, Adhara Lazaro, Wai Han, Raman Velayudhan, Silvia Runge Ranzinger, and Olaf Horstick. 2015. “Community-Effectiveness of Temephos for Dengue Vector Control: A Systematic Literature Review.” PLoS Neglected Tropical Diseases 9 (9): e0004006.
23. Gilles, Jeremie R. L., Marc F. Schetelig, Francesca Scolari, Frantis̆ek Marec, Margareth L. Capurro, Gerald Franz, and Kostas Bourtzis. 2014. “Towards Mosquito Sterile Insect Technique Programmes: Exploring Genetic, Molecular, Mechanical and Behavioural Methods of Sex Separation in Mosquitoes.” Acta Tropica 132 Suppl (April): S178-87.
24. Gratz, Scott J., Alexander M. Cummings, Jennifer N. Nguyen, Danielle C. Hamm, Laura K. Donohue, Melissa M. Harrison, Jill Wildonger, and Kate M. O'Connor-Giles. 2013. “Genome Engineering of Drosophila with the CRISPR RNA-Guided Cas9 Nuclease.” Genetics 194 (4): 1029-35.
25. Hall, Andrew Brantley, Sanjay Basu, Xiaofang Jiang, Yumin Qi, Vladimir A. Timoshevskiy, James K. Biedler, Maria V. Sharakhova, et al. 2015. “Sex Determination. A Male-Determining Factor in the Mosquito Aedes Aegypti.” Science 348 (6240): 1268-70.
26. Hammond, Andrew, Roberto Galizi, Kyros Kyrou, Alekos Simoni, Carla Siniscalchi, Dimitris Katsanos, Matthew Gribble, et al. 2015. “A CRISPR-Cas9 Gene Drive System Targeting Female Reproduction in the Malaria Mosquito Vector Anopheles gambiae.” Nature Biotechnology 34 (December): 78.
27. Harris, Angela F., Andrew R. McKemey, Derric Nimmo, Zoe Curtis, Isaac Black, Sian A. Morgan, Marco Neira Oviedo, et al. 2012. “Successful Suppression of a Field Mosquito Population by Sustained Release of Engineered Male Mosquitoes.” Nature Biotechnology 30 (September): 828.
28. Harris, Angela F., Derric Nimmo, Andrew R. McKemey, Nick Kelly, Sarah Scaife, Christl A. Donnelly, Camilla Beech, William D. Petrie, and Luke Alphey. 2011. “Field Performance of Engineered Male Mosquitoes.” Nature Biotechnology 29 (11): 1034-37.
29. Haye, T., P. Girod, A. G. S. Cuthbertson, X. G. Wang, K. M. Daane, K. A. Hoelmer, C. Baroffio, J. P. Zhang, and N. Desneux. 2016. “Current SWD IPM Tactics and Their Practical Implementation in Fruit Crops across Different Regions around the World.” Journal of Pest Science 89 (3): 643-51.
30. Hemingway, Janet, Linda Field, and John Vontas. 2002. “An Overview of Insecticide Resistance.” Science 298 (5591): 96-97.
31. Hendrichs, J., A. S. Robinson, J. P. Cayol, and W. Enkerlin. 2002. “Medfly Areawide Sterile Insect Technique Programmes for Prevention, Suppression or Eradication: The Importance of Mating Behavior Studies.” The Florida Entomologist 85 (1): 1-13.
32. Hoang, Kim Phuc, Tze Min Teo, Thien Xuan Ho, and Vinh Sy Le. 2016. “Mechanisms of Sex Determination and Transmission Ratio Distortion in Aedes Aegypti.” Parasites & Vectors 9 (January): 49.
33. Hoyle, H. D., J. A. Hutchens, F. R. Turner, and E. C. Raff. 1995. “Regulation of Beta-Tubulin Function and Expression in Drosophila Spermatogenesis.” Developmental Genetics 16 (2): 148-70.
34. Huang, Yuping, Yazhou Chen, Baosheng Zeng, Yajun Wang, Anthony A. James, Geoff M. Gun, Guang Yang, Xijian Lin, Yongping Huang, and Minsheng You. 2016. “CRISPR/Cas9 Mediated Knockout of the Abdominal-A Homeotic Gene in the Global Pest, Diamondback Moth (Plutella Xylostella).” Insect Biochemistry and Molecular Biology 75 (August): 98-106.
35. Hughes, Grant L., Ana Rivero, and Jason L. Rasgon. 2014. “Wolbachia Can Enhance Plasmodium Infection in Mosquitoes: Implications for Malaria Control?” PLoS Pathogens 10 (9): e1004182.
36. Hwa, Jennifer J., Mark A. Hiller, Margaret T. Fuller, and Ansgar Santel. 2002. “Differential Expression of the Drosophila Mitofusin Genes Fuzzy Onions (fzo) and Dmfn.” Mechanisms of Development 116 (1-2): 213-16.
37. Ioriatti, C., V. Walton, D. Dalton, G. Anfora, A. Grassi, S. Maistri, and V. Mazzoni. 2015. “Drosophila Suzukii (Diptera: Drosophilidae) and Its Potential Impact to Wine Grapes During Harvest in Two Cool Climate Wine Grape Production Regions.” Journal of Economic Entomology 108 (3): 1148-55.
38. Joshi, Deepak, Michael J. McFadden, David Bevins, Fengrui Zhang, and Zhiyong Xi. 2014. “Wolbachia Strain wAlbB Confers Both Fitness Costs and Benefit on Anopheles Stephensi.” Parasites & Vectors 7 (July): 336.
39. Kalghatgi, Sameer, Catherine S. Spina, James C. Costello, Marc Liesa, J. Ruben Morones-Ramirez, Shimyn Slomovic, Anthony Molina, Orian S. Shirihai, and James J. Collins. 2013. “Bactericidal Antibiotics Induce Mitochondrial Dysfunction and Oxidative Damage in Mammalian Cells.” Science Translational Medicine 5 (192): 192ra85.
40. Kandul, N. P., Liu, J., Sanchez C., H. M. et al. Transforming insect population control with precision guided sterile males with demonstration in flies. Nat Commun 10, 84 (2019). https://doi.org/10.1038/s41467-018-07964-7
41. Kandul, Nikolay P., Junru Liu, Hector M. Sanchez, Sean L. Wu, John M. Marshall, and Omar S. Akbari. 2018. “Transforming Insect Population Control with Precision Guided Sterile Males.” bioRxiv. https://doi.org/10.1101/377721.
42. KaramiNejadRanjbar, Mohammad, Kolja N. Eckermann, Hassan M. M. Ahmed, Hector M. Sanchez C, Stefan Dippel, John M. Marshall, and Ernst A. Wimmer. 2018. “Consequences of Resistance Evolution in a Cas9-Based Sex Conversion-Suppression Gene Drive for Insect Pest Management.” Proceedings of the National Academy of Sciences of the United States of America 115 (24): 6189-94.
43. Kistler, Kathryn E., Leslie B. Vosshall, and Benjamin J. Matthews. 2015. “Genome Engineering with CRISPR-Cas9 in the Mosquito Aedes Aegypti.” Cell Reports 11 (1): 51-60.
44. Klassen, W., and C. F. Curtis. 2005. “History of the Sterile Insect Technique.” In Sterile Insect Technique: Principles and Practice in Area-Wide Integrated Pest Management, edited by V. A. Dyck, J. Hendrichs, and A. S. Robinson, 3-36. Dordrecht: Springer Netherlands.
45. Knipling, E. F. 1955. “Possibilities of Insect Control or Eradication Through the Use of Sexually Sterile Males.” Journal of Economic Entomology 48 (4): 459-62.
46. Krafsur, E. S., H. Townson, G. Davidson, and C. F. Curtis. 1986. “Screwworm Eradication Is What It Seems.” Nature 323 (6088): 495-96.
47. Kyrou, Kyros, Andrew M. Hammond, Roberto Galizi, Nace Kranjc, Austin Burt, Andrea K. Beaghton, Tony Nolan, and Andrea Crisanti. 2018. “A CRISPR-Cas9 Gene Drive Targeting Doublesex Causes Complete Population Suppression in Caged Anopheles Gambiae Mosquitoes.” Nature Biotechnology, September. https://doi.org/10.1038/nbt.4245.
48. Li, Fang, and Maxwell J. Scott. 2016. “CRISPR/Cas9-Mediated Mutagenesis of the White and Sex Lethal Loci in the Invasive Pest, Drosophila Suzukii.” Biochemical and Biophysical Research Communications 469 (4): 911-16.
49. Li, Ming, Michelle Bui, Ting Yang, Bradley White, and Omar Akbari. 2017. “Germline Cas9 Expression Yields Highly Efficient Genome Engineering in a Major Worldwide Disease Vector, Aedes Aegypti.” https://doi.org/10.1101/156778.
50. Li, Xueyan, Dingding Fan, Wei Zhang, Guichun Liu, Lu Zhang, Li Zhao, Xiaodong Fang, et al. 2015. “Outbred Genome Sequencing and CRISPR/Cas9 Gene Editing in Butterflies.” Nature Communications 6 (September): 8212.
51. Li, Yan, Jie Zhang, Dafeng Chen, Pengcheng Yang, Feng Jiang, Xianhui Wang, and Le Kang. 2016. “CRISPR/Cas9 in Locusts: Successful Establishment of an Olfactory Deficiency Line by Targeting the Mutagenesis of an Odorant Receptor Co-Receptor (Orco).” Insect Biochemistry and Molecular Biology 79 (December): 27-35.
52. Liang, Xiquan, Jason Potter, Shantanu Kumar, Namritha Ravinder, and Jonathan D. Chesnut. 2017. “Enhanced CRISPR/Cas9-Mediated Precise Genome Editing by Improved Design and Delivery of gRNA, Cas9 Nuclease, and Donor DNA.” Journal of Biotechnology 241 (Supplement C): 136-46.
53. Liu, Nannan. 2015. “Insecticide Resistance in Mosquitoes: Impact, Mechanisms, and Research Directions.” Annual Review of Entomology 60 (1): 537-59.
54. Ma, Sanyuan, Jiasong Chang, Xiaogang Wang, Yuanyuan Liu, Jianduo Zhang, Wei Lu, Jie Gao, Run Shi, Ping Zhao, and Qingyou Xia. 2014. “CRISPR/Cas9 Mediated Multiplex Genome Editing and Heritable Mutagenesis of BmKu70 in Bombyx mori.” Scientific Reports 4 (March): 4489.
55. Maciel-de-Freitas, Rafael, Fernando Campos Avendanho, Rosangela Santos, Gabriel Sylvestre, Simone Costa Araújo, José Bento Pereira Lima, Ademir Jesus Martins, Giovanini Evelim Coelho, and Denise Valle. 2014. “Undesirable Consequences of Insecticide Resistance Following Aedes Aegypti Control Activities Due to a Dengue Outbreak.” PloS One 9 (3): e92424.
56. Maruyama, Takeshi, Stephanie K. Dougan, Matthias C. Truttmann, Angelina M. Bilate, Jessica R. Ingram, and Hidde L. Ploegh. 2015. “Increasing the Efficiency of Precise Genome Editing with CRISPR-Cas9 by Inhibition of Nonhomologous End Joining.” Nature Biotechnology 33 (March): 538.
57. Massonnet-Bruneel, B. et al. Fitness of transgenic mosquito Aedes aegypti males carrying a dominant lethal genetic system. PLoS One 8, e62711 (2013).
58. Mazzi, Dominique, Esther Bravin, Manuela Meraner, Robert Finger, and Stefan Kuske. 2017. “Economic Impact of the Introduction and Establishment of Drosophila Suzukii on Sweet Cherry Production in Switzerland.” Insects 8 (1). https://doi.org/10.3390/insects8010018.
59. Moullan, Norman, Laurent Mouchiroud, Xu Wang, Dongryeol Ryu, Evan G. Williams, Adrienne Mottis, Virginija Jovaisaite, et al. 2015. “Tetracyclines Disturb Mitochondrial Function across Eukaryotic Models: A Call for Caution in Biomedical Research.” Cell Reports, March. https://doi.org/10.1016/j.celrep.2015.02.034.
60. Nene, Vishvanath, Jennifer R. Wortman, Daniel Lawson, Brian Haas, Chinnappa Kodira, Zhijian Jake Tu, Brendan Loftus, et al. 2007. “Genome Sequence of Aedes aegypti, a Major Arbovirus Vector.” Science 316 (5832): 1718-23.
61. Oberhofer, Georg, Tobin Ivy, and Bruce A. Hay. 2018. “Behavior of Homing Endonuclease Gene Drives Targeting Genes Required for Viability or Female Fertility with Multiplexed Guide RNAs.” Proceedings of the National Academy of Sciences of the United States of America 115 (40): E9343-52.
62. Papathanos, Philippos A., Hervé C. Bossin, Mark Q. Benedict, Flaminia Catteruccia, Colin A. Malcolm, Luke Alphey, and Andrea Crisanti. 2009. “Sex Separation Strategies: Past Experience and New Approaches.” Malaria Journal 8 Suppl 2 (November): S5.
63. Pluess, Bianca, Frank C. Tanser, Christian Lengeler, and Brian L. Sharp. 2010. “Indoor Residual Spraying for Preventing Malaria.” Cochrane Database of Systematic Reviews, no. 4 (April): CD006657.
64. Port, Fillip, Hui-Min Chen, Tzumin Lee, and Simon L. Bullock. 2014. “Optimized CRISPR/Cas Tools for Efficient Germline and Somatic Genome Engineering in Drosophila.” Proceedings of the National Academy of Sciences of the United States of America 111 (29): E2967-76.
65. Price, Dana C., Andrea Egizi, and Dina M. Fonseca. 2015a. “Characterization of the Doublesex Gene within the Culex pipiens Complex Suggests Regulatory Plasticity at the Base of the Mosquito Sex Determination Cascade.” BMC Evolutionary Biology 15 (June): 108.
66. Price, Dana C., Andrea Egizi, and Dina M. Fonseca. 2015b. “The Ubiquity and Ancestry of Insect Doublesex.” Scientific Reports 5 (August): 13068.
67. Reid, William, and David A. O'Brochta. 2016. “Applications of Genome Editing in Insects.” Current Opinion in Insect Science 13 (February): 43-54.
68. Salvemini, Marco, Rocco D'Amato, Valeria Petrella, Serena Aceto, Derric Nimmo, Marco Neira, Luke Alphey, Lino C. Polito, and Giuseppe Saccone. 2013. “The Orthologue of the Fruitfly Sex Behaviour Gene Fruitless in the Mosquito Aedes Aegypti: Evolution of Genomic Organisation and Alternative Splicing.” PloS One 8 (2): e48554.
69. Salvemini, Marco, Umberto Mauro, Fabrizio Lombardo, Andreina Milano, Vincenzo Zazzaro, Bruno Arca, Lino C. Polito, and Giuseppe Saccone. 2011. “Genomic Organization and Splicing Evolution of the Doublesex Gene, a Drosophila Regulator of Sexual Differentiation, in the Dengue and Yellow Fever Mosquito Aedes aegypti.” BMC Evolutionary Biology 11 (February): 41.
70. Sanchez, Lucas. 2008. “Sex-Determining Mechanisms in Insects.” The International Journal of Developmental Biology 52 (7): 837-56.
71. Sanchez C., H. M., Wu, S. L., Bennett, J. B. & Marshall, J. M. MGDrivE: A modular simulation framework for the spread of gene drives through spatially-explicit mosquito populations. (2018). doi:10.1101/350488
72. Sarno, Francesca, Maria F. Ruiz, José M. Eirin-López, André L. P. Perondini, Denise Selivon, and Lucas Sanchez. 2010. “The Gene Transformer-2 of Anastrepha Fruit Flies (Diptera, Tephritidae) and Its Evolution in Insects.” BMC Evolutionary Biology 10 (May): 140.
73. Sartain, Caroline V., Jun Cui, Richard P. Meisel, and Mariana F. Wolfner. 2011. “The poly(A) Polymerase GLD2 Is Required for Spermatogenesis in Drosophila melanogaster.” Development 138 (8): 1619-29.
74. Scali, Christina, Flaminia Catteruccia, Qiuxiang Li, and Andrea Crisanti. 2005. “Identification of Sex-Specific Transcripts of the Anopheles Gambiae Doublesex Gene.” The Journal of Experimental Biology 208 (Pt 19): 3701-9.
75. Schetelig, M. F., K-Z Lee, S. Otto, L. Talmann, J. Stökl, T. Degenkolb, A. Vilcinskas, and R. Halitschke. 2018. “Environmentally Sustainable Pest Control Options for Drosophila Suzukii.” Journal of Applied Entomology=Zeitschrift Fur Angewandte Entomologie 142 (1-2): 3-17.
76. Schmidt, Tom L., Nicholas H. Barton, Gordana Ras̆ić, Andrew P. Turley, Brian L. Montgomery, Inaki Iturbe-Ormaetxe, Peter E. Cook, et al. 2017. “Local Introduction and Heterogeneous Spatial Spread of Dengue-Suppressing Wolbachia through an Urban Population of Aedes Aegypti.” PLoS Biology 15 (5): e2001894.
77. Sekiné, Kazuki, Tadashi Furusawa, and Masatsugu Hatakeyama. 2015. “The Boule Gene Is Essential for Spermatogenesis of Haploid Insect Male.” Developmental Biology 399 (1): 154-63.
78. Slade, Glen, and Neil Morrison. 2014. “Developing GM Insects for Sustainable Pest Control in Agriculture and Human Health.” BMC Proceedings 8 (4): 043.
79. Stockton, Dara G., Anna K. Wallingford, and Gregory M. Loeb. 2018. “Phenotypic Plasticity Promotes Overwintering Survival in A Globally Invasive Crop Pest, Drosophila Suzukii.” Insects 9 (3). https://doi.org/10.3390/insects9030105.
80. Thailayil, Janis, Kalle Magnusson, H. Charles J. Godfray, Andrea Crisanti, and Flaminia Catteruccia. 2011. “Spermless Males Elicit Large-Scale Female Responses to Mating in the Malaria Mosquito Anopheles gambiae.” Proceedings of the National Academy of Sciences of the United States of America 108 (33): 13677-81.
81. Thomas, D. D., C. A. Donnelly, R. J. Wood, and L. S. Alphey. 2000. “Insect Population Control Using a Dominant, Repressible, Lethal Genetic System.” Science 287 (5462): 2474-76.
82. Van Steenwyk, R. A., and M. P. Bolda. 2014. “Spotted Wing Drosophila: Devastating Effects on Cherry and Berry Pest Management.” In XXIX International Horticultural Congress on Horticulture: Sustaining Lives, Livelihoods and Landscapes (IHC2014): 1105, 11-18.
83. Van Timmeren, Steven, David Mota-Sanchez, John C. Wise, and Rufus Isaacs. 2017. “Baseline Susceptibility of Spotted Wing Drosophila (Drosophila Suzukii) to Four Key Insecticide Classes.” Pest Management Science, August. https://doi.org/10.1002/ps.4702.
84. Vanderplank, F. L. 1944. “Hybridization between Glossina Species and Suggested New Method for Control of Certain Species of Tsetse.” Nature 154 (November): 607.
85. Walsh, Douglas B., Mark P. Bolda, Rachael E. Goodhue, Amy J. Dreves, Jana Lee, Denny J. Bruck, Vaughn M. Walton, Sally D. O'Neal, and Frank G. Zalom. 2011. “Drosophila Suzukii (Diptera: Drosophilidae): Invasive Pest of Ripening Soft Fruit Expanding Its Geographic Range and Damage Potential.” Journal of Integrated Pest Management 2 (1): G1-7.
86. Walton, V. M., H. J. Burrack, D. T. Dalton, R. Isaacs, N. Wiman, and C. Ioriatti. 2014. “Past, Present and Future of Drosophila Suzukii: Distribution, Impact and Management in United States Berry Fruits.” In XXIX International Horticultural Congress on Horticulture: Sustaining Lives, Livelihoods and Landscapes (IHC2014): II 1117, 87-94.
87. Whyard, Steve, Cassidy N. G. Erdelyan, Alison L. Partridge, Aditi D. Singh, Nigel W. Beebe, and Rupert Capina. 2015. “Silencing the Buzz: A New Approach to Population Suppression of Mosquitoes by Feeding Larvae Double-Stranded RNAs.” Parasites & Vectors 8 (February): 96.
88. Wilkinson, T. L. 1998. “The Elimination of Intracellular Microorganisms from Insects: An Analysis of Antibiotic-Treatment in the Pea Aphid (Acyrthosiphon Pisum).” Comparative Biochemistry and Physiology. Part A, Molecular & Integrative Physiology 119 (4): 871-81.
89. Windbichler, Nikolai, Philippos Aris Papathanos, and Andrea Crisanti. 2008. “Targeting the X Chromosome during Spermatogenesis Induces Y Chromosome Transmission Ratio Distortion and Early Dominant Embryo Lethality in Anopheles Gambiae.” PLoS Genetics 4 (12): e1000291.
90. World Health Organization. 2014. “A Global Brief on Vector-Borne Diseases.” http://apps.who.int/iris/bitstream/10665/111008/1/WHO_DCO_WHD_2014.1_eng.pdf.
91. Yaffe, M. P. 1997. “Mitochondrial Morphogenesis: Fusion Factor for Fly Fertility.” Current Biology: CB 7 (12): R782-83.
92. Yakob, Laith, Sebastian Funk, Anton Camacho, Oliver Brady, and W. John Edmunds. 2017. “Aedes Aegypti Control Through Modernized, Integrated Vector Management.” PLoS Currents 9 (January). https://doi.org/10.1371/currents.outbreaks.45 deb 8e03a438c4d088afb4fafae8747.
93. Zeh, Jeanne A., Melvin M. Bonilla, Angelica J. Adrian, Sophia Mesfin, and David W. Zeh. 2012. “From Father to Son: Transgenerational Effect of Tetracycline on Sperm Viability.” Scientific Reports 2 (April): 375.
94. Zetsche, Bernd, Jonathan S. Gootenberg, Omar O. Abudayyeh, Ian M. Slaymaker, Kira S. Makarova, Patrick Essletzbichler, Sara E. Volz, et al. 2015. “Cpf1 Is a Single RNA-Guided Endonuclease of a Class 2 CRISPR-Cas System.” Cell 163 (3): 759-71.

1. A mating system producing a genetically modified progeny that is a sterile male insect of Drosophila suzukii or Aedes aegypti mosquito, comprising a first parental insect and a second parental insect,
wherein the first parental insect comprises at least one nucleic acid sequence that comprises:
(a) at least a first guide RNA targeting a female essential genomic sequence that is required for female-specific viability in Drosophila suzukii or Aedes aegypti, and
(b) at least a second guide RNA targeting a male sterility genomic sequence that is required for male-specific fertility in Drosophila suzukii or Aedes aegypti,
wherein the targeted sequence required for female-specific viability in Aedes aegypti is selected from a sequence of the Intersex or labrum (Lab) gene or wherein the targeted sequence required for male-specific fertility in Aedes aegypti is a sequence of the zero population growth (Zpg)1 gene, optionally wherein the targeted sequence is a sequence in the gene exon, and
wherein the targeted sequence required for male-specific fertility in Drosophila suzukii is selected from a sequence of Cannonball (Can), meiosis I arrest (Mia), P-element induced wimpy testis (PIWI), Heterochromatin Protein 1e (HP1e), zero population growth (Zpg), male sterile (3) K81 (K81), misfire (Mfr), or sneaky (Snky), optionally wherein the targeted sequence is a sequence in the gene exon; and
wherein the second parental insect comprises a nucleic acid that encodes an endonuclease under the control of a regulatory sequence that is suitable for directing protein expression in Drosophila suzukii or Aedes aegypti.

2. The mating system of claim 1,
wherein the targeted sequence required for female-specific viability in Aedes aegypti is selected from:
(Intersex1, SEQ ID NO: 58)
GCGGGATTCGCTCTCGACGA,
(Intersex2, SEQ ID NO: 59)
GGACAACATTTCCAAGGTTA,
(labrum (Lab) 5, SEQ ID NO: 60)
GCACTAGTGGGATATCCTGA,
or
(labrum (Lab) 4, SEQ ID NO: 61)
GTCCAGGAAGCATTTGGTAT;
wherein the targeted sequence required for male-specific fertility in Aedes aegypti is selected from:
(Zpg1, SEQ ID NO: 62)
GTGTAGACTGGCCCTAACGG,
(Zpg2, SEQ ID NO: 63)
TCTCGTTCCCCAAACACTTG,
(Zpg3, SEQ ID NO: 64)
TCATCAAGAACATGTTCAGG,
or
(Zpg4, SEQ ID NO: 65)
ACAGTTGGAACCGAACGCTG;
and
wherein the targeted sequence required for male-specific fertility in Drosophila suzukii is selected from:
(Cannonball (Can), SEQ ID NO: 66)
GCTACAGAGGAATGGCCCAG,
(Cannonball (Can), SEQ ID NO: 67)
GGATCGGGATAACCTGCCGT,
(meiosis I arrest (Mia), SEQ ID NO: 68)
GAGAATCCCCTTGTTGCGGG,
(meiosis I arrest (Mia), SEQ ID NO: 69)
GAGCTCTGACCATCCGCATG,
(P-element induced wimpy testis (PIWI),
SEQ ID NO: 70)
GGTGTTGGACAGCACATCGA,
(P-element induced wimpy testis (PIWI),
SEQ ID NO: 71)
GAGCACACGTGATCGCAAGA,
(Heterochromatin Protein 1e (HP1e), SEQ ID NO: 72)
CAACCTCAAgTTGTaCCAAG,
(Heterochromatin Protein 1e (HP1e), SEQ ID NO: 73)
GGATTATCCAAAACTAAGCA,
(zero population growth (Zpg), SEQ ID NO: 74)
GCAACTGCAAACGCATTCCG,
(zero population growth (Zpg), SEQ ID NO: 75)
GCCCAAGTTGCACCTGCAGG,
(male sterile (3) K81 (K81), SEQ ID NO: 76)
GATCACAGCGAGTTCTTCGG,
(male sterile (3) K81 (K81), SEQ ID NO: 77)
GGAGTTGGGGTCGTCGACAT,
(misfire (Mfr), SEQ ID NO: 78)
GGCGTCAAGTTCAAGAAGCA,
(misfire (Mfr), SEQ ID NO: 79)
GAGAACGGGACACTGGCAGG,
(sneaky (Snky), SEQ ID NO: 80)
GACTTCTCGTAGGTGCGCAA,
and
(sneaky (Snky), SEQ ID NO: 81)
GTAGGTGCGCAATGGTAAGA.

3. The mating system of claim 1,
wherein the regulatory sequence that is suitable for directing protein expression in Drosophila suzukii comprises a promoter selected from: nt 4360 to nt 6582 of SEQ ID NO: 1 or nt 4360 to nt 6600 of SEQ ID NO: 2 (874Z promoter sequence), nt 4366 to nt 4873 of SEQ ID NO: 3 (874S promoter sequence), nt 4366 to nt 7196 of SEQ ID NO: 4 (874R promoter sequence), nt 4366 to nt 6233 of SEQ ID NO: 5 (874W promoter sequence), or nt 4360 to nt 5291 of SEQ ID NO: 6 (874Z1 promoter sequence), optionally wherein the promoter comprises a sequence of nt 4366 to nt 6233 of SEQ ID NO: 5 (874W promoter sequence); and
wherein the regulatory sequence suitable for directing protein expression in Aedes aegypti mosquito comprises a promoter selected from nt 4366 to nt 6497 of SEQ ID NO: 22 (874L promoter sequence), nt 4366 to nt 6865 of SEQ ID NO: 23 or a fragment thereof comprising an AAEL007097 promoter (874M promoter sequence), nt 4366 to nt 7404 of SEQ ID NO: 24 (874N promoter sequence), nt 4366 to nt 7399 of SEQ ID NO: 25 (874P promoter sequence), or nt 4366 to nt 5751 of SEQ ID NO: 26 (874X promoter sequence), or nt 4366 to nt 4786 of SEQ ID NO: 27 (874Y promoter sequence) or nt 4363 to nt 7162 of SEQ ID NO: 28 (874Y3 promoter sequence), optionally wherein the promoter comprises a sequence of nt 4366 to nt 7399 of SEQ ID NO: 25 (874P promoter sequence) or wherein the promoter comprises a sequence of nt 4366 to nt 4786 of SEQ ID NO: 27 (874Y promoter sequence).

4. A mating system producing a genetically modified progeny that is a sterile male insect of Drosophila suzukii or Aedes aegypti mosquito, comprising a first parental insect and a second parental insect,
wherein the first parental insect comprises at least one nucleic acid sequence that comprises:
(a) at least a first guide RNA targeting a female essential genomic sequence that is required for female-specific viability in Drosophila suzukii or Aedes aegypti, and
(b) at least a second guide RNA targeting a male sterility genomic sequence that is required for male-specific fertility in Drosophila suzukii or Aedes aegypti, wherein the targeted sequence required for female-specific viability in Drosophila suzukii is GGCGGCAGCGGCGGGAATGG (Sxl—Max Scott site, SEQ ID NO: 82) or wherein the targeted sequence required for male-specific fertility in Drosophila suzukii is selected from: TGGCGGTACCGGCTCCGGAA (Btub Original site 2, SEQ ID NO: 83), CTTGAGTGTGCACCAGCTGG (Btub site 2, SEQ ID NO: 84), CAATGCGGTAACCAGATCGG (Btub site 3, SEQ ID NO: 85), GCCTCGGGGTCTAAAGATGT (Btub site 4, SEQ ID NO: 86), GCTACAGAGGAATGGCCCAG (Cannonball (Can), SEQ ID NO: 66), GGATCGGGATAACCTGCCGT (Cannonball (Can), SEQ ID NO: 67), GAGAATCCCCTTGTTGCGGG (meiosis I arrest (Mia), SEQ ID NO: 68), GAGCTCTGACCATCCGCATG (meiosis I arrest (Mia), SEQ ID NO: 69), GGTGTTGGACAGCACATCGA (P-element induced wimpy testis (PIWI), SEQ ID NO: 70), GAGCACACGTGATCGCAAGA (P-element induced wimpy testis (PIWI), SEQ ID NO: 71), CAACCTCAAgTTGTaCCAAG (Heterochromatin Protein 1e (HP1e), SEQ ID NO: 72), GGATTATCCAAAACTAAGCA (Heterochromatin Protein 1e (HP1e), SEQ ID NO: 73), GCAACTGCAAACGCATTCCG (zero population growth (Zpg), SEQ ID NO: 74), GCCCAAGTTGCACCTGCAGG (zero population growth (Zpg), SEQ ID NO: 75), GATCACAGCGAGTTCTTCGG (male sterile (3) K81 (K81), SEQ ID NO: 76), GGAGTTGGGGTCGTCGACAT male sterile (3) K81 (K81), SEQ ID NO: 77), GGCGTCAAGTTCAAGAAGCA (misfire (Mfr), SEQ ID NO: 78), GAGAACGGGACACTGGCAGG (misfire (Mfr), SEQ ID NO: 79), GACTTCTCGTAGGTGCGCAA (sneaky (Snky), SEQ ID NO: 80), or GTAGGTGCGCAATGGTAAGA (sneaky (Snky), SEQ ID NO: 81), and
wherein the targeted sequence required for female-specific viability in Aedes aegypti is selected from: AGCGACATGCAGCCATTCTG (double sex (Dsx) 1, SEQ ID NO: 87 in the revised plasmid), CTCACAAGTAGAGCTACACG (Dsx2, SEQ ID NO: 88 in the revised plasmid), GGTAGCTGGCCGTTGCCAAA (Dsx3, SEQ ID NO: 89 in the revised plasmid), CTGTCGTCGTTTTTTTCCGG (Dsx4, SEQ ID NO: 90 in the revised plasmid), GGATATTGGTACGACCCGGG (Dsx5, SEQ ID NO: 91), GCGGGATTCGCTCTCGACGA (Intersex1, SEQ ID NO: 58), GGACAACATTTCCAAGGTTA (Intersex2, SEQ ID NO: 59), GCACTAGTGGGATATCCTGA (labrum (Lab) 5, SEQ ID NO: 60), GTCCAGGAAGCATTTGGTAT (labrum (Lab) 4, SEQ ID NO: 61), GTATTCGATGTTCTCCACC (sister of sex lethal (Slx) 4, SEQ ID NO: 92), GAAGGCATATCAAACATTCG (sister of sex lethal (Slx) 5, SEQ ID NO: 93), GTTAAGAACTCGGCCATCGA (homeotic protein Proboscipedia (Pb) 1, SEQ ID NO: 94), GCAAGTTAAGCCTGAAACAA (homeotic protein Proboscipedia (Pb) 2, SEQ ID NO: 95), or wherein the targeted sequence required for male-specific fertility in Aedes aegypti is selected from: TACTACAACGAGGCTACCGG (Btub1, SEQ ID NO: 96), GTCTCCGCAATACGCCCCGG (Btub2, SEQ ID NO: 97), GATAGGAGCCAAGTTCTGGG (Btub3, SEQ ID NO: 98), CACTATGGATTCGGTCCGAG (Btub4, SEQ ID NO: 99), GTGTAGACTGGCCCTAACGG (zero population growth (Zpg)1, SEQ ID NO: 62), TCTCGTTCCCCAAACACTTG (Zpg2, SEQ ID NO: 63), TCATCAAGAACATGTTCAGG (Zpg3, SEQ ID NO: 64), ACAGTTGGAACCGAACGCTG (Zpg4, SEQ ID NO: 65); and
wherein the second parental insect comprises a nucleic acid that encodes an endonuclease under the control of a regulatory sequence that is suitable for directing protein expression in Drosophila suzukii or Aedes aegypti.

5. The mating system of claim 4,
wherein the regulatory sequence that is suitable for directing protein expression in Drosophila suzukii comprises a promoter selected from: nt 4360 to nt 6582 of SEQ ID NO: 1 or nt 4360 to nt 6600 of SEQ ID NO: 2 (874Z promoter sequence), nt 4366 to nt 4873 of SEQ ID NO: 3 (874S promoter sequence), nt 4366 to nt 7196 of SEQ ID NO: 4 (874R promoter sequence), nt 4366 to nt 6233 of SEQ ID NO: 5 (874W promoter sequence), or nt 4360 to nt 5291 of SEQ ID NO: 6 (874Z1 promoter sequence), optionally wherein the promoter comprises a sequence of nt 4366 to nt 6233 of SEQ ID NO: 5 (874W promoter sequence); and
wherein the regulatory sequence suitable for directing protein expression in Aedes aegypti mosquito comprises a promoter selected from nt 4366 to nt 6497 of SEQ ID NO: 22 (874L promoter sequence), nt 4366 to nt 6865 of SEQ ID NO: 23 or a fragment thereof comprising an AAEL007097 promoter (874M promoter sequence), nt 4366 to nt 7404 of SEQ ID NO: 24 (874N promoter sequence), nt 4366 to nt 7399 of SEQ ID NO: 25 (874P promoter sequence), or nt 4366 to nt 5751 of SEQ ID NO: 26 (874X promoter sequence), or nt 4366 to nt 4786 of SEQ ID NO: 27 (874Y promoter sequence) or nt 4363 to nt 7162 of SEQ ID NO: 28 (874Y3 promoter sequence), optionally wherein the promoter comprises a sequence of nt 4366 to nt 7399 of SEQ ID NO: 25 (874P promoter sequence) or wherein the promoter comprises a sequence of nt 4366 to nt 4786 of SEQ ID NO: 27 (874Y promoter sequence).

6. A mating system producing a genetically modified progeny that is a sterile male insect of Drosophila suzukii, comprising a first parental insect and a second parental insect,
wherein the first parental Drosophila suzukii comprises at least one nucleic acid sequence that comprises:
(a) at least a first guide RNA targeting a female essential genomic sequence that is required for female-specific viability in Drosophila suzukii, and
(b) at least a second guide RNA targeting a male sterility genomic sequence that is required for male-specific fertility in Drosophila suzukii;
wherein the second parental insect comprises a nucleic acid that encodes an endonuclease under the control of a regulatory sequence that is suitable for directing protein expression in Drosophila suzukii, wherein the regulatory sequence comprises a promoter selected from a deadhead (Dhd) promoter or a bicoid (BicC) promoter.

7. The mating system of claim 6,
wherein the targeted sequence required for male-specific fertility in Drosophila suzukii is selected from a sequence of Cannonball (Can), meiosis I arrest (Mia), P-element induced wimpy testis (PIWI), Heterochromatin Protein 1e (HP1e), zero population growth (Zpg), male sterile (3) K81 (K81), misfire (Mfr), or sneaky (Snky).

8. The mating system of claim 6,
wherein the targeted sequence required for female-specific viability in Drosophila suzukii is GGCGGCAGCGGCGGGAATGG (Sxl—Max Scott site, SEQ ID NO: 82) or wherein the targeted sequence required for male-specific fertility in Drosophila suzukii is selected from: TGGCGGTACCGGCTCCGGAA (Btub Original site 2, SEQ ID NO: 83), CTTGAGTGTGCACCAGCTGG (Btub site 2, SEQ ID NO: 84), CAATGCGGTAACCAGATCGG (Btub site 3, SEQ ID NO: 85), GCCTCGGGGTCTAAAGATGT (Btub site 4, SEQ ID NO: 86), GCTACAGAGGAATGGCCCAG (Cannonball (Can), SEQ ID NO: 66), GGATCGGGATAACCTGCCGT (Cannonball (Can), SEQ ID NO: 67), GAGAATCCCCTTGTTGCGGG (meiosis I arrest (Mia), SEQ ID NO: 68), GAGCTCTGACCATCCGCATG (meiosis I arrest (Mia), SEQ ID NO: 69), GGTGTTGGACAGCACATCGA (P-element induced wimpy testis (PIWI), SEQ ID NO: 70), GAGCACACGTGATCGCAAGA (P-element induced wimpy testis (PIWI), SEQ ID NO: 71), CAACCTCAAgTTGTaCCAAG (Heterochromatin Protein 1e (HP1e), SEQ ID NO: 72), GGATTATCCAAAACTAAGCA (Heterochromatin Protein 1e (HP1e), SEQ ID NO: 73), GCAACTGCAAACGCATTCCG (zero population growth (Zpg), SEQ ID NO: 74), GCCCAAGTTGCACCTGCAGG (zero population growth (Zpg), SEQ ID NO: 75), GATCACAGCGAGTTCTTCGG (male sterile (3) K81 (K81), SEQ ID NO: 76), GGAGTTGGGGTCGTCGACAT male sterile (3) K81 (K81), SEQ ID NO: 77), GGCGTCAAGTTCAAGAAGCA (misfire (Mfr), SEQ ID NO: 78), GAGAACGGGACACTGGCAGG (misfire (Mfr), SEQ ID NO: 79), GACTTCTCGTAGGTGCGCAA (sneaky (Snky), SEQ ID NO: 80), or GTAGGTGCGCAATGGTAAGA (sneaky (Snky), SEQ ID NO: 81).

9. The mating system of claim 1, wherein the at least one nucleic acid sequence of the first parental Drosophila suzukii comprises GATTGTCAACTACTTGCCCC (Sex lethal (Sxl)—Nikolai site 1, SEQ ID NO: 100).

10. The mating system of claim 1, wherein the at least one nucleic acid sequence of the first parental Aedes aegypti comprises a set of guide RNAs, wherein the targeted sequences are Dsx1, intersex1, BTub3 and BTub2, or wherein the targeted sequences are Dsx1, Dsx5, BTub3, and BTub2, or wherein the targeted sequences are Zpg3, Zpg4, Dsx1, and Dsx1, or wherein the targeted sequences are Btub3, Btub2, Dsx1, and Dsx1, or wherein the targeted sequences are Dsx1, Dsx2, Dsx3, Dsx4, BTub3, and BTub2, or wherein the targeted sequences are Dsx1, intersex1, BTub3, BTub2, Dsx2, and Dsx4+.

11.-12. (canceled)

13. A method of producing a genetically modified progeny that is a sterile male insect of Drosophila suzukii or Aedes aegypti mosquito, comprising genetically crossing two parental insects of the mating system of claim 1, and producing and collecting the progeny of the parental insects.

14. The method of claim 13, further comprising an introduction step prior to the genetically crossing step, wherein the introduction step comprises one or both of the following:
introducing the at least one nucleic acid sequence to the first parental insect, or
introducing the nucleic acid that encodes an endonuclease under the control of a regulatory sequence to the second parental insect, and
optionally wherein either or both of the introduction integrates the sequence into the insect genome, and optionally wherein the progeny comprising the endonuclease, at least one first guide RNA, and at least one second guide RNA.

15. A progeny produced by the mating system of claim 1, optionally the progeny is an egg, a population of eggs, an insect, or a population of insects, and further optionally wherein the progeny is sterile male.

16. A method of reducing a wild-type insect population comprising introducing the progeny of claim 15 to the wild-type insect population.

17. A polynucleotide comprising a sequence encoding a Cas9 under the control of a deadhead (Dhd) promoter or a bicoid (BicC) promoter, optionally the polynucleotide is isolated or engineered, and further optionally wherein the polynucleotide is for use in the mating system of claim 1.

18. A polynucleotide or a sequence complementary thereto, optionally wherein the polynucleotide is an isolated or engineered polynucleotide, and wherein the polynucleotide comprises one or both of:
(a) at least a first guide RNA targeting a female essential genomic sequence that is required for female-specific viability in Drosophila suzukii or Aedes aegypti, and
(b) at least a second guide RNA targeting a male sterility genomic sequence that is required for male-specific fertility in Drosophila suzukii or Aedes aegypti,
wherein the targeted sequence required for female-specific viability in Aedes aegypti is selected from a sequence of the Intersex or labrum (Lab) gene or wherein the targeted sequence required for male-specific fertility in Aedes aegypti is a sequence of the zero population growth (Zpg)1 gene, optionally wherein the targeted sequence is a sequence in the gene exon, and
wherein the targeted sequence required for male-specific fertility in Drosophila suzukii is selected from a sequence of Cannonball (Can), meiosis I arrest (Mia), P-element induced wimpy testis (PIWI), Heterochromatin Protein 1e (HP1e), zero population growth (Zpg), male sterile (3) K81 (K81), misfire (Mfr), or sneaky (Snky), optionally wherein the targeted sequence is a sequence in the gene exon.

19. A polynucleotide or a sequence complementary thereto, optionally wherein the polynucleotide is an isolated or engineered polynucleotide, and wherein the polynucleotide comprises one or both of:
(a) a first guide RNA targeting a female essential genomic sequence that is required for female-specific viability in Drosophila suzukii or Aedes aegypti, and
(b) a second guide RNA targeting a male sterility genomic sequence that is required for male-specific fertility in Drosophila suzukii or Aedes aegypti,
wherein the targeted sequence required for female-specific viability in Drosophila suzukii is GGCGGCAGCGGCGGGAATGG (Sxl—Max Scott site, SEQ ID NO: 82) or wherein the targeted sequence required for male-specific fertility in Drosophila suzukii is selected from: TGGCGGTACCGGCTCCGGAA (Btub Original site 2, SEQ ID NO: 83), CTTGAGTGTGCACCAGCTGG (Btub site 2, SEQ ID NO: 84), CAATGCGGTAACCAGATCGG (Btub site 3, SEQ ID NO: 85), GCCTCGGGGTCTAAAGATGT (Btub site 4, SEQ ID NO: 86), GCTACAGAGGAATGGCCCAG (Cannonball (Can), SEQ ID NO: 66), GGATCGGGATAACCTGCCGT (Cannonball (Can), SEQ ID NO: 67), GAGAATCCCCTTGTTGCGGG (meiosis I arrest (Mia), SEQ ID NO: 68), GAGCTCTGACCATCCGCATG (meiosis I arrest (Mia), SEQ ID NO: 69), GGTGTTGGACAGCACATCGA (P-element induced wimpy testis (PIWI), SEQ ID NO: 70), GAGCACACGTGATCGCAAGA (P-element induced wimpy testis (PIWI), SEQ ID NO: 71), CAACCTCAAgTTGTaCCAAG (Heterochromatin Protein 1e (HP1e), SEQ ID NO: 72), GGATTATCCAAAACTAAGCA (Heterochromatin Protein 1e (HP1e), SEQ ID NO: 73), GCAACTGCAAACGCATTCCG (zero population growth (Zpg), SEQ ID NO: 74), GCCCAAGTTGCACCTGCAGG (zero population growth (Zpg), SEQ ID NO: 75), GATCACAGCGAGTTCTTCGG (male sterile (3) K81 (K81), SEQ ID NO: 76), GGAGTTGGGGTCGTCGACAT male sterile (3) K81 (K81), SEQ ID NO: 77), GGCGTCAAGTTCAAGAAGCA (misfire (Mfr), SEQ ID NO: 78), GAGAACGGGACACTGGCAGG (misfire (Mfr), SEQ ID NO: 79), GACTTCTCGTAGGTGCGCAA (sneaky (Snky), SEQ ID NO: 80), or GTAGGTGCGCAATGGTAAGA (sneaky (Snky), SEQ ID NO: 81), and
wherein the targeted sequence required for female-specific viability in Aedes aegypti is selected from: AGCGACATGCAGCCATTCTG (double sex (Dsx) 1, SEQ ID NO: 87 in the revised plasmid), CTCACAAGTAGAGCTACACG (Dsx2, SEQ ID NO: 88 in the revised plasmid), GGTAGCTGGCCGTTGCCAAA (Dsx3, SEQ ID NO: 89 in the revised plasmid), CTGTCGTCGTTTTTTTCCGG (Dsx4, SEQ ID NO: 90 in the revised plasmid), GGATATTGGTACGACCCGGG (Dsx5, SEQ ID NO: 91), GCGGGATTCGCTCTCGACGA (Intersex1, SEQ ID NO: 58), GGACAACATTTCCAAGGTTA (Intersex2, SEQ ID NO: 59), GCACTAGTGGGATATCCTGA (labrum (Lab) 5, SEQ ID NO: 60), GTCCAGGAAGCATTTGGTAT (labrum (Lab) 4, SEQ ID NO: 61), GTATTCGATGTTCTCCACC (sister of sex lethal (Slx) 4, SEQ ID NO: 92), GAAGGCATATCAAACATTCG (sister of sex lethal (Slx) 5, SEQ ID NO: 93), GTTAAGAACTCGGCCATCGA (homeotic protein Proboscipedia (Pb) 1, SEQ ID NO: 94), GCAAGTTAAGCCTGAAACAA (homeotic protein Proboscipedia (Pb) 2, SEQ ID NO: 95), or wherein the targeted sequence required for male-specific fertility in Aedes aegypti is selected from: TACTACAACGAGGCTACCGG (Btub1, SEQ ID NO: 96), GTCTCCGCAATACGCCCCGG (Btub2, SEQ ID NO: 97), GATAGGAGCCAAGTTCTGGG (Btub3, SEQ ID NO: 98), CACTATGGATTCGGTCCGAG (Btub4, SEQ ID NO: 99), GTGTAGACTGGCCCTAACGG (zero population growth (Zpg)1, SEQ ID NO: 62), TCTCGTTCCCCAAACACTTG (Zpg2, SEQ ID NO: 63), TCATCAAGAACATGTTCAGG (Zpg3, SEQ ID NO: 64), ACAGTTGGAACCGAACGCTG (Zpg4, SEQ ID NO: 65).

20. (canceled)

21. A cell comprising a polynucleotide of claim 17, optionally wherein the host cell is an insect cell, further optionally, wherein the cell is a Drosophila suzukii or Aedes aegypti mosquito cell, and yet further optionally wherein the host cell is an egg, a sperm or a zygote.

22. A Drosophila suzukii or Aedes aegypti mosquito comprising a polynucleotide of any one of claim 17.

23. A kit comprising a polynucleotide of claim 17.
